(19) United States
(12) Patent Application Publication
(10) Pub. No.: US 2021/0238225 A1
(43) Pub. Date: Aug. 5, 2021

(54) PEPTIDOMIMETIC INHIBITORS OF THE PEPTIDYL-PROLYL CIS/TRANS ISOMERASE (PIN1)

(71) Applicants: DANA-FARBER CANCER INSTITUTE, INC., Boston, MA (US); BETH ISRAEL DEACONESS MEDICAL CENTER, INC., Boston, MA (US)

(72) Inventors: Nathanael GRAY, Boston, MA (US); Benika PINCH, Brookline, MA (US); Sirano DHE-PAGANON, Holliston, MA (US); Hyuk-Soo SEO, Cambridge, MA (US); Chris BROWNE, Brookline, MA (US); Jarrod MARTO, Boston, MA (US); Zainab DOCTOR, Boston, MA (US); Kun Ping LU, Newton, MA (US); Xiao ZHOU, Newton, MA (US); Shingo KOZONO, Brookline, MA (US); Xiaolan LIAN, Fuzhou (CN)

(73) Assignees: DANA-FARBER CANCER INSTITUTE, INC., Boston, MA (US); BETH ISRAEL DEACONESS MEDICAL CENTER, INC., Boston, MA (US)

(21) Appl. No.: 17/251,084

(22) PCT Filed: Jun. 13, 2019
(86) PCT No.: PCT/US2019/036938

§ 371 Date: Dec. 10, 2020

Related U.S. Application Data
(60) Provisional application No. 62/685,110, filed on Jun. 14, 2018.

(51) Int. Cl. C07K 5/107

(52) U.S. Cl. CPC C07K 5/1016; A61K 38/00

(57) ABSTRACT

Disclosed are compounds which inhibit Pin1 activity, methods of making the compounds, pharmaceutical compositions containing the compounds, and methods of using the compounds to treat diseases or disorders characterized or mediated by dysregulated Pin1 activity.

### RELATED APPLICATION

This application is a national stage application, filed under 35 U.S.C. § 371, of International Application No. PCT/US2019/036938, filed Jun. 13, 2019, which claims the benefit of priority under 35 U.S.C. § 119(e) to U.S. Provisional Application No. 62/685,110, filed on Jun. 14, 2018, each of which is incorporated herein by reference in its entirety.

### GOVERNMENT LICENSE RIGHTS

This invention was made with government support under grant number R01 CA205153 awarded by the National Institutes of Health, grant number R01 CA167677 awarded by the National Institutes of Health, grant number F31 CA225066-02 awarded by the National Institutes of Health, grant number 5 T32 GM007306-41 awarded by the National Institutes of Health, and grant number 5 T32 GM095450-04 awarded by the National Institutes of Health. The government has certain rights in the invention.

### BACKGROUND OF THE INVENTION

Proline is unique among the amino acids because it populates both the cis and trans conformations, providing a backbone conformational switch that is controlled by prolyl isomerization. Due to the high energy barrier associated with cis to trans conversion (25-30 kcal/mol), the intrinsic isomerization process is slow (several minutes) relative to biochemical processes, and therefore catalysis by peptidyl prolyl isomerases (PPIases) is required for efficient isomerization.

Proline (Pro)-directed serine/threonine (Ser/Thr) phosphorylation (pSer/Thr-Pro) serves an essential role in cell signaling networks and is often dysregulated in cancer. Numerous oncogenes and tumor suppressors are regulated by Pro-directed phosphorylation and/or are part of signaling pathways involving such phosphorylation.

pSer/Thr-Pro reduces the intrinsically slow cis-trans isomerization process, and also renders the peptide bonds inaccessible for all known peptidyl-prolyl cis-trans isomerases (PPIases), except for peptidyl-prolyl cis-trans isomerase NIMA-interacting 1 (Pin1) and its homologues. Pin1 contains an N-terminal WW domain, which functions as a phosphorylated Ser/Thr-Pro binding module, and a PPIase domain, which catalyzes the cis-trans isomerization. (Zhou et al., Cell. Mol. Life Sci. 56: 788-806 (1999)).

Pin1-catalysed prolyl isomerization regulates the functions of its substrates through multiple different mechanisms, including controlling catalytic activity, turnover, phosphorylation, interactions with DNA, RNA or other proteins, and subcellular localization and processing. Pin1 often functions as a molecular timer that synchronously controls the amplitude and duration of a given cellular process. Pin1 is tightly regulated normally and its deregulation can have a major impact on the development and treatment of cancer and neurodegenerative diseases, such as Alzheimer disease. (Lu and Zhou, Nat. Rev. Mol. Cell Biol. 5:904-16 (2007)).

Pin1 is widely overexpressed and/or overactivated in cancers which correlate with poor clinical prognosis. (Lu and Hunter, Cell Res. 24:1033-49 (2014)). It has also been shown that Pin1 single nucleotide polymorphisms (SNPs) that reduce Pin1 expression are associated with a reduced risk for multiple cancers, and that Pin1-null mice are highly resistant to tumorigenesis, even after the overexpression of oncogenes or after the mutation or ablation of tumor suppressors. (Li et al., PLoS ONE 8:&88148 (2004); Wulf et al., EMBO J. 23:3397-3407 (2004); Girardini et al., Cancer Cell 20:79-91 (2011); Takahashi et al., Oncogene 26:3835-45 (2007)). Further, Pin1-null mice have been shown to develop normally to adulthood with few defects. (Lee et al., Expert Rev. Mol. Med. 73:e21 (2011)). Further, Pin1 overexpression disrupts cell cycle coordination and leads to chromosome instability and tumorigenesis. Pin1 activates and inactivates more than 40 oncogenes and 20 tumor suppressors, respectively. Many of these Pin1 substrates have a role in self-renewal, replicative potential and frequency of cancer stem cells (CSCs). (Zhou and Lu, Nat. Rev. Cancer 16: 463-78 (2016)). Therefore, Pin1 inhibitors may have the desirable ability to simultaneously block multiple cancer-driving pathways and CSC expansion and differentiation with limited toxicity.

### SUMMARY OF THE INVENTION

A first aspect of the present invention is directed to a compound having a structure as represented by formula (I):

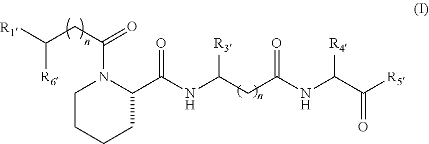

wherein each n is independently 0 or 1;
R₁′ is a phosphorylated alkyl, a hydroxyalkyl, a sulfone, an optionally substituted aralkyl, a carboxylic acid or an ester;
R_{3′} is an optionally substituted aralkyl, a ketone or an optionally substituted heteroaralkyl;
R_{4′} is an alkyl urea, an alkyl guanidine, a hydroxyalkyl, an amide, an optionally substituted heteroaralkyl or an optionally substituted aralkyl; and
R_{5′} is an optionally substituted N-aralkyl, an alkoxy, an optionally substituted N-methyl-aralkyl, an optionally substituted N-methyl-aryl, an optionally substituted N-aryl, an optionally substituted N-cyclyl, an optionally substituted heterocyclyl or an N-alkyl; and
R_{6′} is a sulfonamide or an amide; or a pharmaceutically acceptable salt or stereoisomer thereof, wherein the compound is cell permeable and binds Pin1 with a Ki of less than 1 μM.

In some embodiments, R₁′ is a phosphorylated alkyl, a hydroxyalkyl, a sulfone, an optionally substituted aralkyl, a carboxylic acid or an ester except for

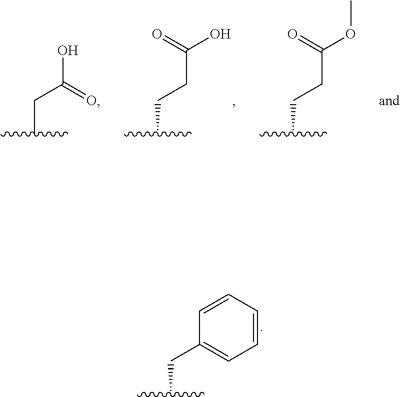

In some embodiments, R_{3′} is an optionally substituted aralkyl, a ketone or an optionally substituted heteroaralkyl except for

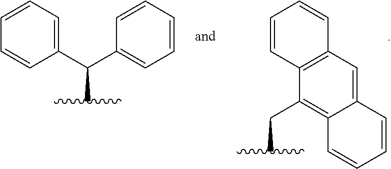

In some embodiments, R_{4′} is an alkyl urea, an alkyl guanidine, a hydroxyalkyl, an amide, an optionally substituted heteroaralkyl or an optionally substituted aralkyl except for

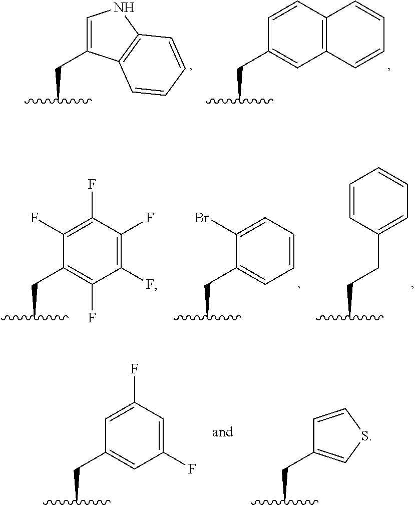

In some embodiments, R_{5′} is an optionally substituted N-aralkyl, an alkoxy, an optionally substituted N-methyl-aralkyl, an optionally substituted N-methyl-aryl, an optionally substituted N-aryl, an optionally substituted N-cyclyl, an optionally substituted heterocyclyl or an N-alkyl except for

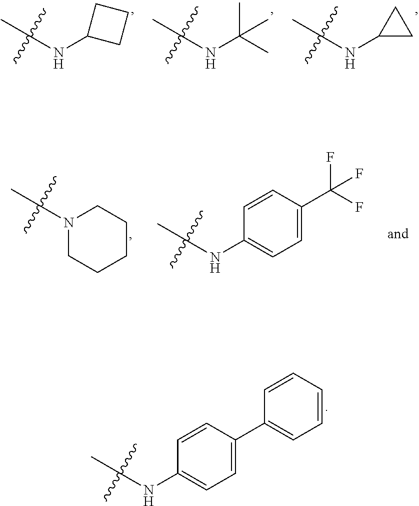

In some embodiments, R_{6′} is a sulfonamide or an amide except for

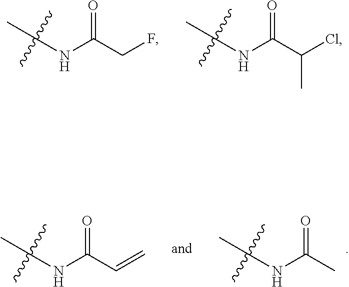

Another aspect of the present invention is directed to a pharmaceutical composition that includes a therapeutically effective amount of a compound of the invention and a pharmaceutically acceptable carrier.

A further aspect of the present invention is directed to a method for making a compound of the invention.

Another aspect of the present invention is directed to a method of treating a disease or disorder mediated by dysregulated Pin1 activity, comprising administering a therapeutically effective amount of the compound of the invention or pharmaceutically acceptable salt or stereoisomer of to a subject in need thereof.

In some embodiments, the disease or disorder is cancer, inflammation, an autoimmune disorder or a neurodegenerative disease

In some embodiments, the autoimmune disease that is treated is lupus, asthma or arthritis.

In some embodiments, the neurodegenerative disease is Alzheimer's disease or Parkinson's disease.

Unlike many previously identified Pin1 inhibitors, many of the Pin1 inhibitors disclosed herein are cell permeable. The present invention provides peptidomimetic inhibitors, many of which irreversibly bind to Pint's cysteine 113, located in the PPIase active site. Thus, the compounds disclosed herein are selective, potent and cell permeable irreversible Pin1 inhibitors.

Without intending to be bound by any particular theory of operation, it is believed that compounds of the present invention exhibit their inhibitory activity by binding to at least one amino residue, e.g. cysteine 113, located in the active site of Pin1. Without intending to be bound by any theory of operation, Applicant believes that the compounds of the present invention exert their therapeutic (e.g., anti-cancer and anti-tumor) effect or benefit at least by restoring the balance of oncogene and tumor suppressor activity in tumors.

### BRIEF DESCRIPTION OF THE DRAWINGS

FIG. 1A is a schematic diagram of a fluorescence polarization assay.

FIG. 1B is a graph of fluorescence polarization at increasing concentrations of compound 18 (Ki=20 nM) as compared to the same concentrations of the Pin1 inhibitor, pTide.

FIG. 2A is a flow chart describing the chymotrypsin-coupled peptidyl-prolyl cis-trans isomerase (PPIase) assay.

FIG. 2B is a graph of Pin1 catalytic activity at increasing concentrations of compound 18 (Ki=48 nM).

FIG. 3A is a graph of Pin1 covalent labeling time course for compound 2b-6 (Ki=170 nM).

FIG. 3B is a pair of mass spectra showing Pin1 alone (DMSO; top), and after a 30-minute room temperature incubation at 1:1 Pin1:compound 18, showing a change of 702 Da indicating 100% covalent labeling of Pin1 by compound 18 upon displacement of the chlorine.

FIG. 4A is a depiction of the 1.8 A resolution x-ray co-crystal structure of compound 2b-6 covalently bound to Pin1 with the compound shown as a stick diagram in the PPIase active site.

FIG. 4B is a depiction of the x-ray co-crystal structure of compound 2b-6 covalently bound to Pin1 with the compound shown as a stick diagram overlayed with an electron density map in the PPIase active site.

FIG. 4C is a table showing the statistics of the crystallographic model for compound 2b-6 bound to Pin1.

FIG. 5A is a photograph of a Western blot showing that compound 2-25 (Ki=20 nM), which corresponds to compound 2b-19 (Ki=30 nM) covalently linked to biotin, was able to pull down Pin1 from the TNBC MDA-MB-231 lysate at a concentration of 1 μM, whereas compound 2-30) (Ki N/A), which is the negative control stereoisomer of compound 2-25, was unable to pull down Pin1 from the lysate.

FIG. 5B is a photograph of a Western blot showing that compound 2-25 (Ki=18 nM), which corresponds to compound 18 (Ki=20 nM) covalently linked to biotin, was able to pull down Pin1 from the TNBC MDA-MB-231 lysate at concentrations of 500 nM and 1 μM.

FIG. 6A is a diagram describing a lysate target engagement experiment via a competition assay with compound 18 and compound 2c (Ki=15 nM), which corresponds to compound 18 covalently linked to desthiobiotin. (o/n=overnight.)

FIG. 6B is a photograph of a Western Blot showing that incubation with compound 18 competes with compound 2c for binding of Pin1 in HEK 293 cell lysate.

FIG. 7A is a diagram depicting a live cell target engagement biotin competition assay in TNBC MDA-MB-231 cells.

FIG. 7B is a photograph of a Western Blot showing that preincubation of live TNBC-MDA-MB-231 cells with increasing concentrations of compound 18 outcompetes compound 2-32 for Pin1 binding, demonstrating cellular target engagement and cell permeability.

FIG. 8A is a diagram depicting the Covalent Inhibitor Target-site Identification (CiTe-ID) experiment, a chemoproteomic method to quantify the dose-dependent binding of covalent inhibitors to cysteine residues proteome-wide.

FIG. 8B is a graph showing that Pin1 C113 is the only site that undergoes dose-dependent covalent modification by compound 18 in HEK 293 cell lysates

FIG. 9 is a photograph of a Western blot of the protein expression of phosphoRb (S780), phosphoRb (S801/811), β-Catenin, cJun, cMyc and phosphoH3 (S10) after incubation of TNBC-MDA-MB 231 cells for 1, 2, 4, 6 and 8 hours with 10 μM of compound 18. Inhibition of Pin1 leads to downstream changes including a decrease in phosphoRb, β-Catenin, cJun, phosphoH3 (S10), and a temporary increase in c-Myc.

FIG. 10 is graph showing that Compound 18 induced dose-dependent and time-dependent defects in cell viability in the pancreatic ductal adenocarcinoma (PDAC) cell line, PATU-8988T.

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

As used herein, “Ki of less than 1 μM” means a Ki value less than 1 μM obtained as measured by a peptidyl-prolyl cis-trans isomerase (PPIase) assay as described in Example 7.

With respect to compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group.

In some embodiments, an alkyl group is a C₁-C₂ alkyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “haloalkyl” refers to an alkyl group as defined herein that is substituted with one or more (e.g., 1, 2, 3, or 4) halo groups.

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-C₁₈ group. In other embodiments, the alkenyl radical is a C₂-C₁₂, C₂-C₁₀, C2-C8, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

As used herein, the term “alkynyl” refers to a linear or branched monovalent hydrocarbon radical with at least one carbon-carbon triple bond. In one example, the alkynyl radical is a C₂-C₁₈ group. In other examples, the alkynyl radical is C₂-C₁₂, C₂-C₁₀, C₂-C₅, C₂-C₆ or C₂-C₃. Examples include ethynyl prop-1-ynyl, prop-2-ynyl, but-1-ynyl, but-2-ynyl and but-3-ynyl.

As used herein, the term “aldehyde” is represented by the formula —C(O)H. The terms “C(O)” and C═O are used interchangeably herein.

The terms “alkoxyl” or “alkoxy” as used herein refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “alkyl urea” is represented by the formula Z¹NHC(O)NH₂, where Z¹ may be an alkyl, halogenated alkyl, alkenyl, alkynyl, aryl, heteroaryl, cycloalkyl, cycloalkenyl, heterocycloalkyl, or heterocycloalkenyl group, all as described herein.

As used herein, the term “alkyl guanidine” is represented by the formula Z¹NHC(NH)NH₂, where Z¹ may be an alkyl, halogenated alkyl, alkenyl, alkynyl, aryl, heteroaryl, cycloalkyl, cycloalkenyl, heterocycloalkyl, or heterocycloalkenyl group, all as described herein.

As used herein, the term “phosphorylated alkyl” is represented by the formula Z¹OP(O)(OH)₂, where Z¹ may be an alkyl, halogenated alkyl, alkenyl, alkynyl, aryl, heteroaryl, cycloalkyl, cycloalkenyl, heterocycloalkyl, or heterocycloalkenyl group, all as described herein.

As used herein, the term “halogen” (or “halo” or “halide”) refers to fluorine, chlorine, bromine, or iodine.

As used herein, the term “carboxylic acid” is represented by the formula —C(O)OH, and a “carboxylate” is represented by the formula —C(O)O—.

As used herein, the term “ester” is represented by the formula —OC(O)Z¹ or —C(O)OZ¹, where Z¹ may be an alkyl, halogenated alkyl, alkenyl, alkynyl, aryl, heteroaryl, cycloalkyl, cycloalkenyl, heterocycloalkyl, or heterocycloalkenyl group, all as described herein.

As used herein, the term “ether” is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group, all as described herein.

As used herein, the term “ketone” is represented by the formula Z¹C(O)Z², where Z¹ and Z² independently represent alkyl, halogenated alkyl, alkenyl, alkynyl, aryl (e.g., benzophenone), heteroaryl, cycloalkyl, cycloalkenyl, heterocycloalkyl, or heterocycloalkenyl group, all as described herein.

As used herein, the term “sulfonyl” refers to the sulfo-oxo group represented by the formula —S(O)₂Z¹, where Z¹ may be hydrogen, an alkyl, halogenated alkyl, alkenyl, alkynyl, aryl, heteroaryl, cycloalkyl, cycloalkenyl, heterocycloalkyl, or heterocycloalkenyl group, all as described herein.

As used herein, the term “sulfonylamino” (or “sulfonamide”) is represented by the formula —S(O)₂NH₂.

As used herein, the term “sulfone” is represented by the formula Z¹S(O)₂Z², where Z¹ and Z² independently represent alkyl, halogenated alkyl, alkenyl, alkynyl, aryl, heteroaryl, cycloalkyl, cycloalkenyl, heterocycloalkyl, or heterocycloalkenyl group, all as described herein.

As used herein, the term “thiol” is represented by the formula —SH.

As used herein, the term “amide” is represented by the formula Z¹C(O)NH₂ wherein Z¹ where Z¹ may be an alkyl, halogenated alkyl, alkenyl, alkynyl, aryl, heteroaryl, cycloalkyl, cycloalkenyl, heterocycloalkyl, or heterocycloalkenyl group, all as described herein.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂.

Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane.

The term carbocyclyl includes aryl ring systems as defined herein. The term carbocycyl also includes cycloalkyl rings (e.g., saturated or partially unsaturated mono-, bi-, or spiro-carbocycles).

The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-Cₓ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and Spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur or oxygen. In some embodiments, heterocyclyl includes 4- to 6-membered monocycles having one or more heteroatoms selected from nitrogen, sulfur or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group.

Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain.

The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Thus, the term aryl embraces aralkyl groups which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 4-6 membered monocyclic aromatic groups where one or more ring atoms is nitrogen, sulfur or oxygen that is independently optionally substituted. In another embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, triazinyl, tetrazinyl, tetrazolo[1,5-b]pyridazinyl, purinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroaryl alkyl groups which as disclosed above refer to a group of the formula —R^{c}-heteroaryl, where R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

Representative substituents include halogens, hydroxyl groups, and any other organic groupings containing any number of carbon atoms, e.g., 1-14 carbon atoms, and which may include one or more (e.g., 1 2 3, or 4) heteroatoms such as oxygen, sulfur, and nitrogen grouped in a linear, branched, or cyclic structural format.

Compounds of the present invention have a structure as represented by formula (I):

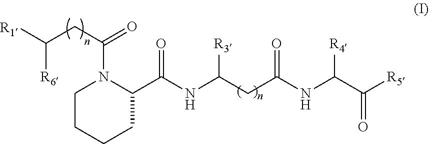

wherein each n is independently 0 or 1;
R₁′ is a phosphorylated alkyl, a hydroxyalkyl, a sulfone, an optionally substituted aralkyl, a carboxylic acid or an ester;
R_{3′} is an optionally substituted aralkyl, a ketone or an optionally substituted heteroaralkyl;
R_{4′} is an alkyl urea, an alkyl guanidine, a hydroxyalkyl, an amide, an optionally substituted heteroaralkyl or an optionally substituted aralkyl;
R_{5′} is an optionally substituted N-aralkyl, an alkoxy, an optionally substituted N-methyl-aralkyl, an optionally substituted N-methyl-aryl, an optionally substituted N-aryl, an optionally substituted N-cyclyl, an optionally substituted heterocyclyl or an N-alkyl; and
R_{6′} is a sulfonamide or an amide; or a pharmaceutically acceptable salt or stereoisomer thereof, wherein the compound is cell permeable and binds Pin1 with a Ki of less than 1 μM.

In some embodiments, R₁′ is a phosphorylated alkyl, a hydroxyalkyl, a sulfone, an optionally substituted aralkyl, a carboxylic acid or an ester except for

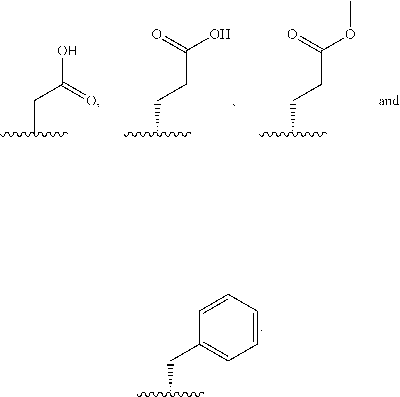

In some embodiments, R_{3′} is an optionally substituted aralkyl, a ketone or an optionally substituted heteroaralkyl except for

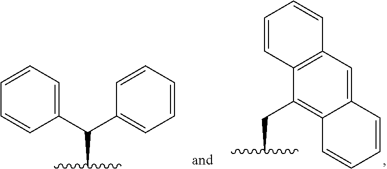

In some embodiments, R_{4′} is an alkyl urea, an alkyl guanidine, a hydroxyalkyl, an amide, an optionally substituted heteroaralkyl or an optionally substituted aralkyl except for

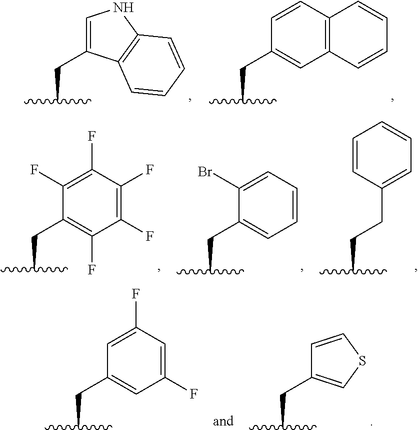

In some embodiments, R_{5′} is an optionally substituted N-aralkyl, an alkoxy, an optionally substituted N-methyl-aralkyl, an optionally substituted N-methyl-aryl, an optionally substituted N-aryl, an optionally substituted N-cyclyl, an optionally substituted heterocyclyl or an N-alkyl except for

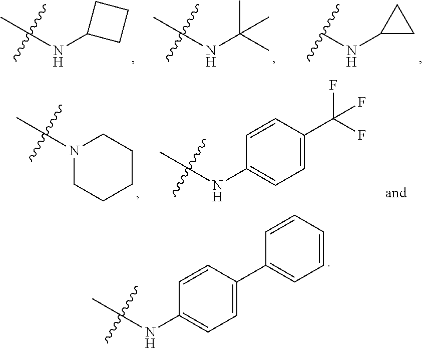

In some embodiments, R_{6′} is a sulfonamide or an amide except for

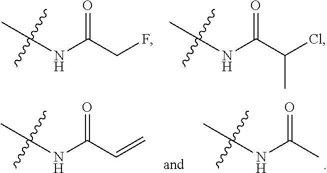

Representative examples of R_{1′} include

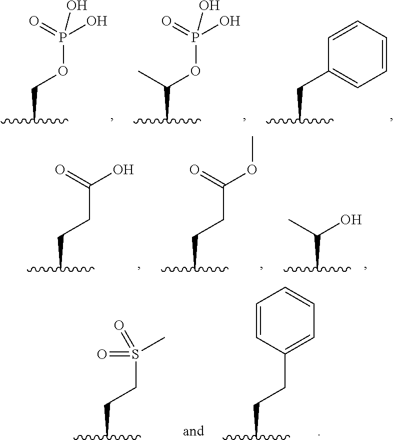

Representative examples of R_{3′} include

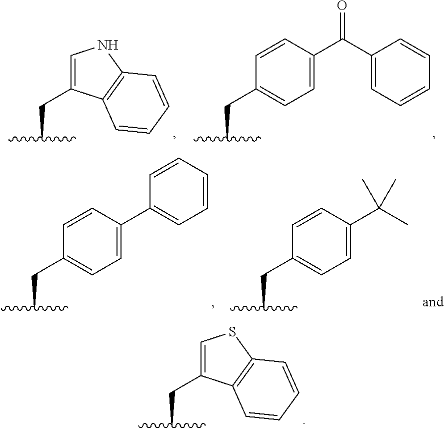

Representative examples of R_{4′} include

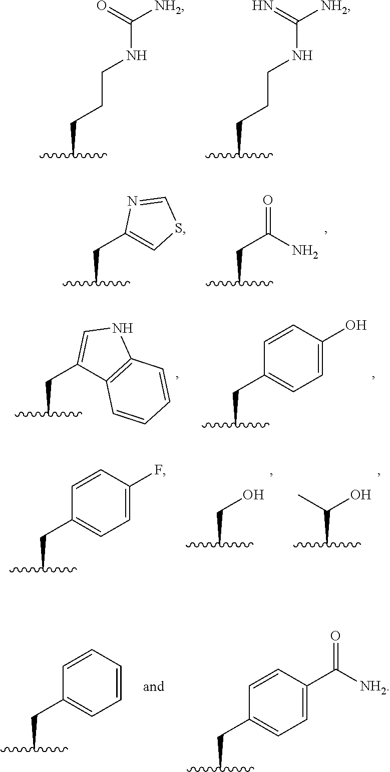

Representative examples of R_{5′} include

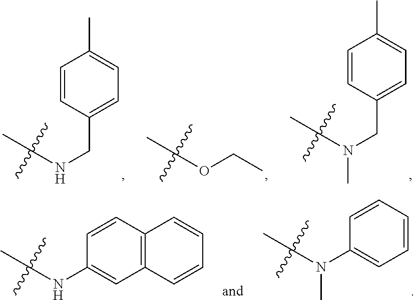

Representative examples of R_{6′} include

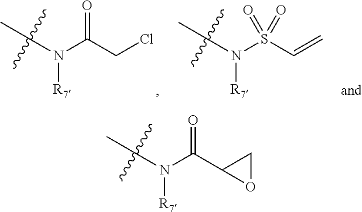

wherein R_{7′} is hydrogen or methyl.

In some embodiments, R_{6′} is chloroacetamide and the inventive compounds are represented by formula (Ia):

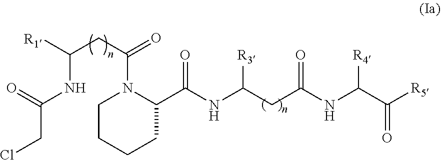

or a pharmaceutically acceptable salt or stereoisomer thereof.

In some embodiments, R_{6′} is N-methyl chloroacetamide and the inventive compounds are represented by formula (Ib):

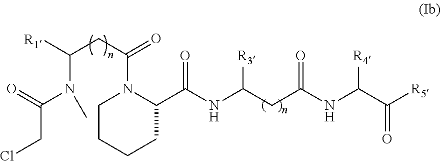

or a pharmaceutically acceptable salt or stereoisomer thereof.

In some embodiments, R_{1′} is benzyl and the inventive compounds are represented by formula (Ic):

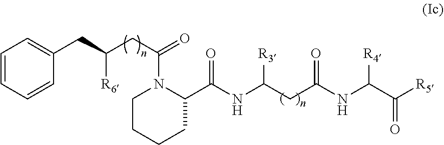

or a pharmaceutically acceptable salt or stereoisomer thereof.

In some embodiments, R_{3′} is an alkyl substituted indole and the inventive compounds are represented by formula (Id):

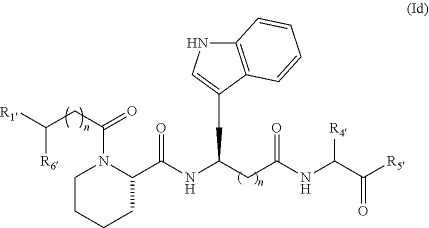

or a pharmaceutically acceptable salt or stereoisomer thereof.

In some embodiments, R_{4′} is an alkyl urea and the inventive compounds are represented by formula (Ie):

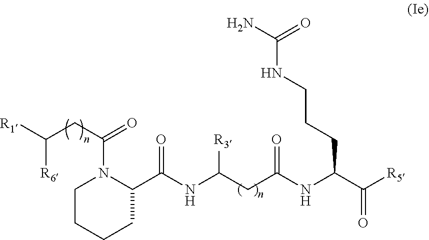

or a pharmaceutically acceptable salt or stereoisomer thereof.

In some embodiments, R_{4′} is an alkyl guanidine and the inventive compounds are represented by formula (If):

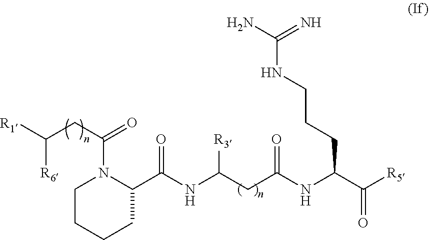

or a pharmaceutically acceptable salt or stereoisomer thereof.

In some embodiments, R_{5′} is alkoxy and the inventive compounds are represented by formula (Ig):

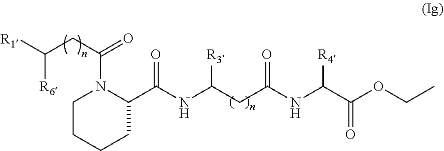

or a pharmaceutically acceptable salt or stereoisomer thereof.

In some embodiments, R_{5′} is methyl substituted N-benzyl and the inventive compounds are represented by formula (Ih):

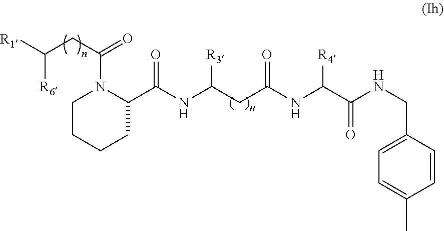

or a pharmaceutically acceptable salt or stereoisomer thereof.

The following substituents are not tolerated at position R_{6′}, i.e. there is a loss of activity and/or cell permeability:

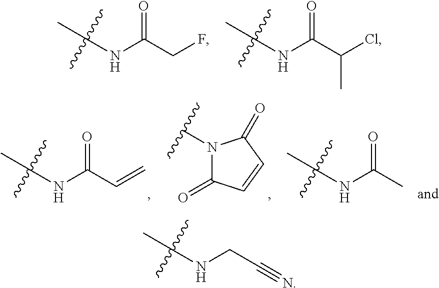

The following substituents are not tolerated at position R_{1′}, i.e. there is a loss of activity and/or cell permeability:

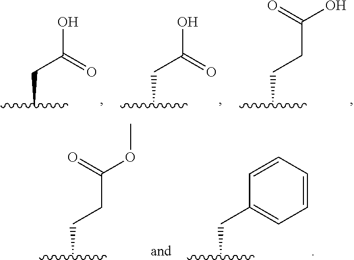

The following substituents are not tolerated at position R_{3′}, i.e. there is a loss of activity and/or cell permeability:

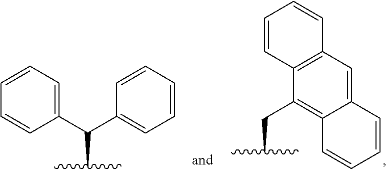

The following substituents are not tolerated at position R_{4′}, i.e. there is a loss of activity and/or cell permeability:

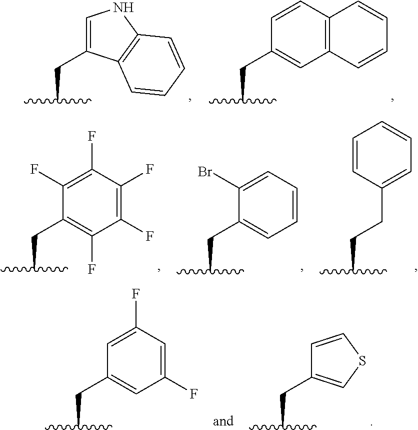

The following substituents are not tolerated at position R_{5′}, i.e. activity is retained, but there is a loss of cell permeability:

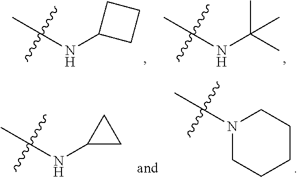

The following substituents are not tolerated at position R_{5′}, i.e. there is a loss of activity and/or cell permeability:

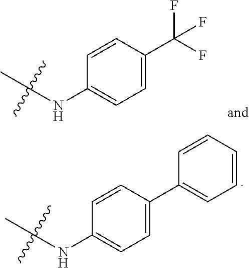 [SMILES: *Nc1ccc(C(F)(F)F)cc1 ; *Nc1ccc(-c2ccccc2)cc1]

In some embodiments, the compounds of the present invention are represented by any of the following structures:

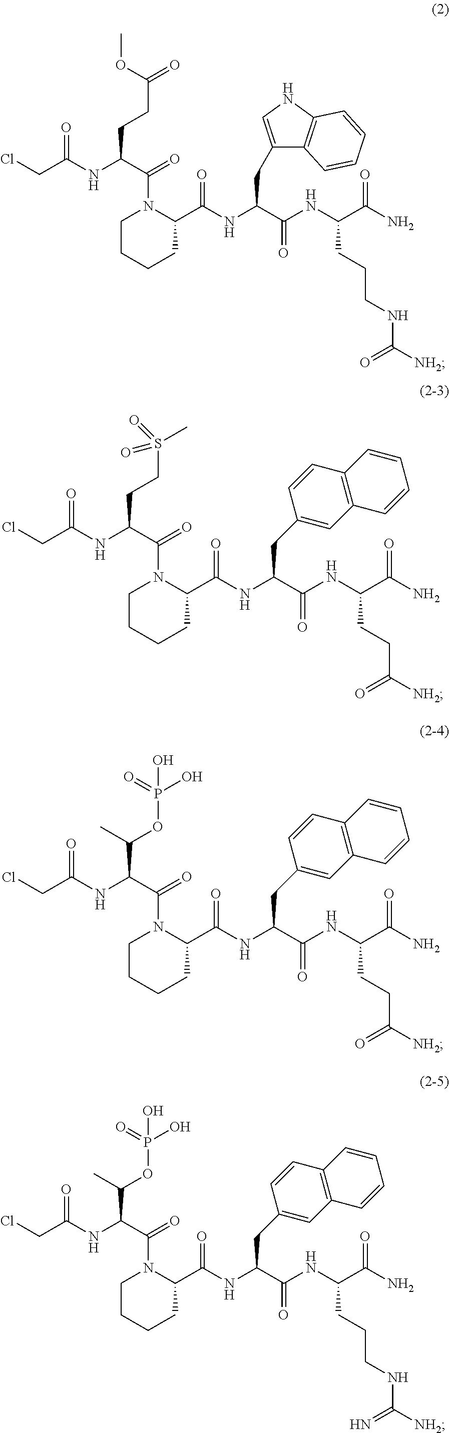 [SMILES: COC(=O)CC[C@H](NC(=O)CCl)C(=O)N1CCCC[C@H]1C(=O)N[C@@H](Cc1c[nH]c2ccccc12)C(=O)N[C@@H](CCCNC(N)=O)C(N)=O ; CS(=O)(=O)CC[C@H](NC(=O)CCl)C(=O)N1CCCC[C@H]1C(=O)N[C@@H](Cc1ccc2ccccc2c1)C(=O)N[C@@H](CCC(N)=O)C(N)=O ; CC(OP(=O)(O)O)[C@H](NC(=O)CCl)C(=O)N1CCCC[C@H]1C(=O)N[C@@H](Cc1ccc2ccccc2c1)C(=O)N[C@@H](CCC(N)=O)C(N)=O ; CC(OP(=O)(O)O)[C@H](NC(=O)CCl)C(=O)N1CCCC[C@H]1C(=O)N[C@@H](Cc1ccc2ccccc2c1)C(=O)N[C@@H](CCCNC(=N)N)C(N)=O]

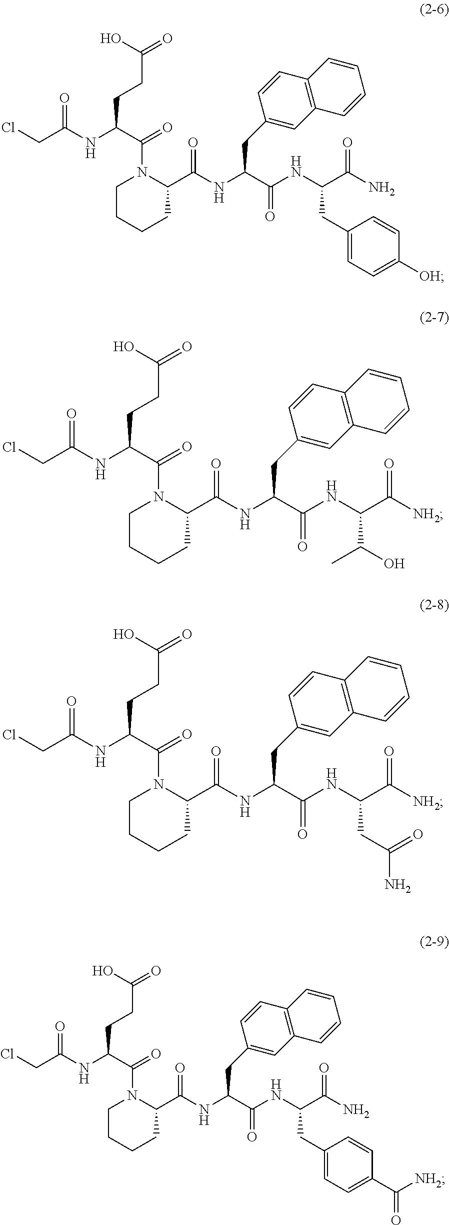 [SMILES: NC(=O)[C@H](Cc1ccc(O)cc1)NC(=O)[C@H](Cc1ccc2ccccc2c1)NC(=O)[C@@H]1CCCCN1C(=O)[C@H](CCC(=O)O)NC(=O)CCl ; CC(O)[C@H](NC(=O)[C@H](Cc1ccc2ccccc2c1)NC(=O)[C@@H]1CCCCN1C(=O)[C@H](CCC(=O)O)NC(=O)CCl)C(N)=O ; NC(=O)C[C@H](NC(=O)[C@H](Cc1ccc2ccccc2c1)NC(=O)[C@@H]1CCCCN1C(=O)[C@H](CCC(=O)O)NC(=O)CCl)C(N)=O ; NC(=O)c1ccc(C[C@H](NC(=O)[C@H](Cc2ccc3ccccc3c2)NC(=O)[C@@H]2CCCCN2C(=O)[C@H](CCC(=O)O)NC(=O)CCl)C(N)=O)cc1]

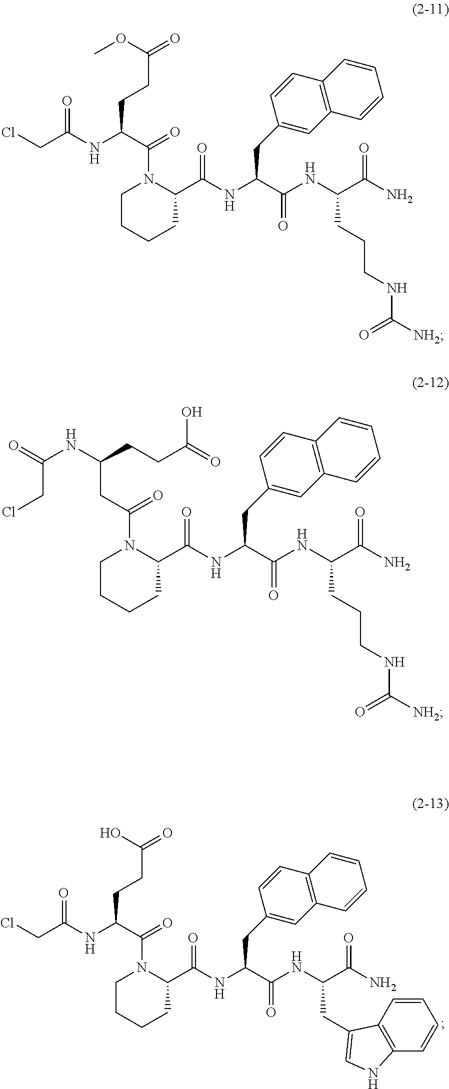 [SMILES: COC(=O)CC[C@H](NC(=O)CCl)C(=O)N1CCCC[C@H]1C(=O)N[C@@H](Cc1ccc2ccccc2c1)C(=O)N[C@@H](CCCNC(N)=O)C(N)=O ; NC(=O)NCCC[C@H](NC(=O)[C@H](Cc1ccc2ccccc2c1)NC(=O)[C@@H]1CCCCN1C(=O)C[C@H](CCC(=O)O)NC(=O)CCl)C(N)=O ; NC(=O)[C@H](Cc1c[nH]c2ccccc12)NC(=O)[C@H](Cc1ccc2ccccc2c1)NC(=O)[C@@H]1CCCCN1C(=O)[C@H](CCC(=O)O)NC(=O)CCl]

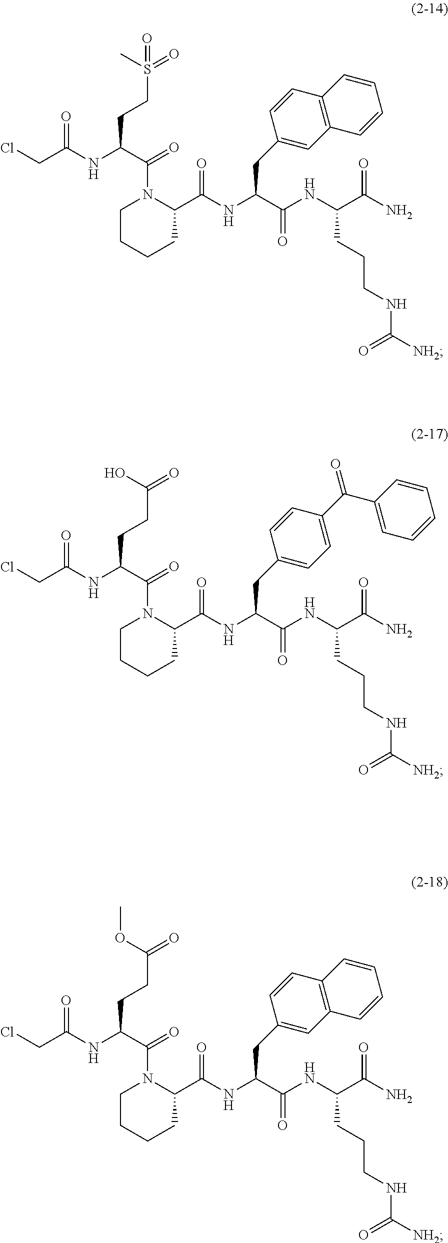 [SMILES: CS(=O)(=O)CC[C@H](NC(=O)CCl)C(=O)N1CCCC[C@H]1C(=O)N[C@@H](Cc1ccc2ccccc2c1)C(=O)N[C@@H](CCCNC(N)=O)C(N)=O ; NC(=O)NCCC[C@H](NC(=O)[C@H](Cc1ccc(C(=O)c2ccccc2)cc1)NC(=O)[C@@H]1CCCCN1C(=O)[C@H](CCC(=O)O)NC(=O)CCl)C(N)=O ; COC(=O)CC[C@H](NC(=O)CCl)C(=O)N1CCCC[C@H]1C(=O)N[C@@H](Cc1ccc2ccccc2c1)C(=O)N[C@@H](CCCNC(N)=O)C(N)=O]

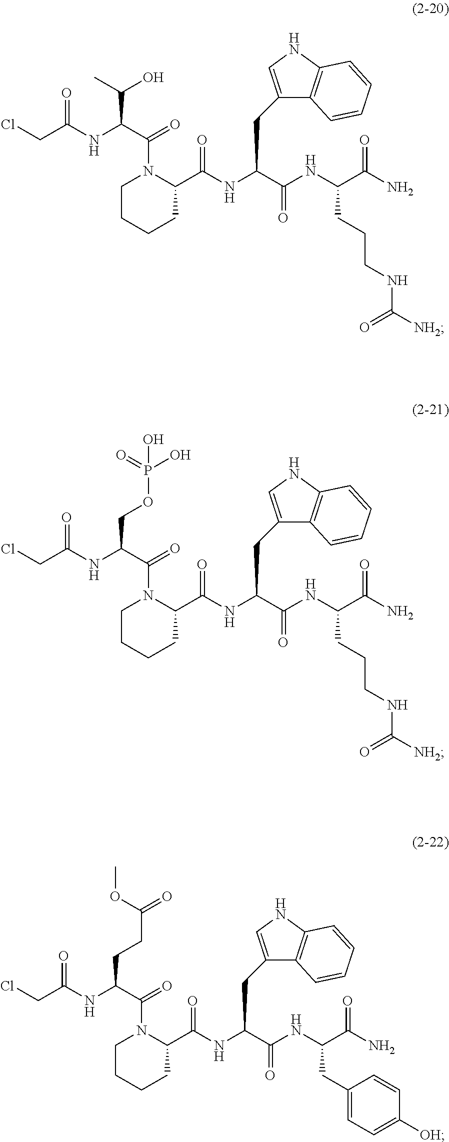 [SMILES: CC(O)[C@H](NC(=O)CCl)C(=O)N1CCCC[C@H]1C(=O)N[C@@H](Cc1c[nH]c2ccccc12)C(=O)N[C@@H](CCCNC(N)=O)C(N)=O ; NC(=O)NCCC[C@H](NC(=O)[C@H](Cc1c[nH]c2ccccc12)NC(=O)[C@@H]1CCCCN1C(=O)[C@H](COP(=O)(O)O)NC(=O)CCl)C(N)=O ; COC(=O)CC[C@H](NC(=O)CCl)C(=O)N1CCCC[C@H]1C(=O)N[C@@H](Cc1c[nH]c2ccccc12)C(=O)N[C@@H](Cc1ccc(O)cc1)C(N)=O]

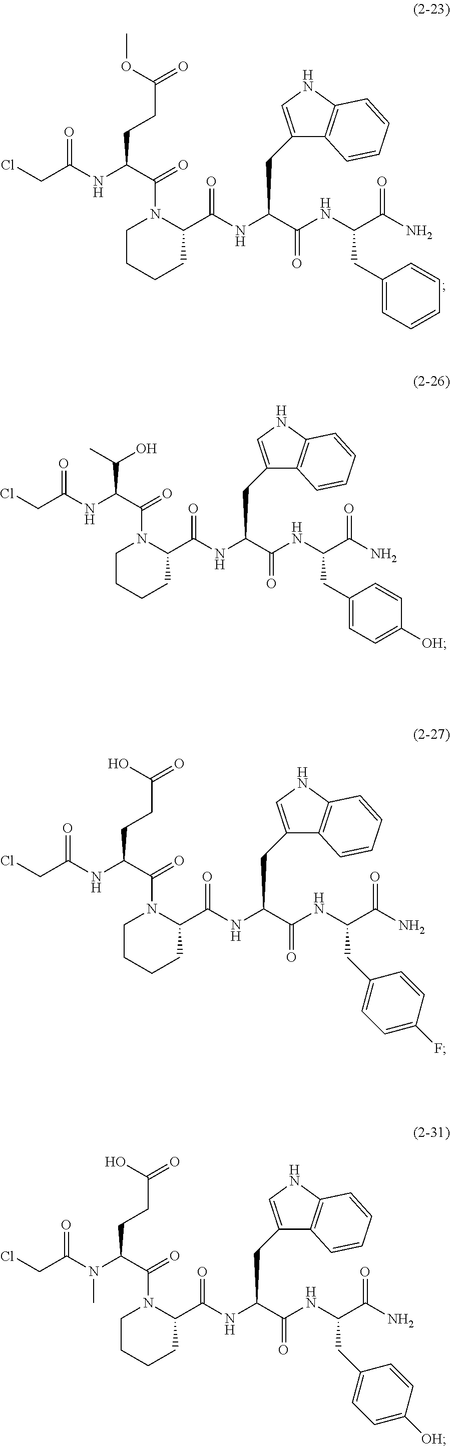 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1ccccc1)C(N)=O ; CC(O)[C@H](NC(=O)CCl)C(=O)N1CCCC[C@H]1C(=O)N[C@@H](Cc1c[nH]c2ccccc12)C(=O)N[C@@H](Cc1ccc(O)cc1)C(N)=O ; NC(=O)[C@H](Cc1ccc(F)cc1)NC(=O)[C@H](Cc1c[nH]c2ccccc12)NC(=O)[C@@H]1CCCCN1C(=O)[C@H](CCC(=O)O)NC(=O)CCl ; CN(C(=O)CCl)[C@@H](CCC(=O)O)C(=O)N1CCCC[C@H]1C(=O)N[C@@H](Cc1c[nH]c2ccccc12)C(=O)N[C@@H](Cc1ccc(O)cc1)C(N)=O]

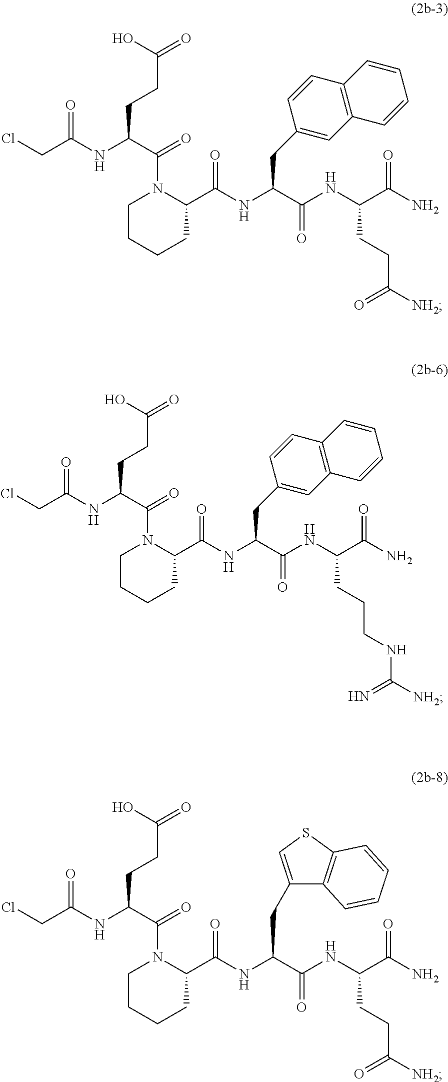 [SMILES: NC(=O)CC[C@H](NC(=O)[C@H](Cc1ccc2ccccc2c1)NC(=O)[C@@H]1CCCCN1C(=O)[C@H](CCC(=O)O)NC(=O)CCl)C(N)=O ; N=C(N)NCCC[C@H](NC(=O)[C@H](Cc1ccc2ccccc2c1)NC(=O)[C@@H]1CCCCN1C(=O)[C@H](CCC(=O)O)NC(=O)CCl)C(N)=O ; NC(=O)CC[C@H](NC(=O)[C@H](Cc1csc2ccccc12)NC(=O)[C@@H]1CCCCN1C(=O)[C@H](CCC(=O)O)NC(=O)CCl)C(N)=O]

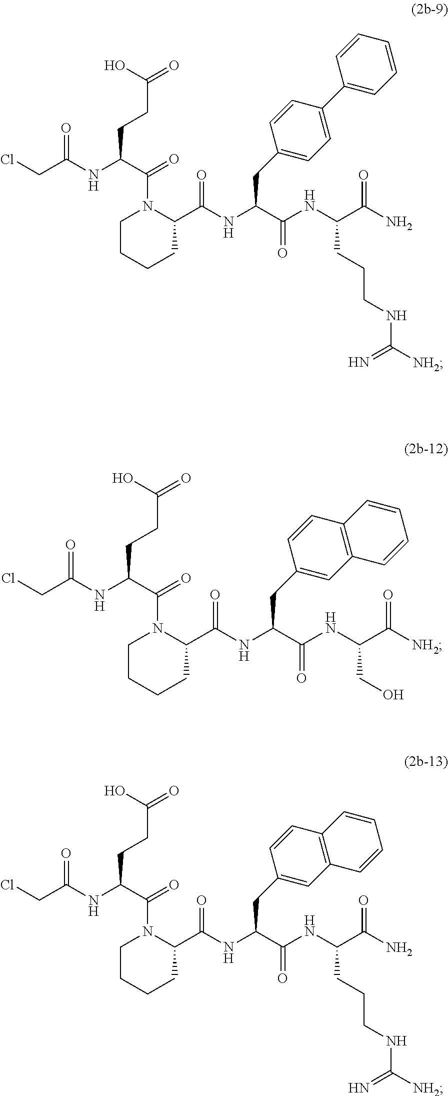 [SMILES: N=C(N)NCCC[C@H](NC(=O)[C@H](Cc1ccc(-c2ccccc2)cc1)NC(=O)[C@@H]1CCCCN1C(=O)[C@H](CCC(=O)O)NC(=O)CCl)C(N)=O ; NC(=O)[C@H](CO)NC(=O)[C@H](Cc1ccc2ccccc2c1)NC(=O)[C@@H]1CCCCN1C(=O)[C@H](CCC(=O)O)NC(=O)CCl ; N=C(N)NCCC[C@H](NC(=O)[C@H](Cc1ccc2ccccc2c1)NC(=O)[C@@H]1CCCCN1C(=O)[C@H](CCC(=O)O)NC(=O)CCl)C(N)=O]

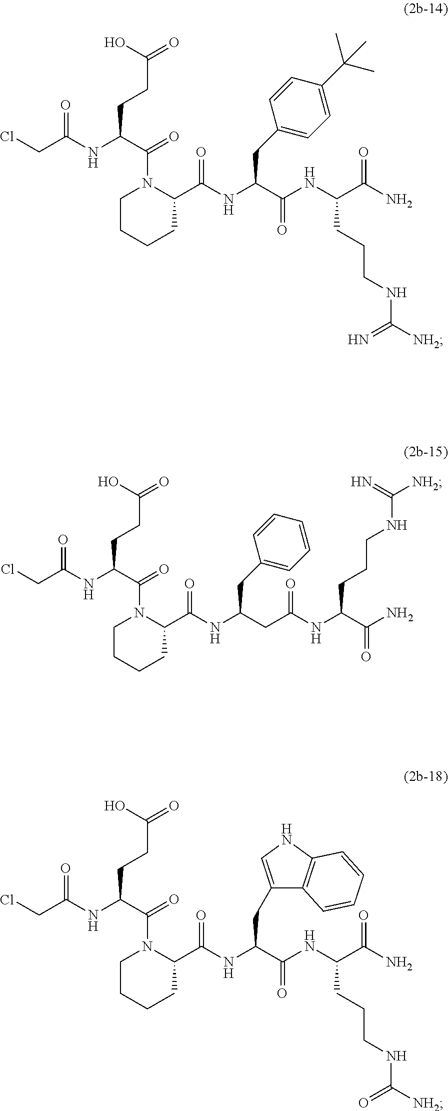 [SMILES: CC(C)(C)c1ccc(C[C@H](NC(=O)[C@@H]2CCCCN2C(=O)[C@H](CCC(=O)O)NC(=O)CCl)C(=O)N[C@@H](CCCNC(=N)N)C(N)=O)cc1 ; N=C(N)NCCC[C@H](NC(=O)C[C@H](Cc1ccccc1)NC(=O)[C@@H]1CCCCN1C(=O)[C@H](CCC(=O)O)NC(=O)CCl)C(N)=O ; NC(=O)NCCC[C@H](NC(=O)[C@H](Cc1c[nH]c2ccccc12)NC(=O)[C@@H]1CCCCN1C(=O)[C@H](CCC(=O)O)NC(=O)CCl)C(N)=O]

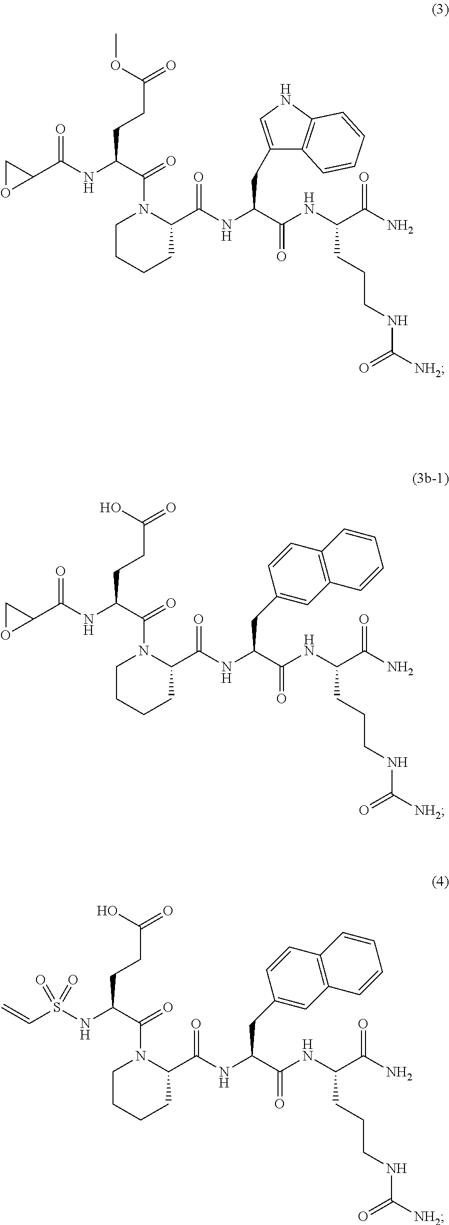 [SMILES: COC(=O)CC[C@H](NC(=O)C1CO1)C(=O)N1CCCC[C@H]1C(=O)N[C@@H](Cc1c[nH]c2ccccc12)C(=O)N[C@@H](CCCNC(N)=O)C(N)=O ; NC(=O)NCCC[C@H](NC(=O)[C@H](Cc1ccc2ccccc2c1)NC(=O)[C@@H]1CCCCN1C(=O)[C@H](CCC(=O)O)NC(=O)C1CO1)C(N)=O ; C=CS(=O)(=O)N[C@@H](CCC(=O)O)C(=O)N1CCCC[C@H]1C(=O)N[C@@H](Cc1ccc2ccccc2c1)C(=O)N[C@@H](CCCNC(N)=O)C(N)=O]

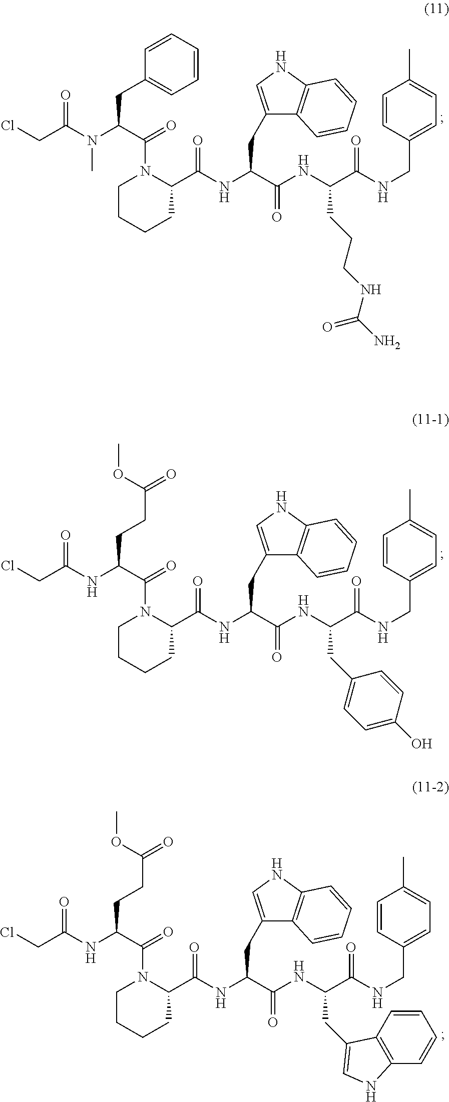 [SMILES: Cc1ccc(CNC(=O)[C@H](CCCNC(N)=O)NC(=O)[C@H](Cc2c[nH]c3ccccc23)NC(=O)[C@@H]2CCCCN2C(=O)[C@H](Cc2ccccc2)N(C)C(=O)CCl)cc1 ; COC(=O)CC[C@H](NC(=O)CCl)C(=O)N1CCCC[C@H]1C(=O)N[C@@H](Cc1c[nH]c2ccccc12)C(=O)N[C@@H](Cc1ccc(O)cc1)C(=O)NCc1ccc(C)cc1 ; COC(=O)CC[C@H](NC(=O)CCl)C(=O)N1CCCC[C@H]1C(=O)N[C@@H](Cc1c[nH]c2ccccc12)C(=O)N[C@@H](Cc1c[nH]c2ccccc12)C(=O)NCc1ccc(C)cc1]

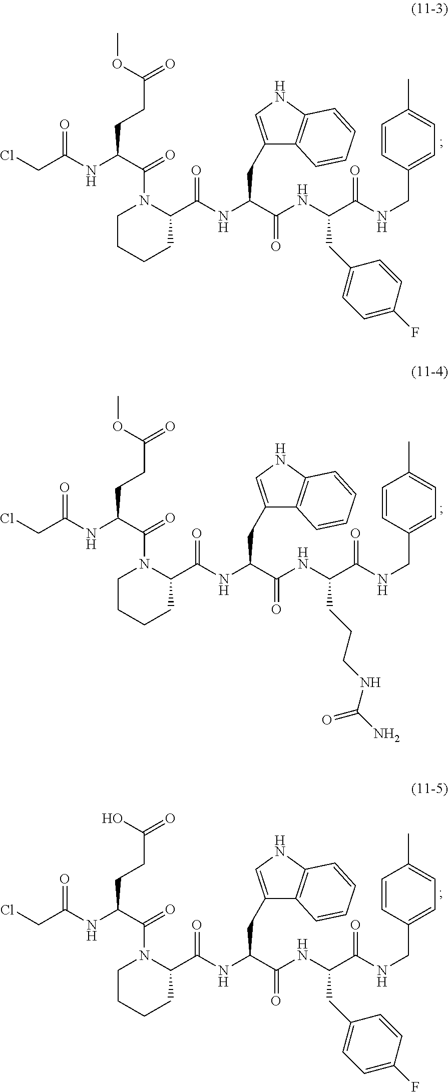 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1ccc(F)cc1)C(=O)NCc1ccc(C)cc1 ; COC(=O)CC[C@H](NC(=O)CCl)C(=O)N1CCCC[C@H]1C(=O)N[C@@H](Cc1c[nH]c2ccccc12)C(=O)N[C@@H](CCCNC(N)=O)C(=O)NCc1ccc(C)cc1 ; Cc1ccc(CNC(=O)[C@H](Cc2ccc(F)cc2)NC(=O)[C@H](Cc2c[nH]c3ccccc23)NC(=O)[C@@H]2CCCCN2C(=O)[C@H](CCC(=O)O)NC(=O)CCl)cc1]

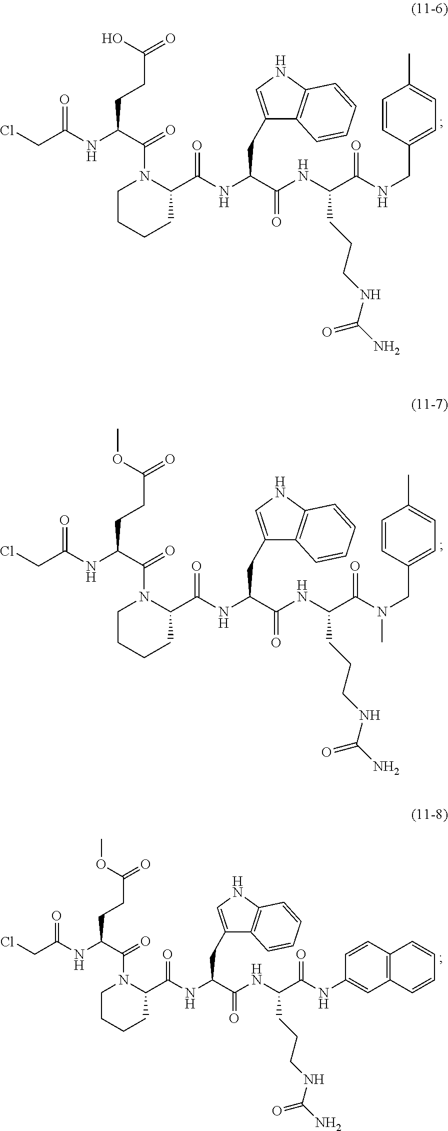 [SMILES: Cc1ccc(CNC(=O)[C@H](CCCNC(N)=O)NC(=O)[C@H](Cc2c[nH]c3ccccc23)NC(=O)[C@@H]2CCCCN2C(=O)[C@H](CCC(=O)O)NC(=O)CCl)cc1 ; COC(=O)CC[C@H](NC(=O)CCl)C(=O)N1CCCC[C@H]1C(=O)N[C@@H](Cc1c[nH]c2ccccc12)C(=O)N[C@@H](CCCNC(N)=O)C(=O)N(C)Cc1ccc(C)cc1 ; COC(=O)CC[C@H](NC(=O)CCl)C(=O)N1CCCC[C@H]1C(=O)N[C@@H](Cc1c[nH]c2ccccc12)C(=O)N[C@@H](CCCNC(N)=O)C(=O)Nc1ccc2ccccc2c1]

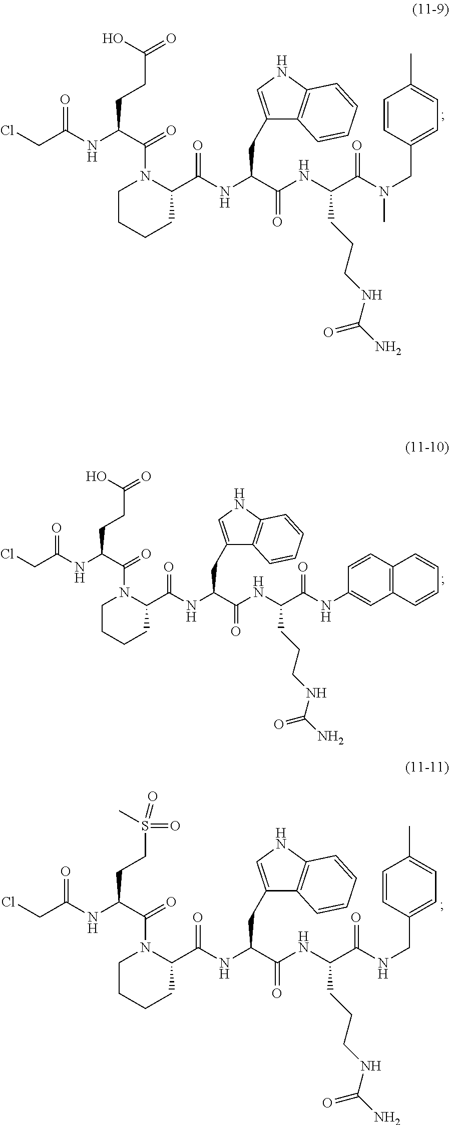 [SMILES: Cc1ccc(CN(C)C(=O)[C@H](CCCNC(N)=O)NC(=O)[C@H](Cc2c[nH]c3ccccc23)NC(=O)[C@@H]2CCCCN2C(=O)[C@H](CCC(=O)O)NC(=O)CCl)cc1 ; NC(=O)NCCC[C@H](NC(=O)[C@H](Cc1c[nH]c2ccccc12)NC(=O)[C@@H]1CCCCN1C(=O)[C@H](CCC(=O)O)NC(=O)CCl)C(=O)Nc1ccc2ccccc2c1 ; Cc1ccc(CNC(=O)[C@H](CCCNC(N)=O)NC(=O)[C@H](Cc2c[nH]c3ccccc23)NC(=O)[C@@H]2CCCCN2C(=O)[C@H](CCS(C)(=O)=O)NC(=O)CCl)cc1]

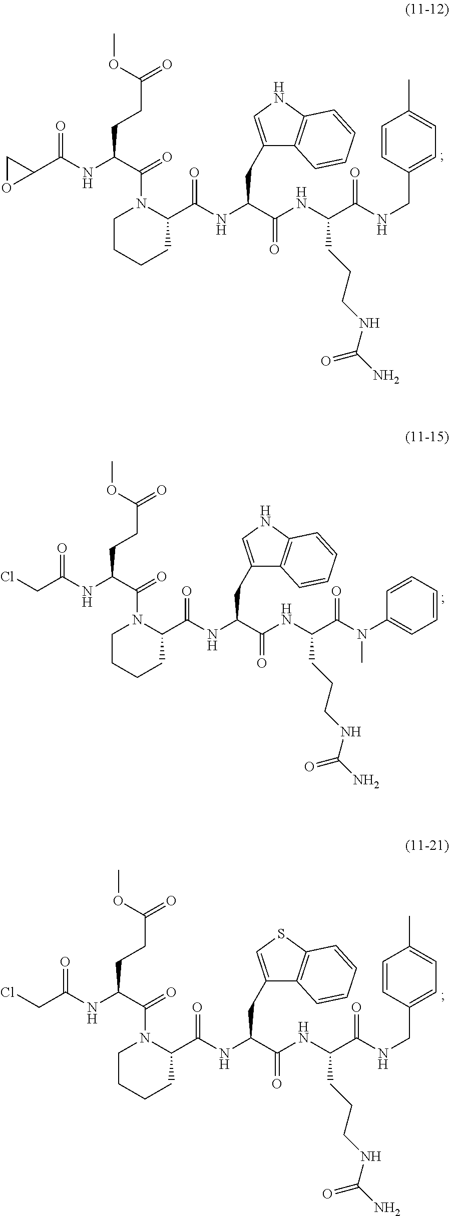 [SMILES: COC(=O)CC[C@H](NC(=O)C1CO1)C(=O)N1CCCC[C@H]1C(=O)N[C@@H](Cc1c[nH]c2ccccc12)C(=O)N[C@@H](CCCNC(N)=O)C(=O)NCc1ccc(C)cc1 ; COC(=O)CC[C@H](NC(=O)CCl)C(=O)N1CCCC[C@H]1C(=O)N[C@@H](Cc1c[nH]c2ccccc12)C(=O)N[C@@H](CCCNC(N)=O)C(=O)N(C)c1ccccc1 ; COC(=O)CC[C@H](NC(=O)CCl)C(=O)N1CCCC[C@H]1C(=O)N[C@@H](Cc1csc2ccccc12)C(=O)N[C@@H](CCCNC(N)=O)C(=O)NCc1ccc(C)cc1]

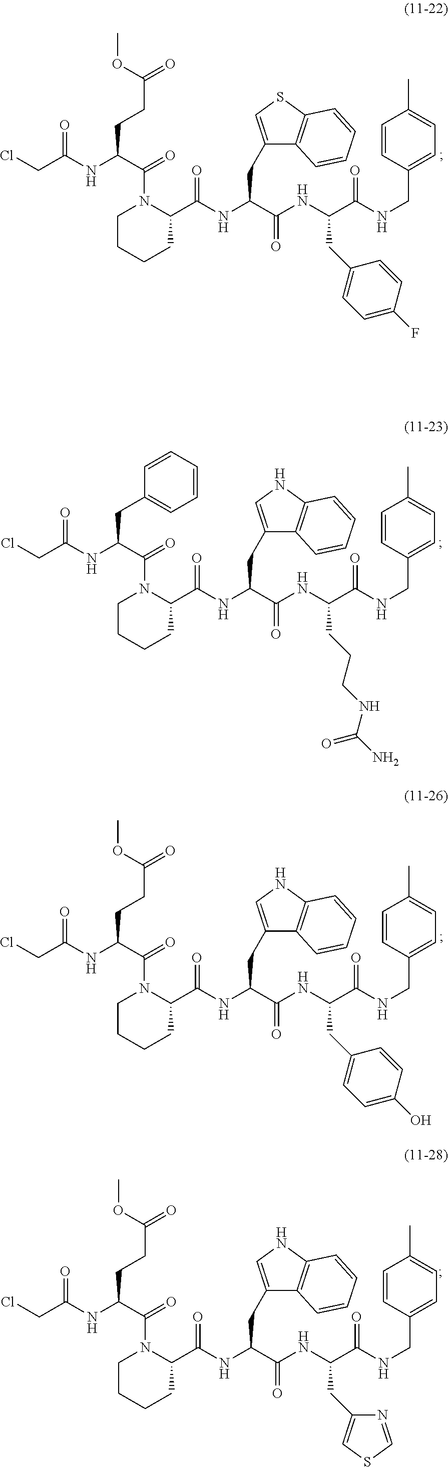 [SMILES: COC(=O)CC[C@H](NC(=O)CCl)C(=O)N1CCCC[C@H]1C(=O)N[C@@H](Cc1csc2ccccc12)C(=O)N[C@@H](Cc1ccc(F)cc1)C(=O)NCc1ccc(C)cc1 ; Cc1ccc(CNC(=O)[C@H](CCCNC(N)=O)NC(=O)[C@H](Cc2c[nH]c3ccccc23)NC(=O)[C@@H]2CCCCN2C(=O)[C@H](Cc2ccccc2)NC(=O)CCl)cc1 ; COC(=O)CC[C@H](NC(=O)CCl)C(=O)N1CCCC[C@H]1C(=O)N[C@@H](Cc1c[nH]c2ccccc12)C(=O)N[C@@H](Cc1ccc(O)cc1)C(=O)NCc1ccc(C)cc1 ; COC(=O)CC[C@H](NC(=O)CCl)C(=O)N1CCCC[C@H]1C(=O)N[C@@H](Cc1c[nH]c2ccccc12)C(=O)N[C@@H](Cc1cscn1)C(=O)NCc1ccc(C)cc1]

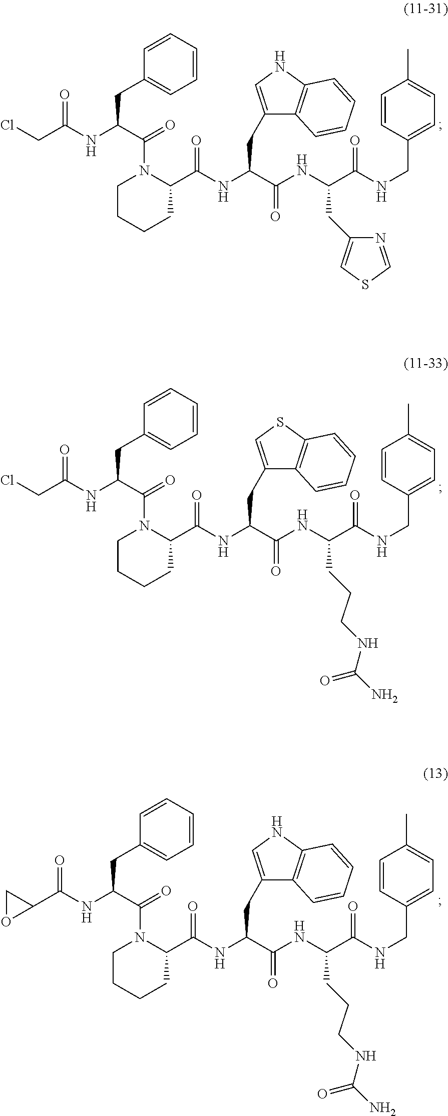 [SMILES: Cc1ccc(CNC(=O)[C@H](Cc2cscn2)NC(=O)[C@H](Cc2c[nH]c3ccccc23)NC(=O)[C@@H]2CCCCN2C(=O)[C@H](Cc2ccccc2)NC(=O)CCl)cc1 ; Cc1ccc(CNC(=O)[C@H](CCCNC(N)=O)NC(=O)[C@H](Cc2csc3ccccc23)NC(=O)[C@@H]2CCCCN2C(=O)[C@H](Cc2ccccc2)NC(=O)CCl)cc1 ; Cc1ccc(CNC(=O)[C@H](CCCNC(N)=O)NC(=O)[C@H](Cc2c[nH]c3ccccc23)NC(=O)[C@@H]2CCCCN2C(=O)[C@H](Cc2ccccc2)NC(=O)C2CO2)cc1]

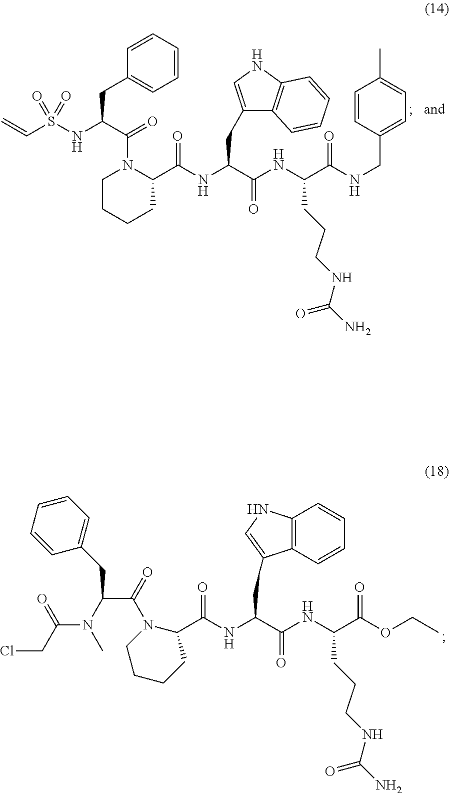 [SMILES: C=CS(=O)(=O)N[C@@H](Cc1ccccc1)C(=O)N1CCCC[C@H]1C(=O)N[C@@H](Cc1c[nH]c2ccccc12)C(=O)N[C@@H](CCCNC(N)=O)C(=O)NCc1ccc(C)cc1 ; CCOC(=O)[C@H](CCCNC(N)=O)NC(=O)[C@H](Cc1c[nH]c2ccccc12)NC(=O)[C@@H]1CCCCN1C(=O)[C@H](Cc1ccccc1)N(C)C(=O)CCl]

or a pharmaceutically acceptable salt or stereoisomer thereof.

Compounds of the present invention may be in the form of a free acid or free base, or a pharmaceutically acceptable salt. As used herein, the term “pharmaceutically acceptable” refers to a material, such as a carrier or diluent, which does not abrogate the biological activity or properties of the compound, and is relatively non-toxic, i.e., the material may be administered to a subject without causing undesirable biological effects (such as dizziness or gastric upset) or interacting in a deleterious manner with any of the components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin.

In some embodiments, the compound of the present application is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

Compounds of the present invention may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present application may be made and used in the form of individual isomers and substantially free of other isomers, or in the form of a mixture of various isomers, e.g., racemic mixtures of stereoisomers.

In addition, the compounds of the present invention embrace the use of N-oxides, crystalline forms (also known as polymorphs), active metabolites of the compounds having the same type of activity, tautomers, and unsolvated as well as solvated forms with pharmaceutically acceptable solvents such as water, ethanol, and the like, of the compounds. The solvated forms of the conjugates presented herein are also considered to be disclosed herein.

### Methods of Synthesis

In another aspect, the present invention is directed to a method for making a compound of formula (I), or a pharmaceutically acceptable salt or stereoisomer thereof. Broadly, the inventive compounds or pharmaceutically-acceptable salts or stereoisomers thereof may be prepared by any process known to be applicable to the preparation of chemically related compounds. The compounds of the present invention will be better understood in connection with the synthetic schemes that described in various working examples and which illustrate nonlimiting methods by which the compounds of the invention may be prepared.

### Pharmaceutical Compositions

Another aspect of the present invention is directed to a pharmaceutical composition that includes a therapeutically effective amount of the compound of formula (I) or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may include one or more pharmaceutically acceptable excipients.

Broadly, compounds of formula (I) may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (z.v.), intramuscular (i.m.), and intrastemal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the compositions are formulated for oral or intravenous administration (e.g., systemic intravenous injection).

Accordingly, compounds of the present invention may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, compounds of the present invention may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, compounds of formula (I) may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody.

In such embodiments, the liposomes are targeted to and taken up selectively by the organ.

The inventive compounds may be formulated for buccal or sublingual administration, examples of which include tablets, lozenges and gels.

The compounds may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichiorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Compounds of formula (I) may be formulated for topical administration which as used herein, refers to administration intradermally by application of the formulation to the epidermis.

These types of compositions are typically in the form of ointments, pastes, creams, lotions, gels, solutions and sprays.

Representative examples of carriers useful in formulating compositions for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols.

Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, 111. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerine, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a compound of formula (I) or a pharmaceutically acceptable salt or a stereoisomer thereof; or a composition including the compound of formula (I) or a pharmaceutically acceptable salt or a stereoisomer thereof, effective in producing the desired therapeutic response in a particular patient suffering from a Pin1-mediated disease or disorder. The term “therapeutically effective amount” includes the amount of the compound of the application or a pharmaceutically acceptable salt or a stereoisomer thereof, when administered, may induce a positive modification in the disease or disorder to be treated (e.g., remission), or is sufficient to prevent development or progression of the disease or disorder, or alleviate to some extent, one or more of the symptoms of the disease or disorder being treated in a subject. In respect of the therapeutic amount of the compound, the amount of the compound used for the treatment of a subject is low enough to avoid undue or severe side effects, within the scope of sound medical judgment can also be considered. The therapeutically effective amount of the compound or composition will be varied with the particular condition being treated, the severity of the condition being treated or prevented, the duration of the treatment, the nature of concurrent therapy, the age and physical condition of the end user, the specific compound or composition employed and the particular pharmaceutically acceptable carrier utilized.

The total daily dosage of the compounds and usage thereof may be decided in accordance with standard medical practice, e.g., by the attending physician using sound medical judgment. The specific therapeutically effective dose for any particular subject will depend upon a variety of factors including the disease or disorder being treated and the severity thereof (e.g., its present status); the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed; and like factors well known in the medical arts (see, for example, Goodman and Gilman's, “The Pharmacological Basis of Therapeutics”, 10th Edition, A. Gilman, J. Hardman and L. Limbird, eds., McGraw-Hill Press, 155-173, 2001).

Compounds of the present invention and their pharmaceutically acceptable salts and stereoisomers may be effective over a wide dosage range. In some embodiments, the total daily dosage (e.g., for adult humans) may range from about 0.001 to about 1000 mg, from 0.01 to about 1000 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, and in yet other embodiments from about 10 to about 30 mg per day. Individual dosage may be formulated to contain the desired dosage amount depending upon the number of times the compound is adminstered per day. By way of example, capsules may be formulated with from about 1 to about 200 mg of compound (e.g., 1, 2, 2.5, 3, 4, 5, 10, 15, 20, 25, 50, 100, 150, and 200 mg). In some embodiments, individual dosages may be formulated to contain the desired dosage amount depending upon the number of times the compound is administered per day.

### Methods of Use

In some aspects, the present invention is directed to methods of treating diseases or disorders involving dysfunctional (e.g., dysregulated) Pin1 activity, that entails administration of a therapeutically effective amount of a compound of formula (I) or a pharmaceutically acceptable salt or stereoisomer thereof, to a subject in need thereof.

The diseases or disorders may be said to be characterized or mediated by dysregulated or dysfunctional Pin1 activity (e.g., elevated levels of Pin1 relative to a non-pathological state). A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the animal's state of health. In some embodiments, compounds of the application may be useful in the treatment of proliferative diseases and disorders (e.g., cancer or benign neoplasms). As used herein, the term “cell proliferative disease or disorder” refers to the conditions characterized by unregulated or abnormal cell growth, or both. Cell proliferative disorders include noncancerous conditions, precancerous conditions, and cancer.

Pin1-catalyzed prolyl isomerization regulates the functions of its substrates through multiple different mechanisms, including controlling catalytic activity, turnover, phosphorylation, interactions with DNA, RNA or other proteins, and subcellular localization and processing. Pin1 is tightly regulated normally and its deregulation can have a major impact on the development and treatment of cancer and neurodegenerative diseases.

Pin1 substrates comprise proteins involved in signal transduction, including RAF1, HER2, eNOS, SMAD2/3, Notch1, Notch3, AKT, FAK, P70S6K, PTP-PEST, MEK1, GRK2, CDK10, FBXW7, PIP4Ks, PKM2 and JNK1; proteins involved in gene transcription including SIN3-RPD3, JUN, β-catenin, CF-2, hSPT5, MYC, NF-κB, FOS, RARα, SRC-3/AIB1, STAT3, MYB, SMRT, FOXO4, KSRP, SF-1, Nanog, PML, Mutant p53, ANp63, Oct4, ERα, PKM2, AR, SUV39H1, RUNX3, KLF10, Osterix and PML-RARα; proteins involved in cell cycle at the G1/S including Cyclin D1, KI67, Cyclin E, p27, LSF and RB1; proteins involved in cell cycle at the G2/M and M including NIMA, RAB4, CDC25, WEE1, PLK1, MYT1, CDC27, CENP-F, INCENP, RPB1, NHERF-1, KRMP1, CK2, TOPIIa, DAB2, p54NRB, SIL, EMU, CEP55, BORA, Survivin, SEPT9, SP1, SWI6, WHI5 and Separase; proteins involved in DNA damage/stress response and apoptosis including p53, BCL-2, p73, BIMEL, p66SHC, DAXX, MCL-1, NUR77, HIPK2, RBBP8, p63, HSF1, HIF-1α, CHE-1 and PGK1; proteins involved in immune response including NFAT, AUF1, IRF3, BTK, BAX, COX-2, p47PHOX, IRAK1, GR and FADD; proteins involved in viral or parasitic infection and transformation including HBX, A3G, v-Rel, Tax, Capsid protein, Integrase, BALF5, RTA, FBXW7 and ORFlp; proteins involved in neuronal survival and degeneration including TAU, APP, Synphilin-1, Gephyrin, mGluR5, REST, GRO/TLE1 and CRMP2A. (Zhou and Lu, “The isomerase Pin1 controls numerous cancer-driving pathways and is a unique drug target” Nature Reviews Cancer 16:463-478; Supplementary Information (2016)).

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

In general, methods of using the compounds of the present invention include administering to a subject in need thereof a therapeutically effective amount of a compound of the present invention.

Exemplary types of non-cancerous diseases or disorders that may be amenable to treatment with the compounds of the present invention include inflammatory diseases and conditions, autoimmune diseases, neurodegenerative diseases, heart diseases, viral diseases, chronic and acute kidney diseases or injuries, obesity, metabolic diseases, allergic and genetic diseases.

Representative examples of specific non-cancerous diseases and disorders include rheumatoid arthritis, alopecia areata, lymphoproliferative conditions, autoimmune hematological disorders (e.g. hemolytic anemia, aplastic anemia, anhidrotic ecodermal dysplasia, pure red cell anemia and idiopathic thrombocytopenia), cholecystitis, acromegaly, rheumatoid spondylitis, osteoarthritis, gout, scleroderma, sepsis, septic shock, dacryoadenitis, cryopyrin associated periodic syndrome (CAPS), endotoxic shock, endometritis, gram-negative sepsis, keratoconjunctivitis sicca, toxic shock syndrome, asthma, adult respiratory distress syndrome, chronic obstructive pulmonary disease, chronic pulmonary inflammation, chronic graft rejection, hidradenitis suppurativa, inflammatory bowel disease, Crohn's disease, Behcet's syndrome, systemic lupus erythematosus, glomerulonephritis, multiple sclerosis, juvenile-onset diabetes, autoimmune uveoretinitis, autoimmune vasculitis, thyroiditis, Addison's disease, lichen planus, appendicitis, bullous pemphigus, pemphigus vulgaris, pemphigus foliaceus, paraneoplastic pemphigus, myasthenia gravis, immunoglobulin A nephropathy, autoimmune thyroiditis or Hashimoto's disease, Sjogren's syndrome, vitiligo, Wegener granulomatosis, granulomatous orchitis, autoimmune oophoritis, sarcoidosis, rheumatic carditis, ankylosing spondylitis, Grave's disease, autoimmune thrombocytopenic purpura, psoriasis, psoriatic arthritis, eczema, dermatitis herpetiformis, ulcerative colitis, pancreatic fibrosis, hepatitis, hepatic fibrosis, CD14 mediated sepsis, non-CD14 mediated sepsis, acute and chronic renal disease, irritable bowel syndrome, pyresis, restenosis, cerebral malaria, cervicitis, stroke and ischemic injury, neural trauma, acute and chronic pain, allergic rhinitis, allergic conjunctivitis, chronic heart failure, congestive heart failure, acute coronary syndrome, cachexia, malaria, leprosy, leishmaniasis, Lyme disease, Reiter's syndrome, acute synovitis, muscle degeneration, bursitis, tendonitis, tenosynovitis, herniated, ruptured, or prolapsed intervertebral disk syndrome, osteopetrosis, rhinosinusitis, thrombosis, silicosis, pulmonary sarcosis, bone resorption diseases, such as osteoporosis, graft-versus-host reaction, fibromyalgia, AIDS and other viral diseases such as Herpes Zoster, Herpes Simplex I or II, influenza virus and cytomegalovirus, diabetes Type I and II, obesity, insulin resistance and diabetic retinopathy, 22q11.2 deletion syndrome, Angelman syndrome, Canavan disease, celiac disease, Charcot-Marie-Tooth disease, color blindness, Cri du chat, Down syndrome, cystic fibrosis, Duchenne muscular dystrophy, haemophilia, Klinefleter's syndrome, neurofibromatosis, phenylketonuria, Prader-Willi syndrome, sudden infant death syndrome, sickle cell disease, Tay-Sachs disease, Turner syndrome, urea cycle disorders, thalassemia, otitis, pancreatitis, parotitis, pericarditis, peritonitis, pharyngitis, pleuritis, phlebitis, pneumonitis, cystic fibrosis, uveitis, polymyositis, proctitis, interstitial lung fibrosis, dermatomyositis, arteriosclerosis, amyotrophic lateral sclerosis, asocality, immune response, varicosis, vaginitis, including chronic recurrent yeast vaginitis, depression, and Sudden Infant Death Syndrome.

In some embodiments, the autoimmune disease that is treated is lupus, asthma or arthritis.

In some embodiments, the neurodegenerative disease is Alzheimer's disease or Parkinson's disease.

In other embodiments, the methods are directed to treating subjects having cancer.

Broadly, the compounds of the present invention may be effective in the treatment of carcinomas (solid tumors including both primary and metastatic tumors), sarcomas, melanomas, and hematological cancers (cancers affecting blood including lymphocytes, bone marrow and/or lymph nodes) including leukemia, lymphoma and multiple myeloma. Adult tumors/cancers and pediatric tumors/cancers are included. The cancers may be vascularized, or not yet substantially vascularized, or non-vascularized tumors.

Representative examples of cancers includes adenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., brain stem glioma, cerebellar astrocytoma, cerebral astrocytoma/malignant glioma, ependymoma, medulloblastoma, supratentorial primitive neuroectodeimal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, acute promyelocytic leukemia, chronic lymphocytic leukemia, chronic myelogenous leukemia, chronic myeloproliferative disorders, anal cancer, colorectal cancer (e.g., colon cancer, rectal cancer), cutaneous T-cell lymphoma, lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), germ cell tumor, ovarian germ cell tumor, gestational trophoblastic tumor glioma, head and neck cancer, hepatocellular (liver) cancer, Hodgkin's lymphoma, non-Hodgkin's lymphoma, hypopharyngeal cancer, intraocular melanoma, ocular cancer, islet cell tumors (endocrine pancreas), renal cancer (e.g., Wilm's Tumor, clear cell renal cell carcinoma), laryngeal cancer, acute lymphoblastic leukemia, acute myeloid leukemia, hairy cell leukemia, lip and oral cavity cancer, liver cancer, lung cancer (e.g., non-small cell lung cancer and small cell lung cancer), primary central nervous system lymphoma, Waldenstrom's macroglobulinema, melanoma, intraocular (eye) melanoma, merkel cell carcinoma, mesothelioma malignant, mesothelioma, metastatic squamous neck cancer, multiple endocrine neoplasia syndrome, mycosis fungoids, myelodysplastic syndromes, myelodyplastic/myeloproliferative diseases, multiple myeloma, chromic myeproliferative disorders, nasopharyngeal cancer, neuroblastoma, oral cancer (e.g., mouth cancer, lip cancer, oral cavity cancer, tongue cancer, oropharyngeal cancer, throat cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, retinoblastoma rhabdomyosarcoma, salivary gland cancer, uterine cancer (e.g., endometrial uterine cancer, uterine sarcoma, uterine corpus cancer), merkel cell skin carcinoma, squamous cell carcinoma, supratentorial primitive neuroectodermal tumors, testicular cancer, thymoma, thymoma and thymic carcinoma, thyroid cancer, transitional cell cancer of the renal pelvis and ureter and other urinary organs, urethral cancer, gestational trophoblastic tumor, vaginal cancer and vulvar cancer.

Sarcomas that may be treatable with compounds of the present invention include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue) and mesenchymous or mixed mesodermal tumor (mixed connective tissue types).

In some embodiments, methods of the present invention entail treatment of subjects having cell proliferative diseases or disorders of the hematological system, liver (hepatocellular), brain, lung, colorectal (e.g., colon), pancreas, prostate, ovary, breast, or skin (e.g., melanoma).

As used herein, “cell proliferative diseases or disorders of the hematologic system” include lymphoma, leukemia, myeloid neoplasms, mast cell neoplasms, myelodysplasia, benign monoclonal gammopathy, lymphomatoid papulosis, polycythemia vera, chronic myelocytic leukemia, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), acute myeloid leukemia (AML), acute promyelocytic leukemia (APL), mantle cell lymphoma (MCL) and ALK+ anaplastic large cell lymphoma) (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, refractory B-cell non-Hodgkin's lymphoma, and relapsed B-cell non-Hodgkin's lymphoma), childhood lymphomas, and lymphomas of lymphocytic and cutaneous origin, e.g., small lymphocytic lymphoma, primary CNS lymphoma (PCNSL), marginal zone lymphoma (MZL), leukemia, including chronic lymphocytic leukemia (CLL), childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the liver (hepatocellular)” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the brain may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain.

Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, a precancer or precancerous condition of the lung, benign growths or lesions of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), squamous cell carcinoma, adenocarcinoma, small cell carcinoma, large cell carcinoma, squamous cell carcinoma, and mesothelioma. Lung cancer can include “scar carcinoma”, bronchioveolar carcinoma, giant cell carcinoma, spindle cell carcinoma, and large cell neuroendocrine carcinoma. Lung cancer includes lung neoplasms having histologic and ultrastructural heterogeneity (e.g., mixed cell types).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer. Colon cancer includes malignant colon neoplasms, carcinoma in situ, typical carcinoid tumors, and atypical carcinoid tumors. Colon cancer includes adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polypopsis, Gardner's syndrome, Peutz-Jeghers syndrome, Turcot's syndrome and juvenile polyposis. Cell proliferative disorders of the colon can be characterized by hyperplasia, metaplasia, and dysplasia of the colon.

As used herein, “cell proliferative diseases or disorders of the pancreas” include all forms of cell proliferative disorders affecting pancreatic cells. Cell proliferative disorders of the pancreas may include pancreatic cancer, an precancer or precancerous condition of the pancreas, hyperplasia of the pancreas, and dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell types).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the prostate.

The compounds of the present invention may be administered to a patient, e.g., a cancer patient, as a monotherapy or by way of combination therapy, and as a front-line therapy or a follow-on therapy for patients who are unresponsive to front line therapy. Therapy may be “first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which have been partially successful but are intolerant to the particular treatment.

Therapy may also be given as an adjuvant treatment, i.e., to prevent reoccurrence of cancer in patients with no currently detectable disease or after surgical removal of a tumor. Thus, in some embodiments, the compound may be administered to a patient who has received another therapy, such as chemotherapy, radioimmunotherapy, surgical therapy, immunotherapy, radiation therapy, targeted therapy or any combination thereof.

The methods of the present invention may entail administration of compounds of the invention or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5 or 6 weeks, and in other embodiments entails a 28-day cycle which includes daily administration for 3 weeks (21 days).

### Combination Therapy

The compounds of the present invention may be used in combination with at least one other active agent, e.g., anti-cancer agent or regimen, in treating diseases and disorders. The term “in combination” in this context means that the agents are co-administered, which includes substantially contemporaneous administration, by the same or separate dosage forms, or sequentially, e.g., as part of the same treatment regimen or by way of successive treatment regimens. Thus, if given sequentially, at the onset of administration of the second compound, the first of the two compounds is in some cases still detectable at effective concentrations at the site of treatment. The sequence and time interval may be determined such that they can act together (e.g., synergistically to provide an increased benefit than if they were administered otherwise). For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a compound of the invention in combination with one or more additional therapeutics. The dosage of the additional therapeutic may be the same or even lower than known or recommended doses. See, Hardman et al., eds., Goodman & Gilman's The Pharmacological Basis Of Basis Of Therapeutics, 10th ed., McGraw-Hill, New York, 2001; Physician's Desk Reference 60th ed., 2006. Anti-cancer agents that may be used in combination with the inventive compounds are known in the art. See, e.g., U.S. Pat. No. 9,101,622 (Section 5.2 thereof). Representative examples of additional active agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bispecific antibodies) and CAR-T therapy.

In some embodiments, the compound of formula (I) and the additional anticancer therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart. The two or more anticancer therapeutics may be administered within the same patient visit.

In some embodiments, the compound of formula (I) and the additional agent or therapeutic (e.g., an anti-cancer therapeutic) are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

The compounds of the present invention may be administered to a patient suffering from a neurodegenerative disease or disorder in combination with another active agent. Representative examples of other active agents known to treat neurodegenerative diseases and disorders include dopaminergic treatments (e.g., Carbidopa-levodopa, pramipexole (Mirapex), ropinirole (Requip) and rotigotine (Neupro, given as a patch)). Apomorphine and monoamine oxidase B (MAO-B) inhibitors (e.g., selegiline (Eldepryl, Zelapar), rasagiline (Azilect) and saflnamide (Xadago)) for Parkinson disease and movement disorders, cholinesterase inhibitors for cognitive disorders (e.g., benztropine (Cogentin) or trihexyphenidyl), antipsychotic drugs for behavioral and psychological symptoms of dementia, as well as agents aimed to slow the development of diseases, such as Riluzole for ALS, cerebellar ataxia and Huntington's disease, non-steroidal anti-inflammatory drugs for Alzheimer's disease, and caffeine A2A receptor antagonists and CERE-120 (adeno-associated virus serotype 2-neurturin) for the neuroprotection of Parkinson's disease.

The compounds of the present invention may be administered to a patient suffering from an autoimmune disease or disorder in combination with another active agent. Representative examples of other active agents known to treat neurodegenerative diseases and disorders include corticosteroids (e.g., prednisone, hydrocortisone, and dexamethasone) immunosuppressant drugs, such as methotrexate, cyclophosphamide, and azathioprine. Other examples include immunosuppressive dugbelimumab (Benlysta®) for severe active lupus nephritis or severe active central nervous system lupus, asthma and arthritis, anti-malarial dugs (e.g., hydroxychloroquine (Plaquenil®) and chloroquine (Aralen®)) for lupus, combinations of corticosteroid and bronchodilator (e.g., fluticasone and salmeterol (Advair Diskus®), budesonide and formoterol (Symbicort®), and fluticasone and vilanterol (BREO)), and analgesics (e.g., acetaminophen), nonsteroidal anti-inflammation drugs (NSAIDs) (e.g., aspirin, ibuprofen, naproxen, indomethacin and celecoxib (Celebrex®)), traditional disease-modifying antirheumatic drugs (DMARDs) (e.g., tumor necrosis factor (TNF) inhibitors or TNF blockers (etanercept (Enbrel®) and adalimumab (Humira®)), Interleukin-6 (IL-6) inhibitors, Interleukin-1 (IL-1) receptor antagonists, B-cell inhibitors, Janus kinases (JAK) inhibitors, phosphodiesterase 4 (PDE 4) inhibitors and costimulation modulators) for treating rheumatoid arthritis (RA), ankylosing spondylitis, psoriatic arthritis, juvenile idiopathic arthritis and lupus.

### Pharmaceutical Kits

The present compositions may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain the compound of the present application or a pharmaceutical composition. The kits or pharmaceutical systems of the invention may also include printed instructions for using the compounds and compositions.

These and other aspects of the present application will be further appreciated upon consideration of the following Examples, which are intended to illustrate certain particular embodiments of the application but are not intended to limit its scope, as defined by the claims.

### EXAMPLES

Broadly, the inventive compounds or pharmaceutically-acceptable salts or stereoisomers thereof, may be prepared by any process known to be applicable to the preparation of chemically related compounds. All solvents and reagents were used as obtained from commercial sources. ¹H and ¹³C NMR spectra were recorded with a Bruker 500 MHz NMR spectrometer, and chemical shifts are reported in parts per million (ppm) downfield from tetramethylsilane (TMS). Coupling constants (J) are reported in Hz. Spin multiplicities are described as s (singlet), d (doublet), t (triplet), dd (doublet of doublets), and m (multiplet). Mass spectra were obtained on a Waters Acquity UPLC®. Preparative HPLC was performed on a Waters Sunfire™ C18 column (19 mm×5 mm) using a gradient of 15-95% methanol in water containing 0.05% trifluoroacetic acid (TFA) over 22 min (28 min run time) at a flow rate of 20 mL/min. Representative schemes for synthesizing the compounds of the present invention are described below.

### General Methods

General Procedure for Synthesis of Peptidic Backbone. Peptidic backbones were prepared according to established solid-phase peptide synthesis (SPPS) protocols with Fmoc-protected amino acids. Wang resin was used on a 0.12 mmol scale, and Fmoc-protected amino acids (0.36 mmol, 3 eq.) were deprotected with 20% piperidine in DMF. Activation of amino acids (0.36 mmol, 3 eq.) for coupling was performed with (1-[Bis(dimethylamino)methylene]-1H-1,2,3-triazolo[4,5-b]pyridinium 3-oxid hexafluorophosphate (HATU) (0.36 mmol, 3 eq.) and N,N-diisopropylethylamine (DIPEA) (8 eq.). Cleavage from the resin was performed with TFA/TIPS/water 95/2.5/2.5. The residual solid was dissolved in H₂O and acetonitrile, and lyophilized to yield the peptidic backbone quantitatively as a white powder.

Equipment Setup. Manual peptide-synthesis apparatus. A vacuum manifold (Vac-Man laboratory manifold, Promega) was placed in a fume hood and connected to a vacuum system through a trap. Three-way stopcocks were placed on the manifold and connected to a line of nitrogen gas. All other unused inlets on the manifold were capped with rubber septa. Poly-Prep® chromatography columns (Bio-Rad) containing the resin were placed on the manifold via the three-way stopcocks. Equipment setup according to literature reference: Kim el al., Nature Protocols 6:761-771 (2011).

Reagents. Wang Resin (Fisher Scientific, 1.45 meq/g), N,N-dimethyl formamide (DMF, Sigma Aldrich), dichloromethane (DCM, Sigma Aldrich), piperidine (Sigma Aldrich), N,N-diisopropylethylamine (DIPEA, Sigma Aldrich) trifluoroacetic acid (TFA, Fisher Scientific), diethyl ether (Fisher Scientific), acetic anhydride (Sigma Aldrich), triisopropylsilane (TIPS, Sigma Aldrich), pyridine (Sigma Aldrich), ninhydrin test kit (Fisher Scientific), standard commercially available unnatural and natural Fmoc-protected amino acids (Fisher Scientific and VWR).

Addition of the First Amino Acid to Wang Resin (applies to Schemes 2 & 3). The resin was suspended in 9:1 v/v CH₂Cl₂/DMF (1 mL per 100 mg of resin) in a Poly-Prep column, and gently agitated with nitrogen gas. In a separate flask, the Fmoc-protected amino acid (3 eq. relative to resin) and HATU (3 eq. relative to resin) were dissolved in DMF (1 mL of DMF per 0.06 mmol Wang resin). To this solution was added DIPEA (8 eq. relative to resin). The resulting amino acid solution was stirred at room temperature until homogeneous, and then added to the resin in the Poly-Prep column. In a separate flask, DMAP (0.1 eq. relative to resin) was dissolved in a minimum amount of DMF. The resulting solution was also added to the resin solution in the Poly-Prep column. The reaction mixture was agitated via nitrogen bubbling for 2-3 hours at room temperature. The solution was then drained by vacuum filtration, and the resin washed 3× with DMF (for each wash step: use enough solvent to slurry the resin, bubble nitrogen for 1 min, then drain solution via vacuum filtration). Then, 2 eq. (relative to the resin) of acetic anhydride and 2 eq. (relative to the resin) of pyridine in DMF (1 mL DMF per 0.12 mmol resin) were added to the Poly-Prep column. The resin was agitated for an additional 30 minutes at room temperature to end cap any unreacted hydroxyl groups on the resin. The solution was then drained by vacuum filtration, and the resin was washed 3× with DMF, 3× with DCM, and 3× with MeOH.

Removal of the FMOC. A solution of 20% piperidine in DMF was prepared and added to the resin (1 mL of solution per 0.12 mmol resin). The resin was agitated via nitrogen bubbling for 15 minutes. The solution was drained using vacuum filtration, then more of the piperidine/DMF solution was added to the resin and bubbled for another 15 minutes. After draining the solution, the resin was washed 3× with DMF.

Coupling of Amino Acids. The resin was suspended in DMF (1 mL per 0.06 mmol resin) and agitated for 5 minutes at room temperature. The DMF was then removed via vacuum filtration. In a separate flask, the Fmoc-protected amino acid (3 eq. relative to resin) and HATU (3 eq. relative to resin) were dissolved in DMF (1 mL of DMF per 0.06 mmol Wang resin). To this solution was added DIPEA (8 eq. relative to resin). The resulting amino acid solution was stirred at room temperature until homogeneous, and then added to the resin in the Poly-Prep column. The reaction mixture was agitated by nitrogen bubbling for 2-3 hours at room temperature. After which the solution was removed with vacuum filtration, and the resin washed 3× with DMF, 3× with DCM, 3× with MeOH. The completion of the coupling was assessed using the Kaiser test. If the test indicated that there was still unreacted N-terminal amine, the coupling step was repeated.

Cleavage from the Resin. After being dried under vacuum, the resin was transferred to an 8 mL reaction vial with screw cap, equipped with a magnetic stir bar. After cooling the flask in an ice bath, a solution of TFA/TIPS/water 95/2.5/2.5 was added (1 mL per 100 mg of resin). The reaction mixture was stirred for 10 minutes at 0° C. Then, the mixture was stirred at room temperature until the cleavage was complete (generally 2-4 hours, but can be left overnight). The resin was filtered using a fine sintered glass funnel and washed 3 times with a minimal amount of TFA. To the filtrate was added 8-10 times the volume of cold diethyl ether. If necessary, the mixture was stored at 4° C. overnight to precipitate the peptide. The peptide was then filtered using a fine sintered glass funnel and washed with cold diethyl ether. Purification by HPLC and subsequent lyophilization yielded the pure peptide chain in quantitative yields.

### Example 1: Identification and Characterization of Compounds Synthesized According to Scheme 1

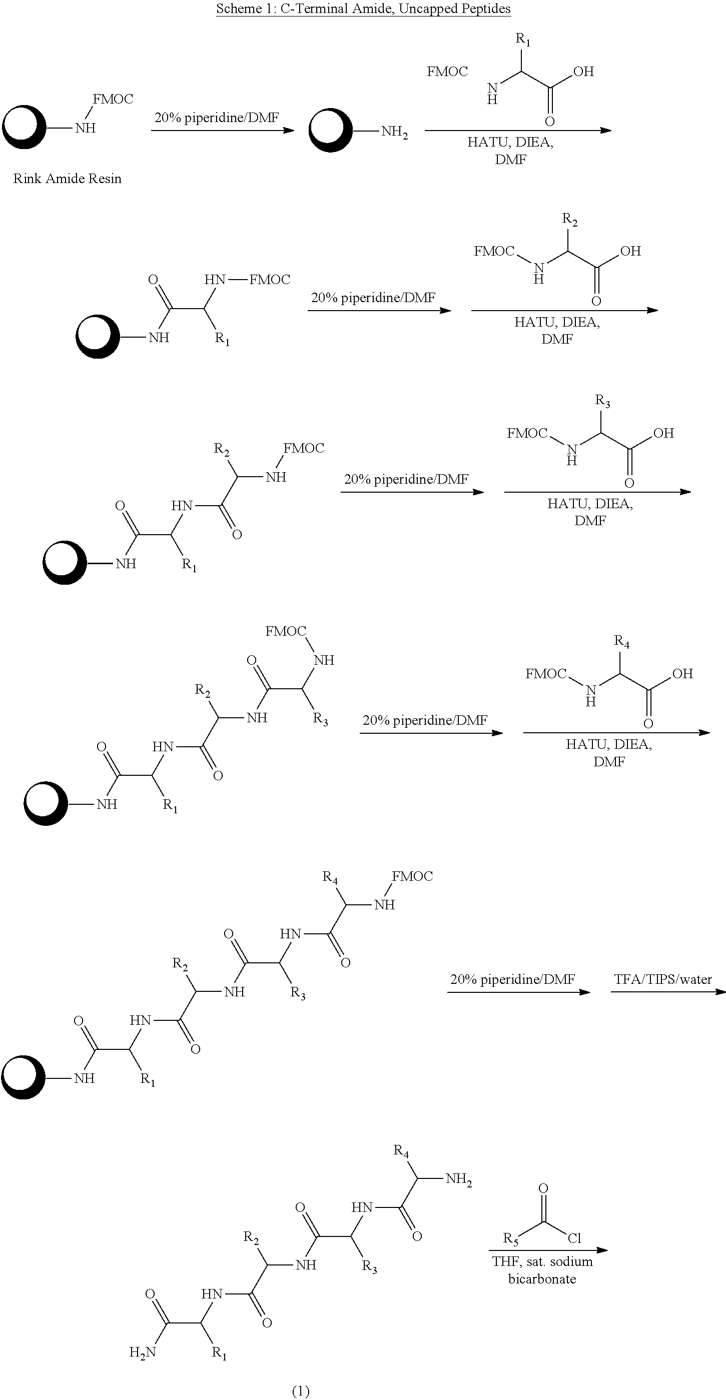

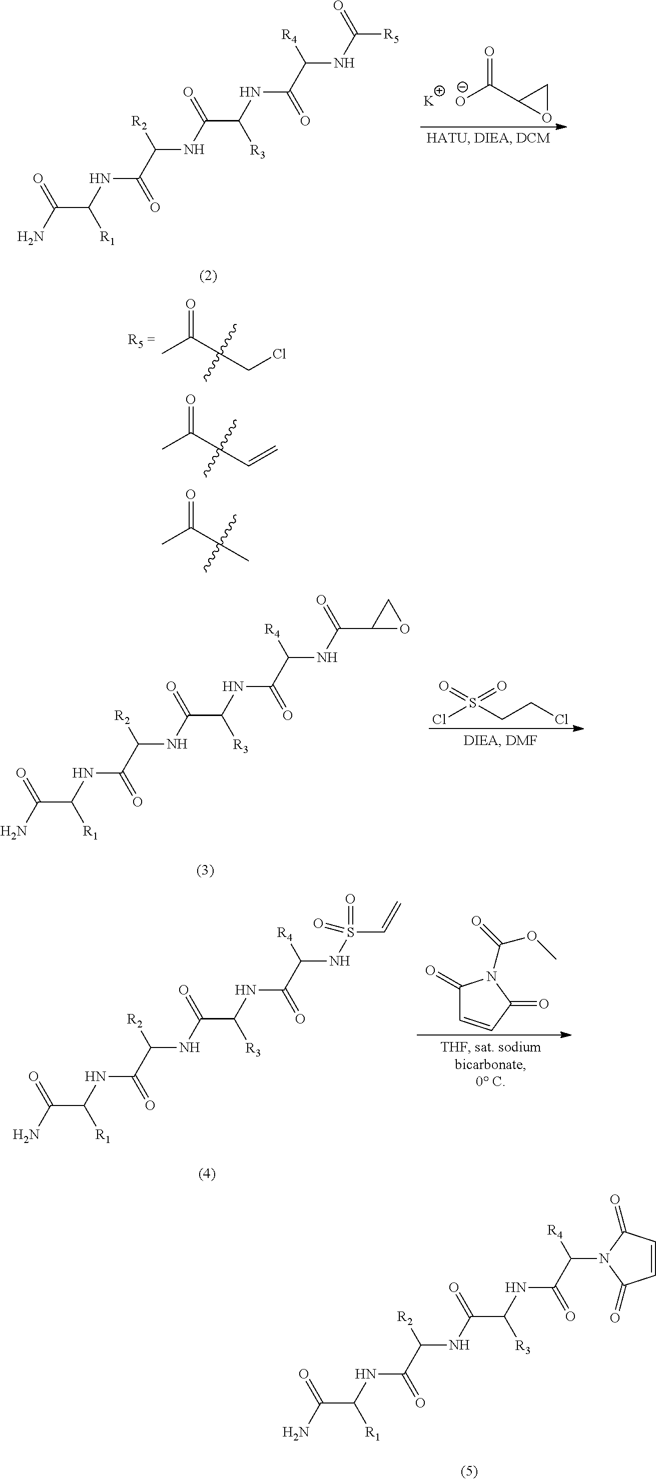

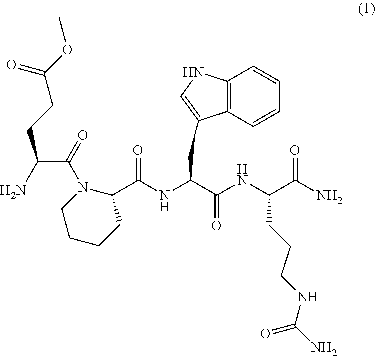 [SMILES: COC(=O)CC[C@H](N)C(=O)N1CCCC[C@H]1C(=O)N[C@@H](Cc1c[nH]c2ccccc12)C(=O)N[C@@H](CCCNC(N)=O)C(N)=O]

### Methyl (S)-4-amino-5-((S)-2-(((S)-1-(((S)-1-amino-1-oxo-5-ureidopentan-2-yl)amino)-3-(1H-indol-3-yl)-1-oxopropan-2-yl)carbamoyl)piperidin-1-yl)-5-oxopentanoate

Peptide 1 was synthesized according to general solid phase peptide synthesis procedures starting from 0.12 mmol of Rink Amide resin. The following amino acids were coupled to the resin: Fmoc-Glu(OMe)-OH, Fmoc-pipecolinic acid, Fmoc-Trp(BOC)-OH, Fmoc-Cit-OH.

¹H NMR (500 MHz, DMSO-d₆): δ=10.71 (s, 1H), 8.02 (s, 3H), 7.86 (d, J=5 Hz, 1H), 7.80 (d, J=5 Hz, 1H), 7.52 (d, J=5 Hz, 1H), 7.24 (d, J=10 Hz, 1H), 7.17 (s, 1H), 7.07 (s, 1H), 6.99-6.95 (m, 2H), 6.90 (t, J=5 Hz, 1H), 5.86 (s, 1H), 4.94 (s, 1H), 4.55-4.51 (m, 1H), 4.37 (s, 1H), 4.16-4.12 (m, 1H), 3.63 (d, J=10 Hz, 1H), 3.54 (s, 3H), 3.52 (s, 1H), 3.10 (dd, J=15, 5 Hz, 1H), 3.01 (t, J=10 Hz, 1H), 2.92-2.83 (m, 3H), 2.13 (d, J=10 Hz, 1H), 1.89-1.84 (m, 2H), 1.78-1.73 (m, 1H), 1.60-1.37 (m, 6H), 1.30-1.23 (m, 4H). ¹³C NMR (126 MHz, DMSO-d₆): δ=173.8, 172.9, 171.5, 170.3, 168.8, 159.3, 158.1, 136.5, 127.8, 124.0, 121.3, 118.9, 118.7, 111.71, 110.39, 53.9, 52.6, 52.5, 52.1, 49.6, 43.3, 30.2, 28.6, 27.7, 27.3, 26.9, 25.4, 25.2, 20.21.

MS (ESI): m/z=[M+1] 615.02.

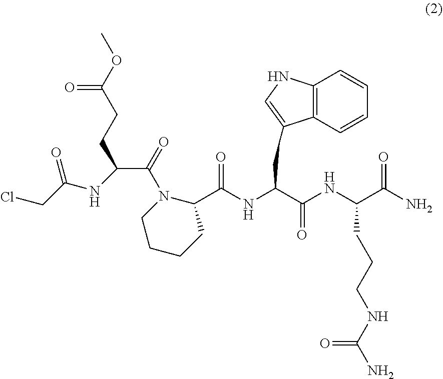 [SMILES: COC(=O)CC[C@H](NC(=O)CCl)C(=O)N1CCCC[C@H]1C(=O)N[C@@H](Cc1c[nH]c2ccccc12)C(=O)N[C@@H](CCCNC(N)=O)C(N)=O]

### Methyl (S)-5-((S)-2-(((S)-1-(((S)-1-amino-1-oxo-5-ureidopentan-2-yl)amino)-3-(1H-indol-3-yl)-1-oxopropan-2-yl)carbamoyl)piperidin-1-yl)-4-(2-chloroacetamido)-5-oxopentanoate

To a mixture of intermediate 1 (49 mg, 0.08 mmol) in 0.2 mL THF and 0.2 mL saturated sodium bicarbonate was added a solution of chloroacetyl chloride (0.012 mL, 0.16 mmol) in 0.2 mL THF. The resulting mixture was stirred in an ice bath for 2 hours. The mixture was then diluted with 0.5 mL of dichloromethane and 0.5 mL of methanol, and filtered through a 0.45 um syringe filter. The solvent was evaporated, the product purified via HPLC and lyophilized to yield compound 2 as a white powder (30 mg, 54%).

¹H NMR (500 MHz, DMSO-d₆): δ=10.80 (s, 1H), 8.47 (d, J=5 Hz, 1H), 7.93 (d, J=5 Hz, 1H), 7.84 (t, J=5 Hz, 1H), 7.64 (d, J=5 Hz, 1H), 7.59 (d, J=5 Hz, 1H), 7.32 (d, J=5 Hz, 1H), 7.26 (s, 1H), 7.14 (s, 1H), 7.05 (d, J=10 Hz, 2H), 6.97 (d, J=10 Hz, 1H), 6.53 (s, 1H), 5.92 (t, J=5 Hz, 1H), 5.39 (s, 2H), 4.97 (d, J=5 Hz, 1H), 4.77-4.74 (m, 1H), 4.66-4.62 (m, 1H), 4.24-4.21 (m, 1H), 4.09 (d, J=5 Hz, 2H), 3.59 (s, 3H), 3.20-3.16 (m, 2H), 3.01-2.91 (m, 3H), 2.84 (t, J=5 Hz, 1H), 2.40 (t, J=5 Hz, 1H), 2.33 (t, J=5 Hz, 2H), 2.12-2.09 (m, 1H), 1.89-1.81 (m, 1H), 1.69-1.63 (m, 2H), 1.53-1.45 (m, 2H), 1.41-1.32 (m, 2H), 1.24-1.17 (m, 1H) ppm.

MS (ESI): m/z=[M+1] 691.83.

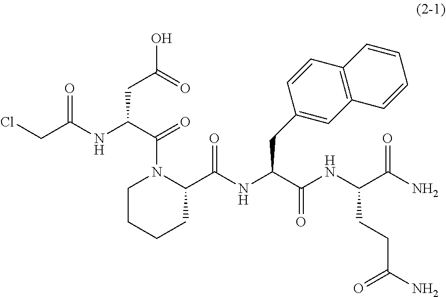 [SMILES: NC(=O)CC[C@H](NC(=O)[C@H](Cc1ccc2ccccc2c1)NC(=O)[C@@H]1CCCCN1C(=O)[C@@H](CC(=O)O)NC(=O)CCl)C(N)=O]

### (R)-3-(2-Chloroacetamido)-4-((S)-2-(((S)-1-(((S)-1,5-diamino-1,5-dioxopentan-2-yl)amino)-3-(naphthalen-2-yl)-1-oxopropan-2-yl)carbamoyl)piperidin-1-yl)-4-oxobutanoic acid

¹H NMR (500 MHz, DMSO-d₆): δ=12.29 (s, 1H), 8.76 (d, J=10 Hz, 1H), 8.15 (t, J=5 Hz, 2H), 7.92-7.84 (m, 2H), 7.72 (d, J=10 Hz, 1H), 7.63 (d, J=10 Hz, 1H), 7.52 (t, J=5 Hz, 1H), 7.46 (t, J=5 Hz, 1H), 7.38-7.29 (m, 2H), 7.21-7.12 (m, 3H), 7.05-6.94 (m, 3H), 6.69 (s, 1H), 6.47 (s, 1H), 4.89-4.82 (m, 1H), 4.68 (t, J=5 Hz, 1H), 4.14-4.11 (m, 1H), 4.00-3.95 (m, 2H), 3.5-3.50 (m, 2H), 2.68-2.62 (m, 1H), 2.07-1.97 (m, 2H), 1.87-1.81 (m, 1H), 1.79-1.68 (m, 1H), 1.31-1.17 (m, 4H), 0.88-0.79 (m, 1H) ppm.

MS (ESI): m/z=[M+1] 645.76.

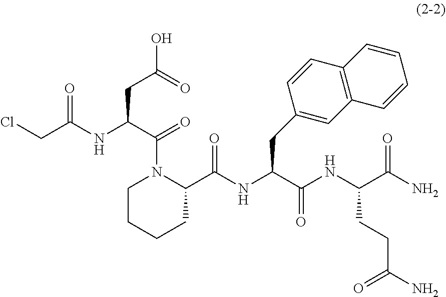 [SMILES: NC(=O)CC[C@H](NC(=O)[C@H](Cc1ccc2ccccc2c1)NC(=O)[C@@H]1CCCCN1C(=O)[C@H](CC(=O)O)NC(=O)CCl)C(N)=O]

### (S)-3-(2-Chloroacetamido)-4-((S)-2-(((S)-1-(((S)-1,5-diamino-1,5-dioxopentan-2-yl)amino)-3-(naphthalen-2-yl)-1-oxopropan-2-yl)carbamoyl)piperidin-1-yl)-4-oxobutanoic acid

¹H NMR (500 MHz, DMSO-d₆): δ=12.39 (s, 1H), 8.70 (d, J=10 Hz, 1H), 8.16 (d, J=10 Hz, 1H), 7.94 (d, J=10 Hz, 1H), 7.85 (d, J=10 Hz, 1H), 7.72 (t, J=5 Hz, 1H), 7.52 (t, J=5 Hz, 1H), 7.46 (t, J=5 Hz, 1H), 7.39-7.34 (m, 2H), 7.21-7.11 (m, 3H), 7.06-6.96 (m, 3H), 6.71 (s, 1H), 6.47 (s, 1H), 4.99-4.95 (m, 1H), 4.87 (s, 1H), 4.70-4.64 (m, 1H), 4.14-4.09 (m, 1H), 4.02-3.95 (m, 2H), 3.59-3.54 (m, 1H), 3.48 (d, J=5 Hz, 1H), 2.73-2.68 (m, 1H), 2.05-2.01 (m, 2H), 1.97-1.94 (m, 1H), 1.89-1.82 (m, 1H), 1.75-1.69 (m, 1H), 1.31-1.08 (m, 4H), 0.90-0.80 (m, 1H) ppm.

MS (ESI): m/z=[M+1] 645.12.

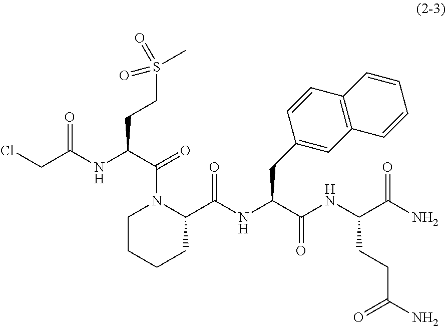 [SMILES: CS(=O)(=O)CC[C@H](NC(=O)CCl)C(=O)N1CCCC[C@H]1C(=O)N[C@@H](Cc1ccc2ccccc2c1)C(=O)N[C@@H](CCC(N)=O)C(N)=O]

### (S)-2-((S)-2-((S)-1-((S)-2-(2-Chloroacetamido)-4-(methylsulfonyl)butanoyl)piperidine-2-carboxamido)-3-(naphthalen-2-yl)propanamido)pentanediamide

¹H NMR (500 MHz, DMSO-d₆): δ=8.69 (d, J=5 Hz, 1H), 8.22 (d, J=10 Hz, 1H), 8.06 (d, J=5 Hz, 1H), 7.93-7.87 (m, 2H), 7.79 (t, J=5 Hz, 1H), 7.59 (t, J=5 Hz, 1H), 7.53 (t, J=5 Hz, 1H), 7.43 (d, J=5 Hz, 1H), 7.27-7.23 (m, 5H), 7.13-7.09 (m, 4H), 7.02 (s, 3H), 6.77 (s, 1H), 6.55 (s, 1H), 4.95 (s, 1H), 4.91-4.88 (m, 1H), 4.22-4.17 (m, 1H), 4.11 (s, 1H), 3.64-3.59 (m, 2H), 2.96 (s, 2H), 2.12-2.08 (m, 2H), 2.00-1.93 (m, 2H), 1.82-1.75 (m, 1H), 1.41-1.24 (m, 4H), 1.07-1.01 (m, 1H) ppm.

MS (ESI): m/z=[M+1] 693.14.

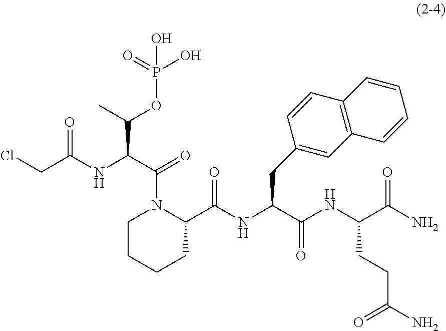 [SMILES: CC(OP(=O)(O)O)[C@H](NC(=O)CCl)C(=O)N1CCCC[C@H]1C(=O)N[C@@H](Cc1ccc2ccccc2c1)C(=O)N[C@@H](CCC(N)=O)C(N)=O]

### (2S,3S)-3-(2-Chloroacetamido)-4-((S)-2-(((S)-1-(((S)-1,5-diamino-1,5-dioxopentan-2-yl)amino)-3-(naphthalen-2-yl)-1-oxopropan-2-yl)carbamoyl)piperidin-1-yl)-4-oxobutan-2-yl dihydrogen phosphate

MS (ESI): m/z=[M+1] 711.09.

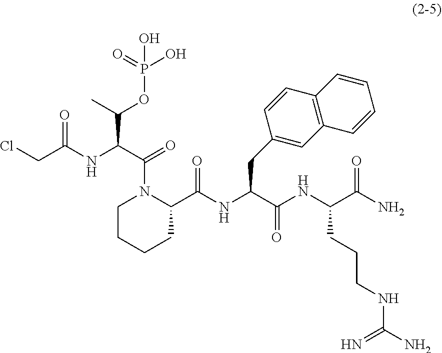 [SMILES: CC(OP(=O)(O)O)[C@H](NC(=O)CCl)C(=O)N1CCCC[C@H]1C(=O)N[C@@H](Cc1ccc2ccccc2c1)C(=O)N[C@@H](CCCNC(=N)N)C(N)=O]

### (2S,3S)-4-((S)-2-(((S)-1-(((S)-1-Amino-5-guanidino-1-oxopentan-2-yl)amino)-3-(naphthalen-2-yl)-1-oxopropan-2-yl)carbamoyl)piperidin-1-yl)-3-(2-chloroacetamido)-4-oxobutan-2-yl dihydrogen phosphate

¹H NMR (500 MHz, DMSO-d₆): δ=8.54-8.49 (m, 1H), 8.22-8.11 (m, 1H), 8.02 (d, J=10 Hz, 1H), 7.86 (s, 1H), 7.81-7.72 (m, 3H), 7.69-7.63 (m, 1H), 7.45-7.37 (m, 4H), 7.25 (s, 1H), 7.15 (s, 1H), 7.05 (s, 1H), 6.99 (s, 1H), 6.63 (d, J=10 Hz, 1H), 6.48 (s, 1H), 4.87 (d, J=5 Hz, 1H), 4.70-4.60 (m, 1H), 4.47-4.40 (m, 2H), 4.22-4.15 (m, 2H), 4.08-4.04 (m, 1H), 3.23-3.18 (m, 3H), 3.09-3.04 (m, 3H), 1.94 (d, J=10 Hz, 1H), 1.69-1.62 (m, 2H), 1.57-1.51 (m, 1H), 1.46-1.40 (m, 2H), 1.20-1.16 (m, 3H), 1.05-0.97 (m, 2H), 0.88-0.84 (m, 1H), 0.77-0.68 (m, 1H) ppm.

MS (ESI): m/z=[M+1] 739.10.

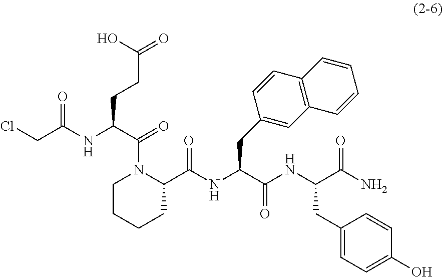 [SMILES: NC(=O)[C@H](Cc1ccc(O)cc1)NC(=O)[C@H](Cc1ccc2ccccc2c1)NC(=O)[C@@H]1CCCCN1C(=O)[C@H](CCC(=O)O)NC(=O)CCl]

### (S)-5-((S)-2-(((S)-1-(((S)-1-Amino-3-(4-hydroxyphenyl)-1-oxopropan-2-yl)amino)-3-(naphthalen-2-yl)-1-oxopropan-2-yl)carbamoyl)piperidin-1-yl)-4-(2-chloroacetamido)-5-oxopentanoic acid

¹H NMR (500 MHz, DMSO-d₆): δ=12.10 (s, 1H), 9.08 (s, 1H), 8.38 (d, J=10 Hz, 1H), 7.95 (d, J=10 Hz, 1H), 7.86 (t, J=5 Hz, 1H), 7.78-7.70 (m, 3H), 7.65-7.60 (m, 1H), 7.41-7.33 (m, 3H), 7.31-7.25 (m, 1H), 7.05-7.00 (m, 1H), 6.94 (d, J=10 Hz, 2H), 6.57 (d, J=10 Hz, 2H), 4.86-4.85 (m, 1H), 4.57-4.49 (m, 2H), 4.33-4.29 (m, 1H), 4.10-4.03 (m, 1H), 4.01 (d, J=5 Hz, 2H), 3.14-3.10 (m, 1H), 2.99-2.90 (m, 1H), 2.84-2.79 (m, 1H), 2.74-2.65 (m, 1H), 2.24 (t, J=5 Hz, 1H), 2.15 (t, J=5 Hz, 2H), 2.02-1.94 (m, 1H), 1.81-1.72 (m, 1H), 1.70-1.64 (m, 1H), 1.58-1.51 (m, 1H), 1.29-1.22 (m, 2H), 1.18-1.09 (m, 1H), 1.00-0.92 (m, 1H) ppm.

MS (ESI): m/z=[M+1] 694.74.

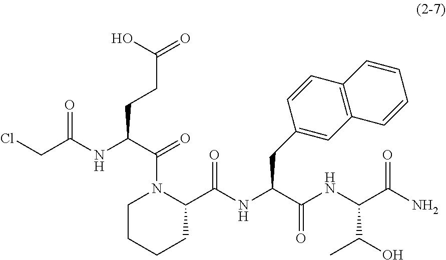 [SMILES: CC(O)[C@H](NC(=O)[C@H](Cc1ccc2ccccc2c1)NC(=O)[C@@H]1CCCCN1C(=O)[C@H](CCC(=O)O)NC(=O)CCl)C(N)=O]

### (S)-5-((S)-2-(((S)-1-(((2S,3S)-1-Amino-3-hydroxy-1-oxobutan-2-yl)amino)-3-(naphthalen-2-yl)-1-oxopropan-2-yl)carbamoyl)piperidin-1-yl)-4-(2-chloroacetamido)-5-oxopentanoic acid

¹H NMR (500 MHz, DMSO-d₆): δ=12.07 (s, 1H), 8.38 (d, J=10 Hz, 1H), 7.83 (d, J=10 Hz, 1H), 7.79-7.69 (m, 5H), 7.41-7.37 (m, 3H), 7.06-7.02 (m, 2H), 4.89 (d, J=5 Hz, 1H), 4.77-4.72 (m, 1H), 4.70-4.63 (m, 2H), 4.05-4.03 (m, 2H), 4.01-3.97 (m, 3H), 3.22-3.19 (m, 1H), 3.02-2.97 (m, 1H), 2.79 (t, J=10 Hz, 1H), 2.23 (t, J=5 Hz, 1H), 2.16 (t, J=5 Hz, 2H), 2.07-1.99 (m, 1H), 1.81-1.73 (m, 1H), 1.59-1.52 (m, 1H), 1.33-1.26 (m, 2H), 1.19-1.11 (m, 2H), 0.96 (d, J=5 Hz, 3H) ppm.

MS (ESI): m/z=[M+1] 632.64.

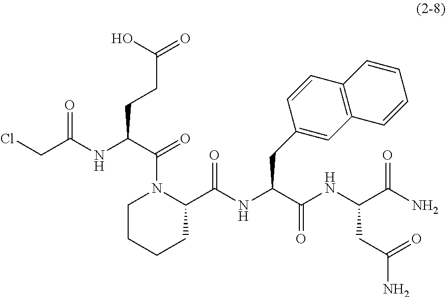 [SMILES: NC(=O)C[C@H](NC(=O)[C@H](Cc1ccc2ccccc2c1)NC(=O)[C@@H]1CCCCN1C(=O)[C@H](CCC(=O)O)NC(=O)CCl)C(N)=O]

### (S)-4-(2-Chloroacetamido)-5-((S)-2-(((S)-1-(((S)-1,4-diamino-1,4-dioxobutan-2-yl)amino)-3-(naphthalen-2-yl)-1-oxopropan-2-yl)carbamoyl)piperidin-1-yl)-5-oxopentanoic acid

¹H NMR (500 MHz, DMSO-d₆): δ=12.10 (s, 1H), 8.39 (d, J=5 Hz, 1H), 8.16 (d, J=5 Hz, 1H), 7.78-7.73 (m, 3H), 7.67-7.63 (m, 1H), 7.40-7.35 (m, 3H), 7.30-7.27 (m, 1H), 7.04-6.98 (m, 2H), 6.83 (s, 2H), 4.89 (d, J=5 Hz, 1H), 4.69-4.56 (m, 3H), 4.42-4.37 (m, 2H), 3.57-3.42 (m, 1H), 3.24-3.17 (m, 2H), 3.04-2.94 (m, 1H), 2.78-2.73 (m, 1H), 2.23 (t, J=5 Hz, 1H), 2.16 (t, J=5 Hz, 2H), 2.07-1.99 (m, 1H), 1.81-1.66 (m, 2H), 1.58-1.53 (m, 1H), 1.33-1.26 (m, 2H), 1.20-1.11 (m, 1H), 1.04-0.95 (m, 1H) ppm.

MS (ESI): m/z=[M+1] 645.75.

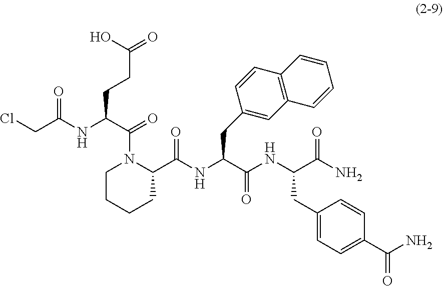 [SMILES: NC(=O)c1ccc(C[C@H](NC(=O)[C@H](Cc2ccc3ccccc3c2)NC(=O)[C@@H]2CCCCN2C(=O)[C@H](CCC(=O)O)NC(=O)CCl)C(N)=O)cc1]

### (S)-5-((S)-2-(((S)-1-(((S)-1-Amino-3-(4-carbamoylphenyl)-1-oxopropan-2-yl)amino)-3-(naphthalen-2-yl)-1-oxopropan-2-yl)carbamoyl)piperidin-1-yl)-4-(2-chloroacetamido)-5-oxopentanoic acid

MS (ESI): m/z=[M+1] 721.77.

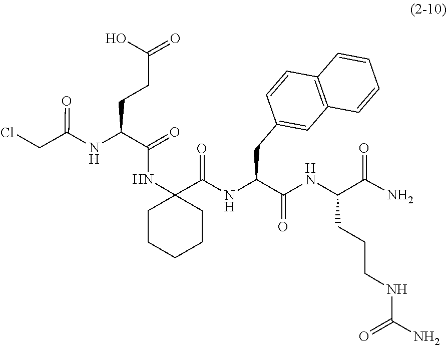 [SMILES: NC(=O)NCCC[C@H](NC(=O)[C@H](Cc1ccc2ccccc2c1)NC(=O)C1(NC(=O)[C@H](CCC(=O)O)NC(=O)CCl)CCCCC1)C(N)=O]

### (S)-5-((1-(((S)-1-(((S)-1-Amino-1-oxo-5-ureidopentan-2-yl)amino)-3-(naphthalen-2-yl)-1-oxopropan-2-yl)carbamoyl)cyclohexyl)amino)-4-(2-chloroacetamido)-5-oxopentanoic acid

MS (ESI): m/z=[M+1] 702.95.

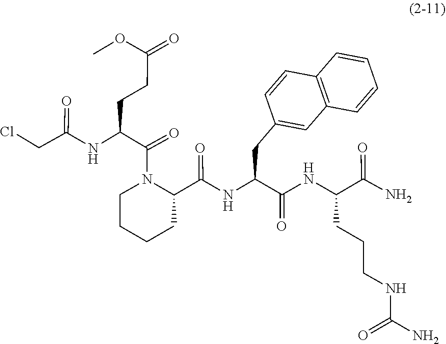 [SMILES: COC(=O)CC[C@H](NC(=O)CCl)C(=O)N1CCCC[C@H]1C(=O)N[C@@H](Cc1ccc2ccccc2c1)C(=O)N[C@@H](CCCNC(N)=O)C(N)=O]

### Methyl (S)-5-((S)-2-(((S)-1-(((S)-1-amino-1-oxo-5-ureidopentan-2-yl)amino)-3-(naphthalen-2-yl)-1-oxopropan-2-yl)carbamoyl)piperidin-1-yl)-4-(2-chloroacetamido)-5-oxopentanoate

¹H NMR (500 MHz, DMSO-d₆): δ=8.39 (d, J=10 Hz, 1H), 7.98 (d, J=5 Hz, 1H), 7.89 (t, J=5 Hz, 1H), 7.78-7.72 (m, 4H), 7.68-7.64 (m, 2H), 7.40-7.36 (m, 3H), 7.26 (s, 1H), 6.99 (s, 1H), 5.89 (s, 1H), 4.88 (d, J=5 Hz, 1H), 4.69-4.63 (m, 2H), 4.18-4.14 (m, 2H), 4.10-4.05 (m, 1H), 4.00 (s, 2H), 3.52 (s, 2H), 3.49 (s, 1H), 3.20-3.17 (m, 1H), 2.99-2.88 (m, 3H), 2.78-2.73 (m, 1H), 2.32 (t, J=5 Hz, 1H), 2.22 (t, J=5 Hz, 2H), 2.05-1.99 (m, 1H), 1.81-1.74 (m, 2H), 1.62-1.56 (m, 2H), 1.47-1.42 (m, 1H), 1.34-1.26 (m, 3H), 1.20-1.11 (m, 1H) ppm.

MS (ESI): m/z=[M+1] 702.62.

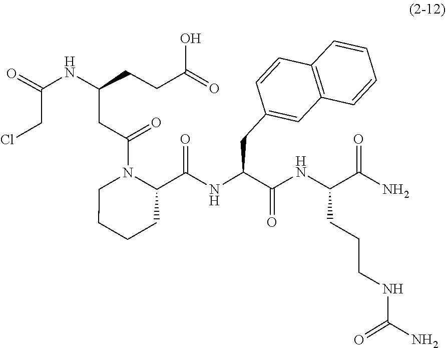 [SMILES: NC(=O)NCCC[C@H](NC(=O)[C@H](Cc1ccc2ccccc2c1)NC(=O)[C@@H]1CCCCN1C(=O)C[C@H](CCC(=O)O)NC(=O)CCl)C(N)=O]

### (S)-6-((S)-2-(((S)-1-(((S)-1-Amino-1-oxo-5-ureidopentan-2-yl)amino)-3-(naphthalen-2-yl)-1-oxopropan-2-yl)carbamoyl)piperidin-1-yl)-4-(2-chloroacetamido)-6-oxohexanoic acid

MS (ESI): m/z=[M+1] 702.85.

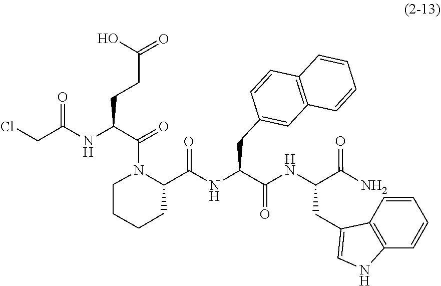 [SMILES: NC(=O)[C@H](Cc1c[nH]c2ccccc12)NC(=O)[C@H](Cc1ccc2ccccc2c1)NC(=O)[C@@H]1CCCCN1C(=O)[C@H](CCC(=O)O)NC(=O)CCl]

### (S)-5-((S)-2-(((S)-1-(((S)-1-Amino-3-(1H-indol-3-yl)-1-oxopropan-2-yl)amino)-3-(naphthalen-2-yl)-1-oxopropan-2-yl)carbamoyl)piperidin-1-yl)-4-(2-chloroacetamido)-5-oxopentanoic acid

¹H NMR (500 MHz, DMSO-d₆): δ=12.09 (s, 1H), 10.72 (s, 1H), 8.38 (d, J=10 Hz, 1H), 8.02 (d, J=10 Hz, 1H), 7.77-7.76 (m, 1H), 7.72-7.69 (m, 1H), 7.65-7.60 (m, 2H), 7.53 (d, J=10 Hz, 1H), 7.38-7.33 (m, 3H), 7.26 (d, J=5 Hz, 2H), 7.08 (s, 1H), 7.01-6.96 (m, 2H), 6.92-6.86 (m, 2H), 4.86 (s, 1H), 4.66-4.55 (m, 2H), 4.44-4.42 (m, 1H), 4.05-4.00 (m, 1H), 3.68-3.55 (m, 1H), 3.55-3.49 (m, 1H), 3.17-3.13 (m, 1H), 3.10-3.04 (m, 1H), 2.97-2.93 (m, 2H), 2.73-2.68 (m, 1H), 2.24 (t, J=5 Hz, 1H), 2.15 (t, J=5 Hz, 2H), 1.98-1.93 (m, 1H), 1.77-1.66 (m, 1H), 1.57-1.51 (m, 1H), 1.27-1.21 (m, 2H), 1.16-1.08 (m, 1H), 0.98-0.91 (m, 1H) ppm.

MS (ESI): m/z=[M+1] 717.86.

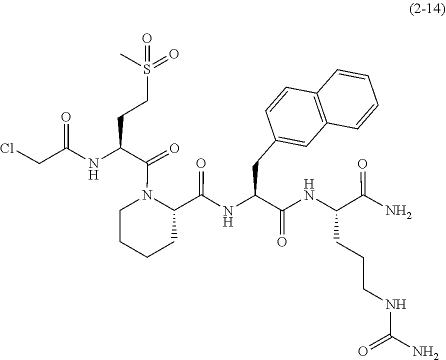 [SMILES: CS(=O)(=O)CC[C@H](NC(=O)CCl)C(=O)N1CCCC[C@H]1C(=O)N[C@@H](Cc1ccc2ccccc2c1)C(=O)N[C@@H](CCCNC(N)=O)C(N)=O]

### (S)—N—((S)-1-(((S)-1-Amino-1-oxo-5-ureidopentan-2-yl)amino)-3-(naphthalen-2-yl)-1-oxopropan-2-yl)-1-((S)-2-(2-chloroacetamido)-4-(methylsulfonyl)butanoyl)piperidine-2-carboxamide

MS (ESI): m/z=[M+1] 722.87.

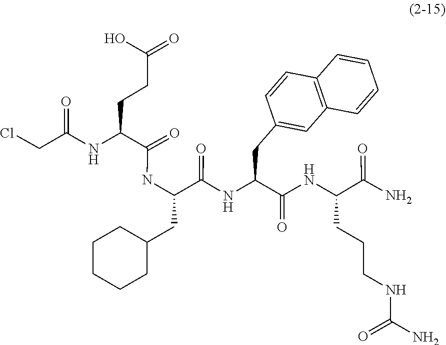 [SMILES: NC(=O)NCCC[C@H](NC(=O)[C@H](Cc1ccc2ccccc2c1)NC(=O)[C@H](CC1CCCCC1)NC(=O)[C@H](CCC(=O)O)NC(=O)CCl)C(N)=O]

### (6S,9S,12S,15S)-1-Amino-6-carbamoyl-15-(2-chloroacetamido)-12-(cyclohexylmethyl)-9-(naphthalen-2-ylmethyl)-1,8,11,14-tetraoxo-2,7,10,13-tetraazaoctadecan-18-oic acid

MS (ESI): m/z=[M+1] 730.90.

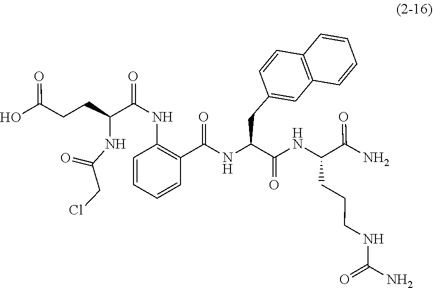 [SMILES: NC(=O)NCCC[C@H](NC(=O)[C@H](Cc1ccc2ccccc2c1)NC(=O)c1ccccc1NC(=O)[C@H](CCC(=O)O)NC(=O)CCl)C(N)=O]

### (S)-5-((2-(((S)-1-(((S)-1-Amino-1-oxo-5-ureidopentan-2-yl)amino)-3-(naphthalen-2-yl)-1-oxopropan-2-yl)carbamoyl)phenyl)amino)-4-(2-chloroacetamido)-5-oxopentanoic acid

MS (ESI): m/z=[M+1] 696.79.

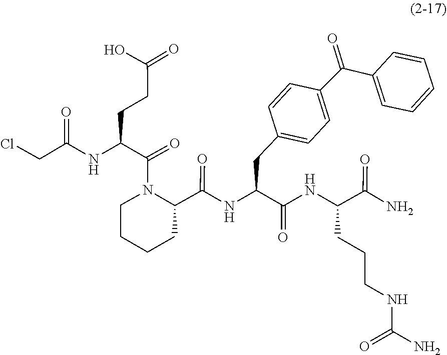 [SMILES: NC(=O)NCCC[C@H](NC(=O)[C@H](Cc1ccc(C(=O)c2ccccc2)cc1)NC(=O)[C@@H]1CCCCN1C(=O)[C@H](CCC(=O)O)NC(=O)CCl)C(N)=O]

### (S)-5-((S)-2-(((S)-1-(((S)-1-Amino-1-oxo-5-ureidopentan-2-yl)amino)-3-(4-benzoylphenyl)-1-oxopropan-2-yl)carbamoyl)piperidin-1-yl)-4-(2-chloroacetamido)-5-oxopentanoic acid

MS (ESI): m/z=[M+1] 742.85.

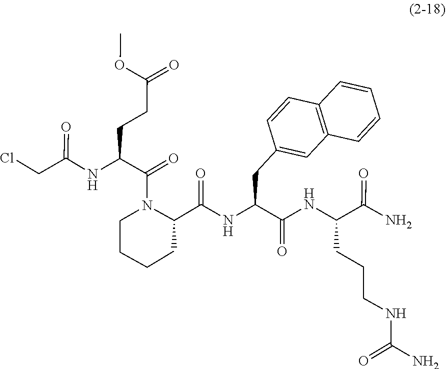 [SMILES: COC(=O)CC[C@H](NC(=O)CCl)C(=O)N1CCCC[C@H]1C(=O)N[C@@H](Cc1ccc2ccccc2c1)C(=O)N[C@@H](CCCNC(N)=O)C(N)=O]

### Methyl (S)-5-((S)-2-(((S)-1-(((S)-1-amino-1-oxo-5-ureidopentan-2-yl)amino)-3-(naphthalen-2-yl)-1-oxopropan-2-yl)carbamoyl)piperidin-1-yl)-4-(2-chloroacetamido)-5-oxopentanoate

MS (ESI): m/z=[M+1] 702.86.

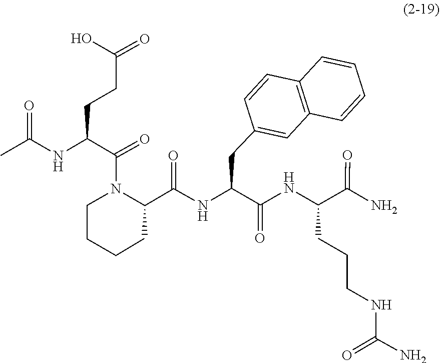 [SMILES: CC(=O)N[C@@H](CCC(=O)O)C(=O)N1CCCC[C@H]1C(=O)N[C@@H](Cc1ccc2ccccc2c1)C(=O)N[C@@H](CCCNC(N)=O)C(N)=O]

### (S)-4-Acetamido-5-((S)-2-(((S)-1-(((S)-1-amino-1-oxo-5-ureidopentan-2-yl)amino)-3-(naphthalen-2-yl)-1-oxopropan-2-yl)carbamoyl)piperidin-1-yl)-5-oxopentanoic acid

¹H NMR (500 MHz, DMSO-d₆): δ=12.10 (s, 1H), 8.03 (d, J=10 Hz, 1H), 7.98 (d, J=10 Hz, 1H), 7.78-7.72 (m, 4H), 7.66-7.60 (m, 2H), 7.39 (t, J=5 Hz, 3H), 7.26 (s, 1H), 6.98-6.96 (m, 1H), 5.90 (s, 1H), 4.87 (d, J=5 Hz, 1H), 4.89-4.61 (m, 2H), 4.18-4.11 (m, 2H), 3.23-3.17 (m, 1H), 3.02-2.86 (m, 4H), 2.71-2.65 (m, 1H), 2.25 (t, J=5 Hz, 1H), 2.14 (t, J=5 Hz, 2H), 1.77 (s, 1H), 1.73 (s, 2H), 1.64-1.50 (m, 3H), 1.46-1.41 (m, 1H), 1.3-1.22 (m, 4H), 1.17-1.09 (m, 1H), 1.00-0.90 (m, 2H) ppm.

MS (ESI): m/z=[M+1] 654.90.

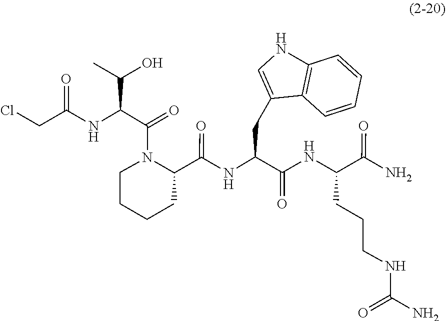 [SMILES: CC(O)[C@H](NC(=O)CCl)C(=O)N1CCCC[C@H]1C(=O)N[C@@H](Cc1c[nH]c2ccccc12)C(=O)N[C@@H](CCCNC(N)=O)C(N)=O]

### (S)—N—((S)-1-(((S)-1-Amino-1-oxo-5-ureidopentan-2-yl)amino)-3-(1H-indol-3-yl)-1-oxopropan-2-yl)-1-((2-chloroacetyl)-L-allothreonyl)piperidine-2-carboxamide

¹H NMR (500 MHz, DMSO-d₆): δ=10.81 (s, 1H), 8.19 (d, J=5 Hz, 1H), 7.95-7.89 (m, 1H), 7.56-7.51 (m, 2H), 7.25-7.24 (m, 2H), 7.05 (s, 1H), 6.99-6.96 (m, 2H), 6.90 (t, J=5 Hz, 1H), 6.48 (s, 1H), 5.94 (s, 1H), 5.36 (s, 2H), 4.94 (d, J=5 Hz, 1H), 4.73-4.66 (m, 2H), 4.20 (s, 1H), 4.15-4.10 (m, 2H), 3.82-3.79 (m, 1H), 3.13-3.09 (m, 1H), 2.94-2.86 (m, 3H), 2.08-2.01 (m, 2H), 1.62-1.55 (m, 2H), 1.47-1.38 (m, 2H), 1.31-1.26 (m, 3H), 1.17 (s, 2H), 0.98 (d, J=5 Hz, 2H), 0.86-0.79 (m, 1H) ppm.

MS (ESI): m/z=[M+1] 649.90.

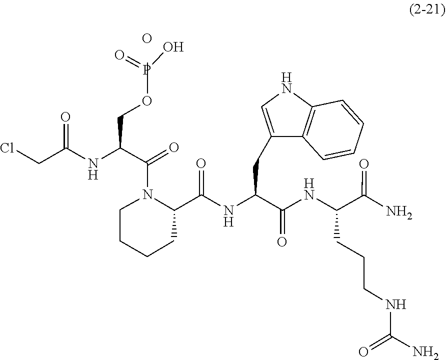

### (S)-3-((S)-2-(((S)-1-(((S)-1-Amino-1-oxo-5-ureidopentan-2-yl)amino)-3-(1H-indol-3-yl)-1-oxopropan-2-yl)carbamoyl)piperidin-1-yl)-2-(2-chloroacetamido)-3-oxopropyl dihydrogen phosphate

¹H NMR (500 MHz, DMSO-d₆): δ=10.77 (s, 1H), 8.64 (d, J=5 Hz, 1H), 7.91 (d, J=5 Hz, 1H), 7.81-7.74 (m, 1H), 7.56 (d, J=5 Hz, 1H), 7.25 (d, J=5 Hz, 1H), 7.12-7.09 (m, 1H), 7.01-6.96 (m, 2H), 6.91 (t, J=5 Hz, 1H), 6.63 (s, 1H), 6.09 (s, 1H), 4.92-4.89 (m, 1H), 4.85 (d, J=5 Hz, 1H), 4.61-4.58 (m, 1H), 4.49-4.43 (m, 1H), 4.15-4.13 (m, 1H), 4.10-4.06 (m, 2H), 4.03 (s, 2H), 3.68 (t, J=5 Hz, 1H), 3.12-3.08 (m, 2H), 2.98-2.92 (m, 2H), 2.90-2.83 (m, 2H), 2.66-2.57 (m, 1H), 2.04-2.01 (m, 1H), 1.78-1.68 (m, 1H), 1.61-1.54 (m, 1H), 1.47-1.40 (m, 1H), 1.36-1.31 (m, 2H), 1.26-1.22 (m, 2H), 1.17-1.10 (m, 2H), 1.02-0.94 (m, 1H) ppm.

MS (ESI): m/z=[M+1] 715.85.

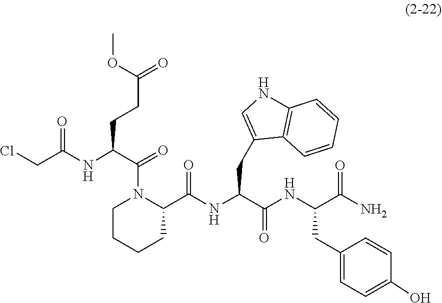 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1ccc(O)cc1)C(N)=O]

### Methyl (S)-5-((S)-2-(((S)-1-(((S)-1-Amino-3-(4-hydroxyphenyl)-1-oxopropan-2-yl)amino)-3-(1H-indol-3-yl)-1-oxopropan-2-yl)carbamoyl)piperidin-1-yl)-4-(2-chloroacetamido)-5-oxopentanoate

¹H NMR (500 MHz, DMSO-d₆): δ=10.73 (s, 1H), 9.08 (s, 1H), 8.38 (d, J=10 Hz, 1H), 7.75 (d, J=10 Hz, 1H), 7.56 (d, J=5 Hz, 1H), 7.49 (d, J=5 Hz, 1H), 7.25-7.19 (m, 2H), 7.04 (s, 1H), 6.99-6.94 (m, 2H), 6.92-6.88 (m, 3H), 6.56 (d, J=5 Hz, 2H), 4.88 (d, J=5 Hz, 1H), 4.69-4.65 (m, 1H), 4.52-4.49 (m, 1H), 4.30-4.26 (m, 1H), 4.02 (s, 2H), 3.51 (s, 3H), 3.05-3.01 (m, 1H), 2.94-2.87 (m, 1H), 2.77-2.73 (m, 1H), 2.69-2.63 (m, 1H), 2.34 (t, J=5 Hz, 1H), 2.25 (t, J=5 Hz, 2H), 2.01-1.96 (m, 1H), 1.80-1.73 (m, 1H), 1.61-1.55 (m, 1H), 1.36-1.29 (m, 3H), 1.19-1.16 (m, 2H), 1.07-0.97 (m, 1H) ppm.

MS (ESI): m/z=[M+1] 697.83.

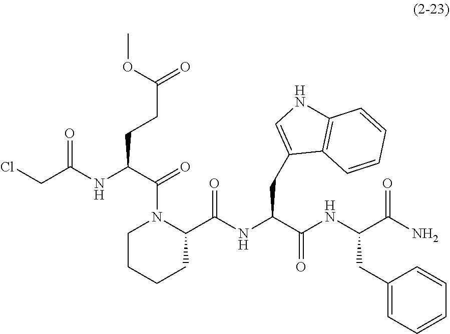 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1ccccc1)C(N)=O]

### Methyl (S)-5-((S)-2-(((S)-1-(((S)-1-Amino-1-oxo-3-phenylpropan-2-yl)amino)-3-(1H-indol-3-yl)-1-oxopropan-2-yl)carbamoyl)piperidin-1-yl)-4-(2-chloroacetamido)-5-oxopentanoate

¹H NMR (500 MHz, DMSO-d₆): δ=10.72 (s, 1H), 8.38 (d, J=5 Hz, 1H), 7.88 (d, J=5 Hz, 1H), 7.52 (d, J=5 Hz, 1H), 7.49 (d, J=5 Hz, 1H), 7.25-7.23 (m, 2H), 2.19-2.16 (m, 3H), 7.12 (d, J=5 Hz, 2H), 7.02 (s, 1H), 6.99-6.95 (m, 1H), 6.90 (t, J=5 Hz, 1H), 4.87 (d, J=5 Hz, 1H), 4.68-4.64 (m, 1H), 4.54-4.49 (m, 1H), 4.41-4.36 (m, 1H), 4.01 (d, J=5 Hz, 2H), 3.51 (s, 3H), 3.03-3.00 (m, 1H), 2.93-2.89 (m, 2H), 2.79-2.74 (m, 1H), 2.74-2.68 (m, 1H), 2.33 (t, J=5 Hz, 1H), 2.25 (t, J=5 Hz, 2H), 2.03-1.97 (m, 1H), 1.80-1.72 (m, 1H), 1.61-1.53 (m, 1H), 1.35-1.38 (m, 3H), 1.19-1.11 (m, 2H), 1.07-1.00 (m, 1H) ppm.

MS (ESI): m/z=[M+1] 681.82.

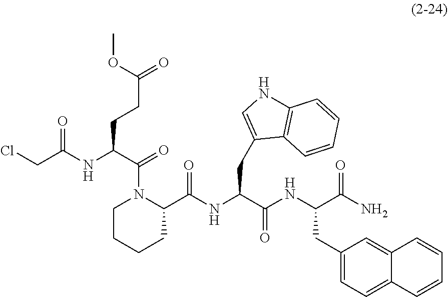 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1ccc2ccccc2c1)C(N)=O]

### Methyl (S)-5-((S)-2-(((S)-1-(((S)-1-Amino-3-(naphthalen-2-yl)-1-oxopropan-2-yl)amino)-3-(1H-indol-3-yl)-1-oxopropan-2-yl)carbamoyl)piperidin-1-yl)-4-(2-chloroacetamido)-5-oxopentanoate

¹H NMR (500 MHz, DMSO-d₆): δ=10.72 (s, 1H), 8.37 (d, J=5 Hz, 1H), 7.91 (d, J=5 Hz, 1H), 7.81-7.77 (m, 2H), 7.75-7.72 (m, 2H), 7.59 (s, 1H), 7.54 (d, J=10 Hz, 1H), 7.44 (d, J=10 Hz, 1H), 7.40-7.37 (m, 2H), 7.31 (d, J=10 Hz, 1H), 7.28 (s, 1H), 7.24 (d, J=5 Hz, 1H), 7.04 (d, J=10 Hz, 1H), 6.99-6.96 (m, 1H), 6.90-6.87 (m, 1H), 4.83 (d, J=5 Hz, 1H), 4.67-4.60 (m, 1H), 4.53-4.46 (m, 2H), 4.01 (s, 1H), 3.51 (s, 3H), 3.11-3.04 (m, 1H), 3.01-2.93 (m, 2H), 2.93-2.86 (m, 1H), 2.73-2.66 (m, 1H), 2.33 (t, J=5 Hz, 1H), 2.24 (t, J=5 Hz, 2H), 1.94-1.88 (m, 1H), 1.79-1.74 (m, 1H), 1.58-1.52 (m, 1H), 1.27-1.21 (m, 3H), 1.15-1.08 (m, 2H), 1.00-0.89 (m, 1H) ppm.

MS (ESI): m/z [M+1] 731.81.

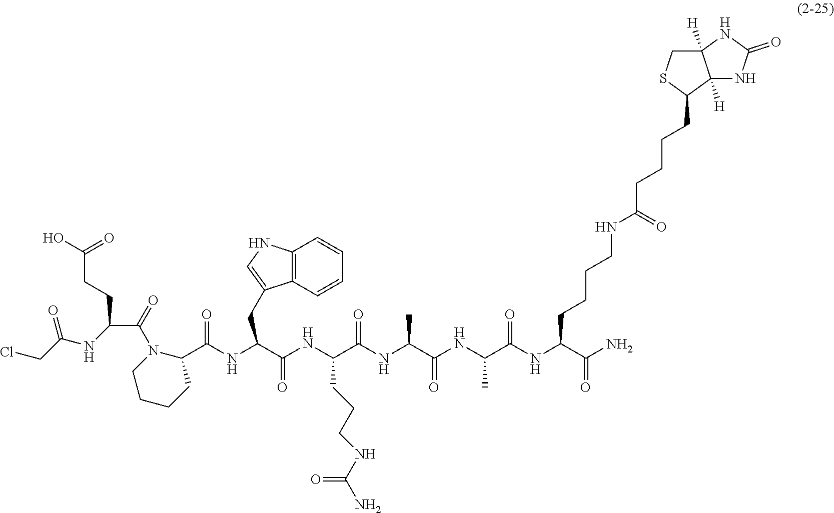 [SMILES: C[C@H](NC(=O)[C@H](C)NC(=O)[C@H](CCCNC(N)=O)NC(=O)[C@H](Cc1c[nH]c2ccccc12)NC(=O)[C@@H]1CCCCN1C(=O)[C@H](CCC(=O)O)NC(=O)CCl)C(=O)N[C@@H](CCCCNC(=O)CCCC[C@H]1SC[C@H]2NC(=O)N[C@H]21)C(N)=O]

### (S)-5-((S)-2-(((2S,5S,8S,11S)-14-Carbamoyl-1-(1H-indol-3-yl)-8,11-dimethyl-3,6,9,12,20-pentaoxo-24-((3aR,4R,6aS)-2-oxohexahydro-1H-thieno[3,4-d]imidazol-4-yl)-5-(3-ureidopropyl)-4,7,10,13,19-pentaazatetracosan-2-yl)carbamoyl)piperidin-1-yl)-4-(2-chloroacetamido)-5-oxopentanoic acid

¹H NMR (500 MHz, DMSO-d₆): δ=12.17 (s, 1H), 10.70 (s, 1H), 8.39 (d, J=10 Hz, 1H), 8.01 (t, J=5 Hz, 2H), 7.92 (d, J=5 Hz, 1H), 7.67 (t, J=5 Hz, 1H), 7.63 (d, J=10 Hz, 1H), 7.53 (t, J=10 Hz, 2H), 7.24 (d, J=10 Hz, 1H), 7.20 (s, 1H), 7.06 (s, 1H), 6.99-6.95 (m, 2H), 6.90 (t, J=5 Hz, 1H), 6.37 (s, 1H), 5.90 (s, 1H), 4.89 (d, J=5 Hz, 1H), 4.69-4.66 (m, 1H), 4.60-4.54 (m, 1H), 4.26-4.21 (m, 1H), 4.20-4.16 (m, 3H), 4.08-4.04 (m, 3H), 4.02 (d, J=5 Hz, 2H), 3.11-3.07 (m, 1H), 3.04-3.00 (m, 1H), 2.94-2.88 (m, 4H), 2.76 (d, J=5 Hz, 1H), 2.74 (d, J=5 Hz, 1H), 2.52 (s, 1H), 2.49 (s, 1H), 2.47 (s, 1H), 2.43 (s, 6H), 2.24 (t, J=5 Hz, 1H), 2.18 (t, J=5 Hz, 2H), 1.97 (t, J=5 Hz, 2H), 1.81-1.73 (m, 2H), 1.61-1.53 (m, 4H), 1.45-1.39 (m, 5H), 1.31-1.27 (m, 6H), 1.25-1.19 (m, 2H), 1.14 (d, J=10 Hz, 4H), 1.07-1.00 (m, 2H) ppm.

MS (ESI): m/z=[M+2] 1174.30.

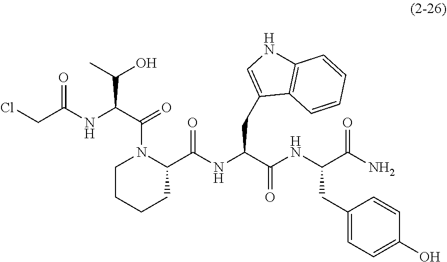 [SMILES: CC(O)[C@H](NC(=O)CCl)C(=O)N1CCCC[C@H]1C(=O)N[C@@H](Cc1c[nH]c2ccccc12)C(=O)N[C@@H](Cc1ccc(O)cc1)C(N)=O]

### (S)—N—((S)-1-(((S)-1-Amino-3-(4-hydroxyphenyl)-1-oxopropan-2-yl)amino)-3-(1H-indol-3-yl)-1-oxopropan-2-yl)-1-((2-chloroacetyl)-L-allothreonyl)piperidine-2-carboxamide

¹H NMR (500 MHz, DMSO-d₆): δ=10.74 (s, 1H), 9.07 (s, 1H), 8.16 (d, J=5 Hz, 1H), 7.78 (d, J=5 Hz, 1H), 7.53-7.47 (m, 2H), 7.25-7.23 (m, 2H), 7.02 (s, 1H), 6.98-6.96 (m, 2H), 6.90 (d, J=10 Hz, 2H), 6.56 (d, J=10 Hz, 2H), 4.94 (d, J=5 Hz, 1H), 4.70-4.66 (m, 1H), 4.65-4.63 (m, 1H), 4.30-4.26 (m, 1H), 4.11 (s, 2H), 3.80-3.78 (m, 1H), 3.05-3.02 (m, 1H), 2.93-2.86 (m, 1H), 2.81-2.75 (m, 1H), 2.69-2.65 (m, 1H), 2.00-1.98 (m, 2H), 1.35-1.26 (m, 2H), 1.21-1.45 (m, 3H), 1.13-1.09 (m, 1H), 1.02-1.00 (m, 2H), 0.97 (d, J=5 Hz, 2H), 0.87-0.79 (m, 1H) ppm.

MS (ESI): m/z=[M+1] 655.75.

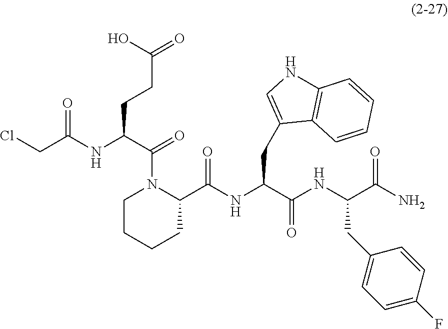 [SMILES: NC(=O)[C@H](Cc1ccc(F)cc1)NC(=O)[C@H](Cc1c[nH]c2ccccc12)NC(=O)[C@@H]1CCCCN1C(=O)[C@H](CCC(=O)O)NC(=O)CCl]

### (S)-5-((S)-2-(((S)-1-(((S)-1-Amino-3-(4-fluorophenyl)-1-oxopropan-2-yl)amino)-3-(1H-indol-3-yl)-1-oxopropan-2-yl)carbamoyl)piperidin-1-yl)-4-(2-chloroacetamido)-5-oxopentanoic acid

¹H NMR (500 MHz, DMSO-d₆): δ=12.07 (s, 1H), 10.71 (s, 1H), 8.38 (d, J=10 Hz, 1H), 7.88 (d, J=10 Hz, 1H), 7.53 (d, J=10 Hz, 1H), 7.49 (d, J=10 Hz, 1H), 7.26-7.22 (m, 2H), 7.17-7.12 (m, 3H), 7.03 (s, 2H), 7.00 (s, 1H), 6.98-6.96 (m, 3H), 6.90 (t, J=5 Hz, 2H), 4.87 (d, J=5 Hz, 1H), 4.69-4.63 (m, 1H), 4.56-4.52 (m, 1H), 0.438-4.34 (m, 1H), 4.02 (d, J=5 Hz, 2H), 3.03-2.99 (m, 1H), 2.91-2.86 (m, 2H), 2.77-2.73 (m, 2H), 2.25 (t, J=5 Hz, 1H), 2.17 (t, J=5 Hz, 2H), 1.78-1.74 (m, 1H), 1.58-1.51 (m, 1H), 1.34-1.27 (m, 2H), 1.20-1.13 (m, 1H) ppm.

MS (ESI): m/z=[M+1] 685.78.

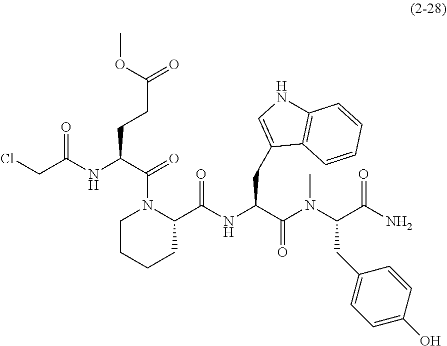 [SMILES: COC(=O)CC[C@H](NC(=O)CCl)C(=O)N1CCCC[C@H]1C(=O)N[C@@H](Cc1c[nH]c2ccccc12)C(=O)N(C)[C@@H](Cc1ccc(O)cc1)C(N)=O]

### Methyl (S)-5-((S)-2-(((S)-1-(((S)-1-amino-3-(4-hydroxyphenyl)-1-oxopropan-2-yl)(methyl)-amino)-3-(1H-indol-3-yl)-1-oxopropan-2-yl)carbamoyl)piperidin-1-yl)-4-(2-chloroacetamido)-5-oxopentanoate

¹H NMR (500 MHz, DMSO-d₆): δ=10.74 (s, 1H), 9.05 (s, 1H), 8.39 (d, J=5 Hz, 2H), 7.81 (d, J=10 Hz, 1H), 7.73 (t, J=5 Hz, 1H), 7.44 (d, J=10 Hz, 2H), 7.35 (d, J=5 Hz, 1H), 7.26-7.23 (m, 2H), 7.03 (s, 1H), 7.01-6.96 (m, 2H), 6.94-6.87 (m, 2H), 6.74 (d, J=10 Hz, 2H), 6.57 (t, J=5 Hz, 2H), 6.48 (s, 1H), 6.46 (s, 2H), 4.92-4.89 (m, 1H), 4.73-4.67 (m, 1H), 4.02 (s, 2H), 4.00 (d, J=5 Hz, 1H), 3.51 (s, 3H), 3.02-2.96 (m, 2H), 2.76 (s, 1H), 2.70 (s, 3H), 2.26 (t, J=5 Hz, 2H), 1.96-1.91 (m, 1H), 1.85-1.77 (m, 1H), 1.63-1.57 (m, 1H), 1.42-1.30 (m, 1H) ppm.

MS (ESI): m/z [M+1] 711.80.

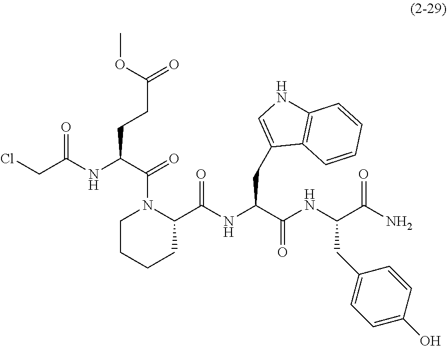 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1ccc(O)cc1)C(N)=O]

### Methyl (S)-5-((S)-2-(((S)-1-(((S)-1-amino-3-(4-hydroxyphenyl)-1-oxopropan-2-yl)amino)-3-(1H-indol-3-yl)-1-oxopropan-2-yl)(methyl)carbamoyl)piperidin-1-yl)-4-(2-chloroacetamido)-5-oxopentanoate

MS (ESI): m/z=[M+1] 711.75.

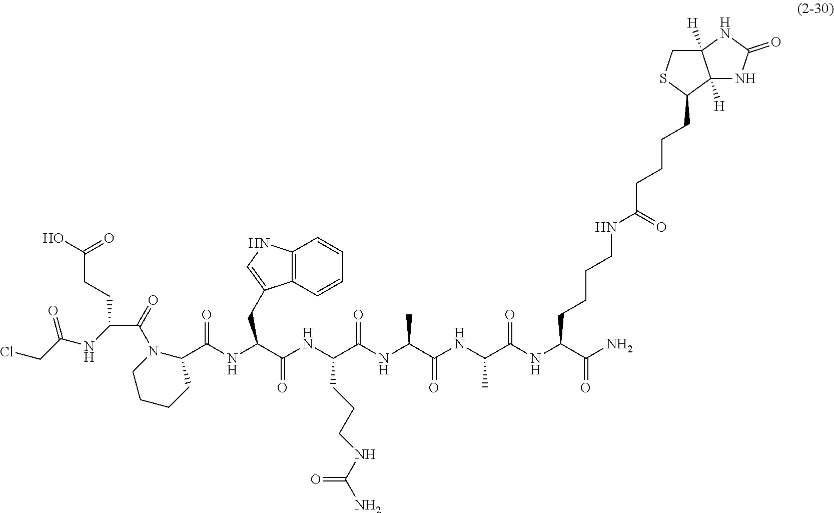 [SMILES: C[C@H](NC(=O)[C@H](C)NC(=O)[C@H](CCCNC(N)=O)NC(=O)[C@H](Cc1c[nH]c2ccccc12)NC(=O)[C@@H]1CCCCN1C(=O)[C@@H](CCC(=O)O)NC(=O)CCl)C(=O)N[C@@H](CCCCNC(=O)CCCC[C@H]1SC[C@H]2NC(=O)N[C@H]21)C(N)=O]

### (R)-5-((S)-2-(((2S,5S,8S,11S)-14-Carbamoyl-1-(1H-indol-3-yl)-8,11-dimethyl-3,6,9,12,20-pentaoxo-24-((3aR,4R,6aS)-2-oxohexahydro-1H-thieno[3,4-d]imidazol-4-yl)-5-(3-ureidopropyl)-4,7,10,13,19-pentaazatetracosan-2-yl)carbamoyl)piperidin-1-yl)-4-(2-chloroacetamido)-5-oxopentanoic acid

¹H NMR (500 MHz, DMSO-d₆): δ=12.28 (s, 1H), 10.76 (s, 1H), 8.52 (d, J=5 Hz, 1H), 8.09-8.03 (m, 2H), 7.99 (d, J=5 Hz, 1H), 7.88-7.85 (m, 1H), 7.75-7.70 (m, 2H), 7.63-7.58 (m, 2H), 7.31-7.27 (m, 2H), 7.16 (d, J=5 Hz, 1H), 7.06-7.01 (m, 2H), 6.98 (d, J=5 Hz, 1H), 6.83-6.79 (m, 1H), 6.72-6.64 (m, 1H), 6.45 (s, 1H), 5.95 (s, 1H), 5.43 (s, 1H), 4.94 (d, J=5 Hz, 1H), 4.80-4.73 (m, 2H), 4.68-4.60 (m, 3H), 4.31-4.28 (m, 3H), 4.26-4.23 (m, 3H), 3.13-3.05 (m, 1H), 3.00-2.94 (m, 4H), 2.84-2.79 (m, 1H), 2.76-2.70 (m, 1H) 2.54 (d, J=10 Hz, 3H), 2.50 (s, 6H), 2.27-2.19 (m, 2H), 2.04 (d, J=10 Hz, 3H), 1.89-1.83 (m, 2H), 1.65-1.59 (m, 4H), 1.49-1.47 (m, 4H), 1.42-1.31 (m, 5H), 1.25-1.15 (m, 4H), 1.12-1.02 (m, 2H), 0.99-0.89 (m, 1H) ppm.

MS (ESI): m/z=[M+2] 1174.40.

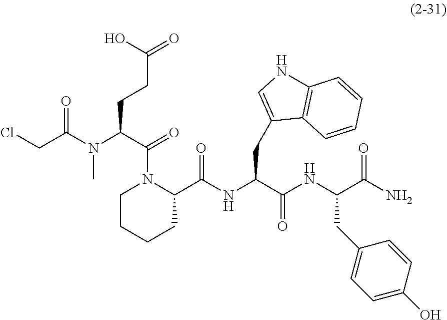 [SMILES: CN(C(=O)CCl)[C@@H](CCC(=O)O)C(=O)N1CCCC[C@H]1C(=O)N[C@@H](Cc1c[nH]c2ccccc12)C(=O)N[C@@H](Cc1ccc(O)cc1)C(N)=O]

### (S)-5-((S)-2-(((S)-1-(((S)-1-Amino-3-(4-hydroxyphenyl)-1-oxopropan-2-yl)amino)-3-(1H-indol-3-yl)-1-oxopropan-2-yl)carbamoyl)piperidin-1-yl)-4-(2-chloro-N-methylacetamido)-5-oxopentanoic acid

MS (ESI): m/z=[M+1] 697.73.

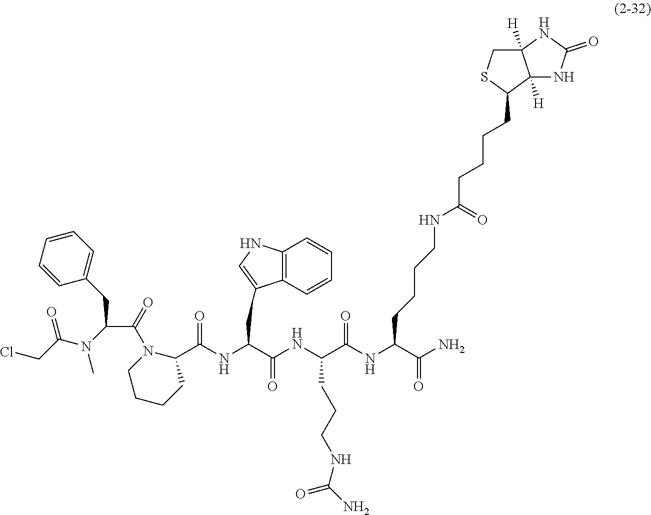 [SMILES: CN(C(=O)CCl)[C@@H](Cc1ccccc1)C(=O)N1CCCC[C@H]1C(=O)N[C@@H](Cc1c[nH]c2ccccc12)C(=O)N[C@@H](CCCNC(N)=O)C(=O)N[C@@H](CCCCNC(=O)CCCC[C@H]1SC[C@H]2NC(=O)N[C@H]21)C(N)=O]

### (S)—N—((S)-1-(((S)-1-(((S)-1-Amino-1-oxo-6-(5-((3aR,4R,6aS)-2-oxohexahydro-1H-thieno[3,4-d]imidazol-4-yl)pentanamido)hexan-2-yl)amino)-1-oxo-5-ureidopentan-2-yl)amino)-3-(1H-indol-3-yl)-1-oxopropan-2-yl)-1-(N-(2-chloroacetyl)-N-methyl-L-phenylalanyl)piperidine-2-carboxamide

MS (ESI): m/z=[M+1] 1063.32.

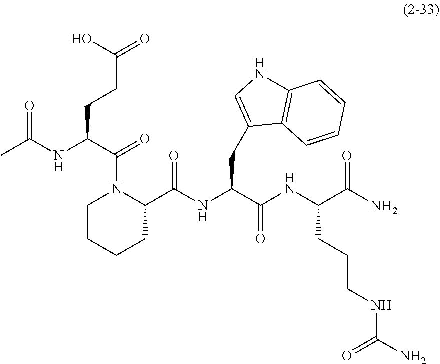 [SMILES: CC(=O)N[C@@H](CCC(=O)O)C(=O)N1CCCC[C@H]1C(=O)N[C@@H](Cc1c[nH]c2ccccc12)C(=O)N[C@@H](CCCNC(N)=O)C(N)=O]

### (S)-4-Acetamido-5-((S)-2-(((S)-1-(((S)-1-amino-1-oxo-5-ureidopentan-2-yl)amino)-3-(1H-indol-3-yl)-1-oxopropan-2-yl)carbamoyl)piperidin-1-yl)-5-oxopentanoic acid

¹H NMR (500 MHz, DMSO-d₆): δ=12.07 (s, 1H), 10.72 (s, 1H), 8.03 (d, J=10 Hz, 1H), 7.86 (d, J=10 Hz, 1H), 7.52 (d, J=10 Hz, 2H), 7.24 (d, J=5 Hz, 1H), 7.19 (s, 1H), 7.06 (s, 1H), 6.97-6.88 (m, 4H), 5.87 (s, 1H), 5.34 (s, 1H), 4.88 (d, J=5 Hz, 1H), 4.66-4.56 (m, 2H), 4.52-4.44 (m, 1H), 4.14-4.12 (m, 1H), 3.12-3.09 (m, 2H), 3.02-2.93 (m, 2H), 2.91-2.84 (m, 3H), 2.73-2.67 (m, 1H), 2.26 (t, J=5 Hz, 1H), 2.16 (t, J=5 Hz, 1H), 2.12-2.08 (m, 1H), 2.03-2.00 (m, 1H), 1.79 (s, 2H), 1.75 (s, 3H), 1.60-1.51 (m, 2H), 1.32-1.21 (m, 2H), 1.17-1.07 (m, 1H) ppm.

MS (ESI): m/z=[M+1] 643.99.

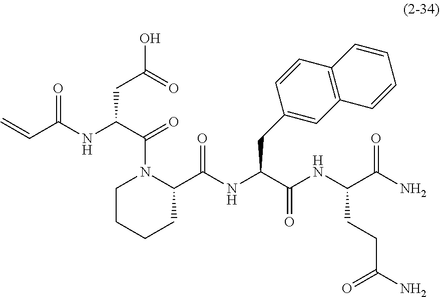 [SMILES: C=CC(=O)N[C@H](CC(=O)O)C(=O)N1CCCC[C@H]1C(=O)N[C@@H](Cc1ccc2ccccc2c1)C(=O)N[C@@H](CCC(N)=O)C(N)=O]

### (R)-3-Acrylamido-4-((S)-2-(((S)-1-(((S)-1,5-diamino-1,5-dioxopentan-2-yl)amino)-3-(naphthalen-2-yl)-1-oxopropan-2-yl)carbamoyl)piperidin-1-yl)-4-oxobutanoic acid

¹H NMR (500 MHz, DMSO-d₆): δ=12.26 (s, 1H), 8.73 (d, J=10 Hz, 1H), 8.16 (d, J=10 Hz, 2H), 7.84 (d, J=10 Hz, 2H), 7.70 (d, J=10 Hz, 2H), 7.52 (t, J=5 Hz, 1H), 7.46 (t, J=5 Hz, 1H), 7.35-7.18 (m, 3H), 7.14 (s, 1H), 7.04 (s, 2H), 6.93 (s, 1H), 6.69 (s, 1H), 6.22-6.11 (m, 2H), 5.57 (t, J=10 Hz, 1H), 4.92-4.88 (m, 2H), 4.76-4.61 (m, 1H), 4.15-4.18 (m, 1H), 3.64-3.61 (m, 1H), 3.50 (d, J=10 Hz, 1H), 2.70-2.65 (m, 1H), 2.07 (t, J=10 Hz, 1H), 2.03-1.97 (m, 2H), 1.86-1.82 (m, 1H), 1.75-1.67 (m, 1H), 1.31-1.15 (m, 4H), 0.80-0.74 (m, 1H) ppm.

MS (ESI): m/z=[M+1] 623.76.

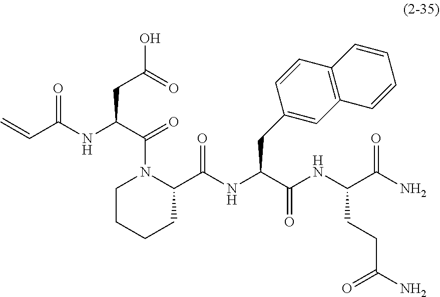 [SMILES: C=CC(=O)N[C@@H](CC(=O)O)C(=O)N1CCCC[C@H]1C(=O)N[C@@H](Cc1ccc2ccccc2c1)C(=O)N[C@@H](CCC(N)=O)C(N)=O]

### (S)-3-Acrylamido-4-((S)-2-(((S)-1-(((S)-1,5-diamino-1,5-dioxopentan-2-yl)amino)-3-(naphthalen-2-yl)-1-oxopropan-2-yl)carbamoyl)piperidin-1-yl)-4-oxobutanoic acid

MS (ESI): m/z=[M+1] 623.80.

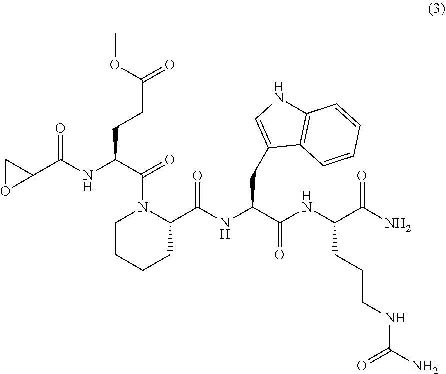 [SMILES: COC(=O)CC[C@H](NC(=O)C1CO1)C(=O)N1CCCC[C@H]1C(=O)N[C@@H](Cc1c[nH]c2ccccc12)C(=O)N[C@@H](CCCNC(N)=O)C(N)=O]

### Methyl (4S)-5-((S)-2-(((S)-1-(((S)-1-amino-1-oxo-5-ureidopentan-2-yl)amino)-3-(1H-indol-3-yl)-1-oxopropan-2-yl)carbamoyl)piperidin-1-yl)-4-(oxirane-2-carboxamido)-5-oxopentanoate (Representative Compound 3)

To a mixture of intermediate 1 (40 mg, 0.065 mmol), HATU (49 mg, 0.13 mmol), and potassium oxirane-2-carboxylate (9.8 mg, 0.078 mmol) in DCM (1 mL) was added N,N-diisopropylethylamine (0.057 mL, 0.33 mmol). The reaction was stirred at room temperature for 3 hrs. The product was then extracted three times with 5 mL of 4:1 chloroform:isopropanol. After washing with water (5 mL) and then brine (5 mL), the solvent was evaporated. The product was isolated via HPLC purification and lyophilized to yield compound 3 as a white powder (13 mg, 29%).

¹H NMR (500 MHz, DMSO-d₆): δ=10.80 (s, 1H), 7.93 (d, J=10 Hz, 1H), 7.59 (d, J=5 Hz, 1H), 7.30 (t, J=5 Hz, 1H), 7.27 (s, 1H), 7.16 (d, J=10 Hz, 1H), 7.13 (s, 1H), 7.05-7.02 (m, 2H), 6.97 (t, J=5 Hz, 1H), 6.56 (s, 1H), 5.95 (s, 1H), 4.96 (s, 1H), 4.79-4.74 (m 1H), 4.65-4.61 (m, 1H), 4.23-4.19 (m, 1H), 3.20-3.16 (m, 2H), 3.04 (s, 3H), 2.91-2.87 (m, 2H), 2.77-2.74 (m, 1H), 2.43-2.40 (m, 1H), 2.34-2.29 (m, 2H), 2.20-2.16 (m, 1H), 2.12-2.09 (m, 1H), 1.87-1.80 (m, 2H), 1.68-1.60 (m, 2H), 1.53-1.44 (m, 2H), 1.43-1.41 (m, 2H), 1.40-1.37 (m, 1H), 1.36-1.30 (m, 3H), 1.24 (s, 2H), 1.16 (s, 1H) ppm.

MS (ESI): m/z=[M+1] 685.88.

To make representative compounds 4 and 5, intermediate 1a was synthesized as described below.

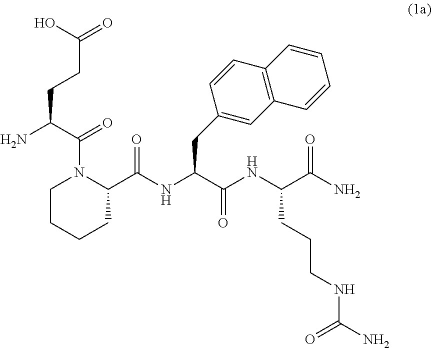 [SMILES: NC(=O)NCCC[C@H](NC(=O)[C@H](Cc1ccc2ccccc2c1)NC(=O)[C@@H]1CCCCN1C(=O)[C@@H](N)CCC(=O)O)C(N)=O]

### (S)-4-Amino-5-((S)-2-(((S)-1-(((S)-1-mino-1-oxo-5-ureidopentan-2-yl)amino)-3-(naphthalen-2-yl)-1-oxopropan-2-yl)carbamoyl)piperidin-1-yl)-5-oxopentanoic acid

Peptide 1a was synthesized according to general solid phase peptide synthesis procedures starting from 0.12 mmol of Rink Amide resin. The following amino acids were coupled to the resin: Fmoc-Glu(OtBu)-OH, Fmoc-pipecolinic acid, Fmoc-2-Nal-OH, Fmoc-Cit-OH.

MS (ESI): m/z=[M+1] 612.88.

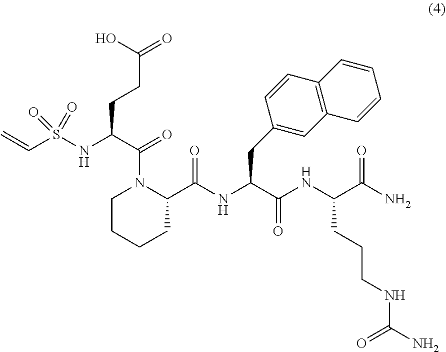 [SMILES: C=CS(=O)(=O)N[C@@H](CCC(=O)O)C(=O)N1CCCC[C@H]1C(=O)N[C@@H](Cc1ccc2ccccc2c1)C(=O)N[C@@H](CCCNC(N)=O)C(N)=O]

### (S)-5-((S)-2-(((S)-1-(((S)-1-Amino-1-oxo-5-ureidopentan-2-yl)amino)-3-(naphthalen-2-yl)-1-oxopropan-2-yl)carbamoyl)piperidin-1-yl)-5-oxo-4(vinylsulfonamido)pentanoic acid

To a solution of 1a (30 mg, 0.05 mmol) in anhydrous DMF (0.5 mL) was added 2-chloroethanesulfonyl chloride (0.008 mL, 0.075 mmol), followed by N,N-diisopropylethylamine (0.035 mL, 0.2 mmol). The resulting solution was stirred at room temperature for 30 minutes. The solution was then diluted with DMSO (2.7 mL) and H₂O (0.8 mL) and the product isolated via HPLC purification and lyophilization to yield compound 4 as a white powder (7 mg, 20%).

MS (ESI): m/z=[M+1] 702.86.

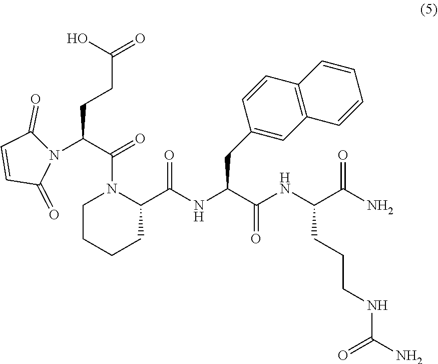 [SMILES: NC(=O)NCCC[C@H](NC(=O)[C@H](Cc1ccc2ccccc2c1)NC(=O)[C@@H]1CCCCN1C(=O)[C@H](CCC(=O)O)N1C(=O)C=CC1=O)C(N)=O]

### (S)-5-((S)-2-(((S)-1-(((S)-1-Amino-1-oxo-5-ureidopentan-2-yl)amino)-3-(naphthalen-2-yl)-1-oxopropan-2-yl)carbamoyl)piperidin-1-yl)-4-(2,5-dioxo-2,5-dihydro-1H-pyrrol-1-yl)-5-oxopentanoic acid

To a solution of 1a (30 mg, 0.05 mmol) in DMF (0.25 mL) was added N,N-diisopropylethylamine (0.035 mL, 0.2 mmol). Methyl 2,5-dioxo-2,5-dihydro-1H-pyrrole-1-carboxylate (8 mg, 0.05 mmol) dissolved in DMF (0.25 mL) was then added to the solution, and the reaction mixture was stirred for 3 hrs at 0° C. The solution was then diluted with DMSO (2.7 mL) and H₂O (0.8 mL) and the product isolated via HPLC purification and lyophilization to yield compound 5 as a white powder (9 mg, 26%).

¹H NMR (500 MHz, DMSO-d₆): δ=12.19 (s, 1H), 8.04 (d, J=5 Hz, 1H), 7.85-7.80 (m, 2H), 7.74 (s, 1H), 7.47-7.44 (m, 2H), 7.33 (s, 1H), 7.19 (s, 1H), 7.09 (s, 1H), 7.06 (s, 1H), 6.99 (s, 1H), 6.53 (s, 1H), 5.94 (t, J=5 Hz, 1H), 5.39 (s, 2H), 4.83-4.76 (m, 2H), 4.26-4.22 (m, 1H), 3.15-3.13 (m, 1H), 3.06-3.02 (m, 1H), 2.99-2.93 (m, 2H), 2.27-2.21 (m, 2H), 2.04-1.98 (m, 2H), 1.70-1.64 (m, 2H), 1.55-1.49 (m, 1H), 1.41-1.34 (m, 2H), 1.28-1.24 (m, 2H), 1.16-1.10 (m, 2H), 1.03-0.98 (m, 2H), 0.98-0.88 (m, 2H), 0.84-0.79 (m, 1H) ppm.

MS (ESI): m/z=[M+1] 692.99.

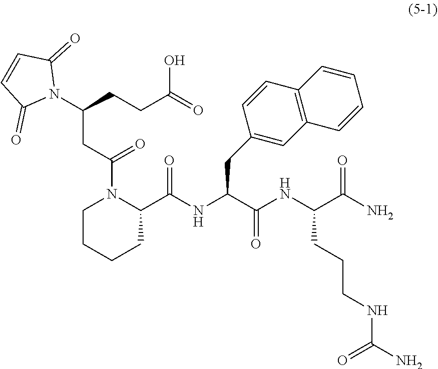 [SMILES: NC(=O)NCCC[C@H](NC(=O)[C@H](Cc1ccc2ccccc2c1)NC(=O)[C@@H]1CCCCN1C(=O)C[C@H](CCC(=O)O)N1C(=O)C=CC1=O)C(N)=O]

### (S)-6-((S)-2-(((S)-1-(((S)-1-Amino-1-oxo-5-ureidopentan-2-yl)amino)-3-(naphthalen-2-yl)-1-oxopropan-2-yl)carbamoyl)piperidin-1-yl)-4-(2,5-dioxo-2,5-dihydro-1H-pyrrol-1-yl)-6-oxohexanoic acid

MS (ESI): m/z=[M+1] 706.89.

### Example 2: Identification and Characterization of Compounds Synthesized According to Scheme 1a

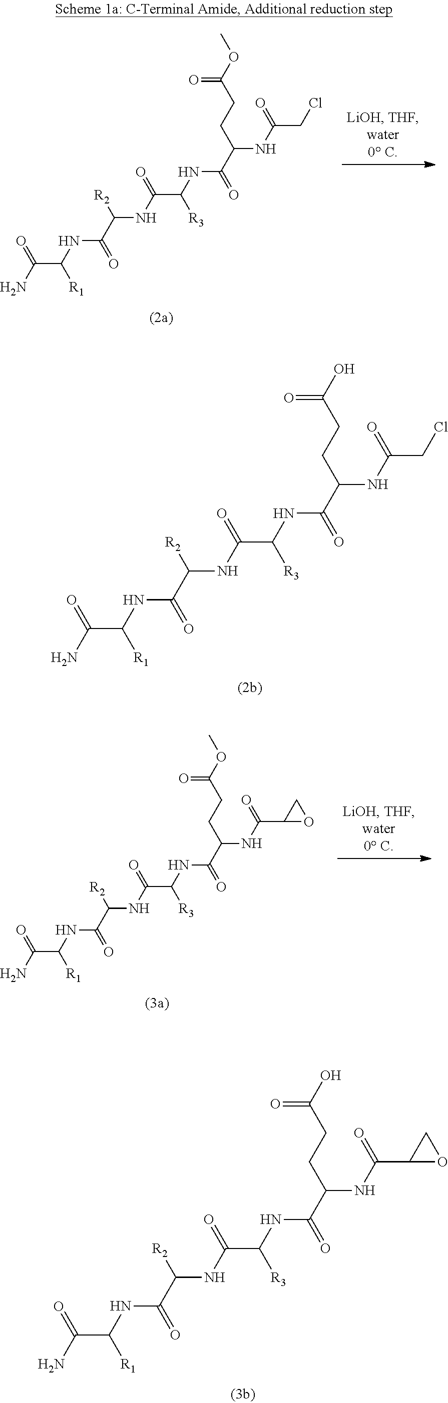

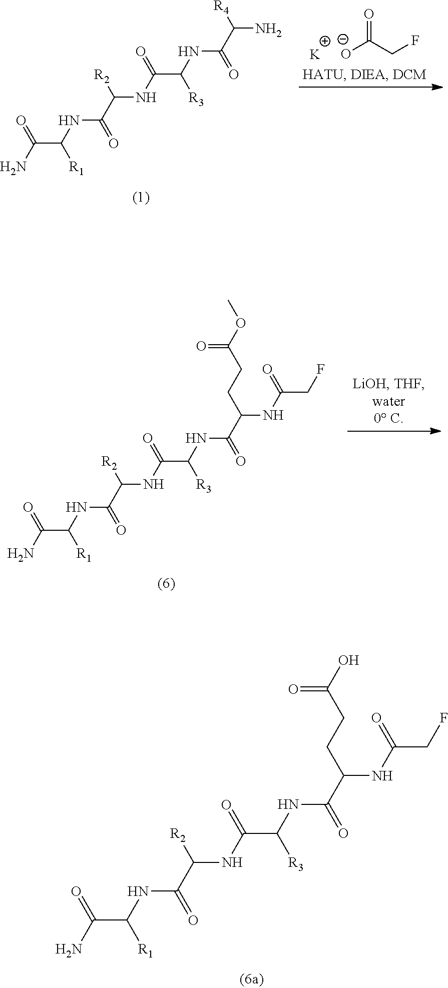

*2a, 3a, and 1 were synthesized according to Scheme 1, where R₄=Glu(Ome)

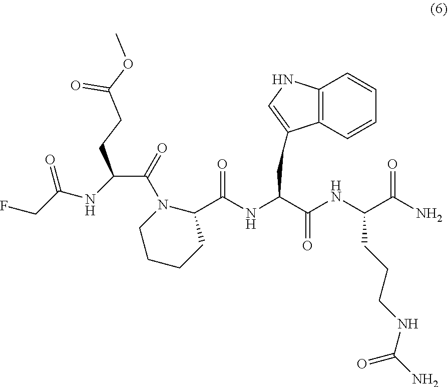 [SMILES: COC(=O)CC[C@H](NC(=O)CF)C(=O)N1CCCC[C@H]1C(=O)N[C@@H](Cc1c[nH]c2ccccc12)C(=O)N[C@@H](CCCNC(N)=O)C(N)=O]

### Methyl (S)-5-((S)-2-(((S)-1-(((S)-1-amino-1-oxo-5-ureidopentan-2-yl)amino)-3-(1H-indol-3-yl)-1-oxopropan-2-yl)carbamoyl)piperidin-1-yl)-4-(2-fluoroacetamido)-5-oxopentanoate (intermediate 6)

To a solution of intermediate 1 (49 mg, 0.08 mmol) in DCM (0.5 mL), was added HATU (59 mg, 0.16 mmol) and potassium fluoroacetate (10 mg, 0.09 mmol), followed by N,N-diisopropylethylamine (0.06 mL, 0.4 mmol). The resulting reaction mixture was stirred at room temperature for 4 hours. The product was then extracted three times with 5 mL of 4:1 chloroform:isopropanol. After washing with water (5 mL) and then brine (5 mL), the solvent was evaporated. The product was isolated via HPLC purification and lyophilized to yield 6 as a white powder (35 mg, 65%).

MS (ESI): m/z [M+1] 675.87.

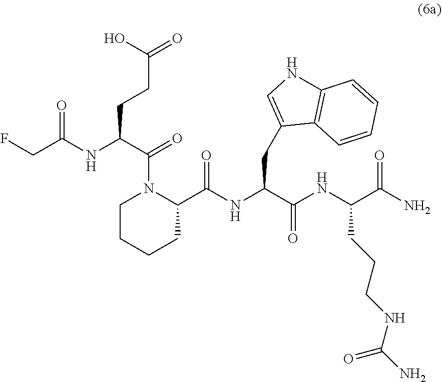 [SMILES: NC(=O)NCCC[C@H](NC(=O)[C@H](Cc1c[nH]c2ccccc12)NC(=O)[C@@H]1CCCCN1C(=O)[C@H](CCC(=O)O)NC(=O)CF)C(N)=O]

### (S)-5-((S)-2-(((S)-1-(((S)-1-Amino-1-oxo-5-ureidopentan-2-yl)amino)-3-(1H-indol-3-yl)-1-oxopropan-2-yl)carbamoyl)piperidin-1-yl)-4-(2-fluoroacetamido)-5-oxopentanoic acid

To a mixture of 6 (25 mg, 0.04 mmol) in THF/water (4:1, 0.5 mL), was added LiOH (1.9 mg, 0.08 mmol). The resulting mixture was stirred at 0° C. in an ice bath for 3 hours. The mixture was then neutralized with 1 N HCl (1.5 equivalents). The solvent was then evaporated and the product directly purified via HPLC purification and lyophilized to yield compound 6a as a white powder (20 mg, 75%).

¹H NMR (500 MHz, DMSO-d₆): δ=12.15 (s, 1H), 10.72 (s, 1H), 8.53-8.40 (m, 1H), 8.19-8.14 (m, 2H), 7.86 (d, J=5 Hz, 2H), 7.59-7.51 (m, 3H), 7.25-7.18 (m, 3H), 7.06 (s, 1H), 6.99-6.89 (m, 3H), 6.47 (s, 2H), 5.88 (s, 1H), 5.33 (s, 3H), 4.89-4.78 (m, 1H), 4.73-4.66 (m, 1H), 4.60-4.50 (m, 1H), 4.17-4.11 (m, 1H), 2.98-2.83 (m, 2H), 2.27-2.16 (m, 1H), 1.84-1.72 (m, 1H), 1.62-1.55 (m, 2H), 1.44-1.24 (m, 6H), 1.17 (s, 2H) ppm.

MS (ESI): m/z=[M+1] 661.91.

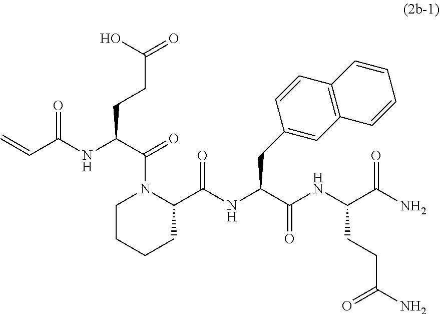 [SMILES: C=CC(=O)N[C@@H](CCC(=O)O)C(=O)N1CCCC[C@H]1C(=O)N[C@@H](Cc1ccc2ccccc2c1)C(=O)N[C@@H](CCC(N)=O)C(N)=O]

### (S)-4-Acrylamido-5-((S)-2-(((S)-1-(((S)-1,5-diamino-1,5-dioxopentan-2-yl)amino)-3-(naphthalen-2-yl)-1-oxopropan-2-yl)carbamoyl)piperidin-1-yl)-5-oxopentanoic acid

¹H NMR (500 MHz, DMSO-d₆): δ=12.07 (s, 1H), 8.41-8.32 (m, 1H), 8.12 (t, J=5 Hz, 1H), 7.95 (d, J=5 Hz, 1H), 7.84 (d, J=10 Hz, 1H), 7.78 (d, J=5 Hz, 1H), 7.71 (t, J=5 Hz, 1H), 7.54-7.44 (m, 2H), 7.35 (d, J=10 Hz, 1H), 7.27-7.16 (m, 3H), 7.05-6.95 (m, 3H), 6.70 (s, 1H), 6.32-6.20 (m, 1H), 6.11-6.01 (m, 1H), 5.59-5.53 (m, 1H), 4.88 (s, 1H), 4.74 (s, 1H), 4.74 (t, J=5 Hz, 1H), 4.67 (t, J=5 Hz, 1H), 4.13 (t, J=10 Hz, 1H), 3.63-3.52 (m, 2H), 2.66 (t, J=5 Hz, 1H), 2.30 (t, J=5 Hz, 1H), 2.19 (t, J=5 Hz, 1H), 2.08-1.96 (m, 2H), 1.87-1.82 (m, 1H), 1.76-1.59 (m, 2H), 1.36-0.98 (m, 5H) ppm.

MS (ESI): m/z=[M+1] 637.82.

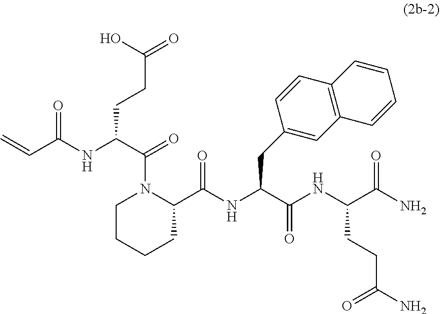 [SMILES: C=CC(=O)N[C@H](CCC(=O)O)C(=O)N1CCCC[C@H]1C(=O)N[C@@H](Cc1ccc2ccccc2c1)C(=O)N[C@@H](CCC(N)=O)C(N)=O]

### (R)-4-Acrylamido-5-((S)-2-(((S)-1-(((S)-1,5-diamino-1,5-dioxopentan-2-yl)amino)-3-(naphthalen-2-yl)-1-oxopropan-2-yl)carbamoyl)piperidin-1-yl)-5-oxopentanoic acid

¹H NMR (500 MHz, DMSO-d₆): δ=12.07 (s, 1H), 8.53 (d, J=5 Hz, 1H), 8.12 (t, J=5 Hz, 1H), 7.85 (d, J=5 Hz, 1H), 7.74-7.64 (m, 2H), 7.53-7.44 (m, 2H), 7.38-7.30 (m, 2H), 7.18-7.10 (m, 4H), 7.06-6.96 (m, 4H), 6.48 (s, 1H), 6.32-6.21 (m, 1H), 6.13-6.04 (m, 1H), 5.57 (d, J=10 Hz, 1H), 4.89 (s, 1H), 4.73 (s, 1H), 4.67-4.63 (m, 2H), 4.15-4.09 (m, 2H), 3.72-3.56 (m, 2H), 2.72 (t, J=5 Hz, 1H), 1.99 (t, J=10 Hz, 1H), 1.86-1.79 (m, 1H), 1.73-1.64 (m, 2H), 1.33-1.17 (m, 4H), 0.81-0.71 (m, 1H) ppm.

MS (ESI): m/z=[M+1] 637.78.

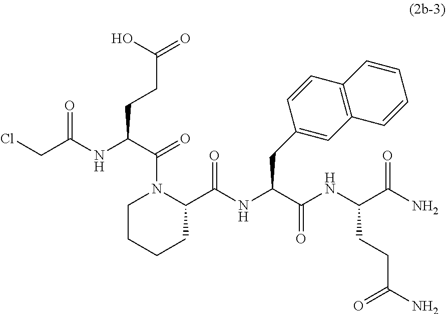 [SMILES: NC(=O)CC[C@H](NC(=O)[C@H](Cc1ccc2ccccc2c1)NC(=O)[C@@H]1CCCCN1C(=O)[C@H](CCC(=O)O)NC(=O)CCl)C(N)=O]

### (S)-4-(2-Chloroacetamido)-5-((S)-2-(((S)-1-(((S)-1,5-diamino-1,5-dioxopentan-2-yl)amino)-3-(naphthalen-2-yl)-1-oxopropan-2-yl)carbamoyl)piperidin-1-yl)-5-oxopentanoic acid

¹H NMR (500 MHz, DMSO-d₆): δ=12.12 (s, 1H), 8.42 (d, J=5 Hz, 1H), 8.13 (d, J=10 Hz, 1H), 7.95-7.90 (m, 1H), 7.85-7.80 (m, 2H), 7.71 (t, J=5 Hz, 1H), 7.51 (t, J=5 Hz, 1H), 7.45 (t, J=5 Hz, 1H), 7.34 (d, J=5 Hz, 1H), 7.31-7.26 (m, 1H), 7.20-7.09 (m, 2H), 7.07-6.96 (m, 3H), 6.70 (s, 1H), 6.48 (s, 1H), 4.88 (s, 1H), 4.72-4.62 (m, 2H), 4.15-4.08 (m, 1H), 4.03 (d, J=5 Hz, 1H), 3.61-3.52 (m, 2H), 2.75-2.69 (m, 1H), 2.19 (t, J=5 Hz, 1H), 2.04-1.97 (m, 3H), 1.87-1.78 (m, 2H), 1.72-1.68 (m, 1H), 1.60-1.56 (m, 1H), 1.37-1.17 (m, 4H), 0.99-0.93 (m, 1H) ppm.

MS (ESI): m/z=[M+1] 659.64.

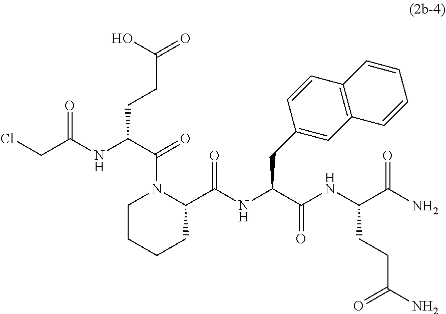 [SMILES: NC(=O)CC[C@H](NC(=O)[C@H](Cc1ccc2ccccc2c1)NC(=O)[C@@H]1CCCCN1C(=O)[C@@H](CCC(=O)O)NC(=O)CCl)C(N)=O]

### (R)-4-(2-Chloroacetamido)-5-((S)-2-(((S)-1-(((S)-1,5-diamino-1,5-dioxopentan-2-yl)amino)-3-(naphthalen-2-yl)-1-oxopropan-2-yl)carbamoyl)piperidin-1-yl)-5-oxopentanoic acid

¹H NMR (500 MHz, DMSO-d₆): δ=12.11 (s, 1H), 8.45 (d, J=10 Hz, 1H), 8.13 (t, J=5 Hz, 1H), 7.84 (t, J=5 Hz, 2H), 7.71 (t, J=5 Hz, 2H), 7.52 (t, J=5 Hz, 1H), 7.45 (t, J=5 Hz, 1H), 7.35-7.32 (m, 2H), 7.16 (s, 2H), 7.06 (s, 2H), 6.95 (s, 2H), 6.69 (s, 1H), 6.48 (s, 1H), 4.36 (s, 1H), 4.76-4.63 (m, 2H), 4.13-4.05 (m, 3H), 3.59-3.53 (m, 1H), 2.68 (t, J=10 Hz, 1H), 2.20-2.18 (m, 2H), 2.04-2.01 (m, 2H), 1.86-1.79 (m, 1H), 1.74-1.68 (m, 1H), 1.65-1.55 (m, 1H), 1.36-1.18 (m, 4H), 1.09-0.95 (m, 1H) ppm.

MS (ESI): m/z=[M+1] 659.08.

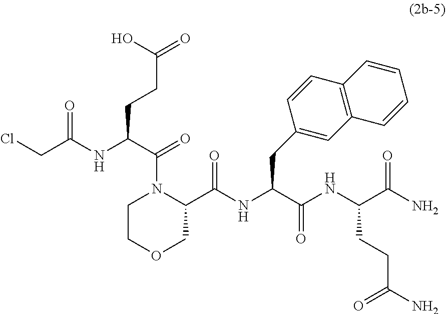 [SMILES: NC(=O)CC[C@H](NC(=O)[C@H](Cc1ccc2ccccc2c1)NC(=O)[C@@H]1COCCN1C(=O)[C@H](CCC(=O)O)NC(=O)CCl)C(N)=O]

### (S)-4-(2-Chloroacetamido)-5-((S)-3-(((S)-1-(((S)-1,5-diamino-1,5-dioxopentan-2-yl)amino)-3-(naphthalen-2-yl)-1-oxopropan-2-yl)carbamoyl)morpholino)-5-oxopentanoic acid

MS (ESI): m/z=[M+1] 661.33.

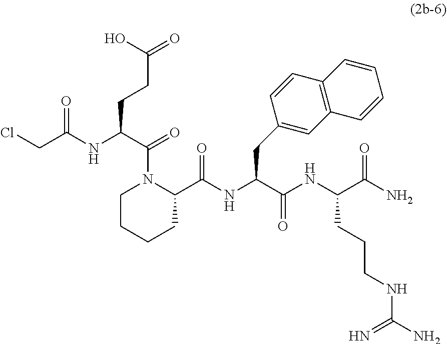 [SMILES: N=C(N)NCCC[C@H](NC(=O)[C@H](Cc1ccc2ccccc2c1)NC(=O)[C@@H]1CCCCN1C(=O)[C@H](CCC(=O)O)NC(=O)CCl)C(N)=O]

### (S)-5-((S)-2-(((S)-1-(((S)-1-Amino-5-guanidino-1-oxopentan-2-yl)amino)-3-(naphthalen-2-yl)-1-oxopropan-2-yl)carbamoyl)piperidin-1-yl)-4-(2-chloroacetamido)-5-oxopentanoic acid

¹H NMR (500 MHz, DMSO-d₆): δ=12.13 (s, 1H), 8.39 (d, J=5 Hz, 1H), 8.08 (d, J=10 Hz, 1H), 7.79-7.74 (m, 3H), 7.70-7.67 (m, 1H), 7.46 (t, J=5 Hz, 1H), 7.39 (t, J=5 Hz, 3H), 7.29-7.27 (m, 1H), 7.06 (d, J=5 Hz, 1H), 6.80 (s, 1H), 4.88 (d, J=5 Hz, 1H), 4.69-4.65 (m, 2H), 4.19-4.15 (m, 1H), 4.11-4.04 (m, 1H), 4.01 (s, 1H), 3.57 (d, J=10 Hz, 1H), 3.23-3.16 (m, 1H), 3.04 (d, J=10 Hz, 2H), 2.99-2.95 (m, 1H), 2.78-2.72 (m, 1H), 2.23 (t, J=5 Hz, 1H), 2.16 (t, J=5 Hz, 1H), 2.03-1.97 (m, 2H), 1.81-1.72 (m, 1H), 1.66-1.62 (m, 1H), 1.59-1.37 (m, 4H), 1.32-1.25 (m, 3H), 1.18-1.12 (m, 2H), 1.01-0.92 (m, 2H) ppm.

MS (ESI): m/z=[M+1] 687.14.

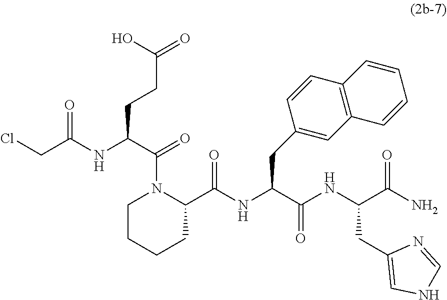 [SMILES: NC(=O)[C@H](Cc1c[nH]cn1)NC(=O)[C@H](Cc1ccc2ccccc2c1)NC(=O)[C@@H]1CCCCN1C(=O)[C@H](CCC(=O)O)NC(=O)CCl]

### (S)-5-((S)-2-(((S)-1-(((S)-1-Amino-3-(1H-imidazol-4-yl)-1-oxopropan-2-yl)amino)-3-(naphthalen-2-yl)-1-oxopropan-2-yl)carbamoyl)piperidin-1-yl)-4-(2-chloroacetamido)-5-oxopentanoic acid

MS (ESI): m/z=[M+1] 668.08.

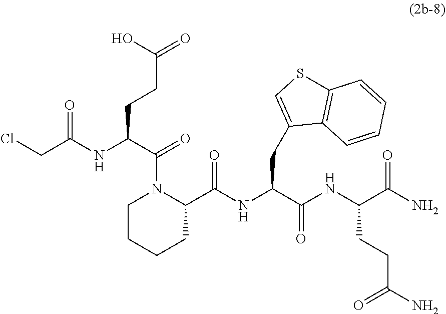 [SMILES: NC(=O)CC[C@H](NC(=O)[C@H](Cc1csc2ccccc12)NC(=O)[C@@H]1CCCCN1C(=O)[C@H](CCC(=O)O)NC(=O)CCl)C(N)=O]

### (S)-5-((S)-2-(((S)-3-(Benzo[6]thiophen-3-yl)-1-(((S)-1,5-diamino-1,5-dioxopentan-2-yl)amino)-1-oxopropan-2-yl)carbamoyl)piperidin-1-yl)-4-(2-chloroacetamido)-5-oxopentanoic acid

MS (ESI): m/z=[M+1] 665.08.

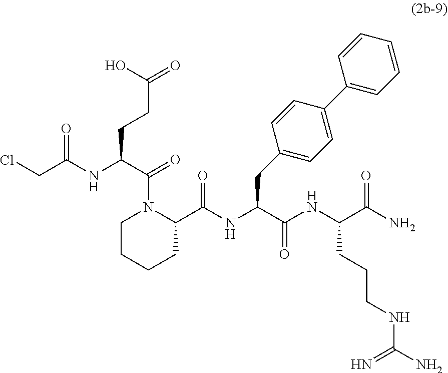 [SMILES: N=C(N)NCCC[C@H](NC(=O)[C@H](Cc1ccc(-c2ccccc2)cc1)NC(=O)[C@@H]1CCCCN1C(=O)[C@H](CCC(=O)O)NC(=O)CCl)C(N)=O]

### (S)-5-((S)-2-(((S)-3-([1,1′-Biphenyl]-4-yl)-1-(((S)-1-amino-5-guanidino-1-oxopentan-2-yl)amino)-1-oxopropan-2-yl)carbamoyl)piperidin-1-yl)-4-(2-chloroacetamido)-5-oxopentanoic acid

¹H NMR (500 MHz, DMSO-d₆): δ=12.10 (s, 1H), 8.42 (d, J=10 Hz, 1H), 8.06 (d, J=5 Hz, 1H), 7.70 (d, J=10 Hz, 1H), 7.56 (t, J=5 Hz, 3H), 7.48 (t, J=5 Hz, 3H), 7.38 (t, J=5 Hz, 2H), 7.30-7.26 (m, 4H), 7.17 (s, 1H), 7.06 (s, 2H), 6.96 (s, 1H), 4.90 (d, J=10 Hz, 1H), 4.74-4.69 (m, 1H), 4.63-4.55 (m, 1H), 4.19-4.16 (m, 1H), 4.11-4.05 (m, 1H), 4.03 (d, J=5 Hz, 1H), 3.05 (d, J=5 Hz, 3H), 2.89-2.81 (m, 1H), 2.72-2.67 (m, 1H), 2.20 (t, J=5 Hz, 1H), 2.06-1.97 (m, 2H), 1.88-1.83 (m, 1H), 1.67-1.59 (m, 2H), 1.50-1.37 (m, 3H), 1.30-1.27 (m, 2H), 1.21-1.15 (m, 2H), 1.11-1.05 (m, 1H) ppm.

MS (ESI): m/z=[M+1] 713.10.

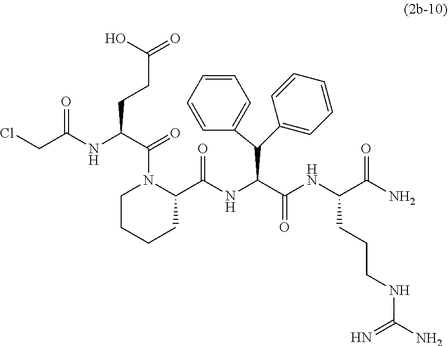 [SMILES: N=C(N)NCCC[C@H](NC(=O)[C@@H](NC(=O)[C@@H]1CCCCN1C(=O)[C@H](CCC(=O)O)NC(=O)CCl)C(c1ccccc1)c1ccccc1)C(N)=O]

### (S)-5-((S)-2-(((S)-1-(((S)-1-Amino-5-guanidino-1-oxopentan-2-yl)amino)-1-oxo-3,3-diphenylpropan-2-yl)carbamoyl)piperidin-1-yl)-4-(2-chloroacetamido)-5-oxopentanoic acid

¹H NMR (500 MHz, DMSO-d₆): δ=12.20 (s, 1H), 8.45 (d, J=5 Hz, 1H), 8.07 (d, J=5 Hz, 1H), 7.70 (d, J=5 Hz, 1H), 7.41 (d, J=5 Hz, 2H), 7.31-7.28 (m, 4H), 7.20-7.15 (m, 5H), 7.08-7.05 (m, 2H), 6.96 (s, 1H), 6.83 (s, 1H), 6.02 (s, 1H), 5.32 (t, J=10 Hz, 1H), 4.81 (s, 1H), 4.65-4.61 (m, 1H), 4.41 (d, J=10 Hz, 1H), 4.16-4.09 (m, 1H), 4.01 (d, J=5 Hz, 1H), 3.90-3.88 (m, 1H), 3.40-3.33 (m, 1H), 2.95 (t, J=5 Hz, 2H), 2.21-2.19 (m, 2H), 2.02-1.98 (m, 1H), 1.83-1.79 (m, 1H), 1.68-1.54 (m, 2H), 1.35-1.26 (m, 3H), 1.25-1.15 (m, 3H), 1.12-105 (m, 2H), 0.97-0.90 (m, 1H) ppm.

MS (ESI): m/z=[M+1] 713.20.

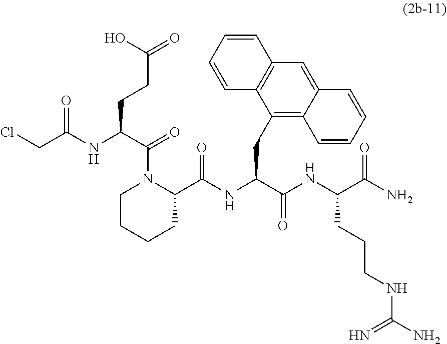 [SMILES: N=C(N)NCCC[C@H](NC(=O)[C@H](Cc1c2ccccc2cc2ccccc12)NC(=O)[C@@H]1CCCCN1C(=O)[C@H](CCC(=O)O)NC(=O)CCl)C(N)=O]

### (S)-5-((S)-2-(((S)-1-(((S)-1-Amino-5-guanidino-1-oxopentan-2-yl)amino)-3-(anthracen-9-yl)-1-oxopropan-2-yl)carbamoyl)piperidin-1-yl)-4-(2-chloroacetamido)-5-oxopentanoic acid

MS (ESI): m/z=[M+1] 737.20.

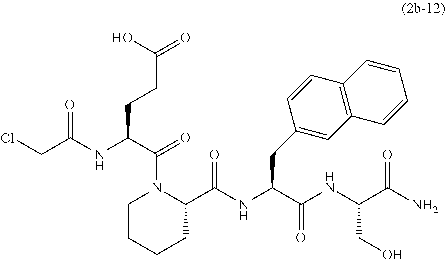 [SMILES: NC(=O)[C@H](CO)NC(=O)[C@H](Cc1ccc2ccccc2c1)NC(=O)[C@@H]1CCCCN1C(=O)[C@H](CCC(=O)O)NC(=O)CCl]

### (S)-5-((S)-2-(((S)-1-(((S)-1-Amino-3-hydroxy-1-oxopropan-2-yl)amino)-3-(naphthalen-2-yl)-1-oxopropan-2-yl)carbamoyl)piperidin-1-yl)-4-(2-chloroacetamido)-5-oxopentanoic acid

¹H NMR (500 MHz, DMO-d₆): δ=12.10 (s, 1H), 8.39 (d, J=5 Hz, 1H), 8.05-7.89 (m, 1H), 7.78-7.65 (m, 6H), 7.40-7.37 (m, 3H), 7.18 (d, J=10 Hz, 1H), 7.14-7.04 (m, 2H), 6.93 (s, 1H), 4.88-4.87 (m, 1H), 4.69-4.55 (m, 2H), 4.17-4.12 (m, 1H), 4.01 (s, 2H), 3.28-3.14 (m, 2H), 3.01-2.95 (m, 1H), 2.86-2.74 (m, 1H), 2.16 (t, J=5 Hz, 2H), 2.06-1.99 (m, 1H), 1.82-1.64 (m, 1H), 1.57-1.53 (m, 1H), 1.34-1.24 (m, 3H), 1.19-1.11 (m, 1H), 1.06-0.92 (m, 1H) ppm.

MS (ESI): m/z=[M+1] 618.02.

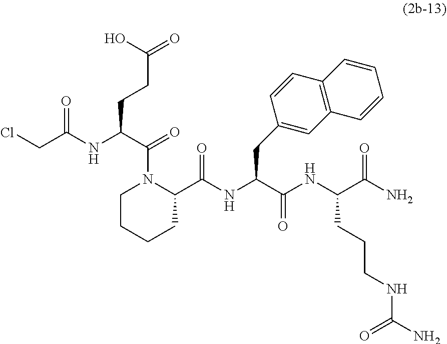 [SMILES: NC(=O)NCCC[C@H](NC(=O)[C@H](Cc1ccc2ccccc2c1)NC(=O)[C@@H]1CCCCN1C(=O)[C@H](CCC(=O)O)NC(=O)CCl)C(N)=O]

### (S)-5-((S)-2-(((S)-1-(((S)-1-Amino-1-oxo-5-ureidopentan-2-yl)amino)-3-(naphthalen-2-yl)-1-oxopropan-2-yl)carbamoyl)piperidin-1-yl)-4-(2-chloroacetamido)-5-oxopentanoic acid

MS (ESI): m/z=[M+1] 688.84.

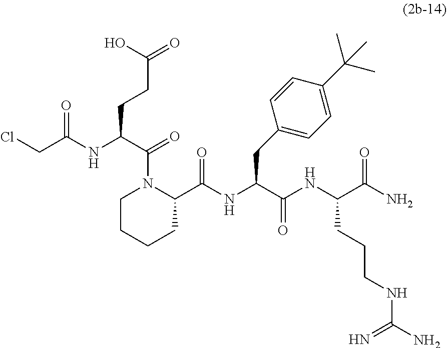 [SMILES: CC(C)(C)c1ccc(C[C@H](NC(=O)[C@@H]2CCCCN2C(=O)[C@H](CCC(=O)O)NC(=O)CCl)C(=O)N[C@@H](CCCNC(=N)N)C(N)=O)cc1]

### (S)-5-((S)-2-(((S)-1-(((S)-1-Amino-5-guanidino-1-oxopentan-2-yl)amino)-3-(4-(tert-butyl)phenyl)-1-oxopropan-2-yl)carbamoyl)piperidin-1-yl)-4-(2-chloroacetamido)-5-oxopentanoic acid

¹H NMR (500 MHz, DMSO-d₆): δ=12.18 (s, 1H), 8.51 (d, J=5 Hz, 1H), 8.10 (d, J=10 Hz, 1H), 7.62 (d, J=10 Hz, 1H), 7.55-7.49 (m, 1H), 7.30-7.28 (m, 2H), 7.27-7.25 (m, 1H), 7.22-7.18 (m, 2H), 7.13-7.11 (m, 1H), 7.01 (s, 1H), 4.95 (d, J=5 Hz, 1H), 4.77-4.74 (m, 1H), 4.65-4.61 (m, 1H), 4.25-4.21 (m, 1H), 4.19-4.12 (m, 1H), 4.10 (d, J=5 Hz, 1H), 3.31 (t, J=5 Hz, 3H), 3.13-3.09 (m, 2H), 3.07-3.03 (m, 1H), 2.83-2.78 (m, 1H), 2.70 (s, 2H), 2.31-2.27 (m, 2H), 2.18 (t, J=5 Hz, 2H), 2.10-2.04 (m, 1H), 1.94-1.88 (m, 3H), 1.76-1.58 (m, 2H), 1.58-1.52 (m, 1H), 1.45-1.38 (m, 1H), 1.25 (s, 9H), 1.11-1.04 (m, 1H) ppm.

MS (ESI): m/z=[M+1] 693.94.

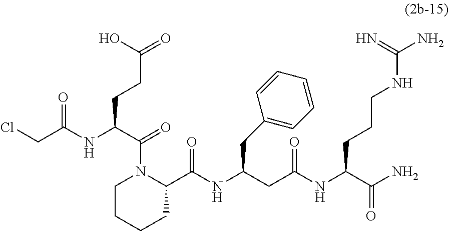 [SMILES: N=C(N)NCCC[C@H](NC(=O)C[C@H](Cc1ccccc1)NC(=O)[C@@H]1CCCCN1C(=O)[C@H](CCC(=O)O)NC(=O)CCl)C(N)=O]

### (S)-5-((S)-2-(((S)-4-(((S)-1-Amino-5-guanidino-1-oxopentan-2-yl)amino)-4-oxo-1-phenylbutan-2-yl)carbamoyl)piperidin-1-yl)-4-(2-chloroacetamido)-5-oxopentanoic acid

MS (ESI): m/z=[M+1] 651.86.

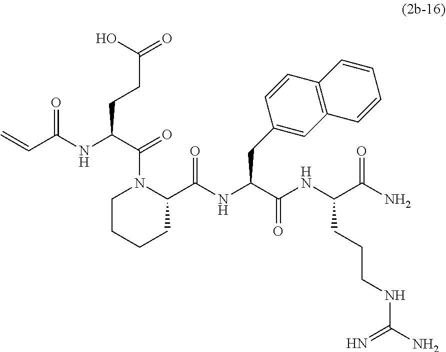 [SMILES: C=CC(=O)N[C@@H](CCC(=O)O)C(=O)N1CCCC[C@H]1C(=O)N[C@@H](Cc1ccc2ccccc2c1)C(=O)N[C@@H](CCCNC(=N)N)C(N)=O]

### (S)-4-Acrylamido-5-((S)-2-(((S)-1-(((S)-1-amino-5-guanidino-1-oxopentan-2-yl)amino)-3-(naphthalen-2-yl)-1-oxopropan-2-yl)carbamoyl)piperidin-1-yl)-5-oxopentanoic acid

MS (ESI): m/z=[M+1] 665.82.

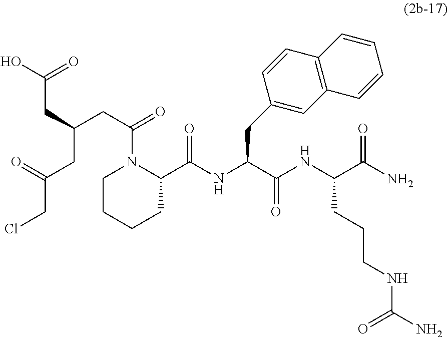 [SMILES: NC(=O)NCCC[C@H](NC(=O)[C@H](Cc1ccc2ccccc2c1)NC(=O)[C@@H]1CCCCN1C(=O)C[C@H](CC(=O)O)CC(=O)CCl)C(N)=O]

### (R)-5-((S)-2-(((S)-1-(((S)-1-Amino-1-oxo-5-ureidopentan-2-yl)amino)-3-(naphthalen-2-yl)-1-oxopropan-2-yl)carbamoyl)piperidin-1-yl)-3-(2-chloroacetamido)-5-oxopentanoic acid

MS (ESI): m/z=[M+1] 688.79.

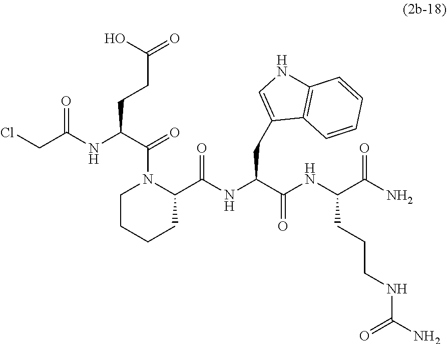 [SMILES: NC(=O)NCCC[C@H](NC(=O)[C@H](Cc1c[nH]c2ccccc12)NC(=O)[C@@H]1CCCCN1C(=O)[C@H](CCC(=O)O)NC(=O)CCl)C(N)=O]

### (S)-5-((S)-2-(((S)-1-(((S)-1-Amino-1-oxo-5-ureidopentan-2-yl)amino)-3-(1H-indol-3-yl)-1-oxopropan-2-yl)carbamoyl)piperidin-1-yl)-4-(2-chloroacetamido)-5-oxopentanoic acid

MS (ESI): m/z=[M+1] 677.73.

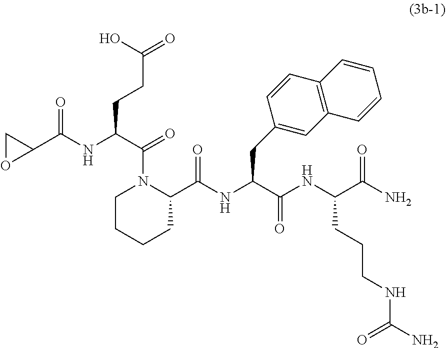 [SMILES: NC(=O)NCCC[C@H](NC(=O)[C@H](Cc1ccc2ccccc2c1)NC(=O)[C@@H]1CCCCN1C(=O)[C@H](CCC(=O)O)NC(=O)C1CO1)C(N)=O]

### (4S)-5-((S)-2-(((S)-1-(((S)-1-Amino-1-oxo-5-ureidopentan-2-yl)amino)-3-(naphthalen-2-yl)-1-oxopropan-2-yl)carbamoyl)piperidin-1-yl)-4-(oxirane-2-carboxamido)-5-oxopentanoic acid

MS (ESI): m/z=[M+1] 682.58.

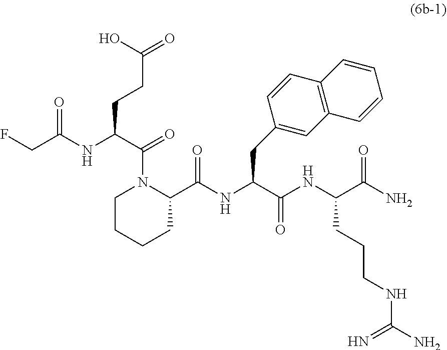 [SMILES: N=C(N)NCCC[C@H](NC(=O)[C@H](Cc1ccc2ccccc2c1)NC(=O)[C@@H]1CCCCN1C(=O)[C@H](CCC(=O)O)NC(=O)CF)C(N)=O]

### (S)-5-((S)-2-(((S)-1-(((S)-1-Amino-5-guanidino-1-oxopentan-2-yl)amino)-3-(naphthalen-2-yl)-1-oxopropan-2-yl)carbamoyl)piperidin-1-yl)-4-(2-fluoroacetamido)-5-oxopentanoic acid

MS (ESI): m/z=[M+1] 671.87.

### Example 3: Identification and Characterization of Compounds Synthesized According to Scheme 1b

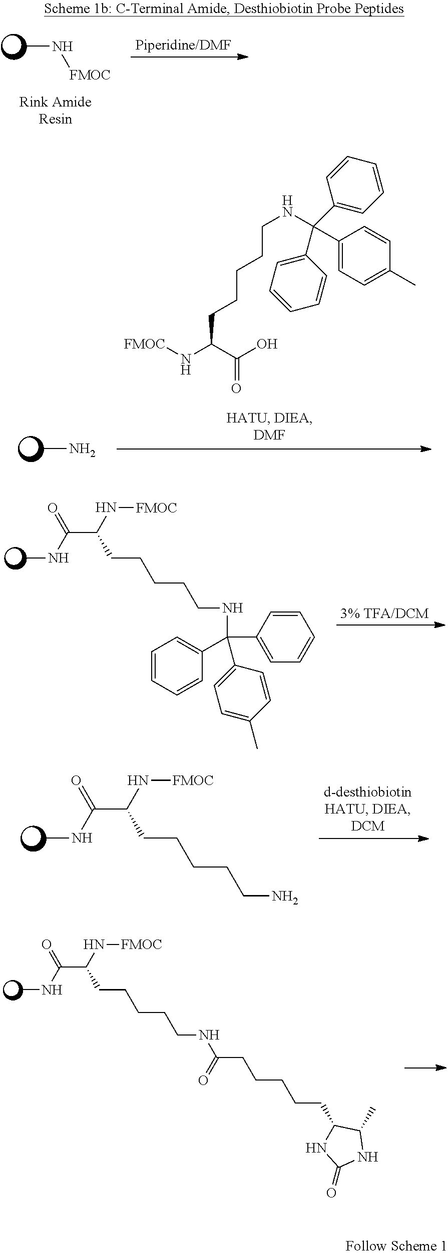

Lys(MTT) Deprotection on Rink Amide Resin: The resin was suspended in DCM (1 mL per 100 mg of resin) in a Poly-Prep column, and gently agitated with nitrogen gas. The DCM was drained by vacuum filtration, and then the resin suspended in 3% TFA in DCM (1 mL per 100 mg of resin), and agitated with nitrogen gas for 10 minutes. The solution was drained by vacuum filtration, and then more 3% TFA in DCM was added, and the resin agitated for another 10 minutes. After draining the solution via vacuum filtration, the resin was washed 2× with DCM, 2× with MeOH, 2× with DCM, 1× with 1% DIPEA in DMF, and then 2× with DMF.

D-Desthiobiotin Coupling to Lys on Rink Amide Resin: To a solution of D-desthiobiotin (10 equivalents relative to resin) in 1:1 DMF:DMSO (0.5 mL volume per 100 mg resin), was added HATU (10 equivalents relative to resin) and DIPEA (26 equivalents relative to resin) in DMF (1 mL volume per 400 mg resin). This solution was then added to the resin, which was agitated with nitrogen gas for 6 hours. After draining the solution via vacuum filtration, the resin was washed 2× with DMF, 2× with DCM, and 2× with MeOH.

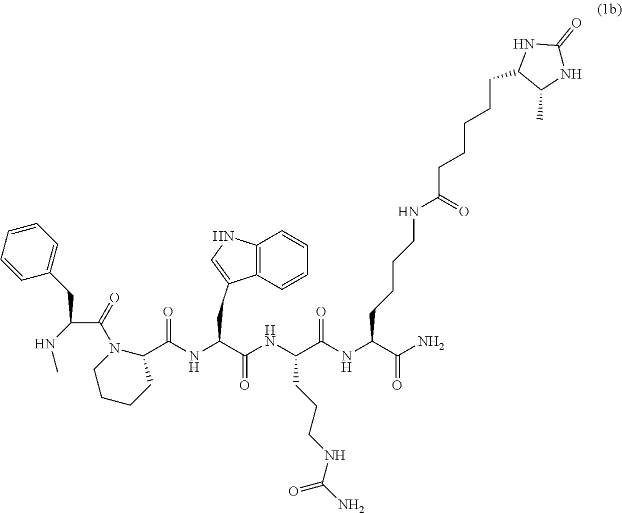

### (S)—N—((S)-1-(((S)-1-(((S)-1-Amino-6-(6-((4S,5R)-5-methyl-2-oxoimidazolidin-4-yl)hexanamido)-1-oxohexan-2-yl)amino)-1-oxo-5-ureidopentan-2-yl)amino)-3-(1H-indol-3-yl)-1-oxopropan-2-yl)-1-(methyl-L-phenylalanyl)piperidine-2-carboxamide (1b)

Peptide 1b was synthesized according to general solid phase peptide synthesis procedures starting from 0.12 mmol of Rink Amide resin (Scheme 1). The following amino acids were coupled to the resin: Fmoc-N-Me-Phe-OH, Fmoc-pipecolinic acid, Fmoc-Trp(BOC)-OH, Fmoc-Cit-OH, Fmoc-Lys(MTT)-OH.

¹H NMR (500 MHz, DMSO-d₆): δ=10.71 (d, J=10 Hz, 1H), 9.10 (s, 1H), 8.78-8.60 (m, 1H), 8.04 (t, J=5 Hz, 1H), 7.81-7.74 (m, 1H), 7.72-7.65 (m, 2H), 7.56-7.52 (m, 1H), 7.30-7.21 (m, 3H), 7.11 (t, J=10 Hz, 2H), 7.06 (s, 1H), 6.99 (t, J=5 Hz, 1H), 6.95-6.89 (m, 2H), 6.22 (s, 1H), 6.04 (s, 1H), 5.87 (s, 1H), 5.31 (s, 1H), 4.96 (s, 1H), 4.63-4.58 (m, 1H), 4.24-4.02 (m, 4H), 3.85 (s, 1H), 3.51 (t, J=5 Hz, 1H), 3.39 (s, 1H), 3.22-3.18 (m, 1H), 3.12-3.05 (m, 1H), 2.90 (s, 3H), 2.85 (s, 2H), 2.71 (t, J=10 Hz, 1H), 2.35 (t, J=5 Hz, 1H), 2.31-2.26 (m, 1H), 2.06 (t, J=5 Hz, 2H), 2.00 (s, 2H), 1.95 (t, J=5 Hz, 2H), 1.63-1.51 (m, 2H), 1.48-1.34 (m, 5H), 1.32-1.20 (m, 7H), 1.18-1.06 (m, 7H), 0.87 (d, J=5 Hz, 3H), 0.78-0.67 (m, 2H) ppm. ¹³C NMR (126 MHz, DMSO-d₆) δ=173.9, 172.5, 171.7, 170.4, 169.1, 163.3, 159.3, 158.6, 158.3, 136.5, 134.3, 134.1, 130.3, 129.2, 128.9, 127.9, 127.8, 124.0, 121.4, 121.3, 118.9, 118.7, 110.6, 58.9, 57.9, 56.3, 55.5, 54.1, 52.9, 52.7, 52.5, 50.7, 38.8, 37.3, 35.8, 35.4, 32.3, 31.7, 31.2, 29.9, 29.4, 29.2, 28.0, 26.9, 26.0, 25.7, 23.2, 19.9, 15.9 ppm.

MS (ESI): m/z=[M+1] 957.25.

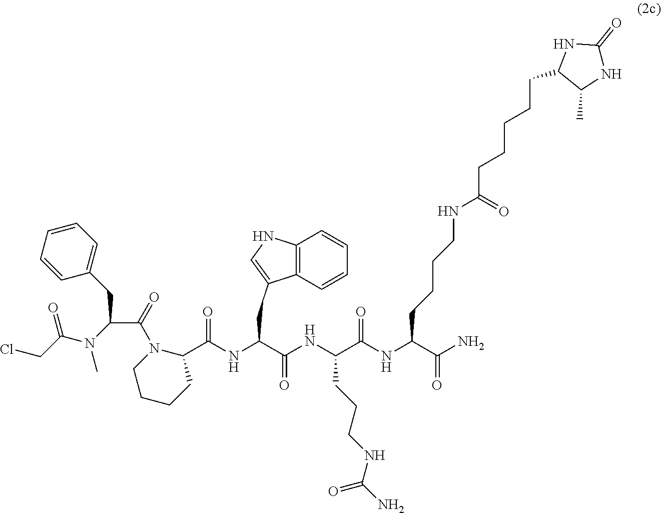

### (S)—N—((S)-1-(((S)-1-(((S)-1-Amino-6-(6-((4S,5R)-5-methyl-2-oxoimidazolidin-4-yl)hexanamido)-1-oxohexan-2-yl)amino)-1-oxo-5-ureidopentan-2-yl)amino)-3-(1H-indol-3-yl)-1-oxopropan-2-yl)-1-(N-(2-chloroacetyl)-N-methyl-L-phenylalanyl)-piperidine-2-carboxamide (Representative compound 2c)

A mixture of peptide 1b (76 mg, 0.08 mmol) in 0.2 mL THF and 0.2 mL saturated sodium bicarbonate was added a solution of chloroacetyl chloride (0.012 mL, 0.16 mmol) in 0.2 mL THF. The resulting mixture was stirred in an ice bath for 2 hours. The mixture was then diluted with 0.5 mL of dichloromethane and 0.5 mL of methanol, and filtered through a 0.45 um syringe filter. The solvent was evaporated, the product purified via HPLC and lyophilized to yield compound 2c as a white powder (58 mg, 70%).

¹H NMR (500 MHz, DMSO-d₆): δ=10.74 (d, J=10 Hz, 1H), 8.08 (d, J=5 Hz, 1H), 7.75 (d, J=5 Hz, 1H), 7.68 (t, J=5 Hz, 1H), 7.58-7.49 (m, 2H), 7.24 (d, J=10 Hz, 2H), 7.17-7.10 (m, 5H), 7.03-6.95 (m, 3H), 6.90 (t, J=5 Hz, 1H), 5.92 (s, 1H), 5.43-5.40 (m, 1H), 5.37-5.34 (m, 1H), 4.89 (s, 1H), 4.66-4.61 (m, 1H), 4.56-4.51 (m, 1H), 4.31 (s, 1H), 4.24-4.18 (m, 2H), 4.11-4.07 (m, 2H), 4.0 (d, J=10 Hz, 1H), 3.75 (d, J=10 Hz, 1H), 3.13-3.10 (m, 2H), 2.98-2.86 (m, 7H), 2.80 (s, 3H), 2.78 (s, 1H), 1.96 (t, J=10 Hz, 3H), 1.54-1.55 (m, 3H), 1.49-1.35 (m, 5H), 1.35-1.21 (m, 7H), 1.19-1.08 (m, 7H), 0.98-0.91 (m, 2H), 0.88 (d, J=5 Hz, 3H) ppm. ¹³C NMR (126 MHz, DMSO) δ=174.0, 172.6, 172.1, 171.7, 170.2, 169.5, 166.0, 163.4, 159.4, 137.8, 136.6, 129.6, 128.6, 127.7, 126.8, 124.2, 121.4, 119.0, 118.7, 111.7, 110.4, 55.5, 54.7, 53.6, 53.1, 52.8, 50.7, 42.3, 40.4, 40.2, 40.0, 39.9, 39.7, 39.5, 39.4, 38.8, 35.8, 34.6, 32.2, 30.6, 29.9, 29.4, 29.2, 27.7, 26.9, 25.9, 25.7, 25.1, 23.2, 20.5, 15.9 ppm.

MS (ESI): m/z=[M+1] 1033.40.

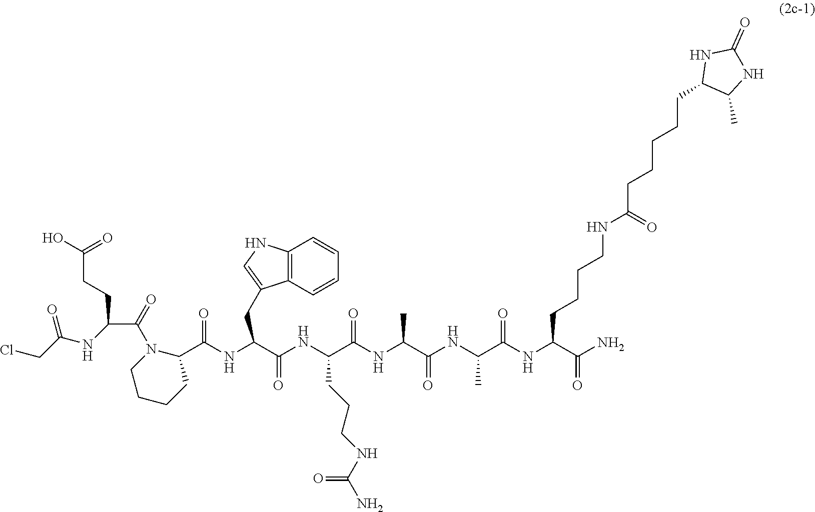 [SMILES: C[C@H](NC(=O)[C@H](C)NC(=O)[C@H](CCCNC(N)=O)NC(=O)[C@H](Cc1c[nH]c2ccccc12)NC(=O)[C@@H]1CCCCN1C(=O)[C@H](CCC(=O)O)NC(=O)CCl)C(=O)N[C@@H](CCCCNC(=O)CCCCC[C@@H]1NC(=O)N[C@@H]1C)C(N)=O]

### (S)-5-((S)-2-(((2S,5S,8S,11S)-14-Carbamoyl-1-(1H-indol-3-yl)-8,11-dimethyl-25-((4S,5R)-5-methyl-2-oxoimidazolidin-4-yl)-3,6,9,12,20-pentaoxo-5-(3-ureidopropyl)-4,7,10,13,19-pentaazapentacosan-2-yl)carbamoyl)piperidin-1-yl)-4-(2-chloroacetamido)-5-oxopentanoic acid

¹H NMR (500 MHz, DMSO-d₆): δ=10.70 (s, 1H), 8.39 (d, J=5 Hz, 1H), 8.02-7.98 (m, 2H), 7.92 (d, J=10 Hz, 1H), 7.65-7.62 (m, 2H), 7.53 (t, J=10 Hz, 2H), 7.24 (d, J=10 Hz, 1H), 7.20 (s, 1H), 7.06 (s, 1H), 6.99-6.94 (m, 2H), 6.90 (t, J=5 Hz, 2H), 5.87 (s, 1H), 4.89 (d, J=5 Hz, 1H), 4.69-4.66 (m, 2H), 4.61-4.55 (m, 2H), 4.21-4.17 (m, 5H), 4.05-3.99 (m, 4H), 3.59 (d, J=10 Hz, 1H), 3.55-3.52 (m, 1H), 3.42-3.39 (m, 1H), 3.11-3.08 (m, 1H), 2.91 (t, J=5 Hz, 6H), 2.77 (t, J=10 Hz, 1H), 2.24 (t, J=5 Hz, 1H), 2.18 (t, J=5 Hz, 2H), 2.04-2.01 (m, 1H), 1.96 (t, J=5 Hz, 3H), 1.80-1.73 (m, 1H), 1.61-1.54 (m, 3H), 1.44-1.38 (m, 6H), 1.31-1.26 (m, 9H), 1.14 (d, J=10 Hz, 8H), 0.89 (d, J=5 Hz, 3H) ppm.

MS (ESI): m/z=[M+2] 1144.26.

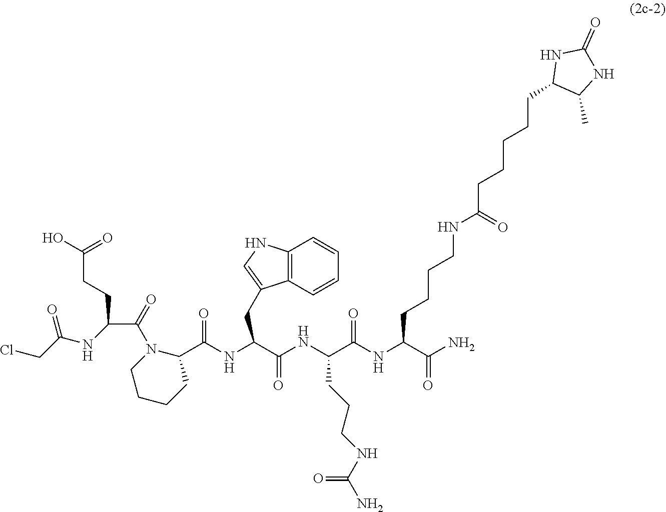 [SMILES: C[C@H]1NC(=O)N[C@H]1CCCCCC(=O)NCCCC[C@H](NC(=O)[C@H](CCCNC(N)=O)NC(=O)[C@H](Cc1c[nH]c2ccccc12)NC(=O)[C@@H]1CCCCN1C(=O)[C@H](CCC(=O)O)NC(=O)CCl)C(N)=O]

### (S)-5-((S)-2-(((S)-1-(((S)-1-(((S)-1-Amino-6-(6-((4S,5R)-5-methyl-2-oxoimidazolidin-4-yl)hexanamido)-1-oxohexan-2-yl)amino)-1-oxo-5-ureidopentan-2-yl)amino)-3-(1H-indol-3-yl)-1-oxopropan-2-yl)carbamoyl)piperidin-1-yl)-4-(2-chloroacetamido)-5-oxopentanoic acid

¹H NMR (500 MHz, DMSO-d₆): δ=10.70 (s, 1H), 8.39 (d, J=10 Hz, 1H), 8.03 (d, J=10 Hz, 1H), 7.73 (d, J=10 Hz, 1H), 7.66 (t, J=5 Hz, 1H), 7.60 (d, J=5 Hz, 1H), 7.54 (d, J=5 Hz, 2H), 7.25-7.22 (m, 2H), 7.07 (d, J=5 Hz, 1H), 7.01-6.93 (m, 2H), 6.90 (t, J=5 Hz, 2H), 5.88 (s, 1H), 4.88 (d, J=5 Hz, 1H), 4.70-4.65 (m, 1H), 4.60-4.56 (m, 1H), 4.21-4.18 (m, 1H), 4.11-4.07 (m, 2H), 4.02 (d, J=5 Hz, 2H), 3.60-3.57 (m, 1H), 3.53 (t, J=5 Hz, 1H), 3.42-3.39 (m, 1H), 3.11-3.07 (m, 1H), 2.94-2.87 (m, 5H), 2.24 (t, J=5 Hz, 1H), 2.17 (t, J=10 Hz, 2H), 2.08-2.02 (m, 1H), 1.96 (t, J=5 Hz, 3H), 1.81-1.73 (m, 1H), 1.62-1.53 (m, 3H), 1.44-1.38 (m, 5H), 1.31-1.25 (m, 9H), 1.18-1.13 (m, 8H), 0.88 (d, J=10 Hz, 3H) ppm.

MS (ESI): m/z=[M+2] 1002.20.

### Example 4: Identification and Characterization of Compounds Synthesized According to Scheme 2

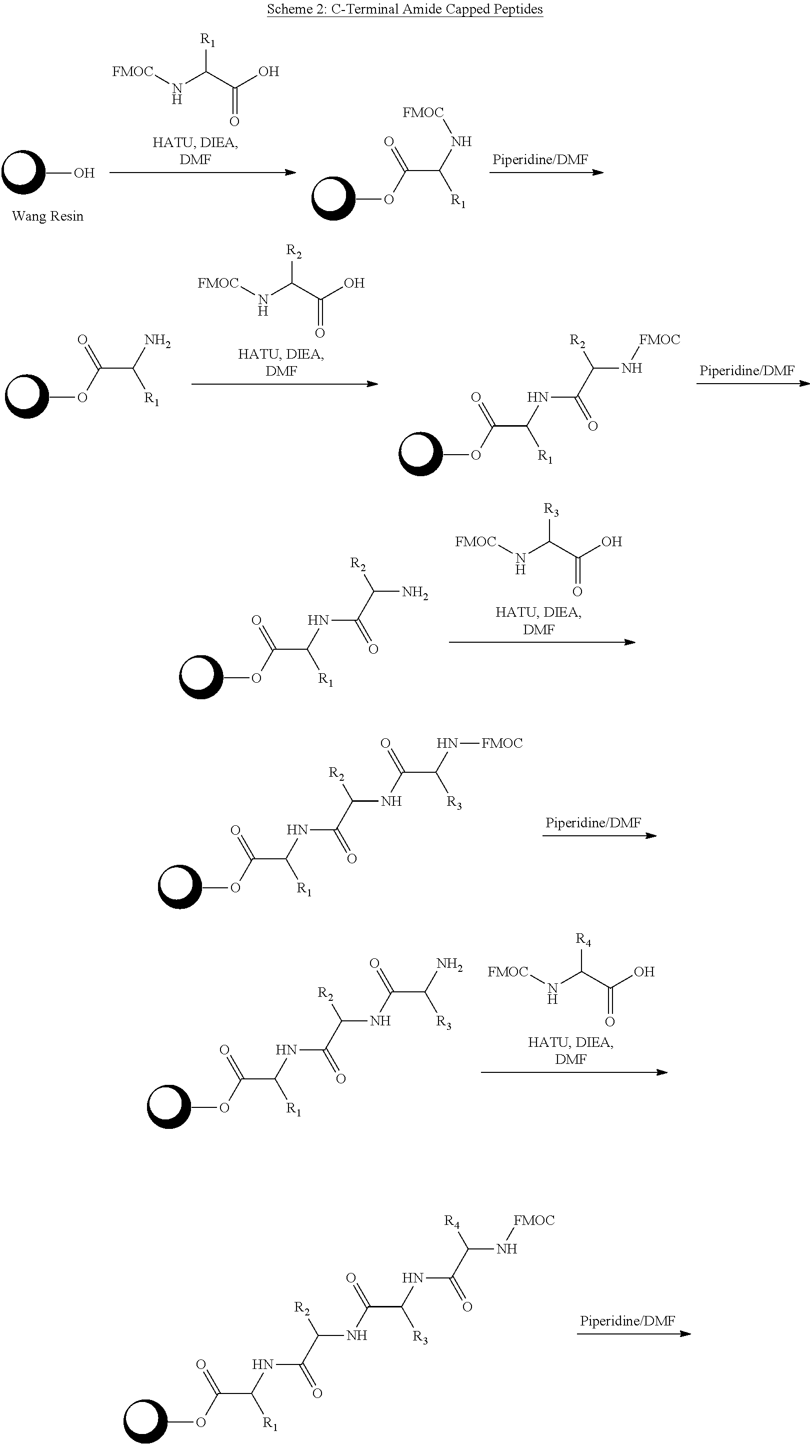

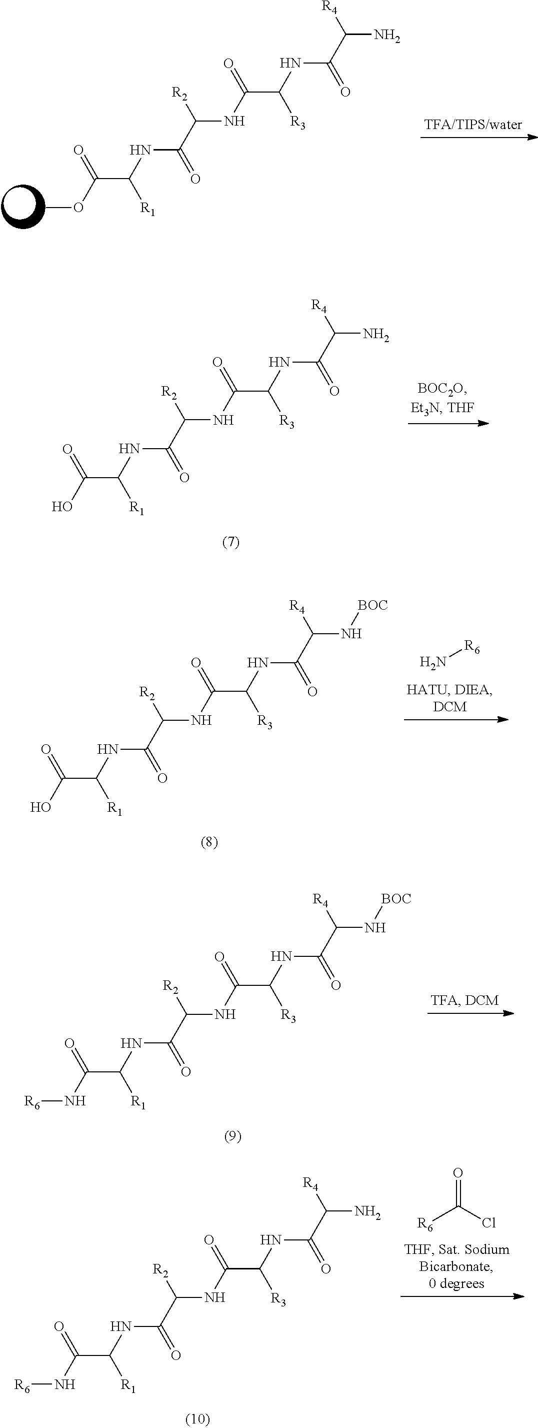

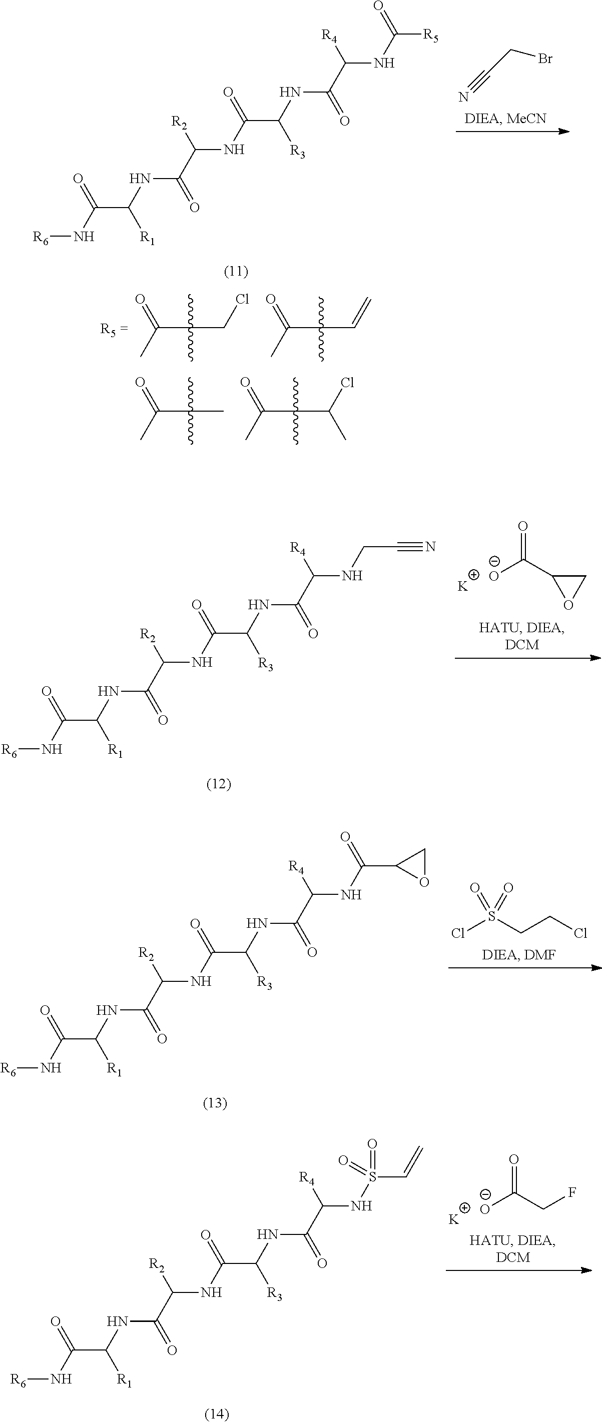

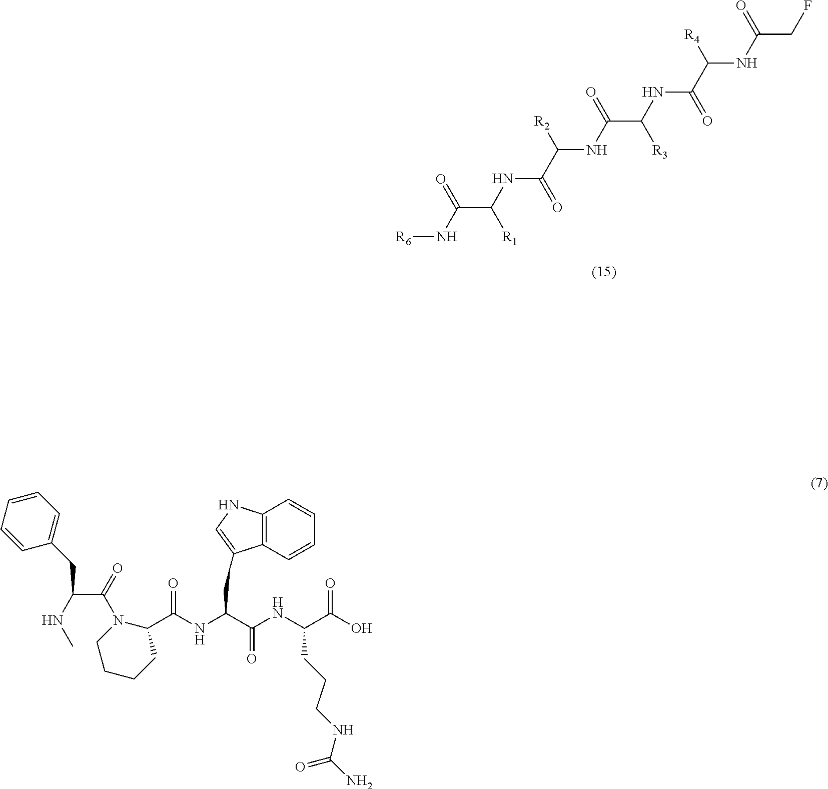

### (S)-2-((S)-3-(1H-Indol-3-yl)-2-((S)-1-(methyl-L-phenylalanyl)piperidine-2-carboxamido) propanamido)-5-ureidopentanoic acid

Peptide 7 was synthesized according to general solid phase peptide synthesis procedures starting from 0.12 mmol of Wang resin. The following amino acids were coupled to the resin: Fmoc-N-Me-Phe-OH, Fmoc-pipecolinic acid, Fmoc-Trp(BOC)-OH, Fmoc-Cit-OH.

¹H NMR (500 MHz, DMSO-d₆): δ=12.57 (s, 1H), 10.75 (d, J=20 Hz, 1H), 9.26 (s, 1H), 8.85-8.75 (m, 1H), 8.61 (s, 1H), 8.26-8.22 (m, 1H), 7.72-7.65 (m, 1H), 7.56 (t, J=5 Hz, 1H), 7.29-7.27 (m, 2H), 7.24 (d, J=10 Hz, 1H), 7.16 (d, J=5 Hz, 1H), 7.12 (d, J=5 Hz, 2H), 7.08 (dd, J=15, 5 Hz, 1H), 7.02-6.96 (m, 1H), 6.91 (dd, J=15, 5 Hz, 1H), 5.91 (d, J=5 Hz, 1H), 5.33 (s, 1H), 4.75-4.8 (m, 1H), 4.66-4.61 (m, 1H), 4.49 (s, 1H), 4.15-4.10 (m, 2H), 4.05-4.02 (m, 1H), 3.86 (d, J=5 Hz, 1H), 2.97-2.93 (m, 1H), 2.88 (s, 3H), 2.35 (t, J=5 Hz, 1H), 2.06 (t, J=5 Hz, 2H), 1.68-1.63 (m, 1H), 1.56-1.40 (m, 4H), 1.34-1.33 (m, 3H), 1.21-1.17 (m, 2H) ppm.

MS (ESI): m/z=[M+1] 634.08.

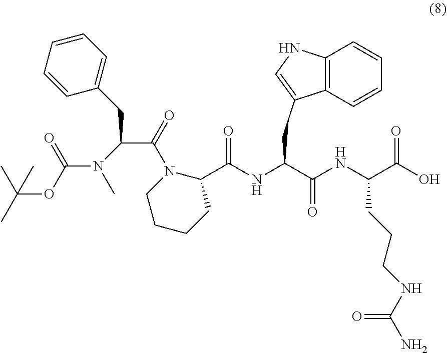 [SMILES: CN(C(=O)OC(C)(C)C)[C@@H](Cc1ccccc1)C(=O)N1CCCC[C@H]1C(=O)N[C@@H](Cc1c[nH]c2ccccc12)C(=O)N[C@@H](CCCNC(N)=O)C(=O)O]

### (S)-2-((S)-2-((S)-1-(N-(tert-Butoxycarbonyl)-N-methyl-L-phenylalanyl)piperidine-2-carboxamido)-3-(1H-indol-3-yl)propanamido)-5-ureidopentanoic acid

To a mixture of peptide 7 (52 mg, 0.08 mmol) in THF (1 mL) was added triethylamine (0.033 mL, 0.24 mmol) and di-tert-butyl dicarbonate (35 mg, 0.16 mmol). The resulting solution was stirred at room temperature for 14 hours. The solvent was evaporated, after which HPLC purification and subsequent lyophilization yielded compound 8 (47 mg, 80%) as a white powder.

¹H NMR (500 MHz, DMSO-d₆): δ=12.64 (s, 1H), 10.75 (s, 1H), 8.26-8.22 (m, 1H), 7.57-7.55 (m, 1H), 7.24-7.19 (m, 4H), 7.16-7.10 (m, 6H), 6.97 (t, J=5 Hz, 1H), 6.8 (t, J=5 Hz, 1H), 5.88 (s, 1H), 5.31 (s, 1H), 4.87 (d, J=5 Hz, 1H), 4.81-4.75 (m, 1H), 4.71-4.67 (m, 1H), 4.16-4.13 (m, 1H), 3.12-3.08 (m, 2H), 2.91-2.85 (m, 4H), 2.54-2.49 (m, 5H), 2.03-2.00 (m, 1H), 1.71-1.67 (m, 1H), 1.57-1.51 (m, 1H), 1.40-1.35 (m, 2H), 1.32-1.26 (m, 1H), 1.22 (s, 3H), 0.98 (s, 9H) ppm. ¹³C NMR (126 MHz, DMSO) δ 173.9, 169.9, 169.7, 159.2, 138.7, 138.7, 136.6, 136.5, 129.9, 129.7, 129.5, 128.7, 128.5, 128.4, 126.7, 126.6, 124.2, 121.2, 119.0, 118.6, 111.6, 79.3, 52.4, 42.7, 40.5, 40.3, 40.2, 40.0, 39.8, 39.7, 39.5, 35.2, 28.4, 28.2, 27.9, 27.9, 27.2, 20.6 ppm.

MS (ESI): m/z=[M+1] 734.07.

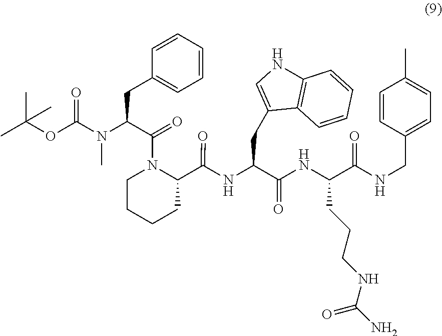 [SMILES: Cc1ccc(CNC(=O)[C@H](CCCNC(N)=O)NC(=O)[C@H](Cc2c[nH]c3ccccc23)NC(=O)[C@@H]2CCCCN2C(=O)[C@H](Cc2ccccc2)N(C)C(=O)OC(C)(C)C)cc1]

### tert-Butyl ((S)-1-((S)-2-(((S)-3-(1H-indol-3-yl)-1-(((S)-1-((4-methylbenzyl)amino)-1-oxo-5-ureidopentan-2-yl)amino)-1-oxopropan-2-yl)carbamoyl)piperidin-1-yl)-1-oxo-3-phenylpropan-2-yl)(methyl)carbamate

A solution of compound 8 (47 mg, 0.064 mmol) and HATU (29 mg, 0.077 mmol) in dichloromethane (1 mL) was stirred at 0° C. for 10 minutes before adding 4-methylbenzylamine (0.009 mL, 0.07 mmol). Then, the solution was stirred for 10 minutes at 0° C. and N,N-Diisopropylethylamine (0.024 mL, 0.14 mmol) was added dropwise. The reaction was then warmed to room temperature and stirred for 4 hours. The mixture was then extracted with ethyl acetate (3×5 mL), washed with water (1×5 mL), brine (1×5 mL), and then dried with sodium sulfate. Evaporation of the solvent followed by HPLC purification and subsequent lyophilization yielded 9 (21 mg, 40%) as a white powder.

¹H NMR (500 MHz, DMSO-d₆): δ=10.75 (s, 1H), 8.35-8.31 (m, 1H), 8.25-8.19 (m, 1H), 8.04-8.00 (m, 1H), 7.66-7.60 (m, 1H), 7.54 (d, J=5 Hz, 1H), 7.24-7.20 (m, 3H), 7.16-7.11 (m, 5H), 7.07-7.03 (m, 7H), 6.97 (t, J=5 Hz, 1H), 6.89 (t, J=5 Hz, 1H), 5.87 (s, 1H), 5.05-5.02 (m, 1H), 4.89 (d, J=5 Hz, 1H), 4.82-4.80 (m, 1H), 4.75-4.71 (m, 1H), 4.65-4.61 (m, 1H), 3.12-3.09 (m, 1H), 3.02-2.95 (m, 1H), 2.90-2.81 (m, 3H), 2.72-2.69 (m, 1H), 2.61 (d, J=10 Hz, 2H), 2.52 (d, J=10 Hz, 2H), 2.20-2.18 (m, 4H), 2.03-2.01 (m, 1H), 1.64-1.58 (m, 1H), 1.51-1.45 (m, 1H), 1.35-1.27 (m, 2H), 1.21 (s, 3H), 1.13 (s, 9H) ppm. ¹³C NMR (126 MHz, DMSO-d₆): δ=171.9, 170.2, 169.8, 159.2, 138.7, 136.7, 136.2, 129.9, 129.3, 129.3, 129.2, 128.5, 128.4, 127.6, 126.6, 121.3, 119.0, 118.6, 111.7, 79.4, 79.3, 61.1, 59.3, 57.3, 55.7, 53.7, 53.1, 42.7, 42.5, 42.3, 40.5, 40.3, 40.2, 40.0, 39.8, 39.7, 39.5, 35.1, 34.8, 30.1, 28.2, 27.9, 27.1, 25.2, 21.1, 20.6 ppm.

MS (ESI): m/z=[M+1] 837.23.

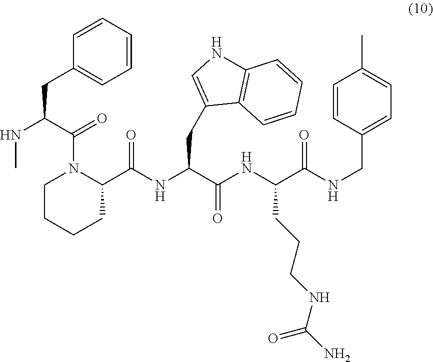 [SMILES: CN[C@@H](Cc1ccccc1)C(=O)N1CCCC[C@H]1C(=O)N[C@@H](Cc1c[nH]c2ccccc12)C(=O)N[C@@H](CCCNC(N)=O)C(=O)NCc1ccc(C)cc1]

### (S)—N—((S)-3-(1H-indol-3-yl)-1-(((S)-1-((4-methylbenzyl)amino)-1-oxo-5-ureidopentan-2-yl)amino)-1-oxopropan-2-yl)-1-(methyl-L-phenylalanyl)piperidine-2-carboxamide

To a mixture of compound 9 (21 mg, 0.025 mmol) in 1 mL dichloromethane was added 10% TFA. The solution was stirred for 14 hours at room temperature, and then concentrated. HPLC purification and subsequent lyophilization yielded compound 10 (15 mg, 80%) as a white powder.

¹H NMR (500 MHz, DMSO-d₆): δ=10.80 (s, 1H), 7.82 (d, J=10 Hz, 1H), 7.54-7.51 (m, 1H), 7.29-7.24 (m, 3H), 7.16-7.12 (m, 2H), 7.10-7.06 (m, 1H), 7.04-7.02 (m, 3H), 7.00-6.98 (m, 1H), 6.91 (t, J=5 Hz, 1H), 5.92 (s, 1H), 4.99 (d, J=5 Hz, 1H), 4.72-4.68 (m, 1H), 4.62-4.58 (m, 1H), 4.25-4.19 (m, 1H), 4.17-4.13 (m, 2H), 3.16-3.09 (m, 1H), 2.99-2.95 (m, 2H), 2.87 (s, 3H), 2.77-2.72 (m, 1H), 2.36 (t, J=5 Hz, 1H), 2.19 (d, J=5 Hz, 3H), 2.08 (t, J=5 Hz, 1H), 1.66-1.58 (m, 2H), 1.47-1.40 (m, 2H), 1.33-1.22 (m, 3H), 1.14-1.07 (m, 1H) ppm.

MS (ESI): m/z=[M+1] 737.43.

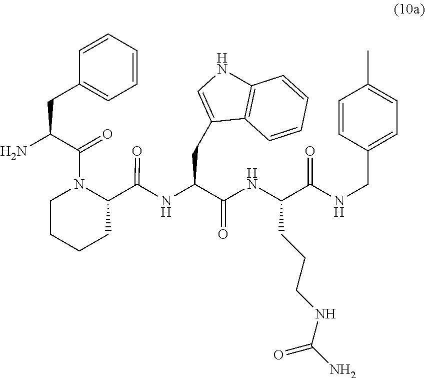 [SMILES: Cc1ccc(CNC(=O)[C@H](CCCNC(N)=O)NC(=O)[C@H](Cc2c[nH]c3ccccc23)NC(=O)[C@@H]2CCCCN2C(=O)[C@@H](N)Cc2ccccc2)cc1]

### (S)—N—((S)-3-(1H-Indol-3-yl)-1-(((S)-1-((4-methylbenzyl)amino)-1-oxo-5-ureidopentan-2-yl) amino)-1-oxopropan-2-yl)-1-(L-phenylalanyl)piperidine-2-carboxamide

Compound 10a was synthesized in an analogous manner to compound 10 but starting with peptidic backbone made from FMOC-N-Me-Phe-OH, Fmoc-pipecolinic acid, Fmoc-Trp(BOC)-OH, Fmoc-Cit-OH.

MS (ESI): m/z=[M+1] 723.96.

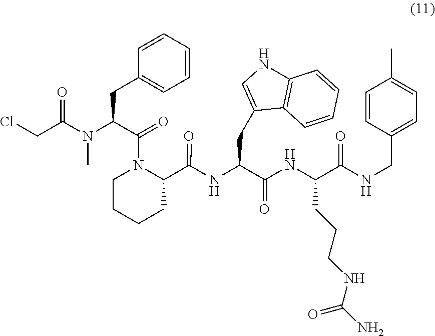 [SMILES: Cc1ccc(CNC(=O)[C@H](CCCNC(N)=O)NC(=O)[C@H](Cc2c[nH]c3ccccc23)NC(=O)[C@@H]2CCCCN2C(=O)[C@H](Cc2ccccc2)N(C)C(=O)CCl)cc1]

### (S)—N—((S)-3-(1H-indol-3-yl)-1-(((S)-1-((4-methylbenzyl)amino)-1-oxo-5-ureidopentan-2-yl)amino)-1-oxopropan-2-yl)-1-(N-(2-chloroacetyl)-N-methyl-L-phenylalanyl)piperidine-2-carboxamide

To a mixture of 10 (15 mg, 0.02 mmol) in 0.2 mL THF and 0.2 mL saturated sodium bicarbonate was added a solution of chloroacetyl chloride (3 μL, 0.04 mmol) in 0.2 mL THF. The resulting mixture was stirred at 0° C. for 2 hours. The mixture was then diluted with 0.5 mL of dichloromethane and 0.5 mL of methanol, and filtered through a 0.45 μm syringe filter. The solvent was evaporated, the product purified via HPLC and lyophilized to yield compound 11 (14 mg, 85%) as a white powder.

¹H NMR (500 MHz, DMSO-d₆): δ=10.82 (s, 1H), 8.27 (t, J=5 Hz, 1H), 8.09 (d, J=5 Hz, 1H), 7.65 (d, J=5 Hz, 1H), 7.59 (d, J=5 Hz, 1H), 7.31 (d, J=5 Hz, 1H), 7.25-7.15 (m, 6H), 7.12 (d, J=5 Hz, 3H), 7.09 (s, 1H), 7.05 (t, J=5 Hz, 1H), 6.97 (t, J=5 Hz, 1H), 5.97 (s, 1H), 5.51-5.47 (m, 1H), 4.97 (d, J=5 Hz, 1H), 4.73-4.69 (m, 1H), 4.30 (s, 2H), 4.23 (t, J=5 Hz, 3H), 4.19 (s, 1H), 4.16 (s, 1H), 3.19-3.15 (m, 2H), 3.02-2.93 (m, 5H), 2.88 (s, 3H), 2.84 (s, 1H), 2.26 (s, 3H), 2.08 (s, 1H), 1.70-1.65 (m, 2H), 1.57-1.53 (m, 1H), 1.40-1.33 (m, 2H), 1.24 (s, 1H), 1.20-1.13 (m, 1H), 1.06 (t, J=5 Hz, 1H), 1.01-0.96 (m, 1H) ppm. ¹³C NMR (126 MHz, DMSO-d₆): δ=171.8, 170.2, 169.5, 166.3, 166.0, 159.3, 137.9, 137.7, 136.7, 136.6, 136.3, 129.7, 129.3, 128.6, 127.7, 127.5, 126.8, 124.4, 124.2, 121.4, 119.0, 118.7, 111.7, 110.4, 56.5, 54.7, 53.7, 53.0, 42.7, 42.3, 42.3, 35.1, 34.6, 30.8, 30.6, 30.1, 29.5, 27.8, 27.1, 26.9, 25.1, 21.1, 20.5.

MS (ESI): m/z [M+1] 813.52.

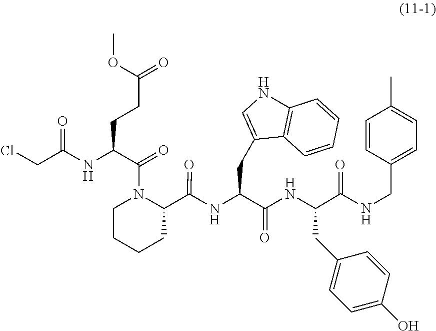 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1ccc(O)cc1)C(=O)NCc1ccc(C)cc1]

### Methyl (S)-4-(2-chloroacetamido)-5-((S)-2-(((S)-1-(((S)-3-(4-hydroxyphenyl)-1-((4-methyl-benzyl) amino)-1-oxopropan-2-yl)amino)-3-(1H-indol-3-yl)-1-oxopropan-2-yl)carbamoyl)piperidin-1-yl)-5-oxopentanoate

MS (ESI): m/z=[M+1] 801.92.

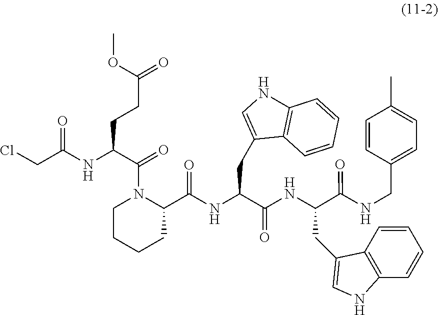 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1c[nH]c2ccccc12)C(=O)NCc1ccc(C)cc1]

### Methyl (S)-5-((S)-2-(((S)-1-(((S)-3-(1H-indol-3-yl)-1-((4-methylbenzyl)amino)-1-oxopropan-2-yl)amino)-3-(1H-indol-3-yl)-1-oxopropan-2-yl)carbamoyl)piperidin-1-yl)-4-(2-chloroacetamido)-5-oxopentanoate

MS (ESI): m/z=[M+1] 824.94.

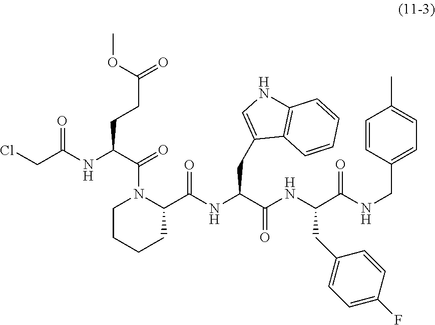 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1ccc(F)cc1)C(=O)NCc1ccc(C)cc1]

### Methyl (S)-4-(2-chloroacetamido)-5-((S)-2-(((S)-1-(((S)-3-(4-fluorophenyl)-1-((4-methylbenzyl) amino)-1-oxopropan-2-yl)amino)-3-(1H-indol-3-yl)-1-oxopropan-2-yl)carbamoyl)piperidin-1-yl)-5-oxopentanoate

¹H NMR (500 MHz, DMSO-d₆): δ=10.82 (s, 1H), 8.47 (d, J=10 Hz, 1H), 8.31 (t, J=5 Hz, 1H), 8.12 (d, J=5 Hz, 1H), 7.62 (d, J=5 Hz, 1H), 7.56 (d, J=5 Hz, 1H), 7.32 (d, J=5 Hz, 1H), 7.21 (t, J=5 Hz, 2H), 7.11-7.07 (m, 3H), 7.05 (s, 2H), 7.04-7.01 (m, 3H), 6.97 (t, J=5 Hz, 1H), 6.54 (s, 1H), 4.94 (d, J=5 Hz, 1H), 4.77-4.72 (m, 1H), 4.65-4.60 (m, 1H), 4.54-4.49 (m, 1H), 4.20 (t, J=5 Hz, 2H), 4.09 (s, 2H), 3.59 (s, 3H), 3.09-3.06 (m, 2H), 2.99-2.93 (m, 2H), 2.88-2.80 (m, 2H), 2.41 (t, J=5 Hz, 1H), 2.32 (t, J=5 Hz, 2H), 2.07-2.02 (m, 1H), 1.89-1.79 (m, 1H), 1.68-1.63 (m, 1H), 1.42-1.34 (m, 3H), 1.27-1.22 (m, 2H), 1.12-1.05 (m, 1H) ppm. ¹³C NMR (126 MHz, DMSO-d₆) δ=173.4, 171.7, 170.9, 170.5, 170.2, 166.2, 162.4, 160.5, 136.5, 136.4, 136.2, 134.1, 131.5, 131.5, 129.2, 127.6, 124.0, 121.3, 118.9, 118.7, 115.3, 115.1, 111.7, 110.3, 54.6, 53.7, 52.5, 51.8, 48.5, 43.1, 42.8, 42.3, 40.5, 37.3, 29.6, 29.5, 27.8, 26.9, 26.8, 25.3, 21.1, 20.4 ppm. ¹⁹F NMR (471 MHz, DMSO-d₆) δ=−73.61.

MS (ESI): m/z=[M+1] 803.97.

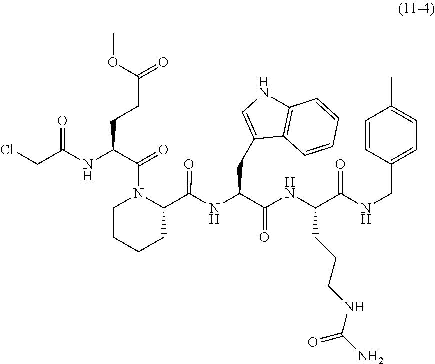 [SMILES: COC(=O)CC[C@H](NC(=O)CCl)C(=O)N1CCCC[C@H]1C(=O)N[C@@H](Cc1c[nH]c2ccccc12)C(=O)N[C@@H](CCCNC(N)=O)C(=O)NCc1ccc(C)cc1]

### Methyl (S)-5-((S)-2-(((S)-3-(1H-indol-3-yl)-1-(((S)-1-((4-methyl benzyl)amino)-1-oxo-5-ureidopentan-2-yl)amino)-1-oxopropan-2-yl)carbamoyl)piperidin-1-yl)-4-(2-chloroacetamido)-5-oxopentanoate

MS (ESI): m/z=[M+1] 795.97.

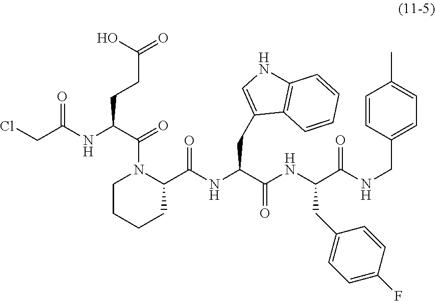 [SMILES: Cc1ccc(CNC(=O)[C@H](Cc2ccc(F)cc2)NC(=O)[C@H](Cc2c[nH]c3ccccc23)NC(=O)[C@@H]2CCCCN2C(=O)[C@H](CCC(=O)O)NC(=O)CCl)cc1]

### (S)-4-(2-chloroacetamido)-5-((S)-2-(((S)-1-(((S)-3-(4-fluorophenyl)-1-((4-methylbenzyl)amino)-1-oxopropan-2-yl)amino)-3-(1H-indol-3-yl)-1-oxopropan-2-yl)carbamoyl)piperidin-1-yl)-5-oxopentanoic acid

MS (ESI): m/z=[M+1] 789.86.

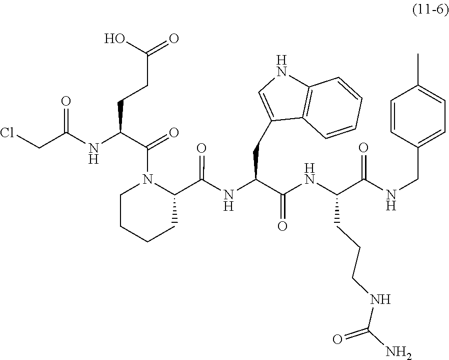 [SMILES: Cc1ccc(CNC(=O)[C@H](CCCNC(N)=O)NC(=O)[C@H](Cc2c[nH]c3ccccc23)NC(=O)[C@@H]2CCCCN2C(=O)[C@H](CCC(=O)O)NC(=O)CCl)cc1]

### (S)-5-((S)-2-(((S)-3-(1H-indol-3-yl)-1-(((S)-1-((4-methylbenzyl)amino)-1-oxo-5-ureidopentan-2-yl)amino)-1-oxopropan-2-yl)carbamoyl)piperidin-1-yl)-4-(2-chloroacetamido)-5-oxopentanoic acid

MS (ESI): m/z=[M+1] 781.95.

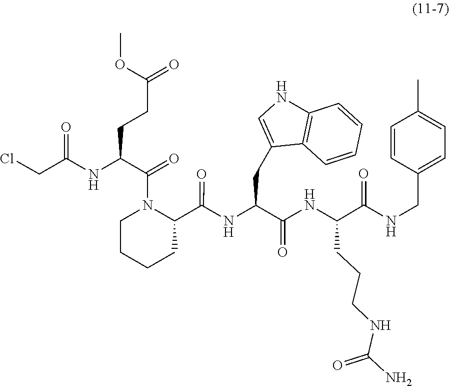 [SMILES: COC(=O)CC[C@H](NC(=O)CCl)C(=O)N1CCCC[C@H]1C(=O)N[C@@H](Cc1c[nH]c2ccccc12)C(=O)N[C@@H](CCCNC(N)=O)C(=O)NCc1ccc(C)cc1]

### Methyl (S)-5-((S)-2-(((S)-3-(1H-indol-3-yl)-1-(((S)-1-(methyl(4-methylbenzyl)amino)-1-oxo-5-ureidopentan-2-yl)amino)-1-oxopropan-2-yl)carbamoyl)piperidin-1-yl)-4-(2-chloroacetamido)-5-oxopentanoate

¹H NMR (500 MHz, DMSO-d₆): δ=10.73 (s, 1H), 8.40 (d, J=10 Hz, 1H), 8.20 (t, J=5 Hz, 1H), 7.60-7.56 (m, 1H), 7.53 (d, J=10 Hz, 1H), 7.24 (t, J=5 Hz, 1H), 7.16 (s, 1H), 7.08-7.05 (m, 2H), 7.03 (d, J=5 Hz, 2H), 7.00-6.98 (m, 2H), 6.90 (t, J=5 Hz, 1H), 5.85-5.80 (m, 1H), 5.35-5.26 (m, 2H), 4.90 (d, J=5 Hz, 1H), 4.72-4.66 (m 1H), 4.62-4.57 (m, 1H), 4.45 (s, 1H), 4.39 (s, 1H), 4.02 (s, 3H), 3.52 (s, 3H), 3.13 (s, 1H), 2.93-2.88 (m, 2H), 2.84 (d, J=5 Hz, 2H), 2.63 (d, J=10 Hz, 1H), 2.27-2.25 (m, 2H), 2.19-2.17 (m, 2H), 2.05-2.01 (m, 1H), 1.82-1.76 (m, 2H), 1.64-1.57 (m, 2H), 1.49-1.42 (m, 2H), 1.37-1.29 (m, 4H), 1.17 (s, 2H), 1.09-1.01 (m, 1H), 0.84-0.76 (m, 1H) ppm.

MS (ESI): m/z=[M+1] 809.98.

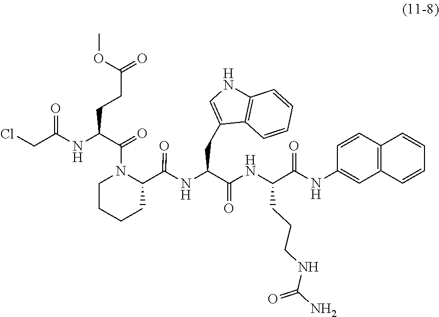 [SMILES: COC(=O)CC[C@H](NC(=O)CCl)C(=O)N1CCCC[C@H]1C(=O)N[C@@H](Cc1c[nH]c2ccccc12)C(=O)N[C@@H](CCCNC(N)=O)C(=O)Nc1ccc2ccccc2c1]

### Methyl (S)-5-((S)-2-(((S)-3-(1H-indol-3-yl)-1-(((S)-1-(naphthalen-2-ylamino)-1-oxo-5-ureidopentan-2-yl)amino)-1-oxopropan-2-yl)carbamoyl)piperidin-1-yl)-4-(2-chloroacetamido)-5-oxopentanoate

¹H NMR (500 MHz, DMSO-d₆): δ=10.73 (s, 1H), 10.14 (s, 1H), 8.39 (d, J=10 Hz, 1H), 8.23-8.20 (m, 1H), 7.81-7.75 (m, 4H), 7.56-7.53 (m, 3H), 7.41 (t, J=5 Hz, 1H), 7.33 (t, J=5 Hz, 1H), 7.23 (d, J=5 Hz, 1H), 7.09 (s, 1H), 6.96 (t, J=5 Hz, 1H), 6.87 (t, J=5 Hz, 1H), 5.94 (s, 1H), 5.40-5.35 (m, 1H), 4.91 (d, J=5 Hz, 1H), 4.72-4.63 (m, 2H), 4.46-4.42 (m, 1H), 4.01 (s, 2H), 3.51 (s, 3H), 3.17-3.13 (m, 1H), 3.00-2.97 (m, 1H), 2.94-2.87 (m, 1H), 2.80-2.73 (m, 1H), 2.35 (t, J=5 Hz, 1H), 2.25 (t, J=5 Hz, 2H), 2.08-2.02 (m, 1H), 1.82-1.75 (m, 2H), 1.72-1.68 (m, 2H), 1.63-1.57 (m, 2H), 1.43-1.37 (m, 2H), 1.43-1.29 (m, 3H), 1.19-1.15 (m, 2H) ppm.

MS (ESI): m/z=[M+1] 817.88.

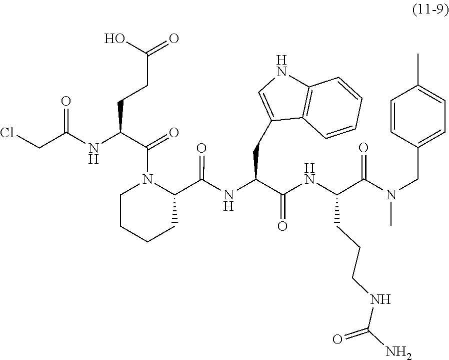 [SMILES: Cc1ccc(CN(C)C(=O)[C@H](CCCNC(N)=O)NC(=O)[C@H](Cc2c[nH]c3ccccc23)NC(=O)[C@@H]2CCCCN2C(=O)[C@H](CCC(=O)O)NC(=O)CCl)cc1]

### (S)-5-((S)-2-(((S)-3-(1H-Indol-3-yl)-1-(((S)-1-(methyl(4-methylbenzyl)amino)-1-oxo-5-ureidopentan-2-yl)amino)-1-oxopropan-2-yl)carbamoyl)piperidin-1-yl)-4-(2-chloroacetamido)-5-oxopentanoic acid

MS (ESI): m/z=[M+1] 795.82.

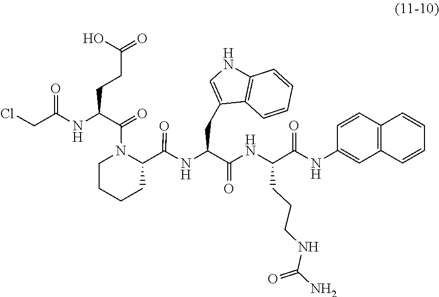 [SMILES: NC(=O)NCCC[C@H](NC(=O)[C@H](Cc1c[nH]c2ccccc12)NC(=O)[C@@H]1CCCCN1C(=O)[C@H](CCC(=O)O)NC(=O)CCl)C(=O)Nc1ccc2ccccc2c1]

### (S)-5-((S)-2-(((S)-3-(1H-Indol-3-yl)-1-(((S)-1-(naphthalen-2-ylamino)-1-oxo-5-ureidopentan-2-yl)amino)-1-oxopropan-2-yl)carbamoyl)piperidin-1-yl)-4-(2-chloroacetamido)-5-oxopentanoic acid

MS (ESI): m/z=[M+1] 803.87.

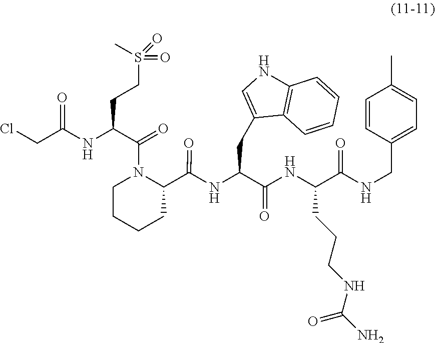 [SMILES: Cc1ccc(CNC(=O)[C@H](CCCNC(N)=O)NC(=O)[C@H](Cc2c[nH]c3ccccc23)NC(=O)[C@@H]2CCCCN2C(=O)[C@H](CCS(C)(=O)=O)NC(=O)CCl)cc1]

### (S)—N—((S)-3-(1H-Indol-3-yl)-1-(((S)-1-((4-methylbenzyl)amino)-1-oxo-5-ureidopentan-2-yl)amino)-1-oxopropan-2-yl)-1-((S)-2-(2-chloroacetamido)-4-(methylsulfonyl)butanoyl)piperidine-2-carboxamide

¹H NMR (500 MHz, DMSO-d₆): δ=10.72 (s, 1H), 8.59 (d, J=10 Hz, 1H), 8.17 (t, J=5 Hz, 1H), 8.03 (d, J=10 Hz, 1H), 7.98 (d, J=5 Hz, 1H), 7.62 (d, J=5 Hz, 1H), 7.54 (d, J=10 Hz, 2H), 7.25 (d, J=10 Hz, 2H), 7.16-7.12 (m, 1H), 7.05-7.03 (m, 2H), 7.00 (d, J=5 Hz, 2H), 6.98-6.95 (m, 2H), 6.90 (t, J=5 Hz, 2H), 5.87 (s, 2H), 5.30 (s, 2H), 4.93-4.87 (m, 1H), 4.82-4.78 (m, 1H), 4.63-4.55 (m, 2H), 4.50-4.41 (m, 2H), 4.23-4.19 (m, 2H), 4.15-4.11 (m, 2H), 4.03 (s, 3H), 3.11-3.01 (m, 2H), 2.89 (s, 2H), 2.19 (s, 3H), 2.06-1.93 (m, 1H), 1.91-1.85 (m, 1H), 1.59-1.44 (m, 2H), 1.33-1.23 (m, 1H), 0.98 (d, J=5 Hz, 3H) ppm.

MS (ESI): m/z=[M+1] 815.93.

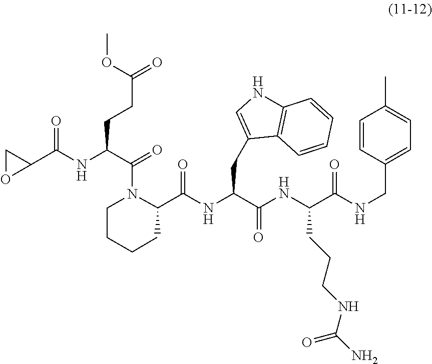 [SMILES: COC(=O)CC[C@H](NC(=O)C1CO1)C(=O)N1CCCC[C@H]1C(=O)N[C@@H](Cc1c[nH]c2ccccc12)C(=O)N[C@@H](CCCNC(N)=O)C(=O)NCc1ccc(C)cc1]

### Methyl (4S)-5-((S)-2-(((S)-3-(1H-indol-3-yl)-1-(((S)-1-((4-methyl benzyl)amino)-1-oxo-5-ureido-pentan-2-yl)amino)-1-oxopropan-2-yl)carbamoyl)piperidin-1-yl)-4-(oxirane-2-carboxamido)-5-oxopentanoate

¹H NMR (500 MHz, DMSO-d₆): δ=10.83 (s, 1H), 8.27 (d, J=10 Hz, 1H), 8.07 (d, J=10 Hz, 1H), 7.71-7.61 (m, 1H), 7.62-7.56 (m, 1H), 7.32 (d, J=5 Hz, 2H), 7.11 (s, 2H), 7.05 (t, J=5 Hz, 1H), 6.96 (t, J=5 Hz, 1H), 6.02 (s, 1H), 4.95 (s, 1H), 4.65 (d, J=5 Hz, 2H), 4.28-4.25 (m, 2H), 4.21 (t, J=5 Hz, 2H), 3.58 (s, 3H), 3.17 (s, 3H), 3.00-2.92 (m, 4H), 2.26 (s, 3H), 2.13-2.07 (m, 2H), 1.84-1.81 (m, 2H), 1.69-1.61 (m, 2H), 1.56-1.49 (m, 2H), 1.45-1.41 (m, 2H), 1.37-1.33 (m, 2H), 1.30 (d, J=10 Hz, 2H), 1.27 (d, J=10 Hz, 2H), 1.24 (s, 1H), 1.05 (d, J=5 Hz, 1H), 0.89-0.83 (m, 2H) ppm.

MS (ESI): m/z=[M+1] 789.91.

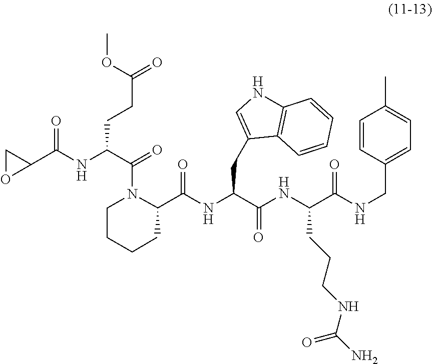 [SMILES: COC(=O)CC[C@@H](NC(=O)C1CO1)C(=O)N1CCCC[C@H]1C(=O)N[C@@H](Cc1c[nH]c2ccccc12)C(=O)N[C@@H](CCCNC(N)=O)C(=O)NCc1ccc(C)cc1]

### Methyl (R)-5-((S)-2-(((S)-3-(1H-indol-3-yl)-1-(((S)-1-((4-methylbenzyl)amino)-1-oxo-5-ureido-pentan-2-yl)amino)-1-oxopropan-2-yl)carbamoyl)piperidin-1-yl)-4-(2-chloroacetamido)-5-oxopentanoate

MS (ESI): m/z=[M+1] 795.52.

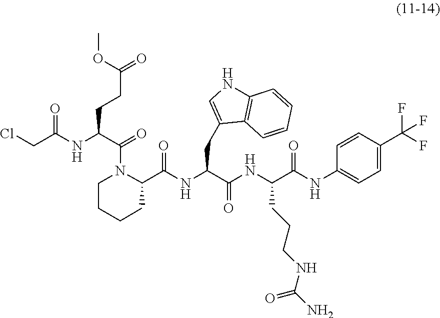 [SMILES: COC(=O)CC[C@H](NC(=O)CCl)C(=O)N1CCCC[C@H]1C(=O)N[C@@H](Cc1c[nH]c2ccccc12)C(=O)N[C@@H](CCCNC(N)=O)C(=O)Nc1ccc(C(F)(F)F)cc1]

### Methyl (S)-5-((S)-2-(((S)-3-(1H-indol-3-yl)-1-oxo-1-(((S)-1-oxo-1-((4-(trifluoromethyl)-phenyl)amino)-5-ureidopentan-2-yl)amino)propan-2-yl)carbamoyl)-piperidin-1-yl)-4-(2-chloroacetamido)-5-oxopentanoate

MS (ESI): m/z=[M+1] 835.60.

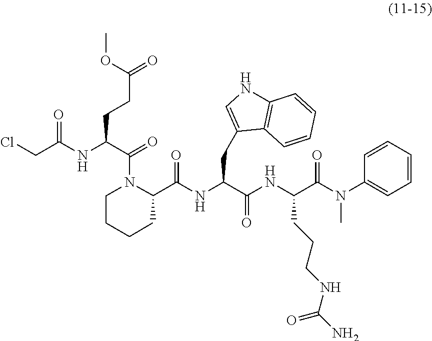 [SMILES: COC(=O)CC[C@H](NC(=O)CCl)C(=O)N1CCCC[C@H]1C(=O)N[C@@H](Cc1c[nH]c2ccccc12)C(=O)N[C@@H](CCCNC(N)=O)C(=O)N(C)c1ccccc1]

### Methyl (S)-5-((S)-2-(((S)-3-(1H-indol-3-yl)-1-(((S)-1-(methyl(phenyl)amino)-1-oxo-5-ureidopentan-2-yl)amino)-1-oxopropan-2-yl)carbamoyl)piperidin-1-yl)-4-(2-chloroacetamido)-5-oxopentanoate

MS (ESI): m/z=[M+1] 781.45.

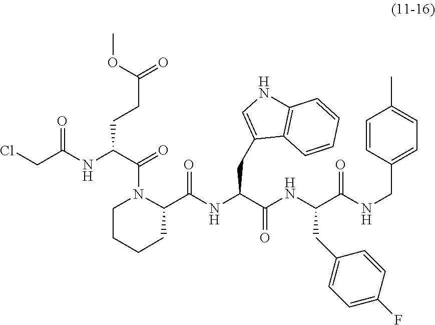 [SMILES: COC(=O)CC[C@@H](NC(=O)CCl)C(=O)N1CCCC[C@H]1C(=O)N[C@@H](Cc1c[nH]c2ccccc12)C(=O)N[C@@H](Cc1ccc(F)cc1)C(=O)NCc1ccc(C)cc1]

### Methyl (R)-4-(2-chloroacetamido)-5-((S)-2-(((S)-1-(((S)-3-(4-fluorophenyl)-1-((4-methyl-benzyl) amino)-1-oxopropan-2-yl)amino)-3-(1H-indol-3-yl)-1-oxopropan-2-yl)carbamoyl)-piperidin-1-yl)-5-oxopentanoate

MS (ESI): m/z=[M+1] 803.52.

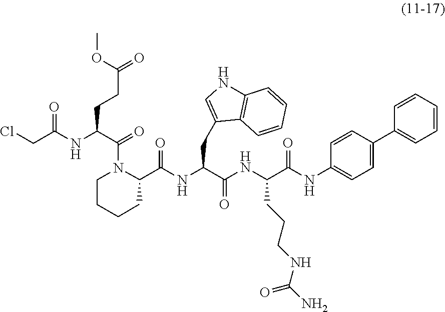 [SMILES: COC(=O)CC[C@H](NC(=O)CCl)C(=O)N1CCCC[C@H]1C(=O)N[C@@H](Cc1c[nH]c2ccccc12)C(=O)N[C@@H](CCCNC(N)=O)C(=O)Nc1ccc(-c2ccccc2)cc1]

### Methyl (S)-5-((S)-2-(((S)-1-(((S)-1-([1,1′-biphenyl]-4-ylamino)-1-oxo-5-ureidopentan-2-yl)amino)-3-(1H-indol-3-yl)-1-oxopropan-2-yl)carbamoyl)piperidin-1-yl)-4-(2-chloroacetamido)-5-oxopentanoate

MS (ESI): m/z=[M+1] 843.61.

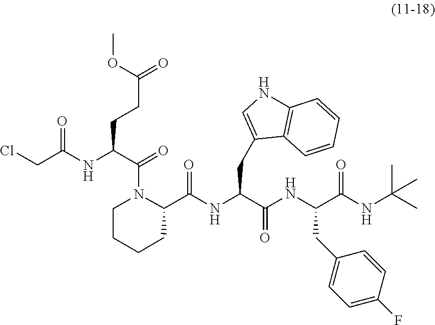 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1ccc(F)cc1)C(=O)NC(C)(C)C]

### Methyl (S)-5-((S)-2-(((S)-1-(((S)-1-(tert-butylamino)-3-(4-fluorophenyl)-1-oxopropan-2-yl)amino)-3-(1H-indol-3-yl)-1-oxopropan-2-yl)carbamoyl)piperidin-1-yl)-4-(2-chloroacetamido)-5-oxopentanoate

¹H NMR (500 MHz, DMSO-d₆): δ=10.82 (s, 1H), 8.47 (d, J=5 Hz, 1H), 7.89 (d, J=10 Hz, 1H), 7.62 (d, J=5 Hz, 1H), 7.55 (d, J=10 Hz, 1H), 7.47-7.45 (m, 1H), 7.31 (d, J=5 Hz, 1H), 7.24-7.19 (m, 2H), 7.10-7.03 (m, 3H), 7.00-6.95 (m, 2H), 4.94 (d, J=5 Hz, 1H), 4.74-4.67 (m, 1H), 4.59-4.55 (m, 1H), 4.48-4.43 (m, 2H), 4.09 (s, 2H), 3.58 (s, 3H), 3.10-3.06 (m, 1H), 3.01-2.94 (m, 1H), 2.88-2.78 (m, 3H), 2.34-2.30 (m, 2H), 2.13-2.04 (m, 2H), 1.87-1.83 (m, 2H), 1.68-1.60 (m, 1H), 1.45-1.36 (m, 3H), 1.19 (s, 9H) ppm.

MS (ESI): m/z=[M+1] 755.53.

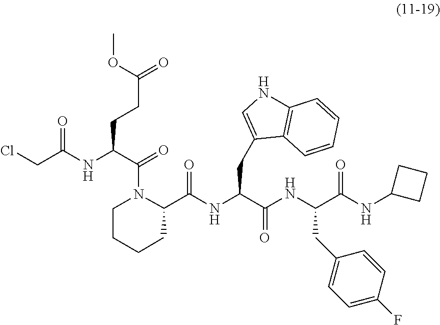 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1ccc(F)cc1)C(=O)NC1CCC1]

### Methyl (S)-4-(2-chloroacetamido)-5-((S)-2-(((S)-1-(((S)-1-(cyclobutylamino)-3-(4-fluorophenyl)-1-oxopropan-2-yl)amino)-3-(1H-indol-3-yl)-1-oxopropan-2-yl)carbamoyl)piperidin-1-yl)-5-oxopentanoate

¹H NMR (500 MHz, DMSO-d₆): δ=10.73 (s, 1H), 8.39 (d, J=5 Hz, 1H), 7.96 (d, J=10 Hz, 1H), 7.92 (d, J=5 Hz, 1H), 7.51 (d, J=10 Hz, 1H), 7.48 (d, J=5 Hz, 1H), 7.24 (d, J=10 Hz, 1H), 7.15-7.10 (m, 2H), 7.00-6.96 (m, 3H), 6.89 (t, J=5 Hz, 2H), 4.88 (d, J=5 Hz, 1H), 4.68-4.66 (m, 1H), 4.53-4.50 (m, 1H), 4.34-4.30 (m, 2H), 4.08-4.03 (m, 2H), 4.02 (d, J=5 Hz, 2H), 3.51 (s, 3H), 3.02-2.98 (m, 2H), 2.91-2.85 (m, 1H), 2.82-2.72 (m, 2H), 2.34 (t, J=5 Hz, 1H), 2.25 (t, J=5 Hz, 2H), 2.07-1.99 (m, 3H), 1.76 (t, J=10 Hz, 2H), 1.65 (t, J=5 Hz, 1H), 1.57-1.50 (m, 2H), 1.36-1.29 (m, 2H), 1.21-1.16 (m, 1H), 1.09-0.99 (m, 1H) ppm.

MS (ESI): m/z=[M+1] 753.48.

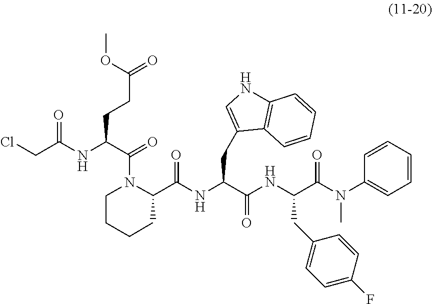 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1ccc(F)cc1)C(=O)N(C)c1ccccc1]

### Methyl (S)-4-(2-chloroacetamido)-5-((S)-2-(((S)-1-(((S)-3-(4-fluorophenyl)-1-(methyl-(phenyl) amino)-1-oxopropan-2-yl)amino)-3-(1H-indol-3-yl)-1-oxopropan-2-yl)carbamoyl)-piperidin-1-yl)-5-oxopentanoate

MS (ESI): m/z=[M+1] 789.56.

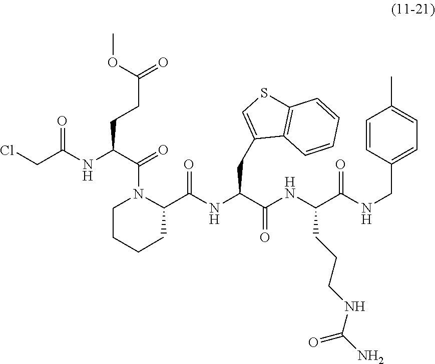 [SMILES: COC(=O)CC[C@H](NC(=O)CCl)C(=O)N1CCCC[C@H]1C(=O)N[C@@H](Cc1csc2ccccc12)C(=O)N[C@@H](CCCNC(N)=O)C(=O)NCc1ccc(C)cc1]

### Methyl (S)-5-((S)-2-(((S)-3-(benzo[&]thiophen-3-yl)-1-(((S)-1-((4-methylbenzyl)amino)-1-oxo-5-ureidopentan-2-yl)amino)-1-oxopropan-2-yl)carbamoyl)piperidin-1-yl)-4-(2-chloroacetamido)-5-oxopentanoate

¹H NMR (500 MHz, DMSO-d₆): δ=8.49 (d, J=10 Hz, 1H), 8.33 (t, J=5 Hz, 1H), 8.19 (d, J=5 Hz, 1H), 7.95 (d, J=10 Hz, 2H), 7.89 (d, J=10 Hz, 2H), 7.41-7.36 (m, 2H), 7.12 (s, 2H), 5.96 (s, 1H), 4.97 (d, J=5 Hz, 1H), 4.79-4.76 (m, 2H), 4.72-4.65 (m, 1H), 4.30-4.21 (m, 4H), 4.09 (s, 2H), 3.59 (s, 3H), 3.30-3.26 (m, 1H), 3.16-3.11 (m, 1H), 2.97-2.95 (m, 3H), 2.91-2.85 (m, 1H), 2.39 (t, J=5 Hz, 1H), 2.33 (t, J=5 Hz, 1H), 2.26 (s, 1H), 2.17-2.13 (m, 1H), 2.09-2.05 (m, 1H), 1.93-1.84 (m, 2H), 1.71-1.65 (m, 2H), 1.59-1.52 (m, 1H), 1.47-1.33 (m, 5H), 1.29-1.23 (m, 1H), 1.20-1.11 (m, 3H) ppm.

MS (ESI): m/z=[M+1] 812.88.

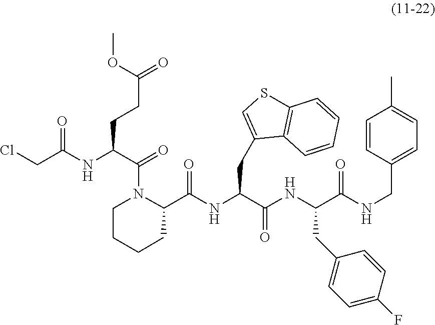 [SMILES: COC(=O)CC[C@H](NC(=O)CCl)C(=O)N1CCCC[C@H]1C(=O)N[C@@H](Cc1csc2ccccc12)C(=O)N[C@@H](Cc1ccc(F)cc1)C(=O)NCc1ccc(C)cc1]

### Methyl (S)-5-((S)-2-(((S)-3-(benzo[b]thiophen-3-yl)-1-(((S)-3-(4-fluorophenyl)-1-((4-methylbenzyl)amino)-1-oxopropan-2-yl)amino)-1-oxopropan-2-yl)carbamoyl)piperidin-1-yl)-4-(2-chloroacetamido)-5-oxopentanoate

¹H NMR (500 MHz, DMSO-d₆): δ=8.49 (d, J=5 Hz, 1H), 8.39 (t, J=5 Hz, 1H), 8.25 (d, J=10 Hz, 1H), 7.96 (d, J=5 Hz, 1H), 7.87 (d, J=10 Hz, 1H), 7.82 (d, J=10 Hz, 1H), 7.41 (t, J=5 Hz, 2H), 7.38 (s, 2H), 7.26-7.23 (m, 2H), 7.10 (d, J=5 Hz, 4H), 7.05 (d, J=5 Hz, 4H), 4.95 (d, J=5 Hz, 1H), 4.80-4.76 (m, 2H), 4.71-4.65 (m, 1H), 4.58-4.53 (m, 1H), 4.23 (d, J=5 Hz, 1H), 4.10 (s, 3H), 3.59 (s, 3H), 3.23-3.18 (m, 2H), 3.13-3.08 (m, 1H), 3.02-2.98 (m, 2H), 2.90-2.84 (m, 2H), 1.91-1.87 (m, 2H), 1.74-1.68 (m, 1H), 1.45-1.39 (m, 2H), 1.27-1.25 (m, 1H), 1.16-1.11 (m, 2H) ppm.

MS (ESI): m/z=[M+1] 820.84.

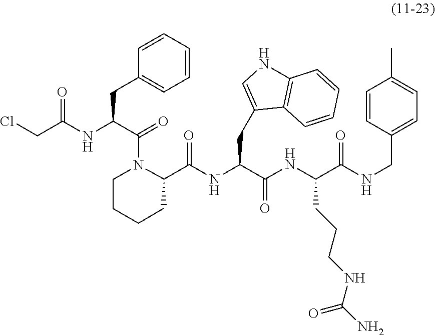 [SMILES: Cc1ccc(CNC(=O)[C@H](CCCNC(N)=O)NC(=O)[C@H](Cc2c[nH]c3ccccc23)NC(=O)[C@@H]2CCCCN2C(=O)[C@H](Cc2ccccc2)NC(=O)CCl)cc1]

### (S)—N—((S)-3-(1H-indol-3-yl)-1-(((S)-1-((4-methylbenzyl)amino)-1-oxo-5-ureidopentan-2-yl)amino)-1-oxopropan-2-yl)-1-((2-chloroacetyl)-L-phenylalanyl)piperidine-2-carboxamide

MS (ESI): m/z=[M+1] 799.97.

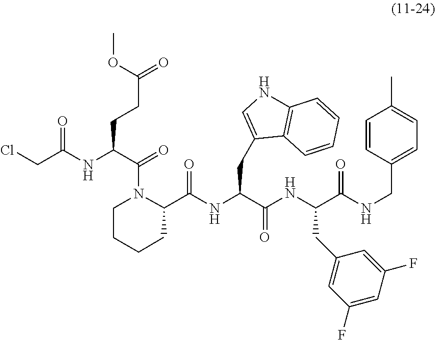 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1cc(F)cc(F)c1)C(=O)NCc1ccc(C)cc1]

### Methyl (S)-4-(2-chloroacetamido)-5-((S)-2-(((S)-1-(((S)-3-(3,5-difluorophenyl)-1-((4-methyl-benzyl)amino)-1-oxopropan-2-yl)amino)-3-(1H-indol-3-yl)-1-oxopropan-2-yl)carbamoyl)piperidin-1-yl)-5-oxopentanoate

¹H NMR (500 MHz, DMSO-d₆): δ=10.74 (s, 1H), 8.38 (d, J=5 Hz, 1H), 8.24 (t, J=5 Hz, 1H), 8.14 (d, J=5 Hz, 1H), 7.55 (d, J=5 Hz, 1H), 7.49 (d, J=5 Hz, 1H), 7.25-7.22 (m, 1H), 7.04-7.01 (m, 4H), 6.98-6.95 (m, 4H), 6.91-6.85 (m, 3H), 4.86 (d, J=5 Hz, 1H), 4.69-4.62 (m, 1H), 4.51 (t, J=5 Hz, 2H), 4.14 (t, J=5 Hz, 1H), 4.01 (s, 2H), 3.51 (s, 3H), 2.99-2.89 (m, 3H), 2.82-2.72 (m, 2H), 2.33 (t, J=5 Hz, 1H), 2.24 (t, J=5 Hz, 2H), 2.19 (s, 3H), 1.99-1.96 (m, 1H), 1.80-1.73 (m, 1H), 1.58-1.54 (m, 1H), 1.35-1.29 (m, 3H), 1.19-1.12 (m, 1H), 1.04-1.00 (m, 1H) ppm.

MS (ESI): m/z=[M+1] 821.84.

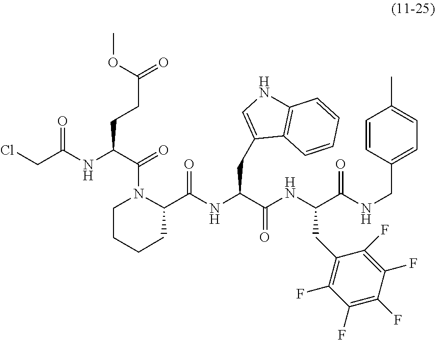 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1c(F)c(F)c(F)c(F)c1F)C(=O)NCc1ccc(C)cc1]

### Methyl (S)-5-((S)-2-(((S)-3-(1H-indol-3-yl)-1-(((S)-1-((4-methyl benzyl)amino)-1-oxo-3-(perfluoro-phenyl)propan-2-yl)amino)-1-oxopropan-2-yl)carbamoyl)piperidin-1-yl)-4-(2-chloroacetamido)-5-oxopentanoate

¹H NMR (500 MHz, DMSO-d₆): δ=10.74 (s, 1H), 8.44 (d, J=5 Hz, 1H), 8.38 (t, J=5 Hz, 1H), 8.34 (d, J=5 Hz, 1H), 8.25 (d, J=5 Hz, 1H), 7.55 (d, J=5 Hz, 1H), 7.50-7.48 (m, 2H), 7.24 (d, J=10 Hz, 2H), 7.03-6.98 (m, 8H), 6.90-6.87 (m, 2H), 4.86 (d, J=5 Hz, 1H), 4.67-4.61 (m, 1H), 4.59-4.51 (m, 1H), 4.17-4.09 (m, 1H), 4.01 (s, 2H), 3.52 (s, 3H), 3.03-2.94 (m, 2H), 2.89-2.81 (m, 2H), 2.25 (t, J=5 Hz, 2H), 2.31 (t, J=5 Hz, 1H), 2.19 (s, 3H), 2.02-1.94 (m, 1H), 1.82-1.71 (m, 1H), 1.60-1.52 (m, 1H), 1.36-1.30 (m, 1H), 1.20-1.16 (m, 1H) ppm.

MS (ESI): m/z=[M+1] 875.68.

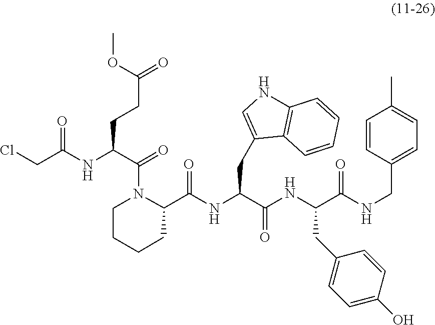 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1ccc(O)cc1)C(=O)NCc1ccc(C)cc1]

### Methyl (S)-4-(2-chloroacetamido)-5-((S)-2-(((S)-1-(((S)-3-(4-hydroxyphenyl)-1-((4-methyl-benzyl)amino)-1-oxopropan-2-yl)amino)-3-(1H-indol-3-yl)-1-oxopropan-2-yl)carbamoyl)piperidin-1-yl)-5-oxopentanoate

MS (ESI): m/z=[M+1] 801.32.

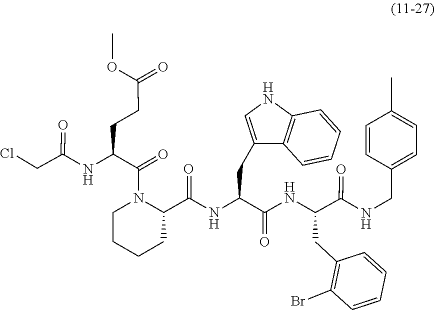 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1ccccc1Br)C(=O)NCc1ccc(C)cc1]

### Methyl (S)-5-((S)-2-(((S)-1-(((S)-3-(2-bromophenyl)-1-((4-methylbenzyl)amino)-1-oxopropan-2-yl)amino)-3-(1H-indol-3-yl)-1-oxopropan-2-yl)carbamoyl)piperidin-1-yl)-4-(2-chloroacetamido)-5-oxopentanoate

MS (ESI): m/z=[M+1] 863.87.

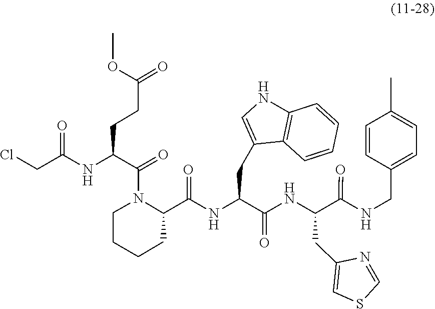 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1cscn1)C(=O)NCc1ccc(C)cc1]

### Methyl (S)-5-((S)-2-(((S)-3-(1H-indol-3-yl)-1-(((S)-1-((4-methyl benzyl)amino)-1-oxo-3-(thiazol-4-yl)propan-2-yl)amino)-1-oxopropan-2-yl)carbamoyl)piperidin-1-yl)-4-(2-chloroacetamido)-5-oxopentanoate

¹H NMR (500 MHz, DMSO-d₆): δ=10.73 (s, 1H), 8.94 (s, 1H), 8.39 (d, J=5 Hz, 1H), 8.18 (d, J=5 Hz, 1H), 8.11 (t, J=5 Hz, 1H), 7.62 (d, J=10 Hz, 1H), 7.49 (d, J=10 Hz, 1H), 7.24 (d, J=10 Hz, 2H), 7.07 (d, J=5 Hz, 2H), 7.00-6.98 (m, 4H), 6.93-6.90 (m, 2H), 4.89 (d, J=5 Hz, 1H), 4.69-4.64 (m, 1H), 4.59-4.55 (m 2H), 4.54-4.49 (m, 1H), 4.12 (d, J=5 Hz, 2H), 4.02 (d, J=5 Hz, 2H), 3.51 (s, 3H), 3.07-3.02 (m, 2H), 2.94-2.89 (m, 1H), 2.79-2.72 (m, 1H), 2.31 (t, J=5 Hz, 1H), 2.25 (t, J=5 Hz, 2H), 2.18 (s, 3H), 2.00-1.97 (m, 1H), 1.81-1.73 (m, 1H), 1.60-1.54 (m, 1H), 1.36-1.27 (m, 2H), 1.20-1.14 (m, 1H), 1.08-1.01 (m, 1H) ppm.

MS (ESI): m/z=[M+1] 792.96.

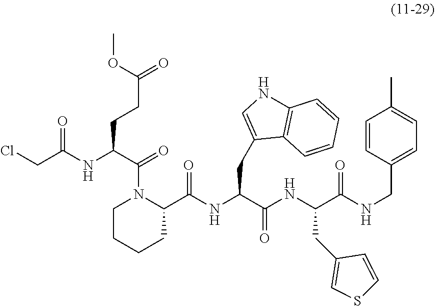 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1ccsc1)C(=O)NCc1ccc(C)cc1]

### Methyl (S)-5-((S)-2-(((S)-3-(1H-indol-3-yl)-1-(((S)-1-((4-methyl benzyl)amino)-1-oxo-3-(thiophen-3-yl)propan-2-yl)amino)-1-oxopropan-2-yl)carbamoyl)piperidin-1-yl)-4-(2-chloroacetamido)-5-oxopentanoate

¹H NMR (500 MHz, DMSO-d₆): δ=10.74 (s, 1H), 8.40 (d, J=5 Hz, 1H), 8.23 (t, J=5 Hz, 1H), 8.09 (d, J=5 Hz, 1H), 7.60 (d, J=10 Hz, 1H), 7.49 (d, J=10 Hz, 1H), 7.26-7.23 (m, 2H), 7.02-6.95 (m, 6H), 6.91 (d, J=5 Hz, 1H), 6.84 (t, J=5 Hz, 1H), 4.90 (d, J=5 Hz, 1H), 4.69-4.65 (m, 1H), 4.59-4.52 (m, 1H), 4.48-4.43 (m, 2H), 4.18-4.13 (m, 2H), 4.02 (d, J=5 Hz, 2H), 3.51 (s, 3H), 3.09-3.04 (m, 2H), 2.95-2.91 (m, 2H), 2.30 (t, J=5 Hz, 1H), 2.25 (t, J=5 Hz, 2H), 2.19 (s, 3H), 2.00-1.98 (m, 1H), 1.82-1.74 (m, 1H), 1.60-1.55 (m, 1H), 1.35-1.28 (m, 2H), 1.19 (q, J=5 Hz, 3H), 1.07-1.01 (m, 1H) ppm.

MS (ESI): m/z=[M+1] 791.91.

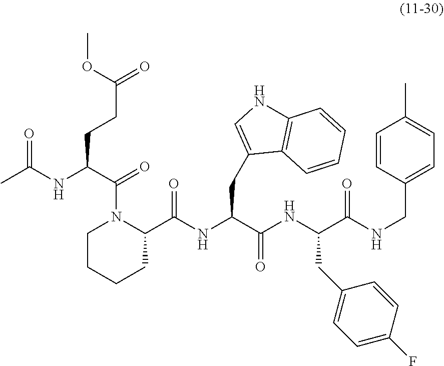 [SMILES: COC(=O)CC[C@H](NC(C)=O)C(=O)N1CCCC[C@H]1C(=O)N[C@@H](Cc1c[nH]c2ccccc12)C(=O)N[C@@H](Cc1ccc(F)cc1)C(=O)NCc1ccc(C)cc1]

### Methyl (S)-4-acetamido-5-((S)-2-(((S)-1-(((S)-3-(4-fluorophenyl)-1-((4-methyl benzyl)-amino)-1-oxopropan-2-yl)amino)-3-(1H-indol-3-yl)-1-oxopropan-2-yl)carbamoyl)piperidin-1-yl)-5-oxopentanoate

MS (ESI): m/z=[M+1] 769.99.

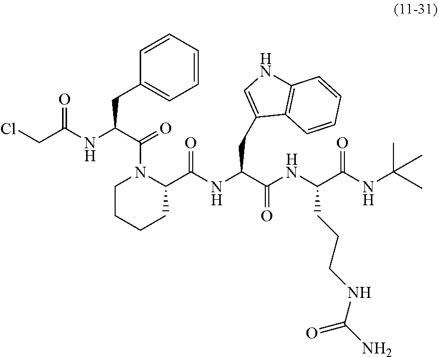 [SMILES: CC(C)(C)NC(=O)[C@H](CCCNC(N)=O)NC(=O)[C@H](Cc1c[nH]c2ccccc12)NC(=O)[C@@H]1CCCCN1C(=O)[C@H](Cc1ccccc1)NC(=O)CCl]

### (S)—N—((S)-1-(((S)-1-(tert-Butylamino)-1-oxo-5-ureidopentan-2-yl)amino)-3-(1H-indol-3-yl)-1-oxopropan-2-yl)-1-((2-chloroacetyl)-L-phenylalanyl)piperidine-2-carboxamide

MS (ESI): m/z=[M+1] 751.43.

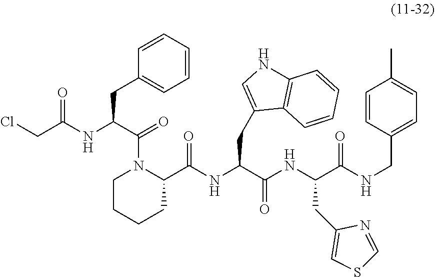 [SMILES: Cc1ccc(CNC(=O)[C@H](Cc2cscn2)NC(=O)[C@H](Cc2c[nH]c3ccccc23)NC(=O)[C@@H]2CCCCN2C(=O)[C@H](Cc2ccccc2)NC(=O)CCl)cc1]

### (S)—N—((S)-3-(1H-Indol-3-yl)-1-(((S)-1-((4-methylbenzyl)amino)-1-oxo-3-(thiazol-4-yl)propan-2-yl)amino)-1-oxopropan-2-yl)-1-((2-chloroacetyl)-L-phenylalanyl)piperidine-2-carboxamide

MS (ESI): m z=[M+] 796.41.

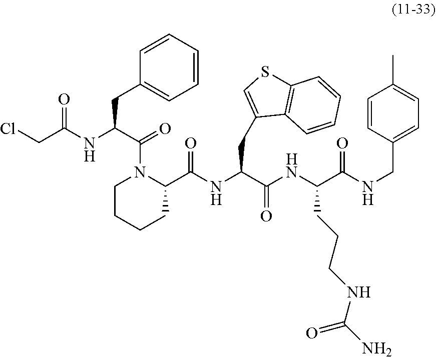 [SMILES: Cc1ccc(CNC(=O)[C@H](CCCNC(N)=O)NC(=O)[C@H](Cc2csc3ccccc23)NC(=O)[C@@H]2CCCCN2C(=O)[C@H](Cc2ccccc2)NC(=O)CCl)cc1]

### (S)—N—((S)-3-(benzo[6]thiophen-3-yl)-1-(((S)-1-((4-methylbenzyl)amino)-1-oxo-5-ureidopentan-2-yl)amino)-1-oxopropan-2-yl)-1-((2-chloroacetyl)-L-phenylalanyl)-piperidine-2-carboxamide

MS (ESI): m/z=[M+1] 816.42.

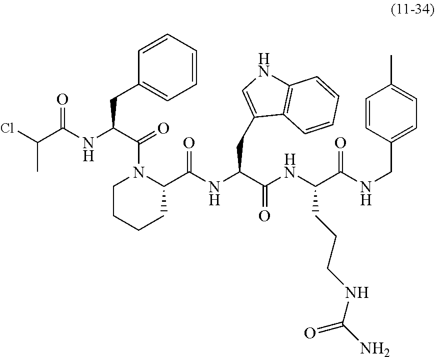 [SMILES: Cc1ccc(CNC(=O)[C@H](CCCNC(N)=O)NC(=O)[C@H](Cc2c[nH]c3ccccc23)NC(=O)[C@@H]2CCCCN2C(=O)[C@H](Cc2ccccc2)NC(=O)C(C)Cl)cc1]

### (2S)—N—((S)-3-(1H-indol-3-yl)-1-(((S)-1-((4-methylbenzyl)amino)-1-oxo-5-ureidopentan-2-yl)amino)-1-oxopropan-2-yl)-1-((2-chloropropanoyl)-L-phenylalanyl)piperidine-2-carboxamide

MS (ESI): m/z=[M+1] 813.47.

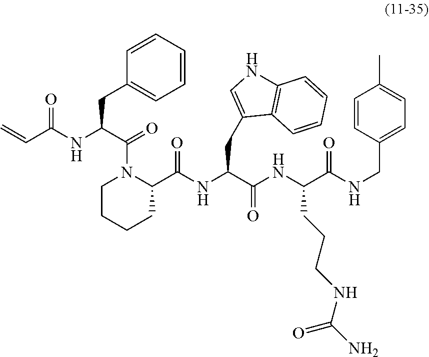 [SMILES: C=CC(=O)N[C@@H](Cc1ccccc1)C(=O)N1CCCC[C@H]1C(=O)N[C@@H](Cc1c[nH]c2ccccc12)C(=O)N[C@@H](CCCNC(N)=O)C(=O)NCc1ccc(C)cc1]

### (S)—N—((S)-3-(1H-indol-3-yl)-1-(((S)-1-((4-methylbenzyl)amino)-1-oxo-5-ureidopentan-2-yl)amino)-1-oxopropan-2-yl)-1-(acryloyl-L-phenylalanyl)piperidine-2-carboxamide

MS (ESI): m/z=[M+1] 777.50.

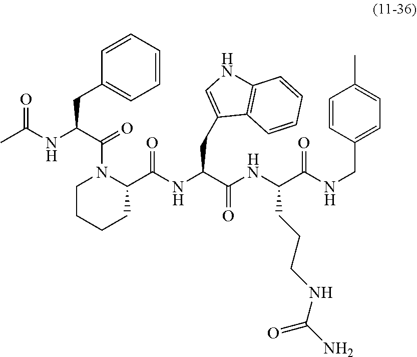 [SMILES: CC(=O)N[C@@H](Cc1ccccc1)C(=O)N1CCCC[C@H]1C(=O)N[C@@H](Cc1c[nH]c2ccccc12)C(=O)N[C@@H](CCCNC(N)=O)C(=O)NCc1ccc(C)cc1]

### (S)—N—((S)-3-(1H-Indol-3-yl)-1-(((S)-1-((4-methylbenzyl)amino)-1-oxo-5-ureidopentan-2-yl)amino)-1-oxopropan-2-yl)-1-(acetyl-L-phenylalanyl)piperidine-2-carboxamide

¹H NMR (500 MHz, DMSO-d₆): δ=10.88 (s, 1H), 8.69 (d, J=5 Hz, 1H), 8.38 (t, J=5 Hz, 1H), 8.34 (t, J=5 Hz, 1H), 8.28 (d, J=10 Hz, 1H), 8.16 (d, J=10 Hz, 1H), 7.89 (d, J=5 Hz, 1H), 7.64 (t, J=5 Hz, 1H), 7.58 (d, J=5 Hz, 1H), 7.38-7.35 (m, 1H), 7.31 (d, J=5 Hz, 3H), 7.28-7.25 (m, 2H), 7.19-7.17 (m, 3H), 7.12-7.18 (m, 2H), 7.03-7.01 (m, 1H), 6.00 (s, 1H), 5.07 (d, J=5 Hz, 1H), 4.95-4.90 (m, 1H), 4.36-4.33 (m, 2H), 4.31-4.27 (m, 2H), 3.27-3.22 (m, 2H), 3.04-2.93 (m, 4H), 2.34-2.29 (m, 3H), 2.16 (s, 1H), 1.90 (s, 3H), 1.77 (s, 3H), 1.46-1.43 (m, 1H), 1.41-1.37 (m, 2H), 1.33 (d, J=5 Hz, 1H), 1.27-1.22 (m, 1H), 1.18-1.15 (m, 2H), 1.13 (t, J=5 Hz, 1H) ppm.

MS (ESI): m/z=[M+1] 765.49.

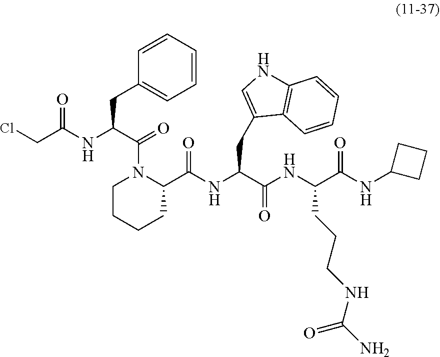 [SMILES: NC(=O)NCCC[C@H](NC(=O)[C@H](Cc1c[nH]c2ccccc12)NC(=O)[C@@H]1CCCCN1C(=O)[C@H](Cc1ccccc1)NC(=O)CCl)C(=O)NC1CCC1]

### (S)-1-((2-Chloroacetyl)-L-phenylalanyl)-N—((S)-1-(((S)-1-(cyclobutylamino)-1-oxo-5-ureidopentan-2-yl)amino)-3-(1H-indol-3-yl)-1-oxopropan-2-yl)piperidine-2-carboxamide

MS (ESI): m/z=[M+1] 749.18.

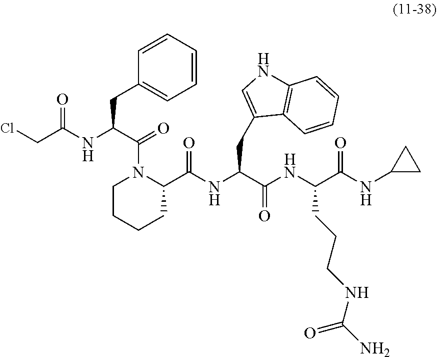 [SMILES: NC(=O)NCCC[C@H](NC(=O)[C@H](Cc1c[nH]c2ccccc12)NC(=O)[C@@H]1CCCCN1C(=O)[C@H](Cc1ccccc1)NC(=O)CCl)C(=O)NC1CC1]

### (S)-1-((2-chloroacetyl)-L-phenylalanyl)-N—((S)-1-(((S)-1-(cyclopropylamino)-1-oxo-5-ureidopentan-2-yl)amino)-3-(1H-indol-3-yl)-1-oxopropan-2-yl)piperidine-2-carboxamide

MS (ESI): m/z=[M+1] 735.37.

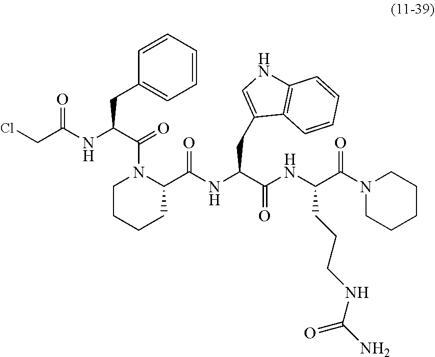 [SMILES: NC(=O)NCCC[C@H](NC(=O)[C@H](Cc1c[nH]c2ccccc12)NC(=O)[C@@H]1CCCCN1C(=O)[C@H](Cc1ccccc1)NC(=O)CCl)C(=O)N1CCCCC1]

### (S)—N—((S)-3-(1H-indol-3-yl)-1-oxo-1-(((S)-1-oxo-1-(piperidin-1-yl)-5-ureidopentan-2-yl)amino) propan-2-yl)-1-((2-chloroacetyl)-L-phenylalanyl)piperidine-2-carboxamide

MS (ESI): m/z=[M+1] 763.14.

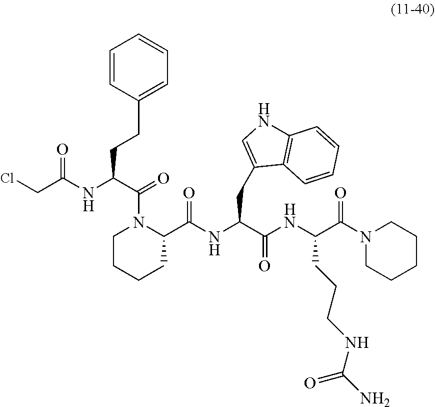 [SMILES: NC(=O)NCCC[C@H](NC(=O)[C@H](Cc1c[nH]c2ccccc12)NC(=O)[C@@H]1CCCCN1C(=O)[C@H](CCc1ccccc1)NC(=O)CCl)C(=O)N1CCCCC1]

### (S)—N—((S)-3-(1H-Indol-3-yl)-1-oxo-1-(((S)-1-oxo-1-(piperidin-1-yl)-5-ureidopentan-2-yl)amino)propan-2-yl)-1-((S)-2-(2-chloroacetamido)-4-phenylbutanoyl)piperidine-2-carboxamide

MS (ESI): m/z=[M+1] 777.10.

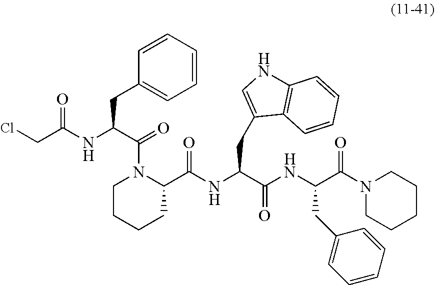 [SMILES: O=C(CCl)N[C@@H](Cc1ccccc1)C(=O)N1CCCC[C@H]1C(=O)N[C@@H](Cc1c[nH]c2ccccc12)C(=O)N[C@@H](Cc1ccccc1)C(=O)N1CCCCC1]

### (S)—N—((S)-3-(1H-Indol-3-yl)-1-oxo-1-(((S)-1-oxo-3-phenyl-1-(piperidin-1-yl)propan-2-yl)amino)propan-2-yl)-1-((2-chloroacetyl)-L-phenylalanyl)piperidine-2-carboxamide

MS (ESI) m/z=[M+1] 753.08.

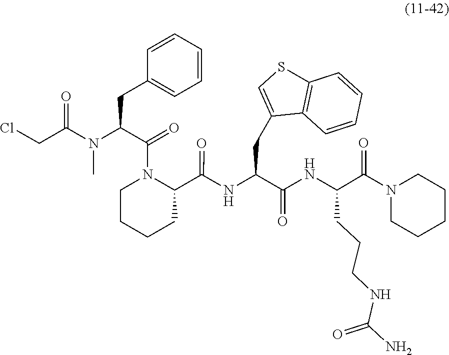 [SMILES: CN(C(=O)CCl)[C@@H](Cc1ccccc1)C(=O)N1CCCC[C@H]1C(=O)N[C@@H](Cc1csc2ccccc12)C(=O)N[C@@H](CCCNC(N)=O)C(=O)N1CCCCC1]

### (S)—N—((S)-3-(Benzo[6]thiophen-3-yl)-1-oxo-1-(((S)-1-oxo-1-(piperidin-1-yl)-5-ureidopentan-2-yl)amino)propan-2-yl)-1-(N-(2-chloroacetyl)-N-methyl-L-phenylalanyl)piperidine-2-carboxamide

¹H NMR (500 MHz, DMSO-d₆): δ=8.21 (d, J=5 Hz, 1H), 7.88 (d, J=5 Hz, 2H), 7.72 (d, J=5 Hz, 1H), 7.37-7.33 (m, 1H), 7.30-7.27 (m, 3H), 7.19-7.10 (m, 7H), 5.84 (s, 1H), 5.44-5.41 (m, 1H), 4.91 (d, J=5 Hz, 1H), 4.78-4.75 (m, 1H), 4.68-4.64 (m, 2H), 4.25 (d, J=10 Hz, 1H), 4.12 (d, J=10 Hz, 2H), 3.23-3.19 (m, 2H), 3.07-3.02 (m, 2H), 2.98-2.95 (m, 1H), 2.82 (s, 3H), 1.99 (d, J=10 Hz, 2H), 1.60-1.56 (m, 1H), 1.53-1.48 (m, 3H), 1.45-1.49 (m, 3H), 1.35-1.32 (m, 3H), 1.31-1.28 (m, 3H), 1.17-1.12 (m, 2H), 0.99-0.93 (3H) ppm.

MS (ESI): m/z=[M+1] 794.06.

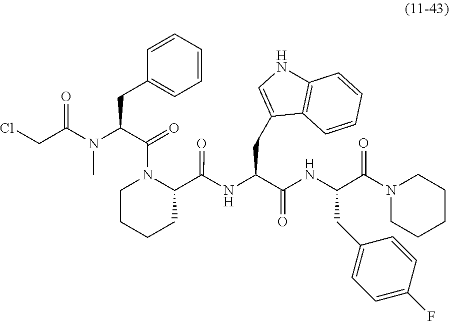 [SMILES: CN(C(=O)CCl)[C@@H](Cc1ccccc1)C(=O)N1CCCC[C@H]1C(=O)N[C@@H](Cc1c[nH]c2ccccc12)C(=O)N[C@@H](Cc1ccc(F)cc1)C(=O)N1CCCCC1]

### (S)-1-(N-(2-Chloroacetyl)-N-methyl-L-phenylalanyl)-N—((S)-1-(((S)-3-(4-fluorophenyl)-1-oxo-1-(piperidin-1-yl)propan-2-yl)amino)-3-(1H-indol-3-yl)-1-oxopropan-2-yl)piperidine-2-carboxamide

¹H NMR (500 MHz, DMSO-d₆): δ=10.75 (s, 1H), 8.28 (d, J=5 Hz, 1H), 7.50 (t, J=10 Hz, 1H), 7.24-7.22 (m, 2H), 7.16-7.11 (m, 6H), 7.01-6.97 (m, 3H), 6.90 (t, J=5 Hz, 2H), 5.44-5.41 (m, 1H), 4.90 (d, J=5 Hz, 1H), 4.86-4.81 (m, 1H), 4.65-4.61 (m, 1H), 4.24 (d, J=10 Hz, 1H), 4.11 (d, J=10 Hz, 1H), 3.32-3.28 (m, 2H), 3.24-3.20 (m, 1H), 3.03-2.99 (m, 1H), 2.94-2.87 (m, 3H), 2.81 (s, 3H), 2.75-2.72 (m, 2H), 2.67-2.63 (m, 1H), 2.02-1.97 (m, 2H), 1.46-1.40 (m, 2H), 1.32-1.27 (m, 3H), 1.20-1.17 (m, 3H), 1.11-1.06 (m, 3H), 0.98-0.90 (m, 2H) ppm.

MS (ESI): m/z=[M+1] 785.00.

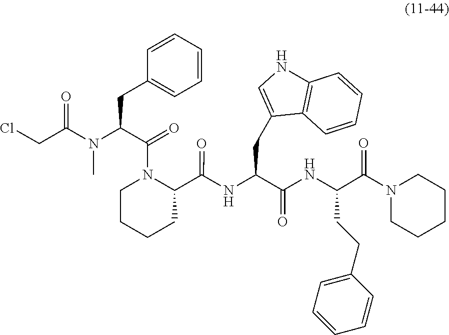 [SMILES: CN(C(=O)CCl)[C@@H](Cc1ccccc1)C(=O)N1CCCC[C@H]1C(=O)N[C@@H](Cc1c[nH]c2ccccc12)C(=O)N[C@@H](CCc1ccccc1)C(=O)N1CCCCC1]

### (S)—N—((S)-3-(1H-Indol-3-yl)-1-oxo-1-(((S)-1-oxo-4-phenyl-1-(piperidin-1-yl)butan-2-yl)amino)propan-2-yl)-1-(N-(2-chloroacetyl)-N-methyl-L-phenylalanyl)piperidine-2-carboxamide

MS (ESI): m/z=[M+1] 781.20.

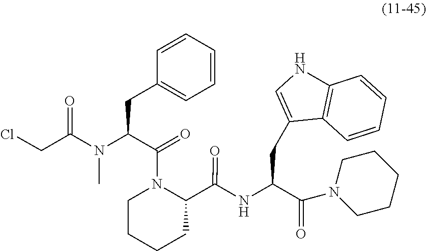 [SMILES: CN(C(=O)CCl)[C@@H](Cc1ccccc1)C(=O)N1CCCC[C@H]1C(=O)N[C@@H](Cc1c[nH]c2ccccc12)C(=O)N1CCCCC1]

### (S)—N—((S)-3-(1H-indol-3-yl)-1-oxo-1-(piperidin-1-yl)propan-2-yl)-1-(N-(2-chloroacetyl)-N-methyl-L-phenylalanyl)piperidine-2-carboxamide

MS (ESI): m/z=[M+1] 620.12.

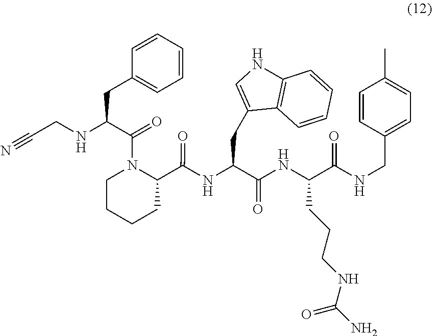 [SMILES: Cc1ccc(CNC(=O)[C@H](CCCNC(N)=O)NC(=O)[C@H](Cc2c[nH]c3ccccc23)NC(=O)[C@@H]2CCCCN2C(=O)[C@H](Cc2ccccc2)NCC#N)cc1]

### (S)—N—((S)-3-(1H-indol-3-yl)-1-(((S)-1-((4-methylbenzyl)amino)-1-oxo-5-ureidopentan-2-yl)amino)-1-oxopropan-2-yl)-1-((cyanomethyl)-L-phenylalanyl)piperidine-2-carboxamide (Representative compound 12)

To a solution of compound 10a (30 mg, 0.04 mmol), N,N-diisopropylethylamine (0.021 mL, 0.12 mmol) in acetonitrile (0.5 mL), was added bromoacetonitrile (0.006 mL, 0.08 mmol).

The reaction mixture was stirred at room temperature for 1 hour. The solvent was then evaporated, the product purified via HPLC and lyophilized to yield compound 12 (18 mg, 58%) as a white powder.

¹H NMR (500 MHz, DMSO-d₆): δ=10.82 (s, 1H), 8.26 (t, J=5 Hz, 1H), 8.12-8.08 (m, 1H), 7.66 (d, J=5 Hz, 1H), 7.61-7.56 (m, 2H), 7.32-7.27 (m, 2H), 7.23-7.16 (m, 3H), 7.12-7.04 (m, 4H), 6.95 (t, J=5 Hz, 2H), 5.94 (d, J=5 Hz, 1H), 5.40 (s, 1H), 5.05 (d, J=5 Hz, 1H), 4.70-4.64 (m, 2H), 4.30-4.26 (m, 2H), 4.21 (d, J=5 Hz, 2H), 4.18 (d, J=5 Hz, 2H), 3.20-3.16 (m, 2H), 3.01-2.92 (m, 2H), 2.86-2.83 (m, 1H), 2.75-2.69 (m, 1H), 2.37 (t, J=5 Hz, 1H), 2.27 (d, J=5 Hz, 2H), 2.25 (s, 3H), 2.10-2.06 (m, 2H), 1.84-1.78 (m, 1H), 1.69-1.63 (m, 1H), 1.54-1.47 (m, 2H), 1.39-1.32 (m, 2H), 1.26-1.23 (m, 1H), 1.18 (d, J=5 Hz, 1H), 0.89-0.84 (m, 1H) ppm.

MS (ESI): m/z=[M+1] 762.09.

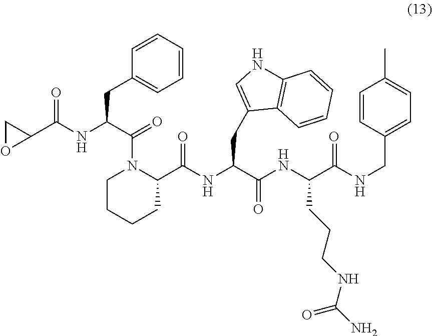 [SMILES: Cc1ccc(CNC(=O)[C@H](CCCNC(N)=O)NC(=O)[C@H](Cc2c[nH]c3ccccc23)NC(=O)[C@@H]2CCCCN2C(=O)[C@H](Cc2ccccc2)NC(=O)C2CO2)cc1]

### (2S)—N—((S)-3-(1H-Indol-3-yl)-1-(((S)-1-((4-methylbenzyl)amino)-1-oxo-5-ureidopentan-2-yl)amino)-1-oxopropan-2-yl)-1-((oxirane-2-carbonyl)-L-phenylalanyl)piperidine-2-carboxamide (Representative Compound 13)

¹H NMR (500 MHz, DMSO-d₆): δ=10.75 (s, 1H), 8.40 (d, J=10 Hz, 1H), 8.19 (t, J=5 Hz, 1H), 8.02 (d, J=5 Hz, 1H), 7.59 (t, J=5 Hz, 1H), 7.52 (d, J=5 Hz, 2H), 7.46 (d, J=5 Hz, 1H), 7.24 (d, J=10 Hz, 1H), 7.17-7.12 (m, 4H), 7.05-7.03 (m, 4H), 6.99-6.95 (m, 2H), 6.89 (t, J=5 Hz, 1H), 5.87 (s, 1H), 5.34 (s, 1H), 4.94 (d, J=5 Hz, 1H), 4.88-4.82 (m, 1H), 4.63-4.59 (m, 1H), 4.23-4.20 (m, 1H), 4.15 (t, J=5 Hz, 2H), 4.12-4.10 (m, 1H), 3.13-3.09 (m, 1H), 2.91-2.84 (m, 4H), 2.75-2.68 (m, 2H), 2.19 (d, J=10 Hz, 3H), 2.05-2.02 (m, 1H), 1.66-1.57 (m, 2H), 1.49-1.41 (m, 2H), 1.39-1.35 (m, 1H), 1.32-1.24 (m, 3H), 1.17 (s, 1H), 1.14 (d, J=5 Hz, 1H), 1.11 (d, J=5 Hz, 1H), 0.80-0.77 (m, 1H) ppm.

MS (ESI): m/z=[M+1] 793.51.

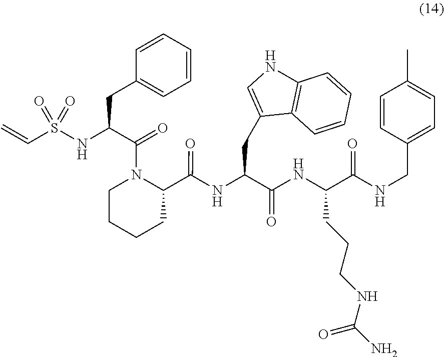 [SMILES: C=CS(=O)(=O)N[C@@H](Cc1ccccc1)C(=O)N1CCCC[C@H]1C(=O)N[C@@H](Cc1c[nH]c2ccccc12)C(=O)N[C@@H](CCCNC(N)=O)C(=O)NCc1ccc(C)cc1]

### (S)—N—((S)-3-(1H-indol-3-yl)-1-(((S)-1-((4-methylbenzyl)amino)-1-oxo-5-ureidopentan-2-yl)amino)-1-oxopropan-2-yl)-1-((vinylsulfonyl)-L-phenylalanyl)piperidine-2-carboxamide (14)

¹H NMR (500 MHz, DMSO-d₆): δ=10.86 (s, 1H), 9.74 (d, J=5 Hz, 1H), 8.75 (t, J=5 Hz, 1H), 8.31 (t, J=5 Hz, 1H), 8.14 (d, J=5 Hz, 1H), 7.80 (d, J=5 Hz, 1H), 7.62 (t, J=5 Hz, 2H), 7.34-7.30 (m, 3H), 7.28-7.25 (m, 3H), 7.18-7.13 (m, 4H), 7.09 (t, J=5 Hz, 1H), 6.99 (t, J=5 Hz, 1H), 6.59 (s, 1H), 5.99 (s, 1H), 5.73 (d, J=10 Hz, 1H), 5.43 (s, 1H), 5.05 (d, J=5 Hz, 1H), 4.72-4.69 (m, 1H), 4.66-4.63 (m, 1H), 4.34-4.30 (m, 3H), 4.26-4.23 (m, 2H), 3.22 (t, J=5 Hz, 1H), 3.02-2.97 (m, 2H), 2.45 (t, J=5 Hz, 1H), 2.42 (t, J=5 Hz, 2H), 2.33 (s, 3H), 2.29 (s, 2H), 2.17-2.14 (m, 1H), 1.73-1.68 (m, 2H), 1.59-1.52 (m, 2H), 1.43-1.39 (m, 2H), 1.31-1.28 (m, 2H) ppm.

MS (ESI): m/z=[M+1] 813.47.

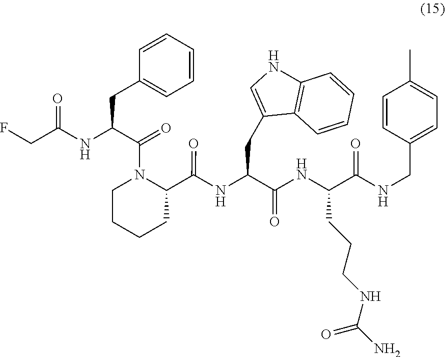 [SMILES: Cc1ccc(CNC(=O)[C@H](CCCNC(N)=O)NC(=O)[C@H](Cc2c[nH]c3ccccc23)NC(=O)[C@@H]2CCCCN2C(=O)[C@H](Cc2ccccc2)NC(=O)CF)cc1]

### (S)—N—((S)-3-(1H-Indol-3-yl)-1-(((S)-1-((4-methylbenzyl)amino)-1-oxo-5-ureidopentan-2-yl)amino)-1-oxopropan-2-yl)-1-((2-fluoroacetyl)-L-phenylalanyl)piperidine-2-carboxamide

### Example 5: Identification and Characterization of Compounds Synthesized According to Scheme 3

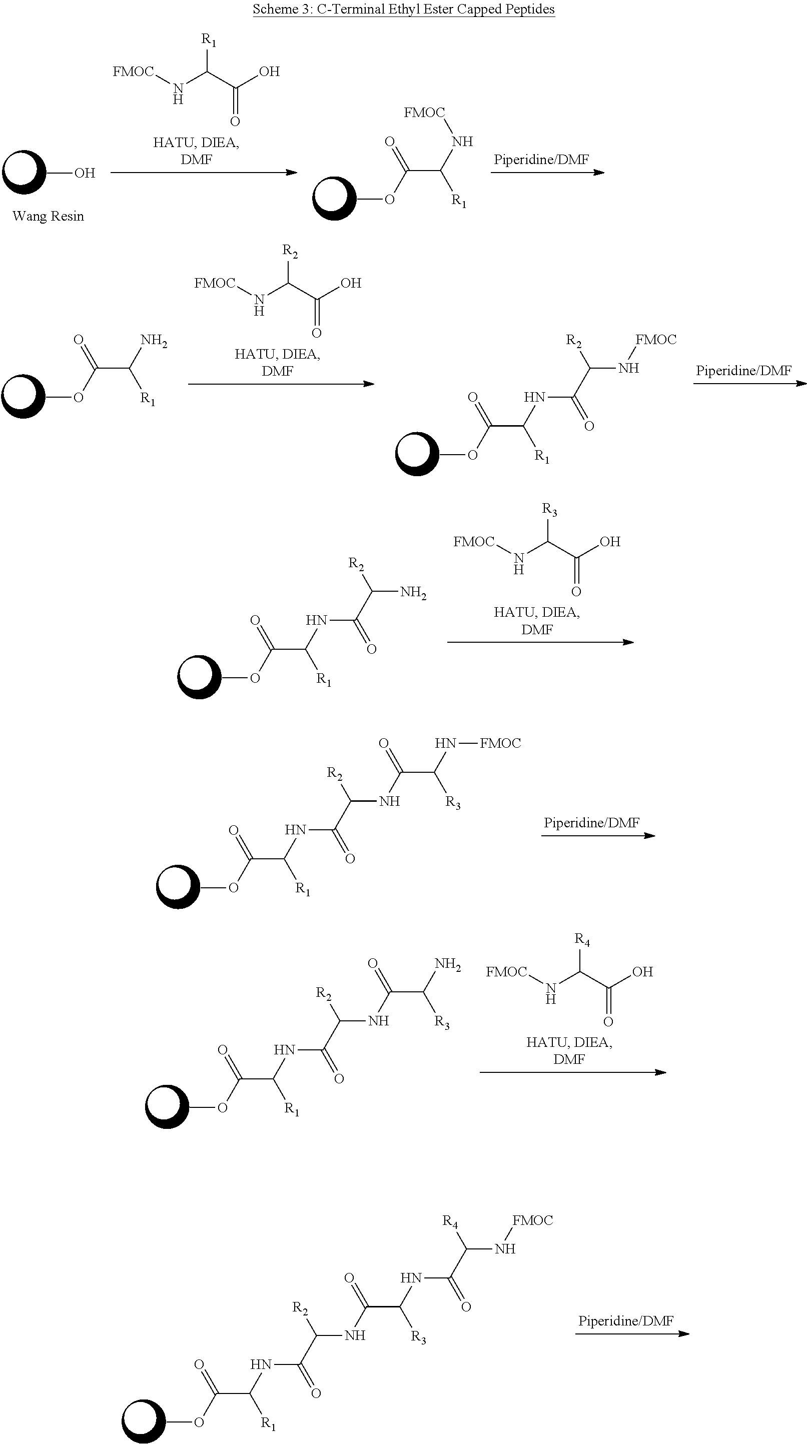

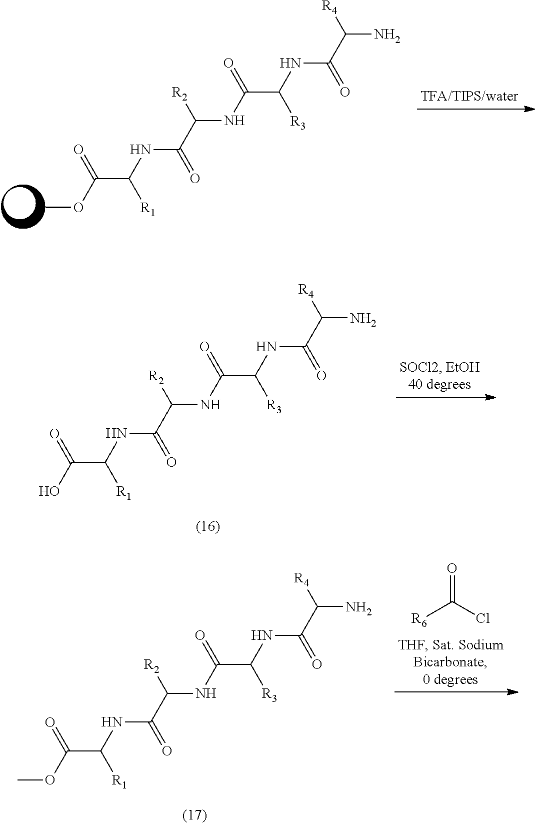

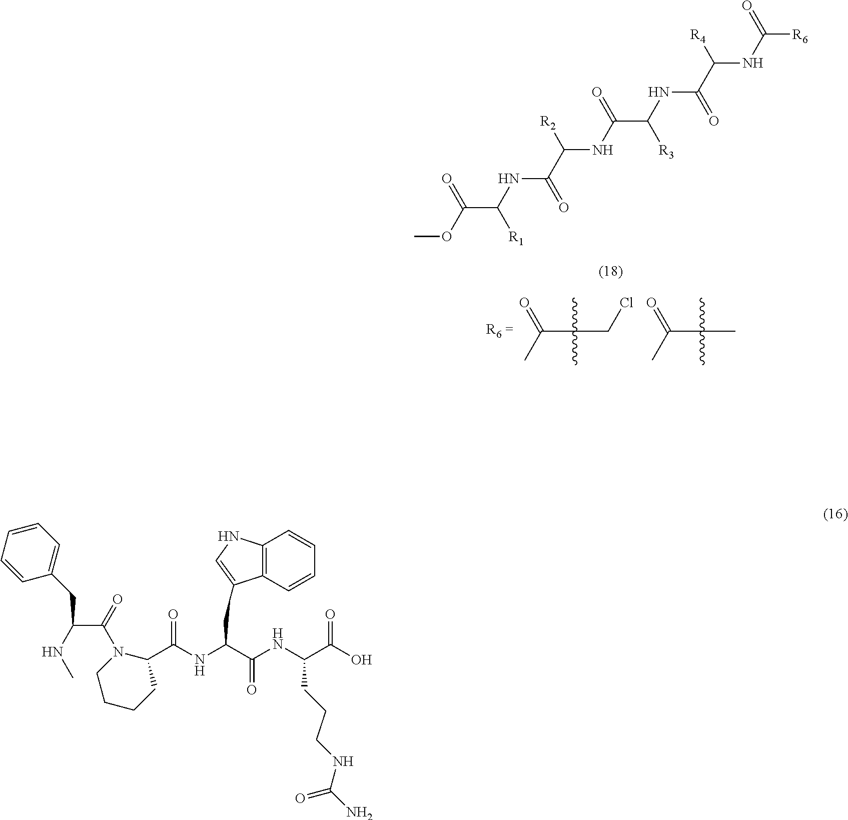

### (S)-2-((S)-3-(1H-indol-3-yl)-2-((S)-1-(methyl-L-phenylalanyl)piperidine-2-carboxamido) propanamido)-5-ureidopentanoic acid (16)

Peptide 16 was synthesized according to general solid phase peptide synthesis procedures starting from 0.12 mmol of Wang resin. The following amino acids were coupled to the resin: Fmoc-N-Me-Phe-OH, Fmoc-pipecolinic acid, Fmoc-Trp(BOC)-OH, Fmoc-Cit-OH.

¹H NMR (500 MHz, DMSO-d₆): δ=12.57 (s, 1H), 10.75 (d, J=20 Hz, 1H), 9.26 (s, 1H), 8.85-8.75 (m, 1H), 8.61 (s, 1H), 8.26-8.22 (m, 1H), 7.72-7.65 (m, 1H), 7.56 (t, J=5 Hz, 1H), 7.29-7.27 (m, 2H), 7.24 (d, J=10 Hz, 1H), 7.16 (d, J=5 Hz, 1H), 7.12 (d, J=5 Hz, 2H), 7.08 (dd, J=15, 5 Hz, 1H), 7.02-6.96 (m, 1H), 6.91 (dd, J=15, 5 Hz, 1H), 5.91 (d, J=5 Hz, 1H), 5.33 (s, 1H), 4.75-4.8 (m, 1H), 4.66-4.61 (m, 1H), 4.49 (s, 1H), 4.15-4.10 (m, 2H), 4.05-4.02 (m, 1H), 3.86 (d, J=5 Hz, 1H), 2.97-2.93 (m, 1H), 2.88 (s, 3H), 2.35 (t, J=5 Hz, 1H), 2.06 (t, J=5 Hz, 2H), 1.68-1.63 (m, 1H), 1.56-1.40 (m, 4H), 1.34-1.33 (m, 3H), 1.21-1.17 (m, 2H) ppm.

MS (ESI): m/z=[M+1] 634.08.

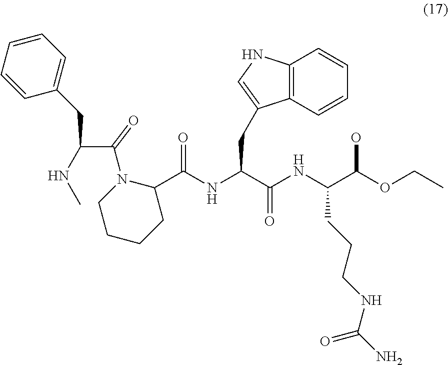 [SMILES: CCO[C@H](O)[C@H](CCCNC(N)=O)NC(=O)[C@H](Cc1c[nH]c2ccccc12)NC(=O)C1CCCCN1C(=O)[C@H](Cc1ccccc1)NC]

### Ethyl (S)-2-((S)-3-(1H-indol-3-yl)-2-((S)-1-(methyl-L-phenylalanyl)piperidine-2-carboxamido)propanamido)-5-ureidopentanoate (17)

To a solution of peptide 16 (50 mg, 0.08 mmol) in ethanol (0.5 mL), was added thionyl chloride (0.02 mL, 0.28 mmol) drop-wise in an ice bath. The resulting solution was stirred at 40° C. for 1 hour. The solvent was then evaporated and the product purified by HPLC purification to yield compound 17 as a white powder (28 mg, 52%).

¹H NMR (500 MHz, DMSO-d₆): δ=10.74 (d, J=10 Hz, 1H), 8.39 (t, J=10 Hz, 1H), 7.70 (dd, J=20, 10 Hz, 1H), 7.54 (t, J=5 Hz, 1H), 7.26 (d, J=10 Hz, 2H), 7.23 (d, J=5 Hz, 1H), 7.15 (d, J=5 Hz, 1H), 7.10 (d, J=10 Hz, 2H), 7.05 (d, J=5 Hz, 1H), 7.01-6.95 (m, 1H), 6.90 (t, J=5 Hz, 1H), 5.89 (s, 1H), 5.33 (s, 1H), 4.69-4.60 (m, 2H), 4.14-4.09 (m, 2H), 4.04-3.97 (m, 3H), 3.84 (s, 1H), 3.10-2.99 (m, 2H), 2.93-2.86 (m, 4H), 2.71 (t, J=10 Hz, 1H), 2.34 (t, J=5 Hz, 2H), 2.04 (t, J=5 Hz, 2H), 1.67-1.62 (m, 2H), 1.53-1.49 (m, 1H), 1.43-1.40 (m, 1H), 1.30-1.13 (m, 4H), 1.09 (t, J=5 Hz, 3H), 0.90-0.85 (m, 1H), 0.76-0.67 (m, 1H) ppm. ¹³C NMR (126 MHz, DMSO-d₆): δ=172.3, 171.9, 170.2, 168.9, 167.6, 159.2, 136.5, 134.3, 130.3, 129.2, 127.9, 124.1, 121.4, 118.9, 111.9, 110.5, 60.9, 58.9, 57.9, 56.3, 53.7, 52.6, 43.3, 37.3, 35.4, 32.3, 31.7, 28.7, 28.2, 27.1, 25.8, 25.4, 24.1, 19.8, 14.5 ppm.

MS (ESI): m/z=[M+1] 662.09.

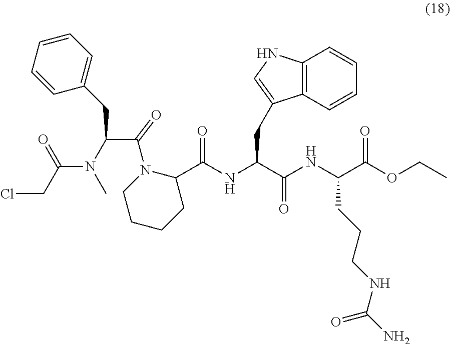 [SMILES: CCOC(=O)[C@H](CCCNC(N)=O)NC(=O)[C@H](Cc1c[nH]c2ccccc12)NC(=O)C1CCCCN1C(=O)[C@H](Cc1ccccc1)N(C)C(=O)CCl]

### Ethyl (S)-2-((S)-2-((S)-1-(N-(2-chloroacetyl)-N-methyl-L-phenylalanyl)piperidine-2-carboxamido)-3-(1H-indol-3-yl)propanamido)-5-ureidopentanoate

To a mixture of compound 17 (28 mg, 0.04 mmol) in 0.2 mL THF and 0.2 mL saturated sodium bicarbonate was added a solution of chloroacetyl chloride (0.006 mL, 0.08 mmol) in 0.2 mL THF. The resulting mixture was stirred in an ice bath for 2 hours. The mixture was then diluted with 0.5 mL of dichloromethane and 0.5 mL of methanol, and filtered through a 0.45 um syringe filter. The solvent was evaporated, the product purified via HPLC and lyophilized to yield compound 18 as a white powder (12 mg, 40%).

¹H NMR (500 MHz, DMSO-d₆): δ=10.75 (s, 1H), 8.40 (d, J=5 Hz, 1H), 7.53 (dd, J=15, 10 Hz, 1H), 7.24 (d, J=5 Hz, 1H), 7.19-7.09 (m, 4H), 7.04-7.01 (m, 1H), 6.98 (t, J=5 Hz, 1H), 6.90 (t, J=5 Hz, 1H), 5.90 (s, 1H), 5.42 (q, J=5 Hz, 1H), 5.34 (t, J=5 Hz, 1H), 4.90 (d, J=5 Hz, 1H), 4.71-4.67 (m, 1H), 4.62-4.57 (m, 1H), 4.23 (d, J=15 Hz, 1H), 4.17-4.13 (m, 1H), 4.10 (d, J=15 Hz, 1H), 4.05-3.98 (m, 2H), 3.76 (d, J=15 Hz, 1H), 3.33 (d, J=10 Hz, 2H), 3.09 (dd, J=15, 5 Hz, 1H), 2.95-2.84 (m, 4H), 2.81 (s, 3H), 2.75 (s, 2H), 2.01 (d, J=10 Hz, 1H), 1.69-1.63 (m, 1H), 1.57-1.53 (m, 1H), 1.38-1.25 (m, 3H), 1.21-1.17 (m, 1H), 1.12 (t, J=5 Hz, 3H), 1.07-1.05 (m, 1H), 0.98-0.85 (m, 2H) ppm. ¹³C NMR (126 MHz, DMSO-d₆): δ=172.4, 172.3, 169.4, 165.9, 159.2, 137.9, 136.6, 129.7, 128.6, 127.7, 126.7, 124.2, 121.3, 119.0, 118.6, 111.7 110.4, 60.9, 54.7, 53.3, 53.0, 52.7, 42.3, 40.5, 40.3, 40.2, 40.0, 39.8, 39.7, 39.5, 34.6, 30.6, 28.7, 27.1, 25.2, 20.6, 14.5.

MS (ESI): m/z [M+1] 738.13.

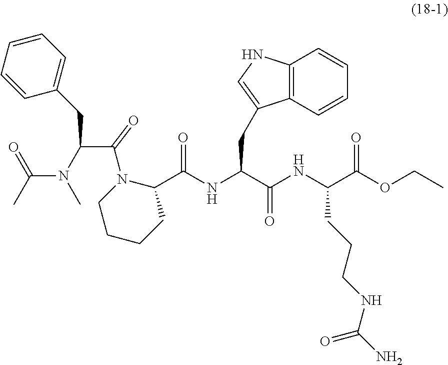 [SMILES: CCOC(=O)[C@H](CCCNC(N)=O)NC(=O)[C@H](Cc1c[nH]c2ccccc12)NC(=O)[C@@H]1CCCCN1C(=O)[C@H](Cc1ccccc1)N(C)C(C)=O]

### Ethyl (S)-2-((S)-2-((S)-1-(N-acetyl-N-methyl-L-phenylalanyl)piperidine-2-carboxamido)-3-(1H-indol-3-yl)propanamido)-5-ureidopentanoate

¹H NMR (500 MHz, DMSO-d₆): δ=10.82 (s, 1H), 8.47 (d, J=5 Hz, 1H), 7.62 (d, J=10 Hz, 1H), 7.58 (d, J=10 Hz, 1H), 7.31 (d, J=10 Hz, 2H), 7.24 (t, J=5 Hz, 2H), 7.21 (s, 1H), 7.17 (t, J=5 Hz, 1H), 7.12-7.09 (m, 1H), 7.05 (t, J=5 Hz, 1H), 6.98 (t, J=5 Hz, 1H), 5.96 (s, 1H), 5.53 (q, J=5 Hz, 1H), 5.39 (s, 1H), 4.98 (d, J=5 Hz, 1H), 4.79-4.74 (m, 1H), 4.67-4.65 (m, 1H), 4.27-4.21 (m, 1H), 4.11 (q, J=5 Hz, 2H), 3.63 (d, J=5 Hz, 1H), 3.19-3.15 (m, 2H), 3.02-2.93 (m, 4H), 2.90-2.86 (m, 1H), 2.81 (s, 3H), 2.76 (s, 1H), 2.67 (s, 1H), 2.09 (s, 3H), 1.81 (s, 1H), 1.76-1.71 (m, 1H), 1.53 (d, J=5 Hz, 1H), 1.46-1.42 (m, 2H), 1.38-1.33 (m, 1H), 1.28-1.24 (m, 2H), 1.20 (t, J=5 Hz, 3H), 1.00-0.94 (m, 2H), ppm. ¹³C NMR (126 MHz, DMSO-d₆): δ=172.4, 170.0, 169.5, 159.2, 138.3, 136.6, 129.7, 128.5, 127.7, 126.6, 124.2, 121.3, 119.0, 118.6, 111.7, 110.4, 60.9, 53.8, 52.9, 52.5, 42.7, 40.5, 40.3, 40.2, 40.0, 39.8, 39.7, 39.5, 34.8, 31.4, 28.7, 28.0, 27.1, 25.1, 21.8, 20.6, 14.5 ppm.

MS (ESI): m/z=[M+1] 704.46.

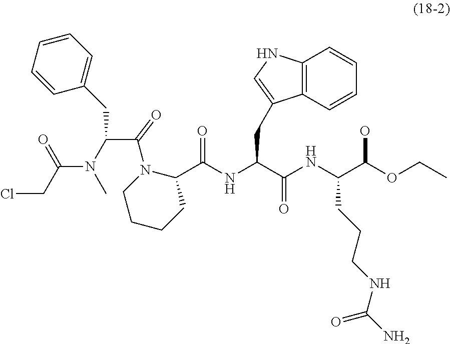 [SMILES: CCO[C@H](O)[C@H](CCCNC(N)=O)NC(=O)[C@H](Cc1c[nH]c2ccccc12)NC(=O)[C@@H]1CCCCN1C(=O)[C@@H](Cc1ccccc1)N(C)C(=O)CCl]

### Ethyl (S)-2-((S)-2-((S)-1-(N-(2-chloroacetyl)-N-methyl-Z)-phenylalanyl)piperidine-2-carboxamido)-3-(1H-indol-3-yl)propanamido)-5-ureidopentanoate)

³H NMR (500 MHz, DMSO-d₆): δ=10.75 (s, 1H), 8.32 (d, J=5 Hz, 1H), 7.53 (d, J=5 Hz, 1H), 7.50 (d, J=5 Hz, 1H), 7.25 (d, J=5 Hz, 1H), 7.28 (d, J=5 Hz, 1H), 7.16 (d, J=5 Hz, 2H), 7.13 (d, J=5 Hz, 2H), 7.10-7.05 (m, 2H), 6.98 (t, J=10 Hz, 1H), 6.90 (t, J=10 Hz, 1H), 5.90 (s, 1H), 5.36 (t, J=5 Hz, 1H), 4.86 (d, J=5 Hz, 1H), 4.60-4.55 (m, 1H), 4.20 (d, J=10 Hz, 2H), 4.16-4.11 (m, 2H), 4.02 (q, J=5 Hz, 2H), 3.37-3.34 (m, 1H), 3.09-3.03 (m, 2H), 2.91-2.84 (m, 4H), 2.81 (s, 3H), 2.74-2.70 (m, 1H), 2.02-1.98 (m, 1H), 1.67-1.60 (m, 1H), 1.56-1.50 (m, 1H), 1.33-1.25 (m, 5H), 1.11 (t, J=5 Hz, 3H), 0.99-0.92 (m, 2H) ppm. ¹³C NMR (126 MHz, DMSO-de): δ=172.3, 170.5, 169.5, 166.4, 159.2, 137.9, 136.5, 129.8, 128.6, 128.5, 127.8, 126.8, 124.0, 121.3, 118.9, 118.6, 111.7, 110.3, 60.9, 55.0, 53.3, 52.6, 42.5, 40.5, 40.3, 40.2, 40.0, 39.8, 39.7, 39.5, 35.1, 30.9, 28.7, 26.9, 25.0, 20.0, 14.5 ppm.

MS (ESI): m/z=[M+1] 738.43.

### Example 6: Fluorescence Polarization (FP) (Binding to Pin1)

Binding affinity to Pin1 was determined using a fluorescence polarization assay to assess competition with an N-terminal fluorescein-labeled peptide (Bth-D-phos.Thr-Pip-Nal), which was synthesized by a peptide synthesis company. The indicated concentrations of candidate compound were pre-incubated for 12 hours at 4° C. with a solution containing 250 nM glutathione S-transferase (GST)-Pin1, 5 nM of fluorescein-labeled peptide probe, 10 μg/ml bovine serum albumin, 0.01% Tween-20 and 1 mM dithiothreitol (DTT) in a buffer of 10 mM 2-hydroxyethyl)-1-piperazineethanesulfonic acid (HEPES), 10 mMNaCl and 1% glycerol (pH 7.4). Measurements of FP were made in black 384-well plates (Corning) using an EnVision reader. Kᵢ values obtained from the FP assay results were derived from the Kenakin Kᵢ equation: Kenakin Kᵢ=(Lb)(EC₅₀)(K_{d})/(Lo)(Ro)+Lb(Ro−Lo+Lb−K_{d}), where K_{d} [M]: K_{d} of the probe, EC₅₀ [M]: obtained from FP assay, total tracer Lo [M]: probe concentration in FP, bound tracer Lb [M]: 85% of probe concentration binds to target protein, total receptor Ro [M]: Pin1 concentration in the FP assay, as described (Auld et al. Receptor binding assays for HTS and drug discovery, in Assay Guidance Manual eds. Sittampalam, G. S., et al. Eli Lilly & Company and the National Center for Advancing Translational Sciences, 2004).

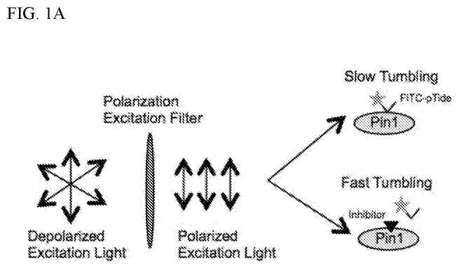

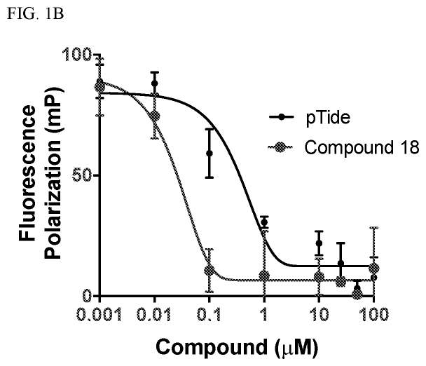

Results, illustrated in FIG. 1B, show that compound 18 is a potent binder of Pin1, with a Ki of 20 nM.

### Example 7: Peptidyl-Prolyl Cis-Trans Isomerase (PPIase) (Inhibition of Isomerase Activity)

Inhibition of Pin1 isomerase activity was determined using the chymotrypsin-coupled PPIase assay, using GST-Pin1 and Suc-Ala-pSer-Pro-Phe-pNA peptide substrate (50 mM), as described previously (Yaffe et al., Science 275:1957-1960 (1997)). GST-Pin1 was pre-incubated with the indicated concentrations of compound for 12 hours at 4° C. in buffer containing 35 mM HEPES (pH 7.8), 0.2 mM DTT, and 0.1 mg/mL bovine serum albumin (BSA). Immediately before the assay was started, chymotrypsin (final concentration 6 mg/mL), followed by the peptide substrate (Suc-Ala-pSer-Pro-Phe-pNA peptide substrate, final concentration 50 mM) was added. The Kᵢ value obtained from the PPIase assay was derived from the Cheng-Prusoff equation, Kᵢ=IC₅₀/(1+S/Kₘ), where Kₘ is the Michaelis constant for the used substrate, S is the initial concentration of the substrate in the assay, and IC₅₀ is the half-minimal inhibitory concentration of the inhibitor.

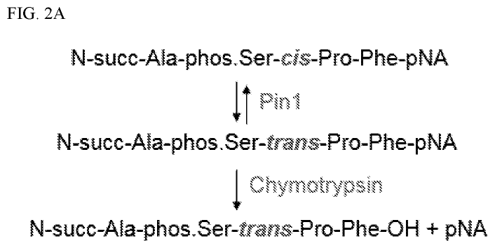

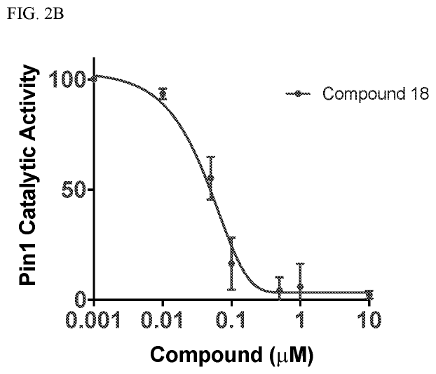

Results, illustrated in FIG. 2B, show that compound 18 potently inhibited Pin1's isomerase activity, with a Ki of 48 nM.

### Example 8: Covalent Labeling (Intact MS)

5 μg of purified Pin1 protein in 50 μL of 20 mM HEPES pH 7.5 and 75 mM NaCl was incubated with 5 μM of respective Pin1 inhibitors for 0-3 hr. A Shimadzu XR HPLC was used to inject the entire sample onto a self-packed reverse-phase column (1/32 in outer diameter×500 μm inner diameter, 5 cm of POROS 50R2 resin). After desalting, protein was eluted with an HPLC gradient (0%-100% B in 4 min, A=0.2 M acetic acid in water, B=0.2 M acetic acid in acetonitrile, flow rate=10 μl/min) into a LTQ XL mass spectrometer (Thermo Fisher Scientific, San Jose, Calif., USA). LTQ XL MS spectra were acquired in centroid mode using the electron multipliers for ion detection. Mass spectra were deconvoluted using MagTran1.03b2 software (Zhang and Marshall, J. Am. Soc. Mass Spectrom. 9:225-233 (1998)).

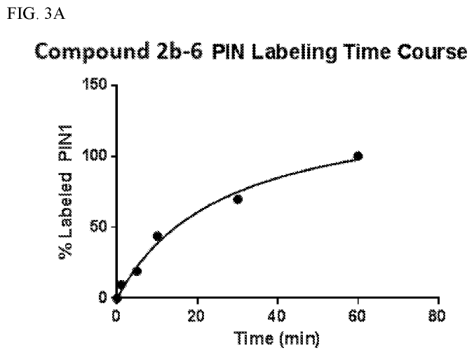

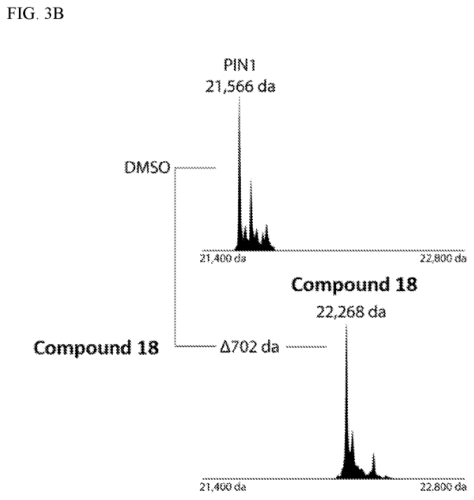

Results are shown in FIG. 3A and FIG. 3B. FIG. 3A shows that compound 2b-6 rapidly labeled Pin1 Cysl 13, over the course of 60 minutes. FIG. 3B shows that compound 18 resulted in 100% covalent labeling of Pin1 Cysl 13 after a 30-minute incubation at room temperature, at 1:1 compound 18: Pin1 ratio.

### Example 9: X-Ray Crystallography

A construct of full-length human Pin1 in a pET28 vector was overexpressed in E. coli BL21 (DE3) in LB medium in the presence of 50 mg/ml of kanamycin. Cells were grown at 37° C. to an OD of 0.8, cooled to 17° C., induced with 500 μM isopropyl-1-thio-D-galactopyranoside, incubated overnight at 17° C., collected by centrifugation, and stored at −80° C. Cell pellets were sonicated in buffer A (50 mM hepes 7.5, 300 mM NaCl, 10% glycerol, 10 mM Imidazole, and 3 mM 2-mercaptoethanol (BME) and the resulting lysate was centrifuged at 30,000×g for 40 min. Ni-NTA beads (Qiagen) were mixed with lysate supernatant for 30 min and washed with buffer A. Beads were transferred to an FPLC-compatible column and the bound protein was washed with 15% buffer B (50 mM hepes 7.5, 300 mM NaCl, 10% glycerol, 300 mM imidazole, and 3 mM BME) and eluted with 100% buffer B. Thrombin was added to the eluted protein and incubated at 4° C. overnight. The sample was concentrated and passed through a Superdex® 200 10/300 column (GE healthcare) in a buffer containing 20 mM hepes 7.5, 150 mMNaCl, 5% glycerol, 3 mM DTT, and 1 mM tris(2-carboxyethyl)phosphine (TCEP). Fractions were pooled, concentrated to approximately 37 mg/ml and frozen at −80° C.

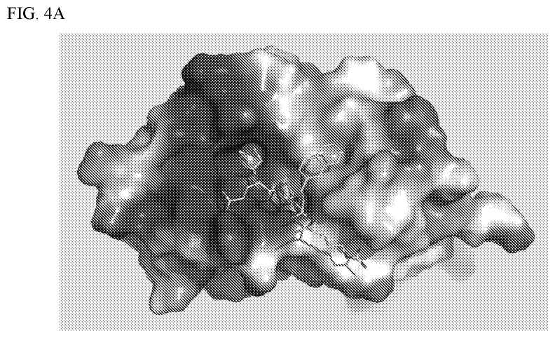

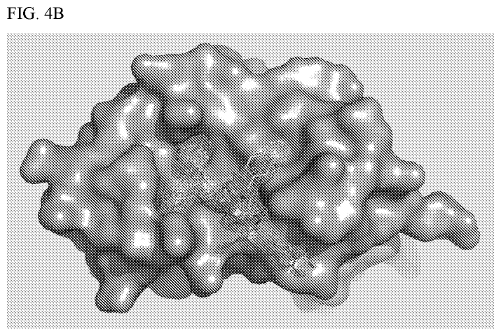

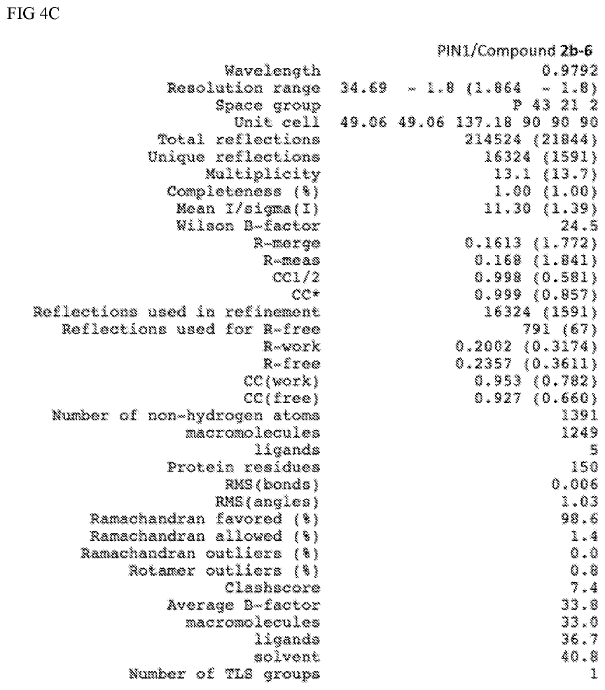

Results are shown in FIG. 4A, FIG. 4B, and FIG. 4C. FIG. 4A is a PDB file showing compound 2b-6 covalently bound to Cys113 in the PPIase active site. FIG. 4B is an electron density map showing the x-ray co-crystallographic structure of compound 2b-6 covalently bound to Pin1 and FIG. 4C is a table showing statistics of the crystallographic model.

Apo protein at a final concentration of 1 mM was crystallized by sitting-drop (200 nL+200 nL) vapor diffusion at 20° C. in the following crystallization buffer: 3 M (NH₄)₂SO₄, 100 mM BisTris-pH 7.0, 1% PEG400, and 1 mM DTT. A volume of 200 nL of 1 mM compound 2b-6 was added directly to crystals for soaking at 20° C. for 16 hrs. Crystals were transferred briefly into crystallization buffer containing 25% glycerol prior to flash-freezing in liquid nitrogen.

Diffraction data from complex crystals were collected at beamline 24ID-E of the NE-CAT at the Advanced Photon Source (Argonne National Laboratory). Data sets were integrated and scaled using XDS (Kabsch, Acta Crystallogr. D Biol. Crystallogr. D66: 133-144 (2010)). Structures were solved by molecular replacement using the program Phaser (McCoy et al., J. Appl. Cryst. 40:658-674 (2007)) and the search model PDB entry 1PIN. The ligand was positioned and preliminarily refined using Buster and Rhofit (Smart et al., Acta Cryst. D68: 368-380 (2012)). Iterative manual model building and refinement using Phenix (Adams et al., Acta Cryst. D66:213-221 (2010)) and Coot (Emsley and Cowtan, Acta Cryst. D60:2126-2132 (2004)) led to a model with excellent statistics (see FIG. 4C). This work was based upon research conducted at the Advanced Photon Source on the Northeastern Collaborative Access Team beamlines (NIGMS P41 GM103403).

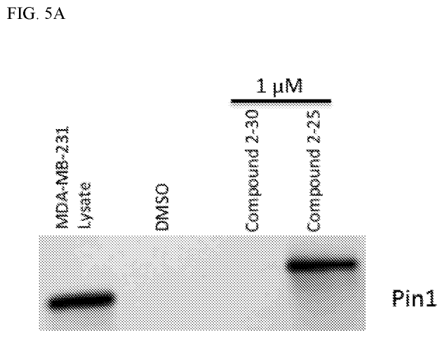

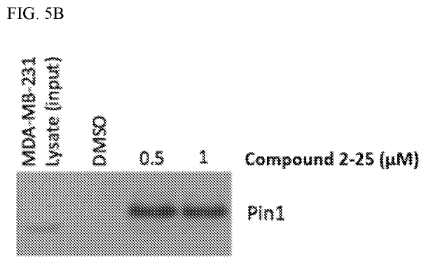

### Example 10: Lysate Target Engagement (Pull Down of Pint with Biotinylated Probes; FIG. 5A)

TNBC-MDA-MB-231 cells were lysed in lysis buffer (50 mM Hepes pH 7.5, 150 mM NaCl, 1 mM ethylenediaminetetraacetic acid (EDTA), 10% v/v glycerol, 0.5% v/v NP-40, protease inhibitors (Roche)). After clarifying by centrifugation (14,000 rpm for 15 min at 4° C.), lysates were incubated with the indicated concentrations of candidate compound for 1 hour at 4° C. (500 μg of protein per sample, as determined by BCA). Lysates were then incubated with streptavidin agarose resin (30 μL of 1:1 beads: lysis buffer slurry) (Thermo Scientific™, cat. #20349) for 2 hours at 4° C. Beads were washed 4 times with 500 μL of washing buffer (50 mM Hepes, pH 7.5, 10 mM NaCl, 1 mM EDTA, 10% glycerol), then pelleted by centrifugation and dried. The beads were then boiled at 95° C. for 5 minutes in 30 μL of 2× LDS+10% β-mercaptoethanol. Lysates were probed for specified proteins by western blotting using the Bolt system (Life Technologies™).

Results are shown in FIG. 5A and FIG. 5B. FIG. 5A shows that a biotin probe, compound 2-25, pulled down Pin1 from TNBC-MDA-MB-231 lysate at a concentration 1 μM, whereas the corresponding negative control, compound 2-30, did not. FIG. 5B shows that a biotin probe, compound 2-32, pulled down Pin1 from TNBCA-MDA-MB-231 lysate at concentrations of 500 nM and 1 μM.

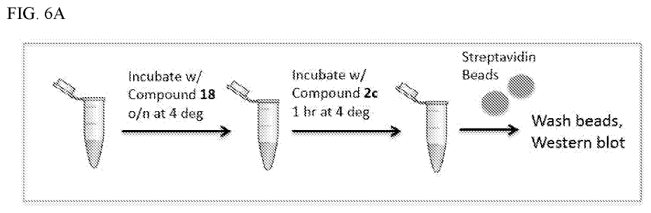

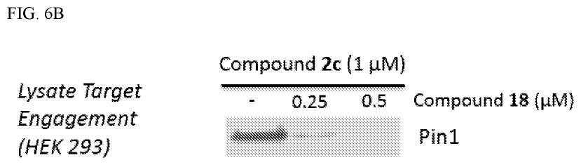

### Example 11: Lysate Target Engagement (Target Engagement Via Competition Assay: FIG. 6A)

TNBC-MDA-MB-231 cells were lysed in lysis buffer (50 mM Hepes pH 7.5, 150 mM NaCl, 1 mM EDTA, 10% v/v glycerol, 0.5% v/v NP-40, protease inhibitors (Roche)). After clarifying by centrifugation, lysates were pre-incubated with the indicated concentrations of compound 18 overnight at 4° C. (500 μg of protein per sample, as determined by BCA). The lysates were then incubated with 1 μM of compound 2c (desthiobiotin probe) for 1 hour at 4° C. Lysates were then incubated with streptavidin agarose resin (30 μL of 1:1 beads: lysis buffer slurry) (Thermo Scientific™, cat. #20349) for 2 hours at 4° C. Beads were washed 4 times with 500 μL of washing buffer (50 mM Hepes, pH 7.5, 10 mM NaCl, 1 mM EDTA, 10% glycerol), then pelleted by centrifugation and dried. The beads were then boiled at 95° C. for 5 minutes in 30 μL of 2× LDS+10% β-mercaptoethanol. Lysates were probed for specified proteins by western blotting using the Bolt system (Life Technologies™).

Results, illustrated in FIG. 6B, show that compound 18 potently engaged Pin1 in TNBC-MDA-MB-231 cell lysate.

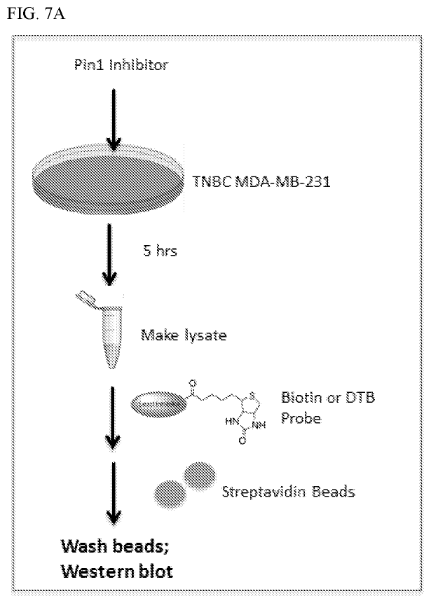

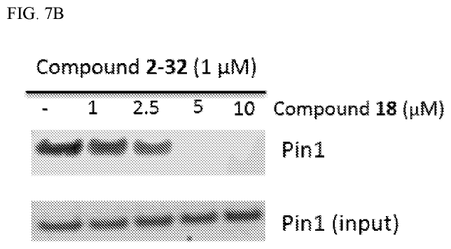

### Example 12: Cellular Target Engagement (Assessment of Cell Permeability; FIG. 7A)

TNBC MDA-MB-231 cells were plated in 10 cm plates with 2.5 million cells per plate in 6 mL of media. The day after plating, cells were treated with the indicated concentrations of compound 18 for 5 hours. The cells were washed 2× with 0.9% NaCl (1 mL per 10 cm plate), and collected by scraping with a cell scraper. Cells were lysed in lysis buffer (50 mM Hepes pH 7.5, 150 mM NaCl, 1 mM EDTA, 10% v/v glycerol, 0.5% v/v NEMO, protease inhibitors (Roche)) using 210 μL of cell lysis buffer per 10 cm plate of cells. After clarifying by centrifugation (14,000 rpm for 15 min at 4° C.), 5 μL of each lysate sample was combined with 5 μL of 2× LDS+10% β-mercaptoethanol, boiled for 5 minutes, and set aside for the input loading control (later to be loaded directly in the gel). Then, 200 μL of each lysate was then incubated with 1 μM of compound 2-32 (biotin probe compound) for 1 hour at 4° C. Lysates were then incubated with streptavidin agarose resin (30 μL of 1:1 beads: lysis buffer slurry) (Thermo Scientific™, cat. #20349) for 2 hours at 4° C. Beads were washed 4 times with 500 μL of washing buffer (50 mM Hepes, pH7.5, 10 mM NaCl, 1 mM EDTA, 10% glycerol), then pelleted by centrifugation and dried. The beads were then boiled at 95° C. for 5 minutes in 30 μL of 2× LDS+10% β-mercaptoethanol. Lysates were probed for specified proteins by western blotting using the Bolt system (Life Technologies™).

Results, illustrated in FIG. 7B, show that compound 18 fully engaged cellular Pin1 by 5 μM.

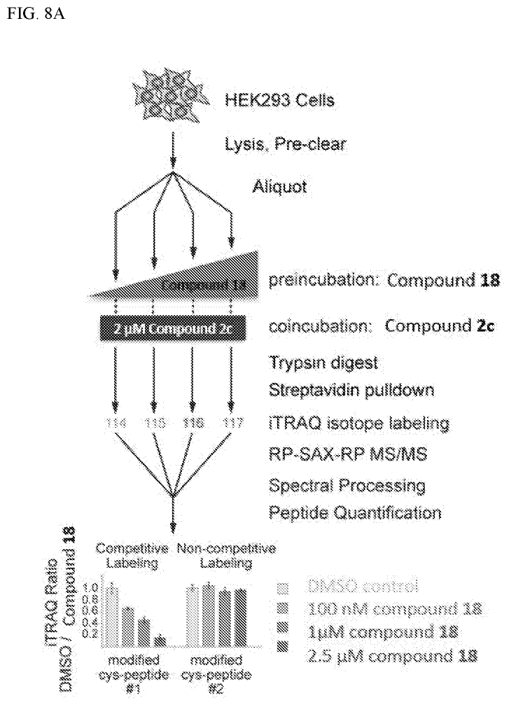

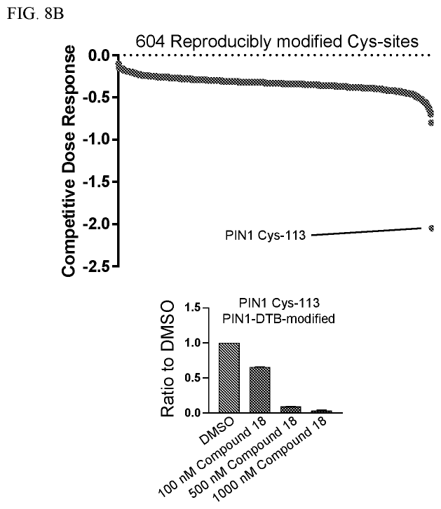

### Example 13: Selectivity Assessment—CiTe-ID (FIG. 8A)

Compound 18 exhibits high selectivity for Pin1 Cys 113. HEK 293 cell pellets were lysed with lysis buffer (50 mM Tris pH 7.5, 150 mM NaCl, 1 mM EDTA, 10% v/v glycerol, 0.5% v/v NP-40, protease inhibitors). After lysate clearance centrifugation, protein concentration was determined by BCA. Samples were precleared with avidin resin for 1 hr. at 4° C. After centrifugation, the supernatant was split in four and pretreated with DMSO or increasing concentrations of compound 18 (100 nM, 1 μM, 2.5 μM) and incubated at room-temperature for 3 hr. 2 μM of the desthiobiotin-tagged analog (2c) was added to all samples and incubated at 4° C. for 18 h. Urea was added to 8 M final concentration followed by cleanup using Zeba™ desalting columns. Samples were diluted to 4 M urea with pH 8 lysis buffer and reduced with 10 mM DTT for 30 min at 56° C. Samples were then alkylated with 20 mM IAA for 30 min at RT. Samples were further diluted to 2 M urea before trypsin digestion at 37° C. overnight. Avidin resin was added to the samples and incubated at RT for 1 h. The beads were sequentially washed with lysis buffer three times, PBS three times, and water two times. Peptides were eluted with four sequential incubations of 50% acetonitrile (MeCN), 0.1% trifluoroacetic acid (TFA) for 3 min at RT. Eluted peptides were concentrated by vacuum centrifugation and resuspended in 0.1% TFA before batch mode C18 cleanup, as described in (Adelmant G O, C. J., Ficarro S B, Sikorski T W, Zhang Y, Marto J A. in Sample Preparation in Biological Mass Spectrometry (ed ⋅ Lazarev A V Ivanov A R) Ch. 22, (Springer). Samples were vacuum dried and resuspended in 30% 0.5M TEAB, 70% EtOH. iTRAQ® stable isotope labeling reagent was added to each corresponding sample. After incubation at room temperature for 1 hr, the samples were combined and acidified before vacuum drying. Samples were reconstituted in 0.1% TFA and desalted using a SOLA desalting plate before vacuum drying. Samples were reconstituted in 25% MeCN, 0.1% TFA and batch mode SCX cleaned, as described in Adelmant, supra. Samples were vacuum dried and reconstituted in 5% DMSO, 100 mM ammonium formate for 3D RP-SAX-RP LC-MS/MS (Zhou et al., Nat. Commun. 4:2171-2181 (2013)). Peptides were separated across 11 fractions using a 90 min 5%-60% MeCN gradient on a Q-Exactive HF mass spectrometer. For data processing, Native .RAW data files from the mass spectrometer were processed using the multiplierz Python-based framework (Parikh et al., BMC bioinformatics 70:364-379 (2009)) to generate .mgf files for input to Mascot (Matrix Science). MS/MS spectra were processed to remove peaks corresponding to inhibitor fragmentation, as described previously (Ficarro et al., Anal. Chem. 55:12248-12254 (2016)). The following inhibitor fragment ion associated m/z values were removed from each spectrum: 110.07, 134.096, 140.07, 147.11, 159.09, 170.06, 197.127, 199.07, 208.08, 234.06, 236.074, 266.19, 280.202, 284.14, 297.23, 315.18, 325.22, 342.25, 344.17, 353.22, 412.233, 438.21, 439.3, 447.27, 455.24, 456.33, 464.3, 465.28, 472.27, 482.307, 499.334, 607.37, 625.38, 642.407, 651.361, 668.389, 685.413, 718.44, 736.45, 753.476, 762.43, 779.456, 796.482. Peptide precursor masses were recalibrated on a per-scan basis by correcting all m/z values based on accurate mass recorded for the Si(CH₃)₂O₆ peak in each spectrum. All data were searched against a forward-reverse human database assembled from the NCBI Refseq database. For de-isotoped HCD spectra, the precursor mass tolerance was set to 10 ppm and the MS/MS fragment ion tolerance was set to 25 mmu. Search parameters included trypsin specificity, with a maximum of two missed cleavages, fixed carbamidomethylation of Cys (+57 Da), variable oxidation on Met (+16 Da with −64 Da neutral loss possible), variable deamidation on Asn and Gin (+1 Da), fixed iTRAQ® 4-plex labeling on Lys and N-termini (+144 Da), variable compound 2c labeling of Cys (+997 Da, with −997 Da neutral loss possible). Reported peptide sequences were filtered based on a 1% false discovery rate. Normalized reporter ion signal for labeled cysteine residues from multiple PSMs was summed and a ratio was generated for each reporter channel by comparing it to the DMSO-treated control channel. Inhibitor concentrations and ratios were used to generate a trend line for each labeled site with the slope being the competitive dose response for the cysteine site.

Results, illustrated in FIG. 8B, show that Pin1 C113 was the only site that underwent dose-dependent covalent modification by compound 18 in HEK 293 cell lysate.

### Example 14: Downstream Signaling

TNBC MDA-MB-231 cells were plated in 6-well plates at a density of 100,000 cells per well in 2 mL media. The day after plating, cells were treated with the indicated concentration of compound 18, and harvested at 1, 2, 4, 6, and 8 hours. To harvest, the cells were washed with PBS, and then lysed in RIPA Lysis Buffer (Thermo Scientific™, cat. #89900, with protease inhibitors (Roche). Lysates were clarified by centrifugation (14,000 rpm for 15 min at 4° C.), and protein concentrations were determined by BCA. The samples were normalized and prepared in 4× LDS+10% β-mercaptoethanol, and were then boiled at 95° C. for 5 minutes. Lysates were probed for specified proteins by western blotting using the Bolt system (Life Technologies™).

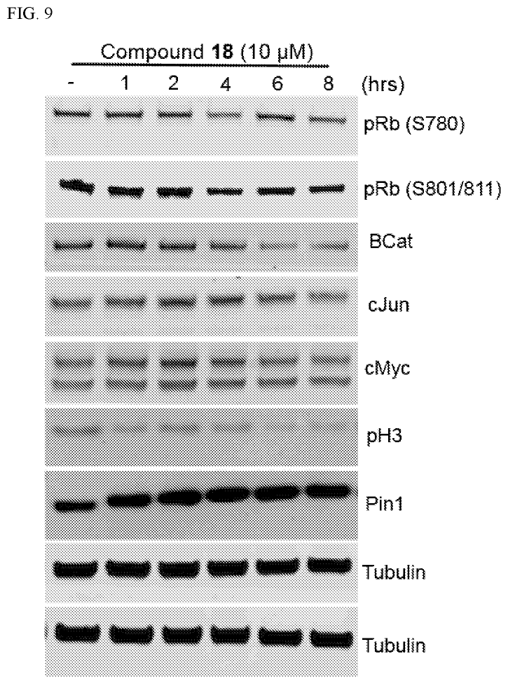

Results, illustrated in FIG. 9, show that compound 18 induced changes in downstream signaling upon Pin1 loss.

### Example 15: Time-Dependent Antiproliferative Activity in PATU-8988T Cells

PATU-8988T cells were plated at a density of 100 cells per well in 100 μL media in a 96-well white clear bottom plate (Corning cat #3903), with at least one plate per time point (Day 0, 2, 4, 6 and 8). Cells were treated the day after plating with 1 μL of DMSO or Compound 18 to give the indicated concentrations, and were then incubated at 37° C. 5% CO₂. Every 48 hours, the media was aspirated and replaced with fresh media containing fresh compound or DMSO. When the indicated time points had been reached, cell viability was evaluated using the CellTiter-Glo Luminescent Cell Viability Assay (Promega cat #G7570) according to the manufacturer's standards, measuring luminescence using an Envision plate-reader. The Day 0 time point plates were read the day after plating, prior to compound treatment. N=3 biological replicates were used for each treatment condition.

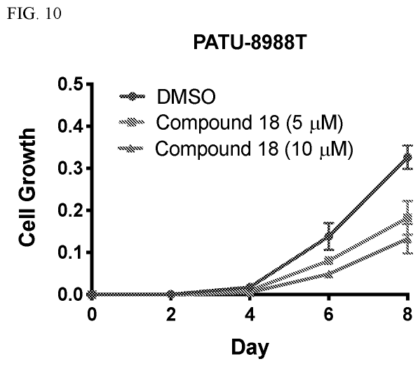

Results, illustrated in FIG. 10, show that Compound 18 induced dose-dependent and time-dependent defects in cell viability in the pancreatic ductal adenocarcinoma (PDAC) cell line, PATU-8988T.

### Example 16: Overview of Compound Characterization Data

Various compounds disclosed herein were analyzed for Pin1 binding (FP assay), inhibition of PPIase activity (chymotrypsin-coupled PPIase assay), covalent labeling to Pin1 Cys 113 (intact MS), lysate and cellular target engagement (as assessed via biotin competition assays, described in Examples 5-7), and x-ray crystallography. The results are shown in Table 1.

TABLE 1
Characterization of inventive compounds.

| Compound | Ki, μM | Ki, μM | Covalent? | Lysate Target | Cellular Target | Selectivity | X-Ray Crystal |
|---|---|---|---|---|---|---|---|
| Name | (FP Assay) | (PPlase Assay) | (In tact MS) | Engagement? | Engagement? | Assessment? | Structure? |
| 2 | 0.074 | | | | | | |
| 2-1 | >10 | | | | | | |
| 2-2 | >10 | | | | | | |
| 2-3 | >10 | | | | | | |
| 2-4 | 1.24 | | | | | | |
| 2-5 | 0.13 | | | | | | |
| 2-6 | 0.28 | | | | No | | |
| 2-7 | 0.3 | | | | | | |
| 2-8 | 0.13 | | | | | | |
| 2-9 | 0.33 | | | | | | |
| 2-10 | >10 | | | | | | |
| 2-11 | 0.06 | | | Yes-complete | No | | |
| | | | | engagement | | | |
| | | | | by 10 μM | | | |
| 2-12 | 0.106 | | | | | | |
| 2-13 | 0.13 | | | | | | |
| 2-14 | 0.13 | | | | | | |
| 2-15 | >10 | | | | | | |
| 2-16 | >10 | | | | | | |
| 2-17 | 0.11 | | | | | | |
| 2-18 | 0.14 | | | | | | |
| 2-19 | >10 | | | | | | |
| 2-20 | 0.122 | | | | | | |
| 2-21 | 0.004 | | | | | | |
| 2-22 | 0.154 | | | Yes-partial | | | |
| | | | | engagement | | | |
| | | | | at 10 μM | | | |
| 2-23 | 0.231 | | | | | | |
| 2-24 | 0.525 | | | | | | |
| 2-25 | 0.02 | | | Yes-(Biotin | | Proteomics | |
| | | | | probe) pulls | | | |
| | | | | down Pin1 | | | |
| | | | | at 1 μM | | | |
| 2-26 | 0.299 | | | | | | |
| 2-27 | 0.075 | | | Yes-partial | No | | |
| | | | | engagement | | | |
| | | | | at 10 μM | | | |
| 2-28 | >10 | | | | | | |
| 2-29 | 4.8 | | | | | | |
| 2-30 | 1.6 | | | | | | |
| 2-31 | 0.07 | | | | | | |
| 2-32 | 0.018 | | | Yes-(Biotin | | | |
| | | | | probe) pulls | | | |
| | | | | down Pin1 | | | |
| | | | | at 0.5 μM | | | |
| 2-33 | >10 | | | | | | |
| 2-34 | >10 | | | | | | |
| 2-35 | >10 | | | | | | |
| 2b-1 | >10 | | | | | | |
| 2b-2 | >10 | | | | | | |
| 2b-3 | 2 | | Yes (81% | | | | Yes |
| | | | labeling) | | | | |
| 2b-4 | >10 | | | | | | |
| 2b-5 | >10 | | | | | | |
| 2b-6 | 0.17 | 0.04 | Yes (100% | No | | | Yes |
| | | | labeling) | | | | |
| 2b-7 | 4.02 | | | | | | |
| 2b-8 | 2.83 | | | | | | |
| 2b-9 | 0.19 | | | | | | |
| 2b-10 | 0.85 | | | | | | |
| 2b-11 | 0.609 | | | | | | |
| 2b-12 | 0.23 | | | | | | |
| 2b-13 | 0.07 | 0.021 | | Yes-Complete | | | |
| | | | | engagement | | | |
| | | | | at 10 μM | | | |
| 2b-14 | 0.19 | | | | | | |
| 2b-15 | >10 | | | | | | |
| 2b-16 | >10 | | | | | | |
| 2b-17 | >10 | | | | | | |
| 2b-18 | 0.03 | | | Yes-Complete | No | | |
| | | | | engagement | | | |
| | | | | at 5 μM | | | |
| 2c | 0.015 | | | Yes-(dtb | | CiTe-ID probe | |
| | | | | probe) pulls | | | |
| | | | | down Pin1 | | | |
| | | | | at 0.5 μM | | | |
| 2c-1 | 0.04 | | | | | | |
| 2c-2 | 0.03 | | | | | | |
| 3 | 4.17 | | | | | | |
| 3b-1 | 1.61 | | Yes (93% | No | No | | |
| | | | labeling) | | | | |
| 4 | 0.35 | | Yes (93% | No | | | |
| | | | labeling) | | | | |
| 5 | >10 | | | | | | |
| 5-1 | 0.61 | | | | | | |
| 6b | >10 | | | | | | |
| 6b-1 | >10 | | | | | | |
| 11 | 0.01 | | | | Yes- | | |
| | | | | | Complete | | |
| | | | | | engagement | | |
| | | | | | at 5 μM | | |
| 11-1 | 0.176 | | | No | No | | |
| 11-2 | >10 | | | No | No | | |
| 11-3 | 0.19 | | | | Yes-Complete | | |
| | | | | | engagement | | |
| | | | | | at 15 μM | | |
| 11-4 | 0.03 | | | | No | | |
| 11-5 | 0.13 | | | | No | | |
| 11-6 | 0.01 | | | | No | | |
| 11-7 | 0.2 | | | | | | |
| 11-8 | 0.15 | | | | | | |
| 11-9 | 0.31 | | | | | | |
| 11-10 | 0.11 | | | | | | |
| 11-11 | 0.17 | | | | | | |
| 11-12 | >10 | | | | | | |
| 11-13 | >10 | | | | | | |
| 11-14 | 1 | | | | | | |
| 11-15 | 0.15 | | | | | | |
| 11-16 | >10 | | | | | | |
| 11-17 | >10 | | | | | | |
| 11-18 | 0.134 | | | | No | | |
| 11-19 | 0.146 | | | | No | | |
| 11-20 | 0.223 | | | | | | |
| 11-21 | 0.015 | | | | | | |
| 11-22 | >10 | | | | | | |
| 11-23 | 0.004 | | Yes (100% | | Yes-Complete | | |
| | | | labeling) | | engagement | | |
| | | | | | at 10 μM | | |
| 11-24 | >10 | | | | | | |
| 11-25 | >10 | | | | | | |
| 11-26 | >10 | | | | | | |
| 11-27 | >10 | | | | | | |
| 11-28 | 0.099 | | | | No | | |
| 11-29 | 1.01 | | | | | | |
| 11-30 | >10 | | | | | | |
| 11-31 | 0.023 | | | | | | |
| 11-32 | 0.121 | | | | | | |
| 11-33 | 0.028 | | | | | | |
| 11-34 | >10 | | | | | | |
| 11-35 | >10 | | | | | | |
| 11-36 | >10 | | | | | | |
| 11-37 | 0.026 | | | | | | |
| 11-38 | 0.018 | | | | No | | |
| 11-39 | 0.017 | | | | No | | |
| 11-40 | 0.087 | | | | | | |
| 11-41 | 0.151 | | | | | | |
| 11-42 | 0.109 | | | | | | |
| 11-43 | 0.172 | | | | | | |
| 11-44 | 1.5 | | | | | | |
| 11-45 | >1 | | | | | | |
| 12 | >10 | | | | | | |
| 13 | 4.43 | | | | | | |
| 14 | 0.143 | | | | | | |
| 15 | >10 | | | | | | |
| 18 | 0.02 | 0.048 | Yes (100% | Yes- | Yes-Complete | | |
| | | | labeling) | Complete | engagement | CiTe-ID | In Progress |
| | | | | engagement | at 5 μM | | |
| | | | | 500 μM | | | |
| 18-1 | 1.9 | | | | No | | |
| 18-2 | >10 | | | | No | | |

These results show that, of the compounds synthesized, peptides with an N-terminal chloroacetamide electrophile and either a glutamic acid or a phenylalanine residue in position R₁ can potently and covalently inhibit Pin1. Capping the C-terminal amide of the peptide with an ethyl ester also leads to a more favorable cell permeability profile.

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A compound having a structure represented by formula (I): 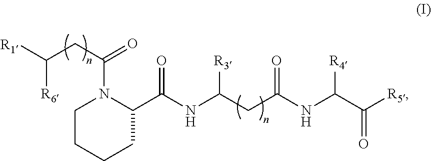
wherein each n is independently 0 or 1;
R₁′ is a phosphorylated alkyl, a hydroxyalkyl, a sulfone, an optionally substituted aralkyl, a carboxylic acid or an ester;
R_{3′} is an optionally substituted aralkyl, a ketone or an optionally substituted heteroaralkyl;
R_{4′} is an alkyl urea, an alkyl guanidine, a hydroxyalkyl, an amide, an optionally substituted heteroaralkyl or an optionally substituted aralkyl;
R_{5′} is an optionally substituted N-aralkyl, an alkoxy, an optionally substituted N-methyl-aralkyl, an optionally substituted N-methyl-aryl, an optionally substituted N-aryl, an optionally substituted N-cyclyl, an optionally substituted heterocyclyl or an N-alkyl; and
R_{6′} is a sulfonamide or an amide; or a pharmaceutically acceptable salt or stereoisomer thereof, wherein the compound is cell permeable and binds Pin1 with a Ki of less than 1 μM.

2. The compound of claim 1, wherein R₁′ is a phosphorylated alkyl, a hydroxyalkyl, a sulfone, an optionally substituted aralkyl, a carboxylic acid or an ester except for

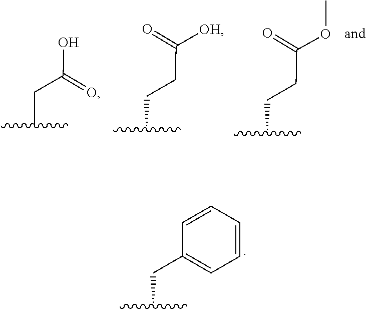

3. The compound of claim 1, wherein R_{3′} is an optionally substituted aralkyl, a ketone or an optionally substituted heteroaralkyl except for

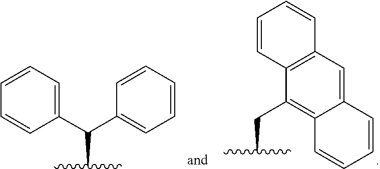

4. The compound of claim 1, wherein R_{4′} is an alkyl urea, an alkyl guanidine, a hydroxyalkyl, an amide, an optionally substituted heteroaralkyl or an optionally substituted aralkyl except for

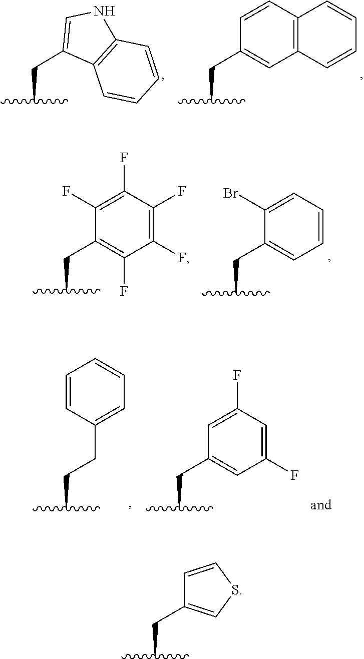

5. The compound of claim 1, wherein R_{5′} is an optionally substituted N-aralkyl, an alkoxy, an optionally substituted N-methyl-aralkyl, an optionally substituted N-methyl-aryl, an optionally substituted N-aryl, an optionally substituted N-cyclyl, an optionally substituted heterocyclyl or an N-alkyl except for

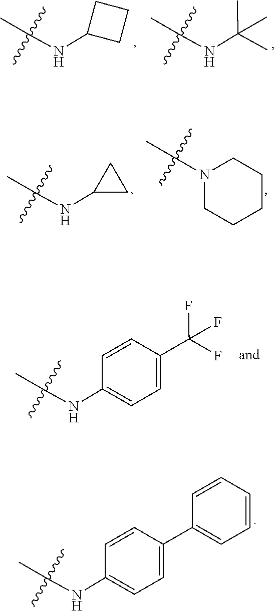

6. The compound of claim 1, wherein R_{6′} is a sulfonamide or an amide except for

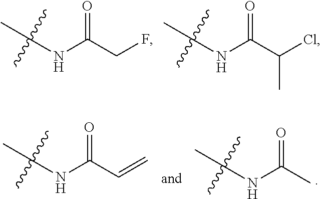

7. The compound of claim 1, wherein R_{6′} is chloroacetamide and the compound has a structure represented by formula (Ia): 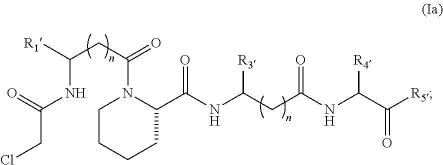
or wherein R_{6′} is N-methyl chloroacetamide and the compound has a structure represented by formula (Ib): 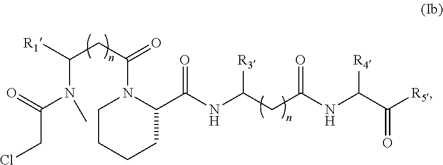
or a pharmaceutically acceptable salt or stereoisomer thereof.

8. (canceled)

9. The compound of claim 1, wherein R_{1′} is benzyl and the compound has a structure represented by formula (Tc): 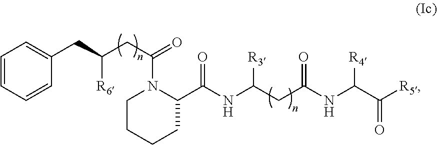
or a pharmaceutically acceptable salt or stereoisomer thereof.

10. The compound of claim 1, wherein R_{3′} is an alkyl substituted indole and the compound has a structure represented by formula (Id): 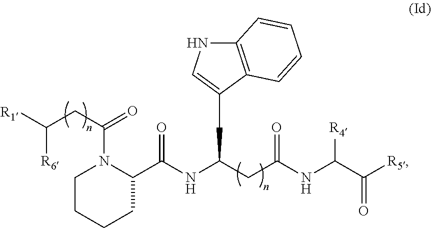
or a pharmaceutically acceptable salt or stereoisomer thereof.

11. The compound of claim 1, wherein R_{4′} is an alkyl urea and the compound has a structure represented by formula (Ie): 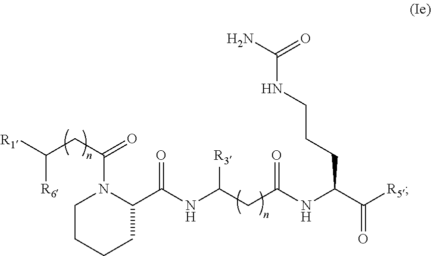
or wherein R_{4′} is an alkyl guanidine and the compound has a structure represented by formula (If): 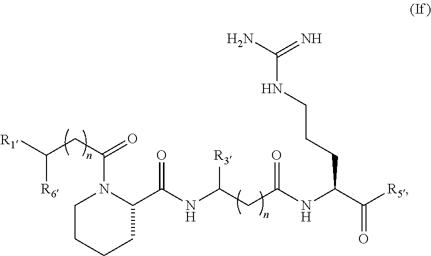
or a pharmaceutically acceptable salt or stereoisomer thereof.

12. (canceled)

13. The compound of claim 1, wherein R_{5′} is alkoxy and the compound has a structure represented by formula (Ig): 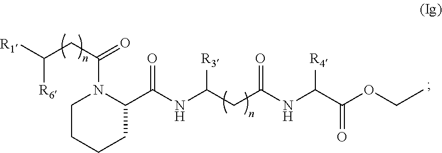
or wherein Ry is methyl substituted N-benzyl and the compound has a structure represented by formula (Ih): 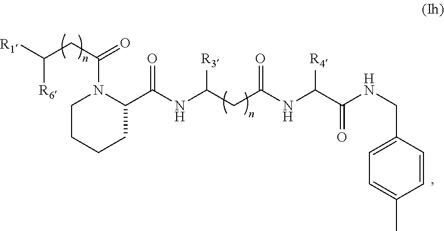
or a pharmaceutically acceptable salt or stereoisomer thereof.

14. (canceled)

15. The compound of claim 1, wherein R_{6′} is 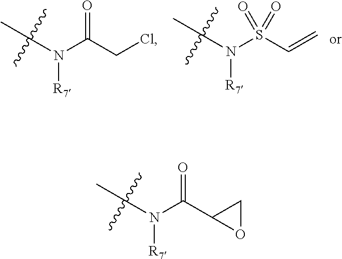
wherein R_{7′} is hydrogen or methyl;
R_{1′} is 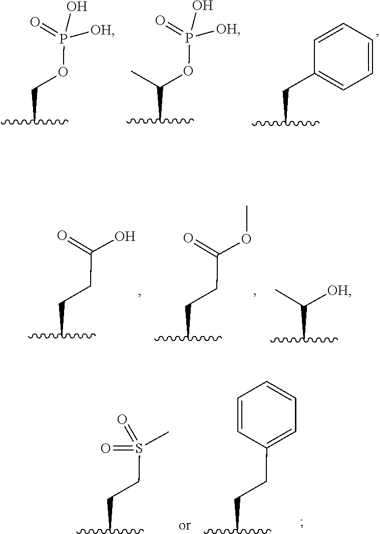
R_{3′} is 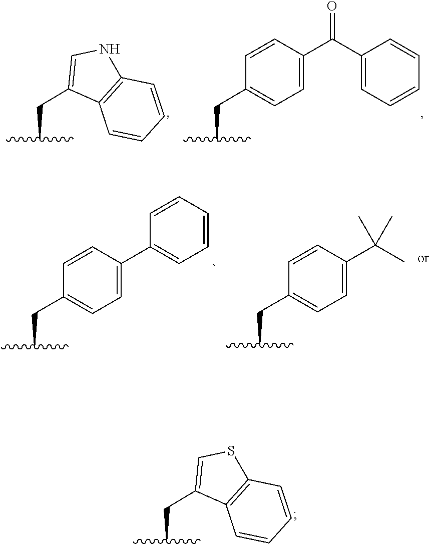
R_{4′} is 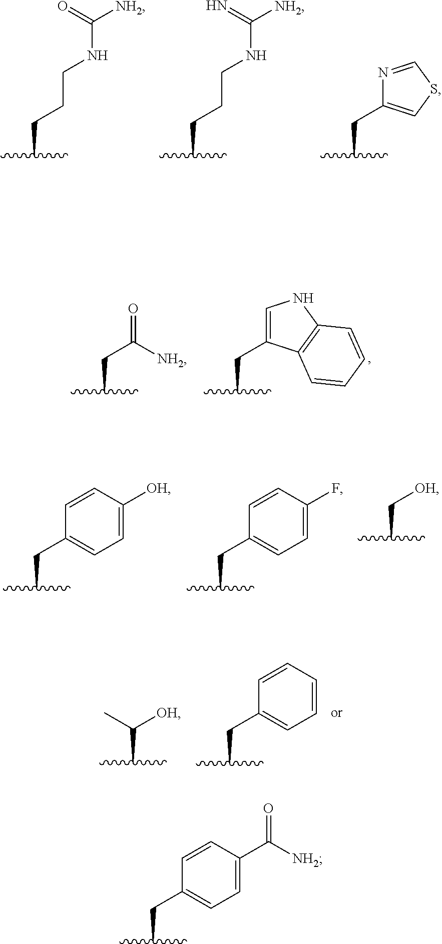
and
R_{5′} is 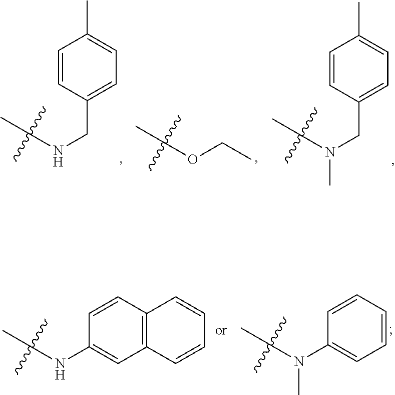
or a pharmaceutically acceptable salt or stereoisomer thereof.

16. The compound of claim 1, wherein the compound is selected from the group consisting of: 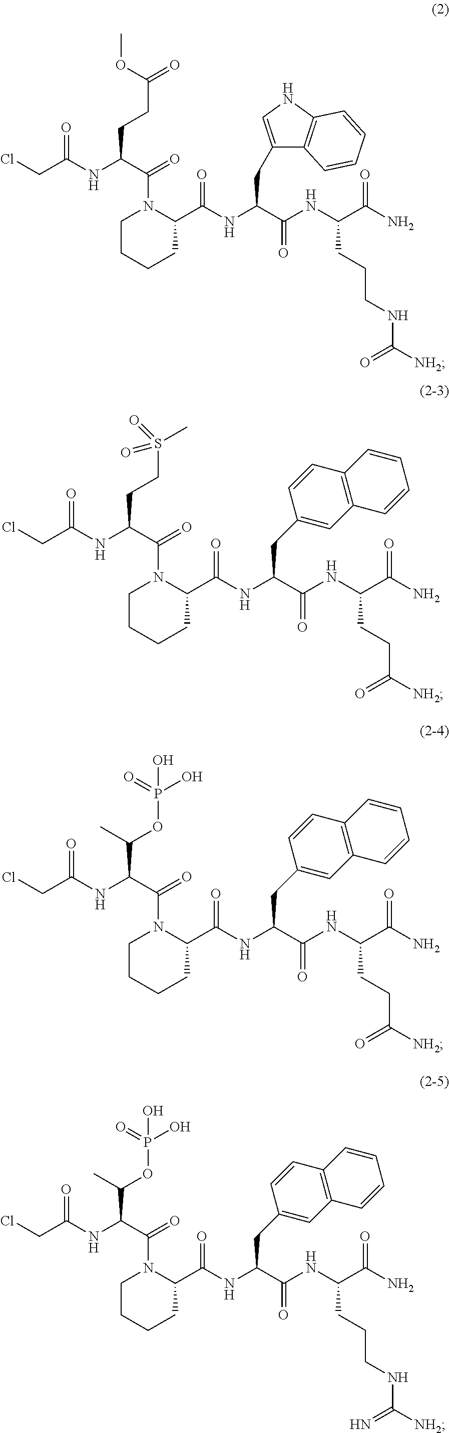 [SMILES: COC(=O)CC[C@H](NC(=O)CCl)C(=O)N1CCCC[C@H]1C(=O)N[C@@H](Cc1c[nH]c2ccccc12)C(=O)N[C@@H](CCCNC(N)=O)C(N)=O ; CS(=O)(=O)CC[C@H](NC(=O)CCl)C(=O)N1CCCC[C@H]1C(=O)N[C@@H](Cc1ccc2ccccc2c1)C(=O)N[C@@H](CCC(N)=O)C(N)=O ; CC(OP(=O)(O)O)[C@H](NC(=O)CCl)C(=O)N1CCCC[C@H]1C(=O)N[C@@H](Cc1ccc2ccccc2c1)C(=O)N[C@@H](CCC(N)=O)C(N)=O ; CC(OP(=O)(O)O)[C@H](NC(=O)CCl)C(=O)N1CCCC[C@H]1C(=O)N[C@@H](Cc1ccc2ccccc2c1)C(=O)N[C@@H](CCCNC(=N)N)C(N)=O] 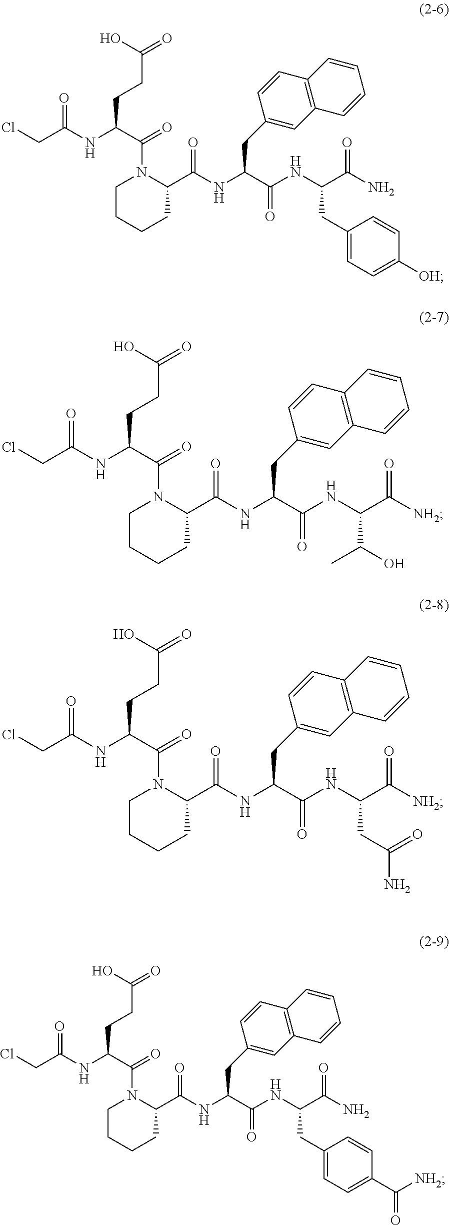 [SMILES: NC(=O)[C@H](Cc1ccc(O)cc1)NC(=O)[C@H](Cc1ccc2ccccc2c1)NC(=O)[C@@H]1CCCCN1C(=O)[C@H](CCC(=O)O)NC(=O)CCl ; CC(O)[C@H](NC(=O)[C@H](Cc1ccc2ccccc2c1)NC(=O)[C@@H]1CCCCN1C(=O)[C@H](CCC(=O)O)NC(=O)CCl)C(N)=O ; NC(=O)C[C@H](NC(=O)[C@H](Cc1ccc2ccccc2c1)NC(=O)[C@@H]1CCCCN1C(=O)[C@H](CCC(=O)O)NC(=O)CCl)C(N)=O ; NC(=O)c1ccc(C[C@H](NC(=O)[C@H](Cc2ccc3ccccc3c2)NC(=O)[C@@H]2CCCCN2C(=O)[C@H](CCC(=O)O)NC(=O)CCl)C(N)=O)cc1] 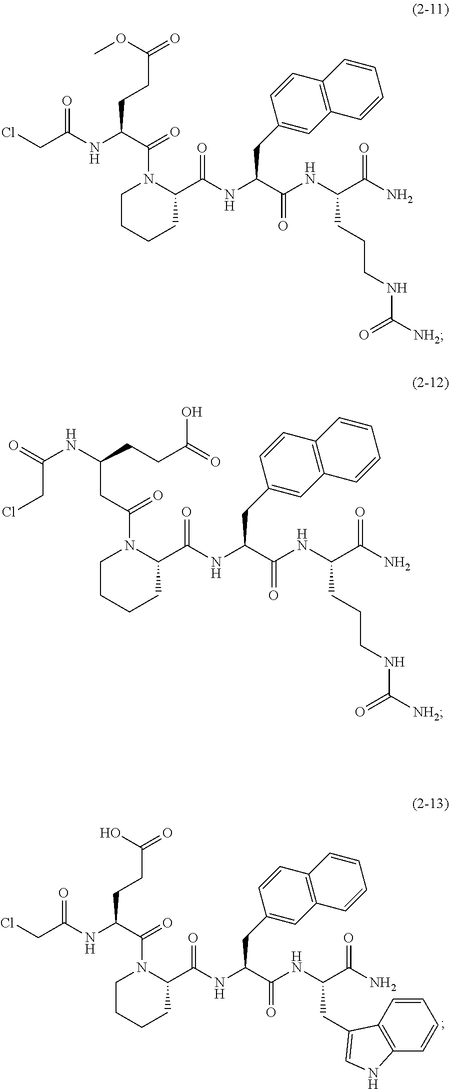 [SMILES: COC(=O)CC[C@H](NC(=O)CCl)C(=O)N1CCCC[C@H]1C(=O)N[C@@H](Cc1ccc2ccccc2c1)C(=O)N[C@@H](CCCNC(N)=O)C(N)=O ; NC(=O)NCCC[C@H](NC(=O)[C@H](Cc1ccc2ccccc2c1)NC(=O)[C@@H]1CCCCN1C(=O)C[C@H](CCC(=O)O)NC(=O)CCl)C(N)=O ; NC(=O)[C@H](Cc1c[nH]c2ccccc12)NC(=O)[C@H](Cc1ccc2ccccc2c1)NC(=O)[C@@H]1CCCCN1C(=O)[C@H](CCC(=O)O)NC(=O)CCl] 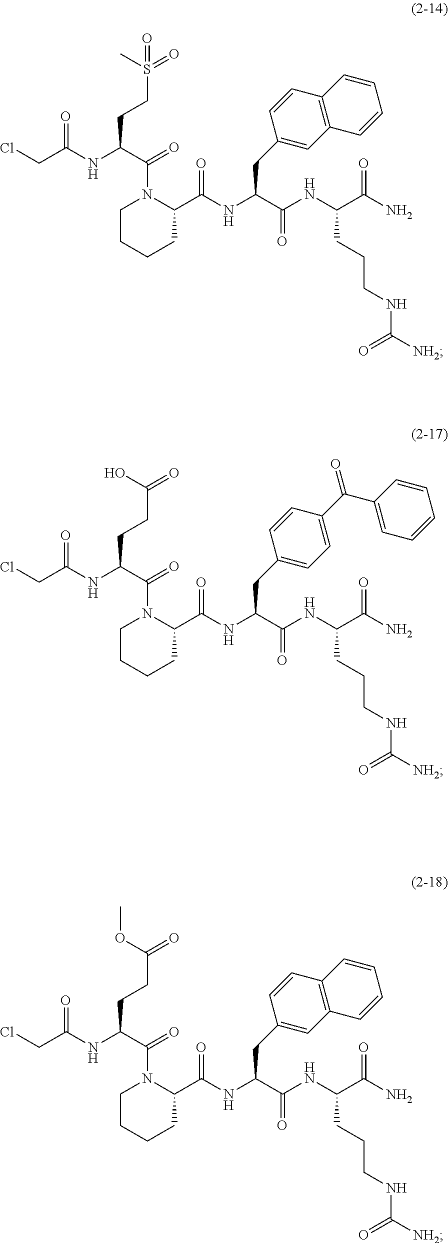 [SMILES: CS(=O)(=O)CC[C@H](NC(=O)CCl)C(=O)N1CCCC[C@H]1C(=O)N[C@@H](Cc1ccc2ccccc2c1)C(=O)N[C@@H](CCCNC(N)=O)C(N)=O ; NC(=O)NCCC[C@H](NC(=O)[C@H](Cc1ccc(C(=O)c2ccccc2)cc1)NC(=O)[C@@H]1CCCCN1C(=O)[C@H](CCC(=O)O)NC(=O)CCl)C(N)=O ; COC(=O)CC[C@H](NC(=O)CCl)C(=O)N1CCCC[C@H]1C(=O)N[C@@H](Cc1ccc2ccccc2c1)C(=O)N[C@@H](CCCNC(N)=O)C(N)=O] 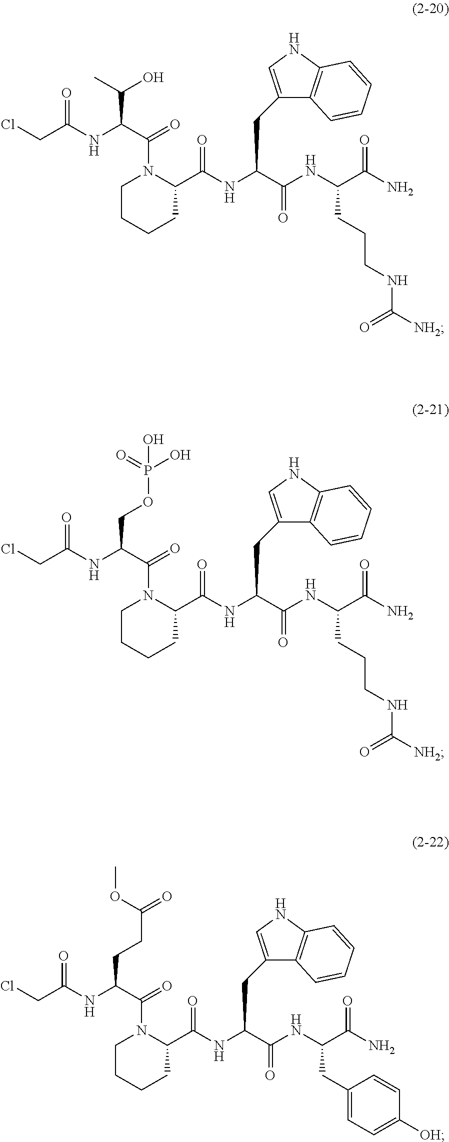 [SMILES: CC(O)[C@H](NC(=O)CCl)C(=O)N1CCCC[C@H]1C(=O)N[C@@H](Cc1c[nH]c2ccccc12)C(=O)N[C@@H](CCCNC(N)=O)C(N)=O ; NC(=O)NCCC[C@H](NC(=O)[C@H](Cc1c[nH]c2ccccc12)NC(=O)[C@@H]1CCCCN1C(=O)[C@H](COP(=O)(O)O)NC(=O)CCl)C(N)=O ; COC(=O)CC[C@H](NC(=O)CCl)C(=O)N1CCCC[C@H]1C(=O)N[C@@H](Cc1c[nH]c2ccccc12)C(=O)N[C@@H](Cc1ccc(O)cc1)C(N)=O] 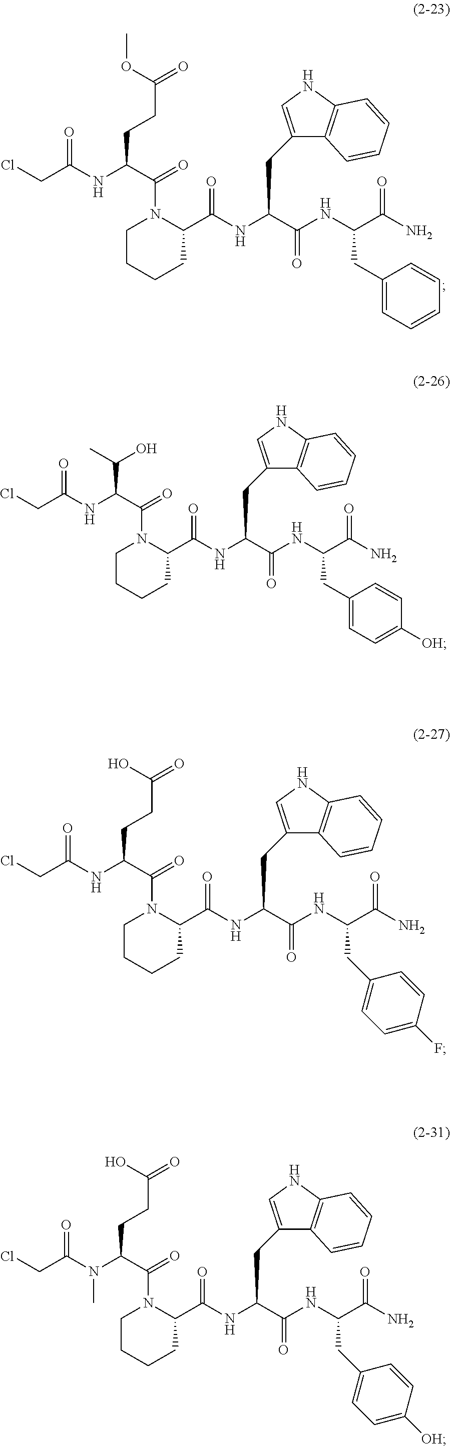 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1ccccc1)C(N)=O ; CC(O)[C@H](NC(=O)CCl)C(=O)N1CCCC[C@H]1C(=O)N[C@@H](Cc1c[nH]c2ccccc12)C(=O)N[C@@H](Cc1ccc(O)cc1)C(N)=O ; NC(=O)[C@H](Cc1ccc(F)cc1)NC(=O)[C@H](Cc1c[nH]c2ccccc12)NC(=O)[C@@H]1CCCCN1C(=O)[C@H](CCC(=O)O)NC(=O)CCl ; CN(C(=O)CCl)[C@@H](CCC(=O)O)C(=O)N1CCCC[C@H]1C(=O)N[C@@H](Cc1c[nH]c2ccccc12)C(=O)N[C@@H](Cc1ccc(O)cc1)C(N)=O] 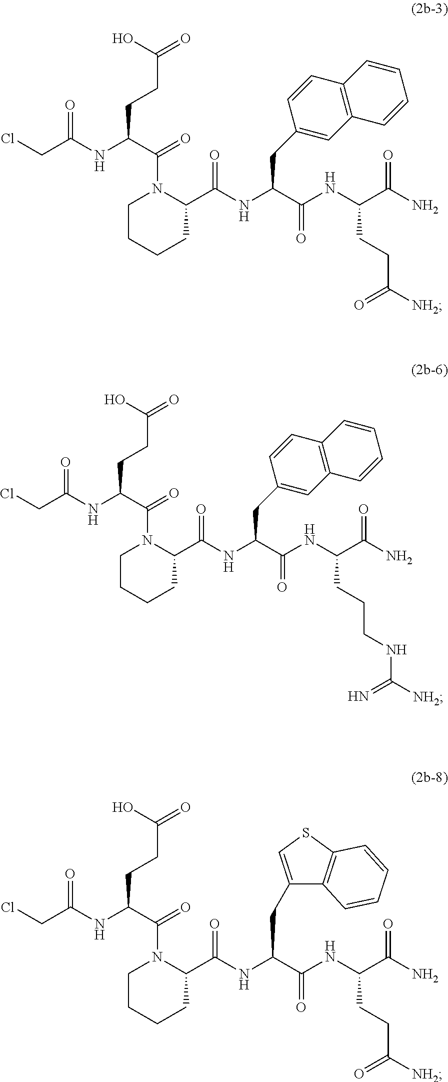 [SMILES: NC(=O)CC[C@H](NC(=O)[C@H](Cc1ccc2ccccc2c1)NC(=O)[C@@H]1CCCCN1C(=O)[C@H](CCC(=O)O)NC(=O)CCl)C(N)=O ; N=C(N)NCCC[C@H](NC(=O)[C@H](Cc1ccc2ccccc2c1)NC(=O)[C@@H]1CCCCN1C(=O)[C@H](CCC(=O)O)NC(=O)CCl)C(N)=O ; NC(=O)CC[C@H](NC(=O)[C@H](Cc1csc2ccccc12)NC(=O)[C@@H]1CCCCN1C(=O)[C@H](CCC(=O)O)NC(=O)CCl)C(N)=O] 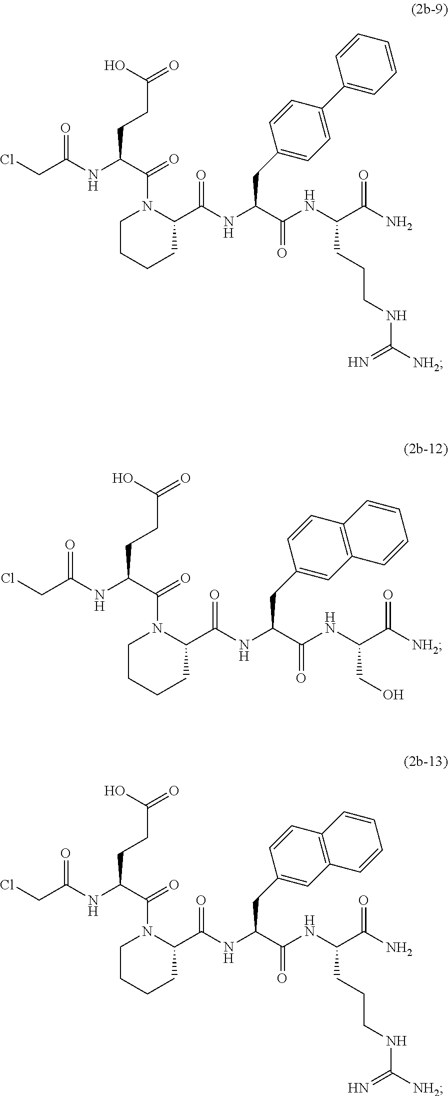 [SMILES: N=C(N)NCCC[C@H](NC(=O)[C@H](Cc1ccc(-c2ccccc2)cc1)NC(=O)[C@@H]1CCCCN1C(=O)[C@H](CCC(=O)O)NC(=O)CCl)C(N)=O ; NC(=O)[C@H](CO)NC(=O)[C@H](Cc1ccc2ccccc2c1)NC(=O)[C@@H]1CCCCN1C(=O)[C@H](CCC(=O)O)NC(=O)CCl ; N=C(N)NCCC[C@H](NC(=O)[C@H](Cc1ccc2ccccc2c1)NC(=O)[C@@H]1CCCCN1C(=O)[C@H](CCC(=O)O)NC(=O)CCl)C(N)=O] 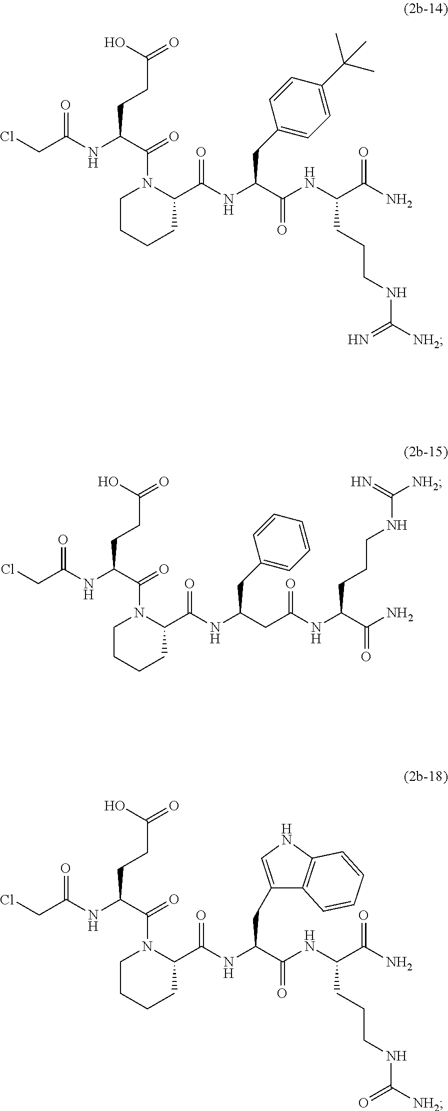 [SMILES: CC(C)(C)c1ccc(C[C@H](NC(=O)[C@@H]2CCCCN2C(=O)[C@H](CCC(=O)O)NC(=O)CCl)C(=O)N[C@@H](CCCNC(=N)N)C(N)=O)cc1 ; N=C(N)NCCC[C@H](NC(=O)C[C@H](Cc1ccccc1)NC(=O)[C@@H]1CCCCN1C(=O)[C@H](CCC(=O)O)NC(=O)CCl)C(N)=O ; NC(=O)NCCC[C@H](NC(=O)[C@H](Cc1c[nH]c2ccccc12)NC(=O)[C@@H]1CCCCN1C(=O)[C@H](CCC(=O)O)NC(=O)CCl)C(N)=O] 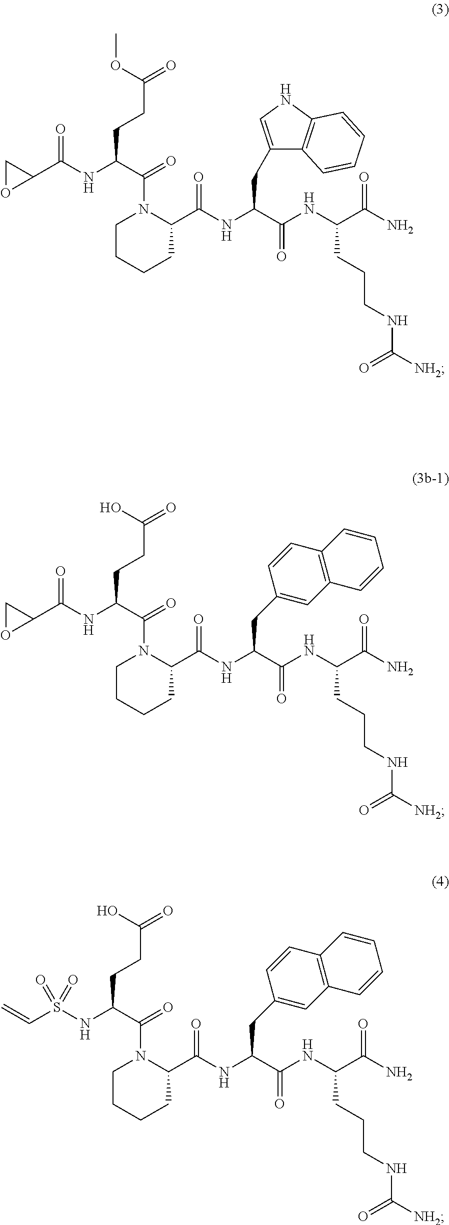 [SMILES: COC(=O)CC[C@H](NC(=O)C1CO1)C(=O)N1CCCC[C@H]1C(=O)N[C@@H](Cc1c[nH]c2ccccc12)C(=O)N[C@@H](CCCNC(N)=O)C(N)=O ; NC(=O)NCCC[C@H](NC(=O)[C@H](Cc1ccc2ccccc2c1)NC(=O)[C@@H]1CCCCN1C(=O)[C@H](CCC(=O)O)NC(=O)C1CO1)C(N)=O ; C=CS(=O)(=O)N[C@@H](CCC(=O)O)C(=O)N1CCCC[C@H]1C(=O)N[C@@H](Cc1ccc2ccccc2c1)C(=O)N[C@@H](CCCNC(N)=O)C(N)=O] 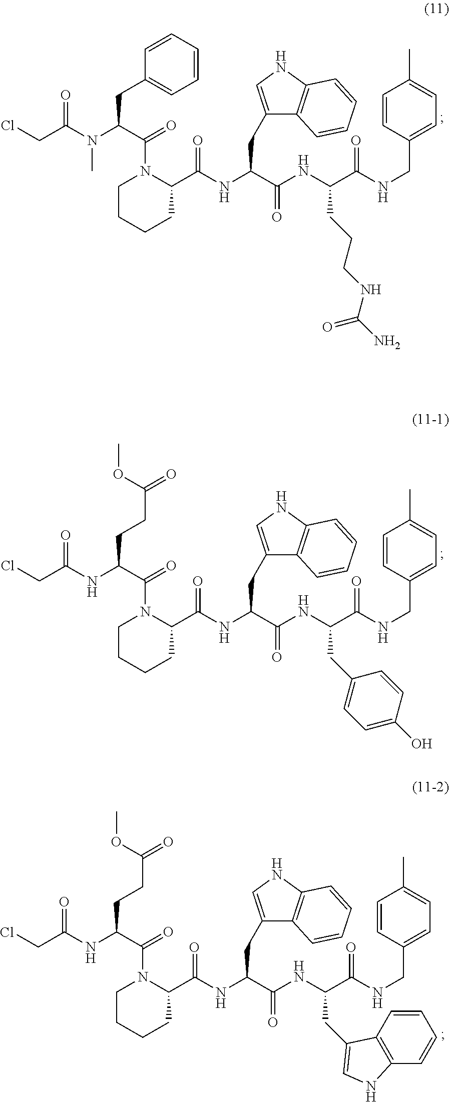 [SMILES: Cc1ccc(CNC(=O)[C@H](CCCNC(N)=O)NC(=O)[C@H](Cc2c[nH]c3ccccc23)NC(=O)[C@@H]2CCCCN2C(=O)[C@H](Cc2ccccc2)N(C)C(=O)CCl)cc1 ; COC(=O)CC[C@H](NC(=O)CCl)C(=O)N1CCCC[C@H]1C(=O)N[C@@H](Cc1c[nH]c2ccccc12)C(=O)N[C@@H](Cc1ccc(O)cc1)C(=O)NCc1ccc(C)cc1 ; COC(=O)CC[C@H](NC(=O)CCl)C(=O)N1CCCC[C@H]1C(=O)N[C@@H](Cc1c[nH]c2ccccc12)C(=O)N[C@@H](Cc1c[nH]c2ccccc12)C(=O)NCc1ccc(C)cc1] 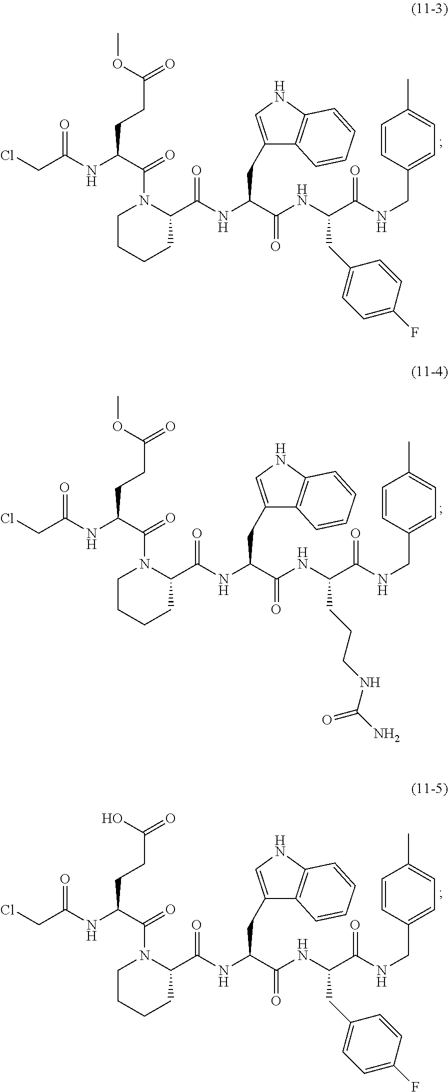 [SMILES: COC(=O)CC[C@H](NC(=O)CCl)C(=O)N1CCCC[C@H]1C(=O)N[C@@H](Cc1c[nH]c2ccccc12)C(=O)N[C@@H](Cc1ccc(F)cc1)C(=O)NCc1ccc(C)cc1 ; COC(=O)CC[C@H](NC(=O)CCl)C(=O)N1CCCC[C@H]1C(=O)N[C@@H](Cc1c[nH]c2ccccc12)C(=O)N[C@@H](CCCNC(N)=O)C(=O)NCc1ccc(C)cc1 ; Cc1ccc(CNC(=O)[C@H](Cc2ccc(F)cc2)NC(=O)[C@H](Cc2c[nH]c3ccccc23)NC(=O)[C@@H]2CCCCN2C(=O)[C@H](CCC(=O)O)NC(=O)CCl)cc1] 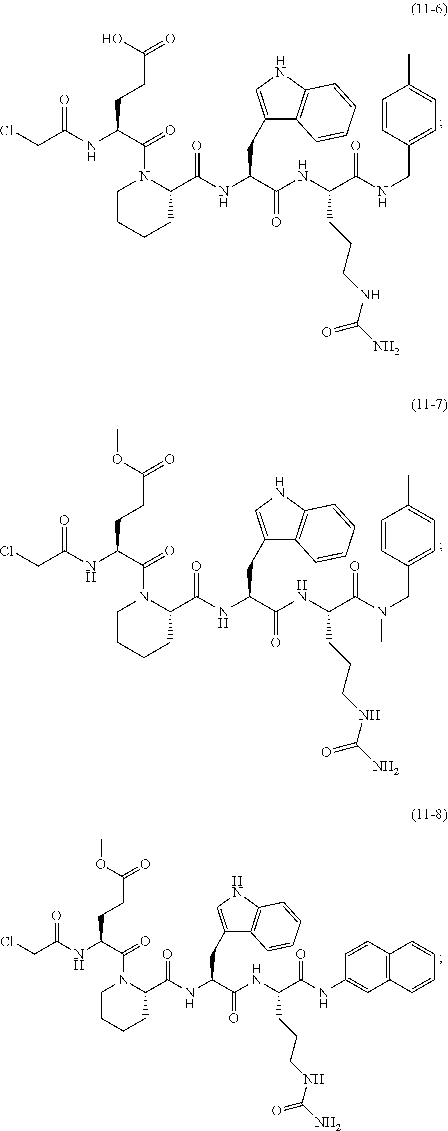 [SMILES: Cc1ccc(CNC(=O)[C@H](CCCNC(N)=O)NC(=O)[C@H](Cc2c[nH]c3ccccc23)NC(=O)[C@@H]2CCCCN2C(=O)[C@H](CCC(=O)O)NC(=O)CCl)cc1 ; COC(=O)CC[C@H](NC(=O)CCl)C(=O)N1CCCC[C@H]1C(=O)N[C@@H](Cc1c[nH]c2ccccc12)C(=O)N[C@@H](CCCNC(N)=O)C(=O)N(C)Cc1ccc(C)cc1 ; COC(=O)CC[C@H](NC(=O)CCl)C(=O)N1CCCC[C@H]1C(=O)N[C@@H](Cc1c[nH]c2ccccc12)C(=O)N[C@@H](CCCNC(N)=O)C(=O)Nc1ccc2ccccc2c1] 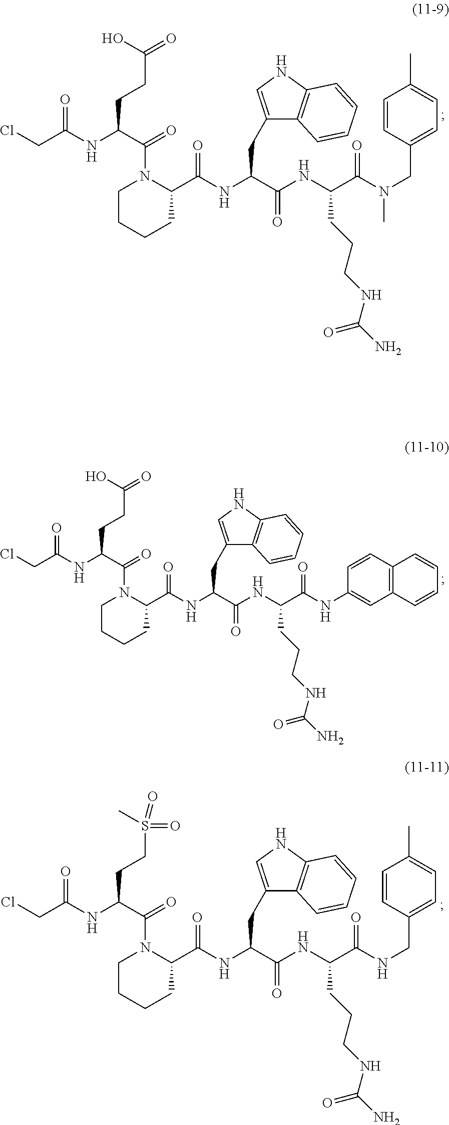 [SMILES: Cc1ccc(CN(C)C(=O)[C@H](CCCNC(N)=O)NC(=O)[C@H](Cc2c[nH]c3ccccc23)NC(=O)[C@@H]2CCCCN2C(=O)[C@H](CCC(=O)O)NC(=O)CCl)cc1 ; NC(=O)NCCC[C@H](NC(=O)[C@H](Cc1c[nH]c2ccccc12)NC(=O)[C@@H]1CCCCN1C(=O)[C@H](CCC(=O)O)NC(=O)CCl)C(=O)Nc1ccc2ccccc2c1 ; Cc1ccc(CNC(=O)[C@H](CCCNC(N)=O)NC(=O)[C@H](Cc2c[nH]c3ccccc23)NC(=O)[C@@H]2CCCCN2C(=O)[C@H](CCS(C)(=O)=O)NC(=O)CCl)cc1] 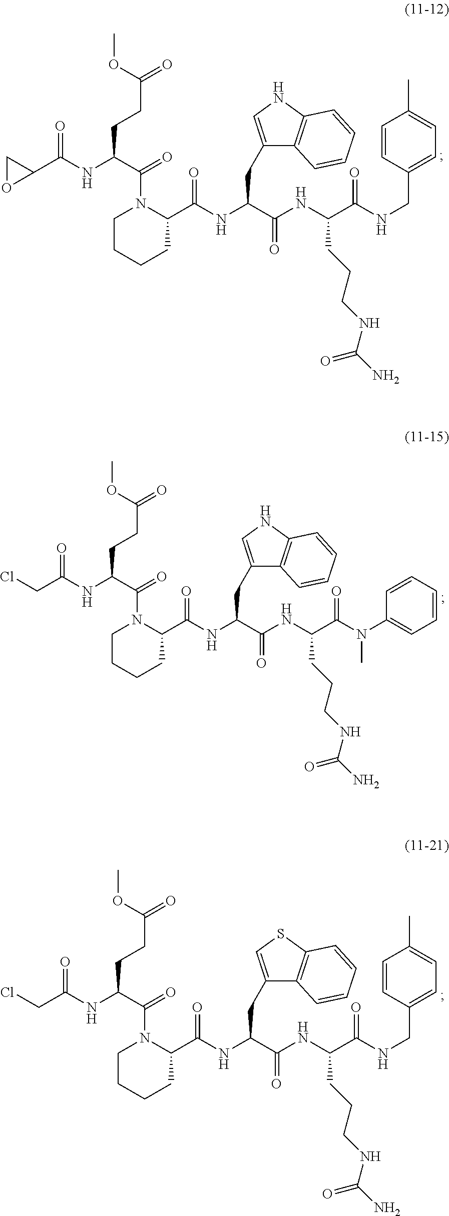 [SMILES: COC(=O)CC[C@H](NC(=O)C1CO1)C(=O)N1CCCC[C@H]1C(=O)N[C@@H](Cc1c[nH]c2ccccc12)C(=O)N[C@@H](CCCNC(N)=O)C(=O)NCc1ccc(C)cc1 ; COC(=O)CC[C@H](NC(=O)CCl)C(=O)N1CCCC[C@H]1C(=O)N[C@@H](Cc1c[nH]c2ccccc12)C(=O)N[C@@H](CCCNC(N)=O)C(=O)N(C)c1ccccc1 ; COC(=O)CC[C@H](NC(=O)CCl)C(=O)N1CCCC[C@H]1C(=O)N[C@@H](Cc1csc2ccccc12)C(=O)N[C@@H](CCCNC(N)=O)C(=O)NCc1ccc(C)cc1] 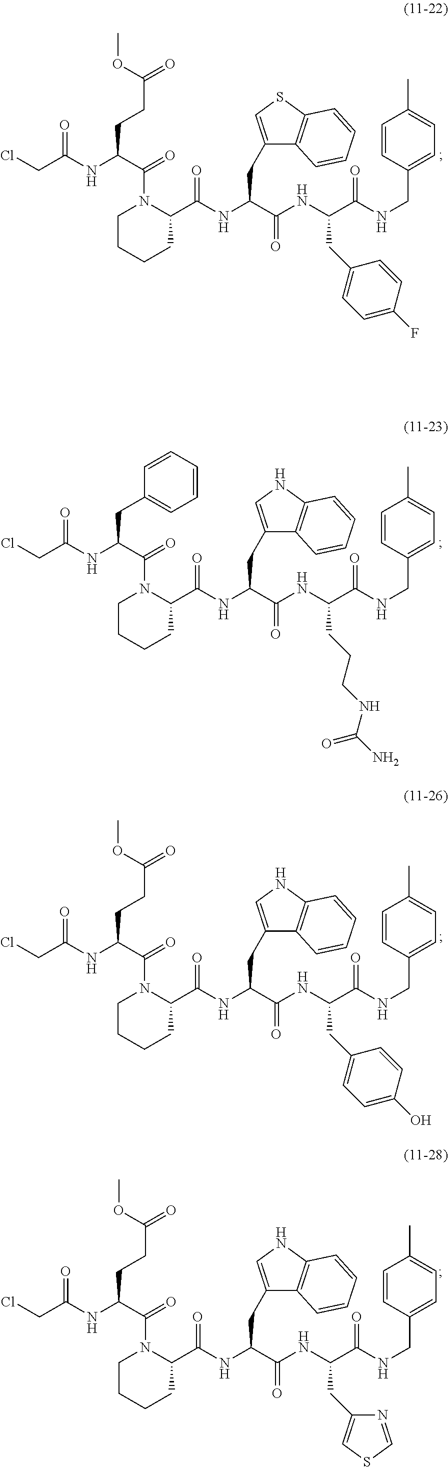 [SMILES: COC(=O)CC[C@H](NC(=O)CCl)C(=O)N1CCCC[C@H]1C(=O)N[C@@H](Cc1csc2ccccc12)C(=O)N[C@@H](Cc1ccc(F)cc1)C(=O)NCc1ccc(C)cc1 ; Cc1ccc(CNC(=O)[C@H](CCCNC(N)=O)NC(=O)[C@H](Cc2c[nH]c3ccccc23)NC(=O)[C@@H]2CCCCN2C(=O)[C@H](Cc2ccccc2)NC(=O)CCl)cc1 ; COC(=O)CC[C@H](NC(=O)CCl)C(=O)N1CCCC[C@H]1C(=O)N[C@@H](Cc1c[nH]c2ccccc12)C(=O)N[C@@H](Cc1ccc(O)cc1)C(=O)NCc1ccc(C)cc1 ; COC(=O)CC[C@H](NC(=O)CCl)C(=O)N1CCCC[C@H]1C(=O)N[C@@H](Cc1c[nH]c2ccccc12)C(=O)N[C@@H](Cc1cscn1)C(=O)NCc1ccc(C)cc1] 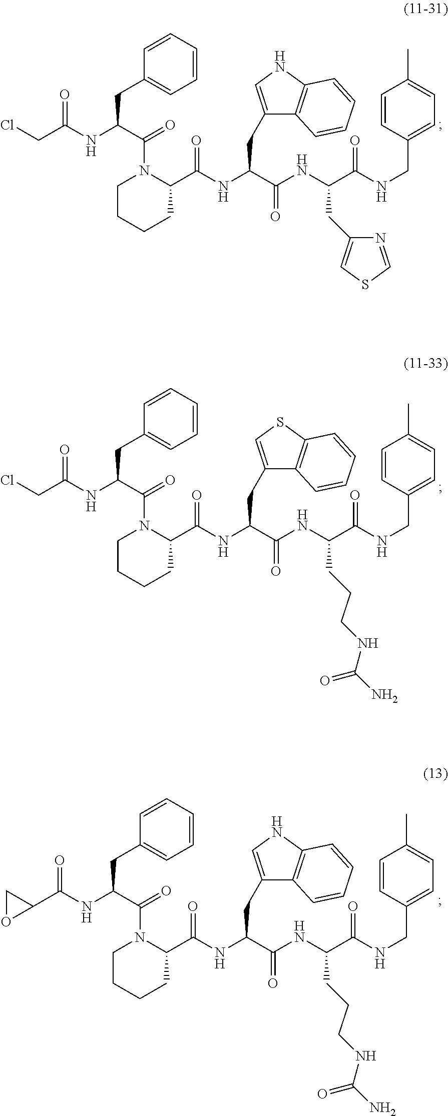 [SMILES: Cc1ccc(CNC(=O)[C@H](Cc2cscn2)NC(=O)[C@H](Cc2c[nH]c3ccccc23)NC(=O)[C@@H]2CCCCN2C(=O)[C@H](Cc2ccccc2)NC(=O)CCl)cc1 ; Cc1ccc(CNC(=O)[C@H](CCCNC(N)=O)NC(=O)[C@H](Cc2csc3ccccc23)NC(=O)[C@@H]2CCCCN2C(=O)[C@H](Cc2ccccc2)NC(=O)CCl)cc1 ; Cc1ccc(CNC(=O)[C@H](CCCNC(N)=O)NC(=O)[C@H](Cc2c[nH]c3ccccc23)NC(=O)[C@@H]2CCCCN2C(=O)[C@H](Cc2ccccc2)NC(=O)C2CO2)cc1] 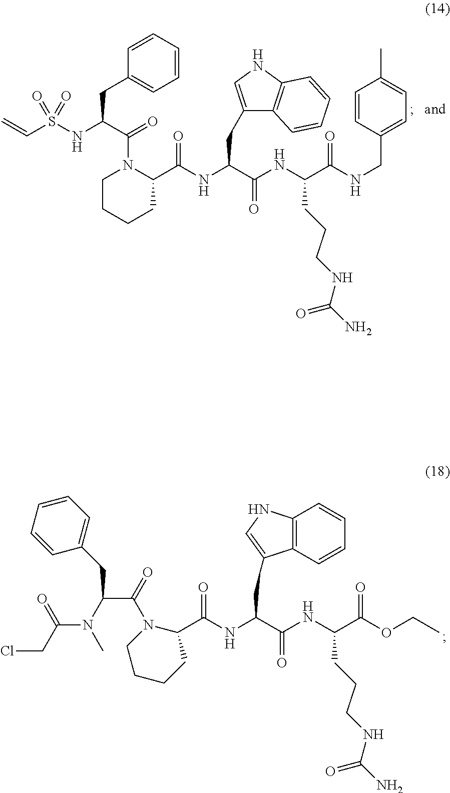 [SMILES: C=CS(=O)(=O)N[C@@H](Cc1ccccc1)C(=O)N1CCCC[C@H]1C(=O)N[C@@H](Cc1c[nH]c2ccccc12)C(=O)N[C@@H](CCCNC(N)=O)C(=O)NCc1ccc(C)cc1 ; CCOC(=O)[C@H](CCCNC(N)=O)NC(=O)[C@H](Cc1c[nH]c2ccccc12)NC(=O)[C@@H]1CCCCN1C(=O)[C@H](Cc1ccccc1)N(C)C(=O)CCl]
or a pharmaceutically acceptable salt or stereoisomer thereof.

12. (canceled)

17. A pharmaceutical composition, comprising a therapeutically effective amount of the compound of claim 1, or a pharmaceutically acceptable salt or stereoisomer thereof, and a pharmaceutically acceptable carrier.

18. The pharmaceutical composition of claim 17, which is in the form of a liquid for oral or parenteral administration.

19. (canceled)

20. A method of treating a disease or disorder mediated by dysregulated Pin1 activity, comprising administering a therapeutically effective amount of the compound or pharmaceutically acceptable salt or stereoisomer of claim 1.

21. The method of claim 20, wherein the disease is cancer, an autoimmune disease or a neurodegenerative disease.

22. The method of claim 21, wherein the cancer is characterized by:
the presence of cancer stem cells (CSCs);
the activation of at least one oncogene selected from the group consisting of: PML-RARα, NF-κB, β-catenin, Notch1, Notch3, OCT4, MYC, MCL1, Cyclin D1, PKM2, JUN, PGK1, FOS, SF1, Tax, HER2, RAB2A, AKT, v53M, Nanog, STAT3, HIF1, HSF1, ER, RAF1, FOXM1, SEPT9, PIP, v-Rel, Survivin, PTP-PEST, AR, MYB, PLK, S6K, AIB1, FAK, NUR77, RSK2, MAP3K8, IRAK1, COX2 and HBx; or
the inactivation of at least one tumour suppressor selected from the group consisting of: PML, AMPK, FBXW7, TRF1, GRK2, SUV39H1, SMRT, RUNX3, RBBP8, ATR, RB1, SMAD, FOXO4, RARα, BTK, CDK10, DAXX, KLF10, BAX and PIP4K.

23.-24. (canceled)

25. The method of claim 21 wherein the autoimmune disease is selected from the group consisting of lupus, asthma and arthritis.

26. The method of claim 21, wherein the neurodegenerative disease is Alzheimer's disease or Parkinson's disease.

27. (canceled)
